SEQUENCE LISTING (141 sequences)

SEQ ID NO: 1 - protein, 297 aa, synthetic construct
MYRMQLLSCIALSLALVTNSAPTQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGGSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGAPGSGLNDIFEAQKIEWHE

SEQ ID NO: 2 - protein, 278 aa, synthetic construct
MYRMQLLSCIALSLALVTNSAPTQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGAPGSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 3 - protein, 299 aa, synthetic construct
MYRMQLLSCIALSLALVTNSAPVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVSGGGGSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGAPGSGLNDIFEAQKIEWE

SEQ ID NO: 4 - protein, 278 aa, synthetic construct
MYRMQLLSCIALSLALVTNSAPQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGAPVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVS

SEQ ID NO: 5 - protein, 275 aa, synthetic construct
MYRMQLLSCIALSLALVTNSAPTQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGAPGSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 6 - protein, 302 aa, synthetic construct
MYRMQLLSCIALSLALVTNSAPVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVSGGGSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGAPGSGLNDIFEAQKIEWHE

SEQ ID NO: 7 - protein, 284 aa, synthetic construct
MYRMQLLSCIALSLALVTNSAPTQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSS

SEQ ID NO: 8 - protein, 280 aa, synthetic construct
MYRMQLLSCIALSLALVTNSAPTQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSQVQLQESGGGSVQAGGSLRLSCAASGYTRSSHCMVWFRQAPGKEREGVALIYTGSGSTYYADSVKGRFTISQDNAKKTLYLQMNSLKPEDTAMYYCAAGTSSSSCPGLLGPPRYYNWGQGTQVTVSS

SEQ ID NO: 9 - protein, 274 aa, synthetic construct
MYRMQLLSCIALSLALVTNSAPTQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSQVQLQESGGGLVQPGGSLRLSCIASGFTFSIMAMSWVRQAPGKGLEWVSTINSDGGKTYYADSVKGRFTISRDNAKNTLYLQLNSLRTEDMAMYYCRRCADIYCSGSGGWTGQGTQVTVSS

SEQ ID NO: 10 - protein, 281 aa, synthetic construct
MYRMQLLSCIALSLALVTNSAPTQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSS

SEQ ID NO: 11 - protein, 256 aa, synthetic construct
MDSYLLMWGLLTFIMVPGCQAGGGSTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGGGSYQVAVAGCVFLLISVLLLSGLTWQRRQRKSRRTI

SEQ ID NO: 12 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 13 - protein, 185 aa, synthetic construct
MYRMQLLSCIALSLALVTNSAPTQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGGGSPKKKIQLHAEHALYDALMILNIVKTNSGGGGSHHHHHHHH

SEQ ID NO: 14 - protein, 237 aa, synthetic construct
MYRMQLLSCIALSLALVTNSAPTQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGGSGGGSTNSPPAEEKLEDYAFNFELILEEIARLFESGDQKDEAEKAKRMKEWMKRIKTTASEDEQEEMANAIITILQSWIFSGGGGSHHHHHHHH

SEQ ID NO: 15 - protein, 193 aa, synthetic construct
MYRMQLLSCIALSLALVTNSAPVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVSGGGSGGGSPKKKIQLHAEHALYDALMILNIVKTNSGGGGSHHHHHHHH

SEQ ID NO: 16 - protein, 285 aa, synthetic construct
MYRMQLLSCIALSLALVTNSAPTQVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSSGGSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSS

SEQ ID NO: 17 - protein, 285 aa, synthetic construct
MYRMQLLSCIALSLALVTNSAPTQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSQVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSS

SEQ ID NO: 18 - protein, 288 aa, synthetic construct
MYRMQLLSCIALSLALVTNSAPTQVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSSGGSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSS

SEQ ID NO: 19 - protein, 288 aa, synthetic construct
MYRMQLLSCIALSLALVTNSAPTQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSQVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSS

SEQ ID NO: 20 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 21 - protein, 383 aa, synthetic construct
MYRMQLLSCIALSLALVTNSAPTQVKLEESGGGLVQAGGSLRLSCAASGRIFSIYDMGWFRQAPGKEREFVSGIRWSGGTSYADSVKGRFTISKDNAKNTIYLQMNNLKAEDTAVYYCGSRGYWGQGTLVTVSSGSGSQVKLEESGGGLVQAGGSLRLSCAASGRIFSIYDMGWFRQAPGKEREFVSGIRWSGGTSYADSVKGRFTISKDNAKNTIYLQMNNLKAEDTAVYYCGSRGYWGQGTLVTVSSGGSGGSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGSHHHHHHHH

SEQ ID NO: 22 - protein, 386 aa, synthetic construct
MYRMQLLSCIALSLALVTNSAPTQVKLEESGGGLVQAGGSLRLSCAASGRIFSIYDMGWFRQAPGKEREFVSGIRWSGGTSYADSVKGRFTISKDNAKNTIYLQMNNLKAEDTAVYYCGSRGYWGQGTLVTVSSGSGSQVKLEESGGGLVQAGGSLRLSCAASGRIFSIYDMGWFRQAPGKEREFVSGIRWSGGTSYADSVKGRFTISKDNAKNTIYLQMNNLKAEDTAVYYCGSRGYWGQGTLVTVSSGGSVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVSGGSHHHHHHHH

SEQ ID NO: 23 - protein, 409 aa, synthetic construct
MYRMQLLSCIALSLALVTNSAPTGQAVVLQEPSLSVSPGGTVTLTCGLSSGSVSTNYYPSWYQQTPGQAPRTLIYYTNTRSSDVPERFSGSIVGNKAALTITGAQPDDESVYFCLLYLGRGIWVFGGGTKLTVLGQPKGGGGSGGGGSGGGGSEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCASSKEKATYYYGMDVWGQGTTVTVSSGGSGGSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGSHHHHHHHH

SEQ ID NO: 24 - protein, 500 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGAPVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTCNQVSLTCLVKGWYPSDIAVEWESNGQPENNYKTTPPVLDSDGSFFLYSKLTVDKSRWQQGNVFSCSVMHEALHNHYTQKSLSLSP

SEQ ID NO: 25 - protein, 518 aa, synthetic construct
MGWSCIILFLVATATGVHSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGGSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGAPGSGLNDIFEAQKIEWHEDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSCEEMTKNQVSLTCLVKGSYASDIAVEWESNGQPENNYKTTPPVLDSDGSFFLYSKLTVVKSRWQQGNVFSCSVMHEALHNHYTQKSLSLSP

SEQ ID NO: 26 - protein, 164 aa, synthetic construct
MYRMQLLSCIALSLALVTNSAPTSSSTKKTQLQLEHLRLDLEMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCTSIIRTLTGGSHHHHHHHH

SEQ ID NO: 27 - protein, 416 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKVEIKRTVGGGGSGGGGSGGGGSQVQLVQSGAEVKKPGSSVKVSCKASGGTFSSDAINWVRQAPGQGLEWMGRIIPIFGVADYAQKFQGRVTLTADKSTSTAYMDLSSLRSEDTAVFYCARERGDYSNFWYFDLWGRGTLVTVSSGGSHHHHHHHH

SEQ ID NO: 28 - protein, 403 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSDIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKRTVGGGGSGGGGSGGGGSQVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSGGSHHHHHHHH

SEQ ID NO: 29 - protein, 411 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKVEIKRTVGGGGSGGGGSGGGGSQVQLVQSGAEVKKPGSSVKVSCKASGGTFSSDAINWVRQAPGQGLEWMGRIIPIFGVADYAQKFQGRVTLTADKSTSTAYMDLSSLRSEDTAVFYCARERGDYSNFWYFDLWGRGTLVTVSSGGSHHHHHHHH

SEQ ID NO: 30 - protein, 401 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGSHHHHHHHH

SEQ ID NO: 31 - protein, 401 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVGGSHHHHHHHH

SEQ ID NO: 32 - protein, 400 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSDIQMTQSPSSVSASVGDRVTITCSASQGISGDLNWYQQKPGKAPKLLIYHTSSLHSGVPSRFSGSGSGTDFTLTISSLQPEDFATYYCQYYSKDLLTFGGGTKLEIKRTVGGGGSGGGGSGGGGSEVQLVQSGAEVKKPGESLKISCKGSGYSFTNFYIHWVRQAPGQRLEWMGSIYPNYGDTAYNQKFKDRFVFSLDTSVSTAYLQISSLKAEDTAVYYCARGYSYAMDYWGQGTTVTVSSGGSHHHHHHHH

SEQ ID NO: 33 - protein, 394 aa, synthetic construct
MGWSCIILFLVATATGVHSQVQLQESGGGLVQPGGSLKLSCAASGFRFSNYGMSWVRQAPGEGLEWVSYINGDGSRTHYADSVKGRFTISRDNAKNTLYLQLNSLKTEDTAMYYCEKGLSRDGWSLSAASRGQGTQVTVSSDIQMTQSPSSVSASVGDRVTITCSASQGISGDLNWYQQKPGKAPKLLIYHTSSLHSGVPSRFSGSGSGTDFTLTISSLQPEDFATYYCQYYSKDLLTFGGGTKLEIKRTVGGGGSGGGGSGGGGSEVQLVQSGAEVKKPGESLKISCKGSGYSFTNFYIHWVRQAPGQRLEWMGSIYPNYGDTAYNQKFKDRFVFSLDTSVSTAYLQISSLKAEDTAVYYCARGYSYAMDYWGQGTTVTVSSGGSHHHHHHHH

SEQ ID NO: 34 - protein, 392 aa, synthetic construct
MYRMQLLSCIALSLALVTNSAPTQVQLQESGGGAVQAGGSLRLSCAASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVGGSHHHHHHHH

SEQ ID NO: 35 - protein, 412 aa, synthetic construct
MYRMQLLSCIALSLALVTNSAPTQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSDIQMTQSPSSVSASVGDRVTITCSASQGISGDLNWYQQKPGKAPKLLIYHTSSLHSGVPSRFSGSGSGTDFTLTISSLQPEDFATYYCQYYSKDLLTFGGGTKLEIKRTVGGGGSGGGGSGGGGSEVQLVQSGAEVKKPGESLKISCKGSGYSFTNFYIHWVRQAPGQRLEWMGSIYPNYGDTAYNQKFKDRFVFSLDTSVSTAYLQISSLKAEDTAVYYCARGYSYAMDYWGQGTTVTVSSGGSHHHHHHHH

SEQ ID NO: 36 - protein, 412 aa, synthetic construct
MYRMQLLSCIALSLALVTNSAPTQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSEVQLVQSGAEVKKPGESLKISCKGSGYSFTNFYIHWVRQAPGQRLEWMGSIYPNYGDTAYNQKFKDRFVFSLDTSVSTAYLQISSLKAEDTAVYYCARGYSYAMDYWGQGTTVTVSSGGGGSGGGGSGGGGSDIQMTQSPSSVSASVGDRVTITCSASQGISGDLNWYQQKPGKAPKLLIYHTSSLHSGVPSRFSGSGSGTDFTLTISSLQPEDFATYYCQYYSKDLLTFGGGTKLEIKRTVGGSHHHHHHHH

SEQ ID NO: 37 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 38 - protein, 462 aa, synthetic construct
MGWSCIILFLVATATGVHSQSVEESGGRLVTPGTPLTLTCTASGFSLSGYYMSWVRQAPGKGLEWIGSISYGGSTYYASWAKGRFTISKTSTTVDLKITSPTTEDTATYFCAAGYTTNTAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 39 - protein, 463 aa, synthetic construct
MGWSCIILFLVATATGVHSQSVEESGGRLVTPGTPLTLTCTVSGFSLSRYYMSWVRQAPGKGLEWIGYIYADSGSTWYASWAKGRFTISKTSTTVDLKMTSLTTEDTATYFCARRADHDGAFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 40 - protein, 463 aa, synthetic construct
MGWSCIILFLVATATGVHSQSVEESGGRLVTPGTPLTLTCTVSGFSLSSYDMSWVRQAPGKGLEYIGIISSSGSTYYASWAKGRFTISKTSTTVDLKITSPTTEDTATYFCARLGYVGYGYADIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 41 - protein, 466 aa, synthetic construct
MGWSCIILFLVATATGVHSQSVEESGGRLVTPGTPLTLTCTVSGFSLSSYHMCWVRQPPGKGLEYIGIISGGAITYYATWAKGRFTISITSSTTVDLKITSPTTEDTATYFCARGGGYGGGDYNFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 42 - protein, 461 aa, synthetic construct
MGWSCIILFLVATATGVHSQSVEESGGRLVTPGTPLTLTCTVSGFSLSYYAMIWVRQAPGKGLEWIGFINSYGKTYYANWAKGRFTISKTSTTVDLKMTSLTSEDTATYFCARERDNYHFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 43 - protein, 476 aa, synthetic construct
MGWSCIILFLVATATGVHSQQHLVESGGGLVKPGASLTLTCKASGFYFSGYYYMSWVRQAPGKGLEWIASIYSGGSGNTYYASWAKGRFTISKTSSTTVTLQMTSLTAADTATYFCARGADYTHDYPADAAAWSFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 44 - protein, 465 aa, synthetic construct
MGWSCIILFLVATATGVHSQSLEESGGGLVKPGASLTLTCTASGFSFSGSYYMCWVRQAPGKGLECIACIYAGSSGATYYASWAKGRFTVSKTSSTTVTLQMTSLTAADTATYFCARHTNGIGLGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 45 - protein, 467 aa, synthetic construct
MGWSCIILFLVATATGVHSQVQLQQSGPELVRPGVSVKISCKGSGHTFTDYALHWVRQSHVKSLEWIGVISSYNGITNYNQRFKGKATMTVDKSSSTAYLELARLTSEDSAIYYCSREEWDVFYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 46 - protein, 467 aa, synthetic construct
MGWSCIILFLVATATGVHSQVQLQQSGAELVRPGASVKLSCKALGDTFTDYEIHWVKQTPVHGLEWIGVIHPGSGGTVYNQKFKGKATLTADKYSSTAYMELSSLTSEDSAVYYCTREGMNTDWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 47 - protein, 465 aa, synthetic construct
MGWSCIILFLVATATGVHSQEQLEESGGDLVKPGASLTLTCTASGFSFNSYWMSWVRQAPGKGLEWIADIGAGGSGSTFYASWAKGRFTISKTSSTTVTLQMTSLTAADTATYFCARYVDGGHSDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 48 - protein, 465 aa, synthetic construct; identical to SEQ ID NO: 47

SEQ ID NO: 49 - protein, 236 aa, synthetic construct
MGWSCIILFLVATATGVHSAFELTQTPSSVEAAVGGTVTIKCQASQSISSYLAWYQQKPGQPPKLLIYGASNLKSGVPSRFKGSGSGTQFTLTISDLEAADAATYYCQSYYYSSSSSYSFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 237 aa, synthetic construct
MGWSCIILFLVATATGVHSAAVLTQTPSPVSAAVGGTVSISCQSSQSVYGKNELSWYQQKPGQPPKLLIYYASTLASGVPSRFSGSGSGTQFALTISGVQADDAATYYCAGGYSSSVDTTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 235 aa, synthetic construct
MGWSCIILFLVATATGVHSIEMTQTPFSVSAAVGGTVTIKCQASEDIYSLLAWYQQKPGQPPKLLIYSASTLASGVPSRFKGSGYGTEFTLTISGVQAADAATYYCQGGYYSSSADNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 237 aa, synthetic construct
MGWSCIILFLVATATGVHSAAVLTQTPSPVSAAVGATVSISCQSSQSVYGNSELAWYQQKPGQRPKLLIFEASKLASGVPSRFSGSGSGTQFSLAISDVQADDAATYYCAGGYSSTSDTTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 238 aa, synthetic construct
MGWSCIILFLVATATGVHSAQVLTQTPSPVSAAVGGTVTISCQSSESVYNNNWLAWYQQKPGQPPKFLIYDASTLASGVPSRFSGSGSGTQFTLTISGVQADDAATYYCQGTYYSSDWYFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 240 aa, synthetic construct
MGWSCIILFLVATATGVHSADIVMTQTPASVEAAVGGIVTIKCQASQSISSYLAWYQQKPGQPPNLLIYRASTLASGVPSRFKGSGSGTQFTLTISDLEAADAATYYCQGYHFSSTSSNYGNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 238 aa, synthetic construct
MGWSCIILFLVATATGVHSDIVMTQTPASVSAAVGGTVSINCQASEDIENFLAWYQQKPGQPPSLLIYETSKLASGVPSRFSGSGSGTEFTLTISDLEAADAATYYCQSYDWSDSSTYDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 238 aa, synthetic construct
MGWSCIILFLVATATGVHSDIMLTQSPLSLPVSLGDQASISCRSSQGIVHSDGNTYLEWYLQKPGQSPKLLIYKVSNRFSGVPDRFSGSGSGTDFILKISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 238 aa, synthetic construct
MGWSCIILFLVATATGVHSDILMTQDELSLPVSLGDQASISCRSSQTIVHTNGNTYLEWYLQKPGQSPKLLIYKVSNRFSGVPDRFSGSGSGTYFTLKISRLEAEDLGVYYCFQGSHVPYTFGGGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 236 aa, synthetic construct
MGWSCIILFLVATATGVHSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 236 aa, synthetic construct; identical to SEQ ID NO: 58

SEQ ID NO: 60 - protein, 372 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 372 aa, synthetic construct; identical to SEQ ID NO: 60

SEQ ID NO: 62 - protein, 373 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSAAVLTQTPSPVSAAVGGTVSISCQSSQSVYGKNELSWYQQKPGQPPKLLIYYASTLASGVPSRFSGSGSGTQFALTISGVQADDAATYYCAGGYSSSVDTTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 373 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSAAVLTQTPSPVSAAVGATVSISCQSSQSVYGNSELAWYQQKPGQRPKLLIFEASKLASGVPSRFSGSGSGTQFSLAISDVQADDAATYYCAGGYSSTSDTTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 374 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSDIMLTQSPLSLPVSLGDQASISCRSSQGIVHSDGNTYLEWYLQKPGQSPKLLIYKVSNRFSGVPDRFSGSGSGTDFILKISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 374 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSDILMTQDELSLPVSLGDQASISCRSSQTIVHTNGNTYLEWYLQKPGQSPKLLIYKVSNRFSGVPDRFSGSGSGTYFTLKISRLEAEDLGVYYCFQGSHVPYTFGGGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 595 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSQSVEESGGRLVTPGTPLTLTCTASGFSLSGYYMSWVRQAPGKGLEWIGSISYGGSTYYASWAKGRFTISKTSTTVDLKITSPTTEDTATYFCAAGYTTNTAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 67 - protein, 596 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSQSVEESGGRLVTPGTPLTLTCTVSGFSLSSYDMSWVRQAPGKGLEYIGIISSSGSTYYASWAKGRFTISKTSTTVDLKITSPTTEDTATYFCARLGYVGYGYADIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 68 - protein, 594 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSQSVEESGGRLVTPGTPLTLTCTVSGFSLSYYAMIWVRQAPGKGLEWIGFINSYGKTYYANWAKGRFTISKTSTTVDLKMTSLTSEDTATYFCARERDNYHFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 69 - protein, 609 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSQQHLVESGGGLVKPGASLTLTCKASGFYFSGYYYMSWVRQAPGKGLEWIASIYSGGSGNTYYASWAKGRFTISKTSSTTVTLQMTSLTAADTATYFCARGADYTHDYPADAAAWSFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 70 - protein, 598 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSQSLEESGGGLVKPGASLTLTCTASGFSFSGSYYMCWVRQAPGKGLECIACIYAGSSGATYYASWAKGRFTVSKTSSTTVTLQMTSLTAADTATYFCARHTNGIGLGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 71 - protein, 369 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 369 aa, synthetic construct; identical to SEQ ID NO: 71

SEQ ID NO: 73 - protein, 370 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSAAVLTQTPSPVSAAVGGTVSISCQSSQSVYGKNELSWYQQKPGQPPKLLIYYASTLASGVPSRFSGSGSGTQFALTISGVQADDAATYYCAGGYSSSVDTTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 370 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSAAVLTQTPSPVSAAVGATVSISCQSSQSVYGNSELAWYQQKPGQRPKLLIFEASKLASGVPSRFSGSGSGTQFSLAISDVQADDAATYYCAGGYSSTSDTTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 371 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSDIMLTQSPLSLPVSLGDQASISCRSSQGIVHSDGNTYLEWYLQKPGQSPKLLIYKVSNRFSGVPDRFSGSGSGTDFILKISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 371 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISMLYDNAKNTLYLQMNSLKPEDTAMYYCAAASPGRCFLPRTALEPALYYNWGQGTQVTVSSGGSDILMTQDELSLPVSLGDQASISCRSSQTIVHTNGNTYLEWYLQKPGQSPKLLIYKVSNRFSGVPDRFSGSGSGTYFTLKISRLEAEDLGVYYCFQGSHVPYTFGGGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 598 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSQSVEESGGRLVTPGTPLTLTCTASGFSLSGYYMSWVRQAPGKGLEWIGSISYGGSTYYASWAKGRFTISKTSTTVDLKITSPTTEDTATYFCAAGYTTNTAFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 78 - protein, 599 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSQSVEESGGRLVTPGTPLTLTCTVSGFSLSSYDMSWVRQAPGKGLEYIGIISSSGSTYYASWAKGRFTISKTSTTVDLKITSPTTEDTATYFCARLGYVGYGYADIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 79 - protein, 597 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSQSVEESGGRLVTPGTPLTLTCTVSGFSLSYYAMIWVRQAPGKGLEWIGFINSYGKTYYANWAKGRFTISKTSTTVDLKMTSLTSEDTATYFCARERDNYHFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 80 - protein, 612 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSQQHLVESGGGLVKPGASLTLTCKASGFYFSGYYYMSWVRQAPGKGLEWIASIYSGGSGNTYYASWAKGRFTISKTSSTTVTLQMTSLTAADTATYFCARGADYTHDYPADAAAWSFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 81 - protein, 601 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSQSLEESGGGLVKPGASLTLTCTASGFSFSGSYYMCWVRQAPGKGLECIACIYAGSSGATYYASWAKGRFTVSKTSSTTVTLQMTSLTAADTATYFCARHTNGIGLGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 82 - protein, 388 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSGGSEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 83 - protein, 386 aa, synthetic construct
MGWSCIILFLVATATGVHSQVQLQESGGGSVQAGGSLRLSCTASGFTFDDSDMGWYRQAPGNECELVSTISSDGNTYYTDSVKGRFTISQDNAKNTVYLQMNSLGPEDTAVYYCAAEPRGYYSNYGGRRECNYWGQGTQVTVSSGGSEVQLVQSGAEVKKPGESLKISCKGSGYSFTNFYIHWVRQAPGQRLEWMGSIYPNYGDTAYNQKFKDRFVFSLDTSVSTAYLQISSLKAEDTAVYYCARGYSYAMDYWGQGTTVTVSSGGGGSGGGGSGGGGSDIQMTQSPSSVSASVGDRVTITCSASQGISGDLNWYQQKPGKAPKLLIYHTSSLHSGVPSRFSGSGSGTDFTLTISSLQPEDFATYYCQYYSKDLLTFGGGTKLEIK

SEQ ID NO: 84 - protein, 587 aa, synthetic construct
MGWSCIILFLVATATGVHSQVQLQESGGGSVQAGGSLRLSCVGSGYTYDTSDMSWYRQAPGKEREFVSDIDSGDWAAYADAVKGRFTISRDNAKKTVYLQMNSLEPEDTAMYYCKASYWKWGKLNNFWGPGTQVTVSSGGSQSLEESGGGLVKPGASLTLTCTASGFSFSGSYYMCWVRQAPGKGLECIACIYAGSSGATYYASWAKGRFTVSKTSSTTVTLQMTSLTAADTATYFCARHTNGIGLGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 85 - protein, 590 aa, synthetic construct
MGWSCIILFLVATATGVHSQVQLQESGGGLVQPGGSLKLSCAASGFRFSNYGMSWVRQAPGEGLEWVSYINGDGSRTHYADSVKGRFTISRDNAKNTLYLQLNSLKTEDTAMYYCEKGLSRDGWSLSAASRGQGTQVTVSSGGSQSLEESGGGLVKPGASLTLTCTASGFSFSGSYYMCWVRQAPGKGLECIACIYAGSSGATYYASWAKGRFTVSKTSSTTVTLQMTSLTAADTATYFCARHTNGIGLGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 86 - protein, 593 aa, synthetic construct
MGWSCIILFLVATATGVHSQVQLQESGGGSVQTGGSLRLSCAVSGYTTYSFNYMGWFRQAPGKEREGVAVIYTGGGSTLYADSVKGRFTISQDNAKNTVYLQMNSLKPEDTAMYYCAADDQRFASPLYAYFGYWGQGTQVTVSSGGSQSLEESGGGLVKPGASLTLTCTASGFSFSGSYYMCWVRQAPGKGLECIACIYAGSSGATYYASWAKGRFTVSKTSSTTVTLQMTSLTAADTATYFCARHTNGIGLGLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 87 - protein, 360 aa, synthetic construct
MGWSCIILFLVATATGVHSQVQLQESGGGSVQAGGSLRLSCAASGFTFDDSDMGWYRQAPGNECDLVSTISSDGSTYYADSVKGRFTISQDNAKNTVYLQMDSVKPEDTAVYYCAADFMIAIQAPGAGCWGQGTQVTVSSGGSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 356 aa, synthetic construct
MGWSCIILFLVATATGVHSQVQLQESGGGSVPAGGSLKLSCAASGFSFSSYPMTWARQAPGKGLEWVSTIASDGGSTAYAASVEGRFTISRDNAKSTLYLQLNSLKTEDTAMYYCTKGYGDGTPAPGQGTQVTVSSGGSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 364 aa, synthetic construct
MGWSCIILFLVATATGVHSQVQLQESGGGSVQAGGSLRLSCTASGFTFDDSDMGWYRQAPGNECELVSTISSDGNTYYTDSVKGRFTISQDNAKNTVYLQMNSLGPEDTAVYYCAAEPRGYYSNYGGRRECNYWGQGTQVTVSSGGSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 359 aa, synthetic construct
MGWSCIILFLVATATGVHSQVQLQESGGGAVQAGGSLRLSCAASGFTFSNAHMSWVRQAPGKGREWISSIYSGGSTWYADSVKGRFTISRDNSKNTLYLQLNSLKTEDTAMYYCAENRLHYYSDDDSLRGQGTQVTVSSGGSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 366 aa, synthetic construct
MGWSCIILFLVATATGVHSQVQLQESGGGSVQAGGSLRLSCVASGYTFSSYCMGWFRQAPGKEREGVAALGGGSTYYADSVKGRFTISQDNAKNTLYLQMNSLKPEDTAMYYCAAAWVACLEFGGSWYDLARYKHWGQGTQVTVSSGGSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 366 aa, synthetic construct
MGWSCIILFLVATATGVHSQVQLQESGGGSVQAGGSLRLSCAASGSIYSSAYIGWFRQAPGKKREGVAGIYTRDGSTAYADSVKGRFTISQDSAKKTVYLQMNSLKPEDTAMYYCAAGRRTKSYVYIFRPEEYNYWGQGTQVTVSSGGSAFELTQTPSSVEAAVGGTVSINCQASEDIERYLAWYQQRPGQPPKLLIYTASSLPSGVPSRFKGSGSGTQFTLTISDLEAADAATYYCQQGWSDANVDNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 586 aa, synthetic construct
MGWSCIILFLVATATGVHSDVQLVESGGGVVQPGGSLRLSCAASGSIASIHAMGWFRQAPGKEREFVAVITVSGGITYYADSVKGRFTISRDPSKNTVYLQMNSLRPEDTALYYCAGDKHQSSFYDYWGQGTLVTVSSGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 708 aa, synthetic construct
MGWSCIILFLVATATGVHSQSVEESGGRLVTPGTPLTLTCTVSGFSLSSYDMSWVRQAPGKGLEYIGIISSSGSTYYASWAKGRFTISKTSTTVDLKITSPTTEDTATYFCARLGYVGYGYADIWGPGTLVTVSSGGGGSGGGGSGGGGSIEMTQTPFSVSAAVGGTVTIKCQASEDIYSLLAWYQQKPGQPPKLLIYSASTLASGVPSRFKGSGYGTEFTLTISGVQAADAATYYCQGGYYSSSADNTFGGGTEVVVKRGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 233 aa, synthetic construct
MGWSCIILFLVATATGVHSDIQMTQSPSSLSASVGDRVTITCRASHYITTWLAWYQQKPGKAPKLLIYDVSSLESGVPSRFSGSGSGTDFTFTISSLQPEDIATYYCQQYESYSPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 233 aa, synthetic construct
MGWSCIILFLVATATGVHSDIQMTQSPSSLSASVGDRVTITCQASQDISNYLNWYQQKPGKAPKLLIYDASNLETGVPSRFSGSGSGTDFTFTISSLQPEDIATYYCQQDANDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 235 aa, synthetic construct
MGWSCIILFLVATATGVHSDIQMTQSPSSLSASVGDRVTITCQASQDIGTYLNWYQQKPGKAPKLLIYEASTLETGVPSRFSGSGSGTDFTFTISSLQPEDIATYYCQQDGARDDYATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 233 aa, synthetic construct
MGWSCIILFLVATATGVHSDIQMTQSPSSLSASVGDRVTITCQASQDIDDYLNWYQQKPGKAPKLLIYDASNLQSGVPSRFSGSGSGTDFTFTISSLQPEDIATYYCQQDSMDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 235 aa, synthetic construct
MGWSCIILFLVATATGVHSDIQMTQSPSSLSASVGDRVTITCRASQSIDEYLNWYQQKPGKAPKLLIYEASKLQSGVPSRFSGSGSGTDFTFTISSLQPEDIATYYCQQDGAMDTYATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 233 aa, synthetic construct
MGWSCIILFLVATATGVHSEIVLTQSPATLSLSPGERATLSCRASQSVDEYLAWYQQKPGQAPRLLIYDASERATGIPARFSGSGSGTDFTLTISSLEPEDFAVYYCQQDATDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 404 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSQVQLVQSGAEVKKPGSSVKVSCKASGGTFSRYPINWVRQAPGQGLEWMGRIIPILGIADYAQRFQGRVTITADKSTNTAYMELSSLRSEDTAVYYCARRDWGDYWGQGTLVTVSSGGGGSGGGGSGGGGSEIVLTQSPGTLSLSPGERATLSCRASQSVSSSFLAWYQQKPGQAPRLLIYGASSRATGIPDRFSGSGSGTDFTLTISRLEPEDFAVYYCQQYSSSPLTFGGGTKVEIKRTVGGSHHHHHHHH

SEQ ID NO: 102 - protein, 404 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSEIVLTQSPGTLSLSPGERATLSCRASQSVSSSFLAWYQQKPGQAPRLLIYGASSRATGIPDRFSGSGSGTDFTLTISRLEPEDFAVYYCQQYSSSPLTFGGGTKVEIKRTVGGGGSGGGGSGGGGSQVQLVQSGAEVKKPGSSVKVSCKASGGTFSRYPINWVRQAPGQGLEWMGRIIPILGIADYAQRFQGRVTITADKSTNTAYMELSSLRSEDTAVYYCARRDWGDYWGQGTLVTVSSGGSHHHHHHHH

SEQ ID NO: 103 - protein, 416 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSQVQLVQSGAEVKKPGSSVKVSCKASGGTFSSDAINWVRQAPGQGLEWMGRIIPIFGVADYAQKFQGRVTLTADKSTSTAYMDLSSLRSEDTAVFYCARERGDYSNFWYFDLWGRGTLVTVSSGGGGSGGGGSGGGGSDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKVEIKRTVGGSHHHHHHHH

SEQ ID NO: 104 - protein, 416 aa, synthetic construct; identical to SEQ ID NO: 27

SEQ ID NO: 105 - protein, 405 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSQVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIKRTVGGSHHHHHHHH

SEQ ID NO: 106 - protein, 411 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSQVQLVQSGAEVKKPGSSVKVSCKASGGTFSSDAINWVRQAPGQGLEWMGRIIPIFGVADYAQKFQGRVTLTADKSTSTAYMDLSSLRSEDTAVFYCARERGDYSNFWYFDLWGRGTLVTVSSGGGGSGGGGSGGGGSDIQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPYTFGQGTKVEIKRTVGGSHHHHHHHH

SEQ ID NO: 107 - protein, 402 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSQIVLSQSPAILSASPGERVTMTCRASSSVSFMHWLQQKPGSSPKPWIYATSNLASGVSARFSGSGSGTSYSLTITRVEAEDAATYYCQQWSSNPPAFGGGTKLEIKGGGGSGGGGSGGGGSEVQLVESGGDLVQPRGSLKLSCAASGFTFSSYGMSWVRQTPDKRLELVATINGYGDTTYYPDSVKGRFTISRDNAKNTLYLQMSSLKSEDTAMYFCARDRDYGNSYYYALDYWGQGTSVTVSSGGSHHHHHHHH

SEQ ID NO: 108 - protein, 399 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSQVQLVQSGAEVKKPGSSVKVSCKASGGTFSRYPINWVRQAPGQGLEWMGRIIPILGIADYAQRFQGRVTITADKSTNTAYMELSSLRSEDTAVYYCARRDWGDYWGQGTLVTVSSGGGGSGGGGSGGGGSEIVLTQSPGTLSLSPGERATLSCRASQSVSSSFLAWYQQKPGQAPRLLIYGASSRATGIPDRFSGSGSGTDFTLTISRLEPEDFAVYYCQQYSSSPLTFGGGTKVEIKRTVGGSHHHHHHHH

SEQ ID NO: 109 - protein, 398 aa, synthetic construct
MGWSCIILFLVATATGVHSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSSQVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSGGGGSGGGGSGGGGSDIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKRTVGGSHHHHHHHH

SEQ ID NO: 110 - protein, 403 aa, synthetic construct
MGWSCIILFLVATATGVHSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGGSEVQLVESGGGLVKPGGSLRLSCVASELTFSNVWMTWVRQAPGKGLEWVGRIKSKADGGTTDYAAPVKGRFTISRDDSKNTLYLQMNSLKTEDTAVYYCTTGGYSYGDNWFDPWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKHHHHHHHH

SEQ ID NO: 111 - protein, 403 aa, synthetic construct
MGWSCIILFLVATATGVHSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKGGGGSGGGGSGGGGSEVQLVESGGGLVKPGGSLRLSCVASELTFSNVWMTWVRQAPGKGLEWVGRIKSKADGGTTDYAAPVKGRFTISRDDSKNTLYLQMNSLKTEDTAVYYCTTGGYSYGDNWFDPWGQGTLVTVSSHHHHHHHH

SEQ ID NO: 112 - protein, 406 aa, synthetic construct
MGWSCIILFLVATATGVHSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGGSAIQLTQSPSSLSASVGDRVTITCRASQGISSALAWYQQKPGKAPKLLIYDASSLESGVPSRFSGSGSGTDFTLTISSLQPEDFATYYCQQFNSYPITFGQGTRLEIKGGGGSGGGGSGGGGSEVQLVESGGGLVQPGRSLRLSCAASGFTFGDYAMHWVRQAPGKGLEWVSGISWNSGIIGYADSVKGRFTISRDNAKNSLYLQMNSLRAEDTALYYCVKDEGSSGWYDFNYFGMDVWGQGTTVTVSSHHHHHHHH

SEQ ID NO: 113 - protein, 403 aa, synthetic construct
MGWSCIILFLVATATGVHSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKGGGGSGGGGSGGGGSEVQLVESGGGLVKPGGSLRLSCVASELTFSNVWMTWVRQAPGKGLEWVGRIKSKADGGTTDYAAPVKGRFTISRDDSKNTLYLQMNSLKTEDTAVYYCTTGGYSYGDNWFDPWGQGTLVTVSSGGSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSHHHHHHHH

SEQ ID NO: 114 - protein, 400 aa, synthetic construct
MGWSCIILFLVATATGVHSQVQLVESGGGLVKPGGSLRLSCAASGFTFSDYYMSWIRQAPGKGLEWVSFISSSGIIIYYADSVKGRFTISRDNAKNSLYLQMNSLRAEDTAVYYCARRGYSGYDYYYYGLDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSTFGGGTKVEIKGSQVQLQESGGGSVQTGGSLRLSCTASTSIYSNNYMAWFSQSPGKGREGVAAVYMDDGRPYYADSVKGRFTISLDSAKNTMYLQMNSLKPEDTAMYYCAAAPGPLSRNYWYTSANYDYWGQGTQVTVSSHHHHHHHH

SEQ ID NO: 115 - protein, 400 aa, synthetic construct
MGWSCIILFLVATATGVHSEIVLTQSPGTLSLSPGERATLSCRASQSVSSSYLAWYQQKPGQAPRLLIYGASSRATGIPDRFSGSGSGTDFTLTISRLEPEDFAVYYCQQYGSTFGGGTKVEIKGGGGSGGGGSGGGGSQVQLVESGGGLVKPGGSLRLSCAASGFTFSDYYMSWIRQAPGKGLEWVSFISSSGIIIYYADSVKGRFTISRDNAKNSLYLQMNSLRAEDTAVYYCARRGYSGYDYYYYGLDVWGQGTTVTVSSGSQVQLQESGGGSVQTGGSLRLSCTASTSIYSNNYMAWFSQSPGKGREGVAAVYMDDGRPYYADSVKGRFTISLDSAKNTMYLQMNSLKPEDTAMYYCAAAPGPLSRNYWYTSANYDYWGQGTQVTVSSHHHHHHHH

SEQ ID NO: 116 - protein, 407 aa, synthetic construct
MGWSCIILFLVATATGVHSQVQLVQSGAEVKEPGASVKISCKASGYTFTNDYMHWVRQAPGQGLEWMGIINPTGGGTSYAQKFRGRVTMTRDTSTSTVYMELSSLRSEDTAVFYCARERYYGSGTYFNIGLWYFDLWGRGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKGSQVQLQESGGGSVQTGGSLRLSCTASTSIYSNNYMAWFSQSPGKGREGVAAVYMDDGRPYYADSVKGRFTISLDSAKNTMYLQMNSLKPEDTAMYYCAAAPGPLSRNYWYTSANYDYWGQGTQVTVSSHHHHHHHH

SEQ ID NO: 117 - protein, 407 aa, synthetic construct
MGWSCIILFLVATATGVHSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKGGGGSGGGGSGGGGSQVQLVQSGAEVKEPGASVKISCKASGYTFTNDYMHWVRQAPGQGLEWMGIINPTGGGTSYAQKFRGRVTMTRDTSTSTVYMELSSLRSEDTAVFYCARERYYGSGTYFNIGLWYFDLWGRGTLVTVSSGSQVQLQESGGGSVQTGGSLRLSCTASTSIYSNNYMAWFSQSPGKGREGVAAVYMDDGRPYYADSVKGRFTISLDSAKNTMYLQMNSLKPEDTAMYYCAAAPGPLSRNYWYTSANYDYWGQGTQVTVSSHHHHHHHH

SEQ ID NO: 118 - protein, 251 aa, synthetic construct
MGWSCIILFLVATATGVHSQLQLQESGPGLVKPSETLSLTCTVSGGSISSSVYYWSWIRQPPGKGLEWIGVIYPSGNTYYSPSLKSRVTISVDTSKNQFSLKLSSVTAADTAVYYCARTIYDLFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHHH

SEQ ID NO: 119 - protein, 353 aa, synthetic construct
MGWSCIILFLVATATGVHSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGSDIQMTQSPSSVSASVGDRVTITCRASQGISSWLAWYQQKPGKAPKLLIYAASSLQSGVPSRFSGSGSGTDFTLTISSLQPEDFATYYCLQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 359 aa, synthetic construct
MGWSCIILFLVATATGVHSVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVSGGSDIQMTQSPSSVSASVGDRVTITCRASQGISSWLAWYQQKPGKAPKLLIYAASSLQSGVPSRFSGSGSGTDFTLTISSLQPEDFATYYCLQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 707 aa, synthetic construct
MDMRVPAQLLGLLLLWLRGARCEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCASSKEKATYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCASSKEKATYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 122 - protein, 489 aa, synthetic construct
MDMRVPAQLLGLLLLWLRGARCQVKLEESGGGLVQAGGSLRLSCAASGRIFSIYDMGWFRQAPGKEREFVSGIRWSGGTSYADSVKGRFTISKDNAKNTIYLQMNNLKAEDTAVYYCGSRGYWGQGTLVTVSSGGGGSGGGGSQVKLEESGGGLVQAGGSLRLSCAASGRIFSIYDMGWFRQAPGKEREFVSGIRWSGGTSYADSVKGRFTISKDNAKNTIYLQMNNLKAEDTAVYYCGSRGYWGQGTLVTVSSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHNHYTQKSLSLSP

SEQ ID NO: 123 - protein, 238 aa, synthetic construct
MDMRVPAQLLGLLLLWLRGARCQAVVLQEPSLSVSPGGTVTLTCGLSSGSVSTNYYPSWYQQTPGQAPRTLIYYTNTRSSDVPERFSGSIVGNKAALTITGAQPDDESVYFCLLYLGRGIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 124 - protein, 463 aa, synthetic construct
MGWSCIILFLVATATGVHSEVKLVESGGGLVAPGGSLKLSCAASGFTFSSYPMSWVRQTPEKRLEWVAYINNGGGNPYYPDTVKGRFTISRDNAKNTLYLQMSSLKSEDTAIYYCIRQYYGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 125 - protein, 374 aa, synthetic construct
MGWSCIILFLVATATGVHSEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 126 - protein, 466 aa, synthetic construct
MGWSCIILFLVATATGVHSEVKLVESGGGLVKPGGSLTLPCAASGFTFSRYAMSWVRQTPEKRLEWVASISSGGDTYYPDSVKGRFTVSRDNARNILFLQMSSLRSEDTGMYYCARVNYYDSSFLDWWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHNHYTQESLSLSP

SEQ ID NO: 127 - protein, 238 aa, synthetic construct
MGWSCIILFLVATATGVHSDVLMTQTPLSLPVSLGDQASISCRSSQTIVHSNGKIYLEWYLQKPGQSPKLLIYRVSKRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 234 aa, synthetic construct
MGWSCIILFLVATATGVHSDIVMTQSQRFMSTLEGDRVSVTCKASQNVGTNVAWYQQKPGQSPKALIYSASYRYSGVPDRFTGSGSGTDFTLTISNVQSEDLAEYFCQQYNNYPLYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 130 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 131 - protein, 279 aa, synthetic construct
MGWSCIILFLVATATGVHSQVQLQESGGGSVQAGGSLRLSCAASGYTYRDYYMGWFRQAPGREREGVASIYTRGSREGSTRYSSSVEGRFTITLDTAKNTLYLQMNSLKPEDTAMYYCAADDRTWLPRVQLGGPRENEYNYWGQGTQVTVSSGGSQLQLVESGGGLVQAGGSLRLSCAASGRGFSSYTMGWFRQAPGKEREFVSAIRWSGSITYYADSVKGRFTISRDNAKNTMYLQMNSLKPEDTAVYYCAASPVAGWGTSPAWYDYWGQGTQVTVSS

SEQ ID NO: 132 - protein, 275 aa, synthetic construct
MGWSCIILFLVATATGVHSQVQLQESGGGSVQAGGSLRLSCAASGYTYSKNWYMGWFRQTPGKEREGVAVIAYDDWPTYADSVKGRFTISKDNTKNTLYLQMNSLKPEDTAMYYCAARQLGGDYCYFPNLSRFCYNYWGQGTQVTVSSGGSQLQLVESGGGLVQAGGSLRLSCAASGRGFSSYTMGWFRQAPGKEREFVSAIRWSGSITYYADSVKGRFTISRDNAKNTMYLQMNSLKPEDTAVYYCAASPVAGWGTSPAWYDYWGQGTQVTVSS

SEQ ID NO: 133 - protein, 264 aa, synthetic construct
MGWSCIILFLVATATGVHSQVQLQESGGGSVQAGGSLRLSCTASGFTFNEANHMGWYRQAPGNECELVSTISSDGTTYYPDSVKGRFTISQDNAKKTAFLQMNSLKPEDTAVYYCAADQSRRGSLCLGQGTQVTVSSGGSQLQLVESGGGLVQAGGSLRLSCAASGRGFSSYTMGWFRQAPGKEREFVSAIRWSGSITYYADSVKGRFTISRDNAKNTMYLQMNSLKPEDTAVYYCAASPVAGWGTSPAWYDYWGQGTQVTVSS

SEQ ID NO: 134 - protein, 275 aa, synthetic construct
MGWSCIILFLVATATGVHSQLQLVESGGGLVQPGGSLTLSCAASGRSFSSYGMGWFRQAPGKEREFVATISWNSGSIYYTDSVKGRFTISRDDAKNTMSLQMNSLRPEDTAVYSCAAGPRDWGNMRKFEEYEYRGQGTQVTVSSGGSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 135 - protein, 275 aa, synthetic construct
MGWSCIILFLVATATGVHSQVQLVESGGGLVQPGGSLTLSCAASGRSFSEYGMGWFRQAPGKEREFVATISWDSDSIYYTDSVKGRFTISRDNAKNTMSLQMNSLKPEDTAVYSCAARPRDWGNMRRFEAYEYRGQGTQVTVSSGGSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 136 - protein, 273 aa, synthetic construct
MGWSCIILFLVATATGVHSQVQLVESGGGLVQTGGSLRLSCAASEPTFSDYAGSWFRQAPGKEREFLASINWRGDTTYYADSVKGRFTISRDNAKNMMYLEMNNLEPEDTAVYRCAAKATDWSSTLYEYDYWGQGTQVTVSSGGSQVQLQESGGGSVQAGGSLRLSCAASSYTISSVCMGWFRQAPGKEREGVAGIAPDGSTGYGDSVKGRFTISKDNAKNTLYLQMNSLKPEDTAMYYCAAASPGRCFLPRTALEPALYYNWGQGTQVTVSS

SEQ ID NO: 137 - protein, 272 aa, synthetic construct
MGWSCIILFLVATATGVHSQLQLVESGGGLVQPGGSLTLSCAASGRSFSSYGMGWFRQAPGKEREFVATISWNSGSIYYTDSVKGRFTISRDDAKNTMSLQMNSLRPEDTAVYSCAAGPRDWGNMRKFEEYEYRGQGTQVTVSSGGSQVQLQESGGGSVQAGGSLRLSCVGSGYTYDTSDMSWYRQAPGKEREFVSDIDSGDWAAYADAVKGRFTISRDNAKKTVYLQMNSLEPEDTAMYYCKASYWKWGKLNNFWGPGTQVTVSSHHHHHH

SEQ ID NO: 138 - protein, 275 aa, synthetic construct
MGWSCIILFLVATATGVHSQLQLVESGGGLVQPGGSLTLSCAASGRSFSSYGMGWFRQAPGKEREFVATISWNSGSIYYTDSVKGRFTISRDDAKNTMSLQMNSLRPEDTAVYSCAAGPRDWGNMRKFEEYEYRGQGTQVTVSSGGSQVQLQESGGGLVQPGGSLKLSCAASGFRFSNYGMSWVRQAPGEGLEWVSYINGDGSRTHYADSVKGRFTISRDNAKNTLYLQLNSLKTEDTAMYYCEKGLSRDGWSLSAASRGQGTQVTVSSHHHHHH

SEQ ID NO: 139 - protein, 279 aa, synthetic construct
MGWSCIILFLVATATGVHSQLQLVESGGGLVQPGGSLTLSCAASGRSFSSYGMGWFRQAPGKEREFVATISWNSGSIYYTDSVKGRFTISRDDAKNTMSLQMNSLRPEDTAVYSCAAGPRDWGNMRKFEEYEYRGQGTQVTVSSGGSQVQLQESGGGSVQAGGSLRLSCVTSGYTYSSANMAWFRQAPGKEREGVAIITPSGRATTYADSVKGRFTISRDNAANTLYLQMNSLKPEDTAMYYCAADTPPYSGLWYAERTYNYWGQGTQVTVSSHHHHHH

SEQ ID NO: 140 - protein, 281 aa, synthetic construct
MGWSCIILFLVATATGVHSQLQLVESGGGLVQPGGSLTLSCAASGRSFSSYGMGWFRQAPGKEREFVATISWNSGSIYYTDSVKGRFTISRDDAKNTMSLQMNSLRPEDTAVYSCAAGPRDWGNMRKFEEYEYRGQGTQVTVSSGGSQVQLQESGGGSVQAGGSLRLSCTASGFTFDDEDMGWYRQAPGNECELVSSLGSLGRRYYADSVKDRFAISQDNAKNTVYLQMNSLKPEDTAVYYCAATKGGSWLDSILASCQGAFGYWGQGTQVTVSSHHHHHH

SEQ ID NO: 141 - protein, 273 aa, synthetic construct
MGWSCIILFLVATATGVHSQLQLVESGGGLVQPGGSLTLSCAASGRSFSSYGMGWFRQAPGKEREFVATISWNSGSIYYTDSVKGRFTISRDDAKNTMSLQMNSLRPEDTAVYSCAAGPRDWGNMRKFEEYEYRGQGTQVTVSSGGSQVQLQESGGGSVQAGGSLRLSCAASGSTSCSSVMRWYRQAPGKEREFVSSINSDRRTVYADSVKGRFTISQDNAKSTLYLQMNSLKAEDTATYYCQRELYGDSWCQGNYWGQGTQVTVSSHHHHHH

(19) United States
(12) Patent Application Publication
(10) Pub. No.: US 2026/0265386 A1
(43) Pub. Date: Sep. 10, 2026

(54) CONDITIONALLY ACTIVATED RECEPTOR SIGNALING BY USING SCAFFOLD PROTEIN

(71) Applicant: CHUGAI SEIYAKU KABUSHIKI KAISHA, Tokyo (JP)

(72) Inventor: Akihiko MIZOROKI, Singapore (SG)

(21) Appl. No.: 19/162,561

(22) PCT Filed: Mar. 6, 2024
(86) PCT No.: PCT/JP2024/008456

§ 371 Date: Sep. 5, 2025

(30) Foreign Application Priority Data
Mar. 7, 2023 (EP) 23160546.0

(51) Int. Cl. C07K 16/28; C12N 15/62

(52) U.S. Cl. CPC C07K 16/2866; C07K 16/2818; C07K 16/2827; C07K 16/2863; C07K 16/2875; C12N 15/62; C07K 2317/14; C07K 2317/31; C07K 2317/52; C07K 2317/569; C07K 2317/622; C07K 2317/76; C07K 2319/03; C07K 2319/31

(57) ABSTRACT

The present invention relates to one or more binding molecules that are capable of inducing receptor signaling of a receptor complex, conditioned upon non-competitive and/or biparatopic binding to a scaffold protein. The present invention also relates to the therapeutic use of the one or more binding molecules in cancer and autoimmune disease.

### TECHNICAL FIELD

The present invention relates to one or more target-binding molecules that are capable of inducing receptor signaling of a receptor complex, conditioned upon binding to a scaffold protein.

### BACKGROUND ART

Because of the risk of toxicity, most of receptor ligands including but not limited to agonistic ligands, for example cytokines, have limitation to be used for therapy. Localizing agonistic activity of ligands is a promising approach to improve the efficacy and safety of therapeutic molecules. Currently, there are several approaches being developed, like protease-activated ligands (Nature Communications 2021; 12: 2768), antibody-ligand conjugation (Nature 2022; 610: 161-172) and split cytokine (WO2020/106708 A1). However, the selectivity of these approaches is still limited. In addition, many receptors including but not limited to cytokine receptors induce signaling by forming homo-, hetero-dimer or oligomerization on the cell surface for effective signaling. The present disclosure, which is concerned with target-binding molecules that induce effective signaling of a receptor complex dependent on the binding to a scaffold protein, addresses these needs.

### CITATION LIST

### Patent Literature

[PTL 1] WO 2020/106708 A1

### Non Patent Literature

[NPL 1] Hsu, E. J., Cao, X., Moon, B. et al. A cytokine receptor-masked IL2 prodrug selectively activates tumor-infiltrating lymphocytes for potent antitumor therapy. Nat Commun 12, 2768 (2021)
[NPL 2] Deak, L. C., Nicolini, V., Hashimoto, M., et al. PD1-cis-IL-2R agonism yields better effectors from stem-like CD8+T cells. Nature 610, 161-172 (2022)
[NPL 3] Wang, X., Rickert, M., Garcia, K. C. Structure of the quaternary complex of interleukin-2 with its alpha, beta, and gammac receptors. Science 310, 1159-1163 (2005)
[NPL 4] Mitra, S., Ring, A. M., Amarnath, S., et al. Interleukin-2 activity can be fine tuned with engineered receptor signaling clamps. Immunity 42, 826-838 (2015)
[NPL 5] Song, D., Liu, X., Dong, C., et al. Two novel human anti-CD25 antibodies with antitumor activity inversely related to their affinity and in vitro activity. Scientific Reports 11, 22966 (2021).
[NPL 6] Yen, M., Ren, J., Liu, Q., et al. Facile discovery of surrogate cytokine agonists. Cell 185, 1414-1430.e19 (2022)
[NPL 7] Sun, D., Sang, Z., Kim, Y. J., et al. Potent neutralizing nanobodies resist convergent circulating variants of SARS-CoV-2 by targeting diverse and conserved epitopes. Nature Communications 12, 4676 (2021)
[NPL 8] Fenwick, C., Loredo-Varela, J-L., Joo, V., et al. Tumor suppression of novel anti-PD1 antibodies mediated through CD28 costimulatory pathway. Journal of Experimental Medicine 216, 1525-1541 (2019)
[NPL9] Tan, S., Zhang, H., Chai, Y., et al. An unexpected N-terminal loop in PD-1 dominates binding by nivolumab. Nature Communications 8, 14369 (2017)
[NPL10] Wang, L., Bi, X., Zhu, Y., et al. IL-2R alpha up-regulation is mediated by latent membrane protein 1 and promotes lymphomagenesis and chemotherapy resistance in natural killer/T-cell lymphoma. Cancer Communications 38, 62 (2018)

### SUMMARY OF INVENTION

### Technical Problem

Effective receptor signaling is a key requirement for the effectiveness of agonistic ligands. Well-known examples are cytokines, which are key immune mediators residing in many lesion sites whose effects, when harnessed, can significantly improve immune responses. This also applies to interleukin-2 (IL-2). While many therapies with agonistic ligands have been developed, the issue of high toxicity and low efficacy remains of concern.

### Solution to Problem

The present inventors have thought that the ability to deliver an agonist capable of site-specifically activating receptor signaling would overcome systemic toxicity and low efficacy issue. To achieve such site-specific activation of receptor signaling, the present inventors have inter alia developed combinations of target-binding molecules or protein complexes, which, in the presence of a scaffold protein, are capable of inducing receptor signaling of a receptor complex. The combination of target-binding molecules or the protein complex of the present invention comprises a first binding domain, and a third binding domain, which are each capable of non-competitively binding to a scaffold protein. The combination of target-binding molecules or the protein complex of the present invention comprises a second binding domain, which is capable of binding to a first receptor protein, and a fourth binding domain, which is capable of binding to a second receptor protein. The first receptor protein and the second receptor protein are each receptor subunits that can associate to form a receptor complex. Upon binding of all four binding domains, the receptor complex is activated and can induce signaling. By making the presence of the scaffold protein a condition of the activation of the receptor complex, and of the specific binding of the present invention, the receptor signaling can be conditionally induced, thus significantly increasing the specificity and lowering the toxicity.

### EXEMPLARY EMBODIMENTS

The present invention is based on such findings, and specifically includes the exemplary aspects and embodiments described below.
[A-1] The present invention is in particular concerned with a combination of target-binding molecules, comprising
   a first target-binding molecule, comprising a first binding domain that is capable of binding to a scaffold protein and a second binding domain that is capable of binding to a first receptor protein, and
   a second target-binding molecule, comprising a third binding domain that is capable of binding to the scaffold protein and a fourth binding domain that is capable of binding to a second receptor protein,
   wherein the first target-binding molecule and the second target-binding molecule are capable of non-competitively binding to the scaffold protein, and
   wherein the first receptor protein and the second receptor protein are each receptor subunits that can associate to form a receptor complex, wherein the combination of target-binding molecules is capable of inducing receptor signaling of the receptor complex.
[A-1a] The present invention is concerned with a combination of target-binding molecules, comprising
   a first target-binding molecule, comprising a first binding domain that is capable of binding to a scaffold protein and a second binding domain that is capable of binding to a first receptor protein, and
   a second target-binding molecule, comprising a third binding domain that is capable of binding to the scaffold protein and a fourth binding domain that is capable of binding to a second receptor protein,
   wherein the first target-binding molecule and the second target-binding molecule are capable of non-competitively binding to the scaffold protein, and
   wherein the first receptor protein and the second receptor protein are each receptor subunits that can associate to form a receptor complex. Upon binding of the four binding domains as defined above, the receptor complex induces signalling.
[A-2] In a preferred embodiment of the combination of target-binding molecules of [A-1] or [A-1a], the first binding domain and the third binding domain are capable of binding the scaffold protein biparatopically.
[A-3] In a preferred embodiment of the combination of target-binding molecules of any one of [A-1] to [A-2], the combination of target-binding molecules induces receptor signaling in a first concentration or amount of the scaffold protein and does not induce receptor signaling in a second concentration or amount of the scaffold protein, wherein when the first concentration or amount of the scaffold protein is increased by at least 10%, 20%, 30%, 40%, 50%, 60%, 70%, 80%, 90%, 100%, 150%, 200%, 250%, 300%, 350%, 400%, 450% or 500% as compared to the second concentration or amount of the scaffold protein, receptor signaling is increased by at least 10%, 20%, 30%, 40%, 50%, 60%, 70%, 80%, 90%, 100%, 150%, 200%, 250%, 300%, 350%, 400%, 450% or 500%.
[A-4] In a preferred embodiment of the combination of target-binding molecules of any one of [A-1] to [A-3], the first binding domain is connected to the second binding domain, optionally via a linker, and/or the third binding domain is connected to the fourth binding domain, optionally via a linker. In a preferred embodiment, the linker is 20, 15, 10 amino acids or less. In an even more preferred embodiment, the linker is 10 amino acids or less.
[A-5] In a preferred embodiment of the combination of target-binding molecules of any one of [A-1] to [A-4], the first binding domain and the third binding domain are capable of binding the scaffold protein biparatopically within a domain of the scaffold protein.
[A-5a] In a preferred embodiment of the combination of target-binding molecules of any one of [A-1] to [A-4], the first binding domain and the third binding domain are each selected from the group consisting of a neutralizing binding domain and a non-neutralizing binding domain. Preferably, the first binding domain is a neutralizing binding domain and the third binding domain is a non-neutralizing binding domain or vice versa.
[A-6] In a preferred embodiment of the combination of target-binding molecules of any one of [A-1] to [A-5a], the scaffold protein comprises two, three, four, or more subunits.
[A-7] In a preferred embodiment of the combination of target-binding molecules of any one of [A-1] to [A-6] the first binding domain and the third binding domain each are capable of binding subunits of the scaffold protein which can associate to form the scaffold protein.
[A-8] In a preferred embodiment of the combination of target-binding molecules of any one of [A-1] to [A-7], the scaffold protein is a membrane-bound protein, a soluble protein, or an insoluble protein deposition.
[A-9] In a preferred embodiment of the combination of target-binding molecules of any one of [A-1] to [A-8], the scaffold protein is a cell surface marker, an immune cell surface marker, a T cell exhaustion marker, tumor-specific marker, tumor-associated marker, or a protein whose expression is characteristic of a disease or disorder.
[A-10] In a preferred embodiment of the combination of target-binding molecules of any one of [A-1] to [A-9], the scaffold protein is selected from the group consisting of PD1, PDL1, CSF2RB, CD4, CD5, CD6, CD7, CD8, CD9, CCR4, CD8, CD25, CD27, CD38, CD39, CD45, CD62L, CCR6, CD69, CD103, CD73, CD127, GITR, LRRC32, ICOS, TIGIT, RBD, MUC1, CEA, CTLA4, IL2R alpha, CXCR5, Neuropilin-1, TIM3, LAG3, TNF alpha, CD19, CD20, CD22, CD30, CD33, Glycoprotein NMB, CD56, CD70, CD79A, CD79B, CD138, PSCA, PSMA, BCMA, FcRH5, GPRC5D, FAP, LRRC15, E-selectin, EphB2, Melanotransferrin, HER2, TROP2, Nectine-4, EGFRvIII, IL13RA2, TMEFF2, Muc16, EpCAM, FcRH2, AFP, PSA, Amyloid beta, MBP and ASGPR. In an even more preferred embodiment, the scaffold protein is selected from the group consisting of PD1, PDL1, TNF alpha, CD25, MUC1, CEA and CD8.
[A-11] In a preferred embodiment of the combination of target-binding molecules of any one of [A-1] to [A-10], the first and second receptor proteins are each independently selected from the group consisting of IL2R beta, IL2R gamma, IL4R, IL13RA1, IL7R, IL9R, IL21R, TSLPR, IL3RA, CSF2RB, IL5RA, CSF2RA, IL6R, gp130, IL11RA, IL12RB1, IL12RB2, IL27RA, IL31RA, OSMR, CNTFR, LIFR, IL10RA, IL10RB, IL20RA, IL20RB, IL22RA1, IL28RA, IFNAR1, IFNAR2, IFNGR1, IFNGR2, IL1R1, IL1RAP, IL18R1, IL18RAP, ST2, IL17RA, IL17RC, TLR3, TLR4, TLR7, TLR9, CSF1R, TNFR1, TNFR2, LTBR, HVEM, FAS, CD28, cMET, DR3, DR4, DR5, NGFR, RANK, FN14, CD40, 4-1BB, OX40, GITR, TGFBR1, TGFBR2, ACVRL1, ACVR2A, BMPR2, ACVR2B, ACVR1B, ACVR1C, ACVR1, AMHR2, BMPR1A, BMPR1B, BMPR2, TRA, TRB, CD3E, CD16, TREM2, FGFR1, FGFR2, FGFR3, FGFR4, Fzd1, Fzd2, Fzd3, Fzd4, Fzd5, Fzd6, Fzd7, Fzd8 Fzd9, Fzd10, LRP5, LRP6 and LGR5. In an even more preferred embodiment, the first and second receptor proteins are each independently selected from the group consisting of IL2R beta, IL2R gamma, Fzd1, Fzd2, Fzd3, Fzd4, Fzd5, Fzd6, Fzd7, Fzd8 Fzd9, Fzd10, LRP5, LRP6, CSF2RA, CSF2RB and cMET.
[A-11a] In a preferred embodiment of the combination of target-binding molecules of any one of [A-1] to [A-10], the scaffold protein is PD1, the first receptor protein is IL2R beta, and the second receptor protein is IL2R gamma.
[A-11b] In a preferred embodiment of the combination of target-binding molecules of any one of [A-1] to [A-10], the scaffold protein is IL2R alpha, the first receptor protein is IL2R beta, and the second receptor protein is IL2R gamma.
[A-11c] In a preferred embodiment of the combination of target-binding molecules of any one of [A-1] to [A-10], the target binding molecules compete for binding with or binds to the same epitope as any one of the target-binding molecules or protein complex of any one of Tables 1-17.
[A-11d] In a preferred embodiment of the combination of target-binding molecules of any one of [A-1] to [A-10], the scaffold protein, the first receptor protein, and the second receptor protein is a combination chosen from any of (i) to (vi) below:
   (i) the scaffold protein is PDL1, the first receptor protein is IL2R beta, and the second receptor protein is IL2R gamma;
   (ii) the scaffold protein is TNFa, the first receptor protein is IL2R beta, and the second receptor protein is IL2R gamma;
   (iii) the scaffold protein is CD25, the first receptor protein is IL2R beta, and the second receptor protein is IL2R gamma;
   (iv) the scaffold protein is PDL1, the first receptor protein is CSF2RA, and the second receptor protein is CSF2RB;
   (v) the scaffold protein is MUC1 or CEA, the first receptor protein is a Fzd receptor, and the second receptor protein is a Lrp; or
   (vi) the scaffold protein is CD8, the first receptor protein is IL2R beta, and the second receptor protein is IL2R gamma.
[A-11e] In a preferred embodiment of the combination of target-binding molecules of any one of [A-1] to [A-11d], the receptor complex is IL-2R. In a more preferred embodiment, inducing IL-2 receptor complex signaling comprises evaluating IL-2 receptor complex activation using a colorimetric enzyme assay that determines alkaline phosphatase activity. In a even more preferred embodiment, alkaline phosphatase activity is determined by measuring optical density optionally at 620 nm. In a most preferred embodiment, receptor signaling of the receptor complex is induced when there is an increase in optical density by at least 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500% in the presence of the combination of target-binding molecules of [A-1] to [A-11c] as compared to in the absence of the combination of target-binding molecules of [A-1] to [A-11c].
[A-12] In a preferred embodiment of the combination of target-binding molecules of any one of [A-1] to [A-11e], each binding domain is an antigen binding domain comprising VH and VL, sdAb, VHH, scFv, Fab, scFab, Fab′, Fab′-SH, F(ab′)2, diabody, triabody, Fv, aptamer, Affibody, a cytokine, a ligand, or a split cytokine.
[A-13] In a preferred embodiment of the combination of target-binding molecules of any one of [A-1] to [A-12], the first target-binding molecule and the second target-binding molecule are connected, optionally via a linker. In this embodiment, the combination is a single protein or protein complex.
[A-14] In a preferred embodiment of the combination of target-binding molecules of any one of [A-1] to [A-13], the first target-binding molecule and the second target-binding molecule are each connected to a half-life extending domain, optionally via a linker.
[A-15] In a preferred embodiment of the combination of target-binding molecules of [A-14], the first target-binding molecule is connected to the half-life extending domain at the first binding domain and the second target-binding molecule is connected to the half-life extending domain at the third binding domain.
[A-16] In a preferred embodiment of the combination of target-binding molecules of [A-14], the half-life extending domain comprises an Fc domain, FcRn-binding domain, an albumin-binding domain, an albumin or variant thereof, or polyethylene glycol.
[A-17] In a preferred embodiment of the combination of target-binding molecules of [A-14], the half-life extending domain comprises an Fc domain, optionally wherein the Fc domain comprises modifications that diminish antibody-dependent cellular cytotoxicity (ADCC), diminish complement-dependent cytotoxicity (CDC), increase FcRn binding, increase pI, or promote heterodimerization.
[A-18] The present invention is also concerned with a nucleic acid molecule encoding the combination of target-binding molecules of any one of [A-1] to [A-17].
[A-19] The present invention is also concerned with a plurality of nucleic acid molecules encoding the combination of target-binding molecules of any one of [A-1] to [A-17], wherein the plurality comprises at least one first nucleic acid molecule and at least one second nucleic acid molecule, wherein the first nucleic acid molecule encodes the first target-binding molecule as defined in any one of [A-1] to [A-17], and wherein the second nucleic acid molecule encodes the second target-binding molecule as defined in any one of [A-1] to [A-17].
[A-20] The present invention is also concerned with a vector comprising the nucleic acid molecule of [A-18], or the plurality of nucleic acid molecules of [A-19].
[A-20a] The present invention is also concerned with a first vector comprising the first nucleic acid molecule of [A-19] and a second vector comprising the second nucleic acid molecule of [A-19].
[A-21] The present invention is also concerned with a host cell comprising the nucleic acid molecule of [A-18], or the plurality of nucleic acid molecules of [A-19], or the vector of [A-20].
[A-21a] The present invention is also concerned with a first host cell comprising the first nucleic acid molecule of [A-19] or the first vector of [A-20a] and a second host cell comprising the second nucleic acid molecule of [A-19] or the second vector of [A-20a].
[A-22] The present invention is also concerned with a method for producing a combination of target-binding molecules, comprising the steps of:
   (i) culturing the host cell of [A-21] or the host cells of [A-21a] under conditions suitable for protein expression;
   (ii) optionally lysing the host cell; and
   (iii) isolating the combination of target-binding molecules.
[A-23] The present invention is also concerned with a method for producing a combination of target-binding molecules, comprising the steps of:
   (i) culturing the host cell of [A-21] or first host cell of [A-21a] under conditions suitable for protein expression to obtain the first target-binding molecule;
   (ii) culturing the host cell of [A-21] or second host cell of [A-21a] under conditions suitable for protein expression to obtain the second target-binding molecule;
   (iii) isolating the first target molecule and the second target molecule from the host cells; and
   (iv) combining the first target-binding molecule and the second target-binding molecule to obtain the combination of target-binding molecules.
[A-24] The present invention is also concerned with a pharmaceutical composition comprising the combination of target-binding molecules of any one of claims [A-1] to [A-17].
[A-25] The present invention is also concerned with a method for treating a disease in a subject, the method comprising administering the pharmaceutical composition of [A-24] to a subject in need thereof.
[A-26] In a preferred embodiment of the method for treating a disease in a subject of [A-25], the disease is a cancer or an autoimmune disease.
[A-26a] In a preferred embodiment the cancer or autoimmune disease of [A-25] is a cancer or autoimmune disease that is susceptible of being improved or prevented by an increase or enhancement in IL-2R signalling.
[A-27] The present invention is also concerned with the pharmaceutical composition of [A-24] for use in therapy.
[A-28] In a preferred embodiment of the pharmaceutical composition for use of [A-27], the therapy is cancer immunotherapy or autoimmune disease immunotherapy.
[A-28a] In a preferred embodiment the cancer or autoimmune disease of [A-28] is a cancer or autoimmune disease that is susceptible of being improved or prevented by an increase or enhancement in IL-2R signalling.
[A-29] The present invention is also concerned with the use of the pharmaceutical composition of [A-24] in the manufacture of a medicament for treating a disease.
[A-30] In a preferred embodiment of the use of claim [A-29], the disease is a cancer or an autoimmune disease.
[A-30a] In a preferred embodiment the cancer or autoimmune disease of [A-30] is a cancer or autoimmune disease that is susceptible of being improved or prevented by an increase or enhancement in IL-2R signalling.
[A-31] In a preferred embodiment of the combination of target-binding molecules of any one of [A-1] to [A-17], the method of any one of [A-25] or [A-26], the pharmaceutical composition for use of any one of [A-27] or [A-28], or the use of any one of [A-29], [A-30] or [A30a], the scaffold protein is PD-1, the first receptor protein is IL-2R beta, and the second receptor protein is IL-2R gamma.
[A-31a] In a preferred embodiment of the combination of target-binding molecules of any one of [A-1] to [A-17], the method of any one of [A-25] or [A-26], the pharmaceutical composition for use of any one of [A-27] or [A-28], or the use of any one of [A-29], [A-30] or [A-30a], the scaffold protein is IL-2R alpha, the first receptor protein is IL-2R beta, and the second receptor protein is IL-2R gamma.
[A-32] In a preferred embodiment of the combination of target-binding molecules of any one of [A-1] to [A-17], the method of claim [A-26], the pharmaceutical composition for use of [A-28], or the use of [A-30] or [A-30a], the cancer or autoimmune disease is characterized by PD1, IL2R alpha, IL-2R beta, or IL-2R gamma expression.
[A-33] The present invention is also concerned with the first target-binding molecule as defined in any one of [A-1] to [A-17] for use in therapy, wherein therapy comprises administering the first target-binding molecule in combination with the second target-binding molecule as defined in any one of [A-1] to [A-17], optionally wherein the first target-binding molecule and the second target-binding molecule are to be administered simultaneously, sequentially, or separately.
[A-34] The present invention is also concerned with the second target-binding molecule as defined in any one of [A-1] to [A-17] for use in therapy, wherein therapy comprises administering the second-target binding molecule in combination with the first target-binding molecule as defined in any one of [A-1] to [A-17], optionally wherein the first target-binding molecule and the second target-binding molecule are to be administered simultaneously, sequentially, or separately.
[A-35] In a preferred embodiment of the first target-binding molecule for use of [A-33] or the second target-binding molecule for use of [A-34], the disease is a cancer or an autoimmune disease.
[A-35a] In a preferred embodiment the cancer or autoimmune disease of [A-35] is a cancer or autoimmune disease that is susceptible of being improved or prevented by an increase or enhancement in IL-2R signalling.
[A-36] In a preferred embodiment of the first target-binding molecule or the second target-binding molecule for use of [A-35], the cancer or autoimmune disease is characterized by PD1, IL2R alpha, IL-2R beta, or IL-2R gamma expression.
[A-37] In a preferred embodiment of the first target-binding molecule or the second target-binding molecule for use of any one of [A-33] to [A-36], the scaffold protein is PD1, the first receptor protein is IL-2R beta, and the second receptor protein is IL-2R gamma.
[A-37a] In a preferred embodiment of the first target-binding molecule or the second target-binding molecule for use of any one of [A-33] to [A-36], the scaffold protein is IL2R alpha, the first receptor protein is IL-2R beta, and the second receptor protein is IL-2R gamma.
[A-38] The present invention is also concerned with a protein complex comprising a first target-binding molecule, comprising a first binding domain that is capable of binding to a scaffold protein and a second binding domain that is capable of binding to a first receptor protein, and a second target-binding molecule, comprising a third binding domain that is capable of binding to the scaffold protein and a fourth binding domain that is capable of binding to a second receptor protein,
   wherein the first target-binding molecule and the second target-binding molecule are capable of non-competitively binding to the scaffold protein,
   wherein the first receptor protein and the second receptor protein are each receptor subunits that can associate to form a receptor complex,
   wherein the protein complex is capable of inducing receptor signaling of the receptor complex.
[A-38a] The present invention is also concerned with a protein complex comprising a first target-binding molecule, comprising a first binding domain that is capable of binding to a scaffold protein and a second binding domain that is capable of binding to a first receptor protein, and a second target-binding molecule, comprising a third binding domain that is capable of binding to the scaffold protein and a fourth binding domain that is capable of binding to a second receptor protein,
   wherein the first target-binding molecule and the second target-binding molecule are capable of non-competitively binding to the scaffold protein,
   wherein the first receptor protein and the second receptor protein are each receptor subunits that can associate to form a receptor complex.
   Upon binding of the four binding domains as defined above, the receptor complex induces signalling.
[A-39] The present invention is also concerned with a protein complex comprising a first binding domain, a second binding domain, a third binding domain and a fourth binding domain, wherein the first binding domain is capable of binding to a scaffold protein, the second binding domain is capable of binding to a first receptor protein, the third binding domain is capable of binding to the scaffold protein, and the fourth binding domain is capable of binding to a second receptor protein,
   wherein the first binding domain and the third binding domain are capable of non-competitively binding to the scaffold protein,
   wherein the first receptor protein and the second receptor protein are each receptor subunits that can associate to form a receptor complex,
   wherein the protein complex is capable of inducing receptor signaling of the receptor complex.
[A-39a] The present invention is also concerned with a protein complex comprising a first binding domain, a second binding domain, a third binding domain and a fourth binding domain, wherein the first binding domain is capable of binding to a scaffold protein, the second binding domain is capable of binding to a first receptor protein, the third binding domain is capable of binding to the scaffold protein, and the fourth binding domain is capable of binding to a second receptor protein,
   wherein the first binding domain and the third binding domain are capable of non-competitively binding to the scaffold protein,
   wherein the first receptor protein and the second receptor protein are each receptor subunits that can associate to form a receptor complex,
   Upon binding of the four binding domains as defined above, the receptor complex induces signalling.
[A-40] In a preferred embodiment of the protein complex of any one of [A-38] to [A-39a], the first binding domain and the third binding domain are capable of binding the scaffold protein biparatopically.
[A-41] In a preferred embodiment of the protein complex of any one of [A-38] to [A-40], the protein complex induces receptor signaling in a first concentration or amount of the scaffold protein and does not induce receptor signaling in a second concentration or amount of the scaffold protein, wherein when the first concentration or amount of the scaffold protein is increased by at least 10%, 20%, 30%, 40%, 50%, 60%, 70%, 80%, 90%, 100%, 150%, 200%, 250%, 300%, 350%, 400%, 450% or 500% as compared to the second concentration or amount of the scaffold protein, receptor signaling is increased by at least 10%, 20%, 30%, 40%, 50%, 60%, 70%, 80%, 90%, 100%, 150%, 200%, 250%, 300%, 350%, 400%, 450% or 500%.
[A-42] In a preferred embodiment of the protein complex of any one of [A-38] to [A-41], the first binding domain is connected to the second binding domain, optionally via a linker, and/or the third binding domain is connected to the fourth binding domain, optionally via a linker. In a preferred embodiment, the linker is 20, 15, 10 amino acids or less. In an even more preferred embodiment, the linker is 10 amino acids or less.
[A-43] In a preferred embodiment of the protein complex of any one of [A-38] to [A-42], the first binding domain and the third binding domain are capable of binding the scaffold protein biparatopically within a domain of the scaffold protein.
[A-43a] In a preferred embodiment of the protein complex of any one of [A-38] to [A-42], the first binding domain and the third binding domain are each selected from the group consisting of a neutralizing binding domain and a non-neutralizing binding domain. Preferably, the first binding domain is a neutralizing binding domain and the third binding domain is a non-neutralizing binding domain or vice versa.
[A-44] In a preferred embodiment of the protein complex of any one of [A-38] to [A-43a], the scaffold protein comprises two, three, four, or more subunits.
[A-45] In a preferred embodiment of the protein complex of any one of [A-38] to [A-44], the first binding domain and the third binding domain each are capable of binding subunits of the scaffold protein which can associate to form the scaffold protein.
[A-46] In a preferred embodiment of the protein complex of any one of [A-38] to [A-45], the scaffold protein is a membrane-bound protein, a soluble protein, or an insoluble protein deposition.
[A-47] In a preferred embodiment of the protein complex of any one of [A-38] to [A-46], the scaffold protein is a cell surface marker, an immune cell surface marker, a T cell exhaustion marker, tumor-specific marker, tumor-associated marker, or a protein whose expression is characteristic of a disease or disorder.
[A-48] In a preferred embodiment of the protein complex of any one of [A-38] to [A-47], the scaffold protein is selected from the group consisting of PD1, PDL1, CSF2RB, CD4, CD5, CD6, CD7, CD8, CD9, CCR4, CD8, CD25, CD27, CD38, CD39, CD45, CD62L, CCR6, CD69, CD103, CD73, CD127, GITR, LRRC32, ICOS, TIGIT, RBD, MUC1, CEA, CTLA4, IL2R alpha, CXCR5, Neuropilin-1, TIM3, LAG3, TNF alpha, CD19, CD20, CD22, CD30, CD33, Glycoprotein NMB, CD56, CD70, CD79A, CD79B, CD138, PSCA, PSMA, BCMA, FcRH5, GPRC5D, FAP, LRRC15, E-selectin, EphB2, Melanotransferrin, HER2, TROP2, Nectine-4, EGFRvIII, IL13RA2, TMEFF2, Muc16, EpCAM, FcRH2, AFP, PSA, Amyloid beta, MBP and ASGPR. In an even more preferred embodiment, the scaffold protein is selected from the group consisting of PD1, PDL1, TNF alpha, CD25, MUC1, CEA and CD8.
[A-49] In a preferred embodiment of the protein complex of any one of [A-38] to [A-48], wherein the first and second receptor proteins are each independently selected from the group consisting of IL2R beta, IL2R gamma, IL4R, IL13RA1, IL7R, IL9R, IL21R, TSLPR, IL3RA, CSF2RB, IL5RA, CSF2RA, IL6R, gp130, IL11RA, IL12RB1, IL12RB2, IL27RA, IL31RA, OSMR, CNTFR, LIFR, IL10RA, IL10RB, IL20RA, IL20RB, IL22RA1, IL28RA, IFNAR1, IFNAR2, IFNGR1, IFNGR2, IL1R1, IL1RAP, IL18R1, IL18RAP, ST2, IL17RA, IL17RC, TLR3, TLR4, TLR7, TLR9, CSF1R, TNFR1, TNFR2, LTBR, HVEM, FAS, CD28, cMET, DR3, DR4, DR5, NGFR, RANK, FN14, CD40, 4-1BB, OX40, GITR, TGFBR1, TGFBR2, ACVRL1, ACVR2A, BMPR2, ACVR2B, ACVR1B, ACVR1C, ACVR1, AMHR2, BMPR1A, BMPR1B, BMPR2, TRA, TRB, CD3E, CD16, TREM2, FGFR1, FGFR2, FGFR3, FGFR4, Fzd1, Fzd2, Fzd3, Fzd4, Fzd5, Fzd6, Fzd7, Fzd8 Fzd9, Fzd10, LRP5, LRP6 and LGR5. In an even more preferred embodiment, the first and second receptor proteins are each independently selected from the group consisting of IL2R beta, IL2R gamma, Fzd1, Fzd2, Fzd3, Fzd4, Fzd5, Fzd6, Fzd7, Fzd8 Fzd9, Fzd10, LRP5, LRP6, CSF2RA, CSF2RB and cMET.
[A-49a] In a preferred embodiment of the protein complex of any one of [A-38] to [A-49], the scaffold protein is PD1, the first receptor protein is IL2R beta, and the second receptor protein is IL2R gamma.
[A-49b] In a preferred embodiment of the protein complex of any one of [A-38] to [A-49], the scaffold protein is IL2R alpha, the first receptor protein is IL2R beta, and the second receptor protein is IL2R gamma.
[A-49c] In a preferred embodiment of the protein complex of any one of [A-38] to [A-49], the protein complex competes for binding with or binds to the same epitope as any one of the target-binding molecules or protein complex of any one of Tables 1-17.
[A-49d] In a preferred embodiment of the protein complex of any one of [A-38] to [A-49], the scaffold protein, the first receptor protein, and the second receptor protein is a combination chosen from any of (i) to (vi) below:
   (i) the scaffold protein is PDL1, the first receptor protein is IL2R beta, and the second receptor protein is IL2R gamma;
   (ii) the scaffold protein is TNFa, the first receptor protein is IL2R beta, and the second receptor protein is IL2R gamma;
   (iii) the scaffold protein is CD25, the first receptor protein is IL2R beta, and the second receptor protein is IL2R gamma;
   (iv) the scaffold protein is PDL1, the first receptor protein is CSF2RA, and the second receptor protein is CSF2RB;
   (v) the scaffold protein is MUC1 or CEA, the first receptor protein is a Fzd receptor, and the second receptor protein is a Lrp; or
   (vi) the scaffold protein is CD8, the first receptor protein is IL2R beta, and the second receptor protein is IL2R gamma.
[A-49e] In a preferred embodiment of the protein complex of [A-38] to [A-49d], the receptor complex is IL-2R. In a more preferred embodiment, inducing IL-2 receptor complex signaling comprises evaluating IL-2 receptor complex activation using a colorimetric enzyme assay that determines alkaline phosphatase activity. In a even more preferred embodiment, alkaline phosphatase activity is determined by measuring optical density optionally at 620 nm. In a most preferred embodiment, receptor signaling of the receptor complex is induced when there is an increase in optical density by at least 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500% in the presence of the protein complex of [A-38] to [A-49c] as compared to in the absence of the protein complex of [A-38] to [A-49c].
[A-50] In a preferred embodiment of the protein complex of any one of [A-38] to [A-49e], each binding domain is an antigen binding domain comprising VH and VL, sdAb, VHH, scFv, Fab, scFab, Fab′, Fab′-SH, F(ab′)2, diabody, triabody, Fv, aptamer, Affibody, a cytokine, a ligand, or a split cytokine.
[A-51] In a preferred embodiment of the protein complex of any one of [A-38] to [A-50], the protein complex further comprises a half-life extending domain.
[A-52] In a preferred embodiment of the protein complex of any one of [A-38] and [A-40] to [A-51], the first target-binding molecule is connected to the half-life extending domain at the first binding domain and the second target-binding molecule is connected to the half-life extending domain at the third binding domain.
[A-53] In a preferred embodiment of the protein complex of any one of [A-51] to [A-52], the half-life extending domain comprises an Fc domain, FcRn-binding domain, an albumin-binding domain, an albumin or variant thereof, or polyethylene glycol.
[A-54] In a preferred embodiment of the protein complex of [A-53], the half-life extending domain comprises an Fc domain, optionally wherein the Fc domain comprises modifications that diminish antibody-dependent cellular cytotoxicity (ADCC), diminish complement-dependent cytotoxicity (CDC), increase FcRn binding, increase pI, or promote heterodimerization.
[A-55] The present invention is also concerned with a nucleic acid molecule or a plurality of nucleic acid molecules encoding the protein complex of any one of [A-38] to [A-54].
[A-56] The present invention is also concerned with a vector comprising the nucleic acid molecule or the plurality of nucleic acid molecules of [A-55].
[A-57] The present invention is also concerned with a host cell comprising the nucleic acid molecule or the plurality of nucleic acid molecules of [A-55] or the vector of [A-56].
[A-58] The present invention is also concerned with a method for producing a protein complex, comprising the steps of:
   (i) culturing the host cell of [A-57] under conditions suitable for protein expression
   (ii) optionally lysing the host cell; and
   (iii) isolating the combination of target-binding molecules.
[A-59] The present invention is also concerned with a pharmaceutical composition comprising the protein complex of any one of [A-38] to [A-54].
[A-60] The present invention is also concerned with a method for treating a disease in a subject, the method comprising administering the pharmaceutical composition of [A-59] to a subject in need thereof.
[A-61] In a preferred embodiment of the method for treating a disease in a subject of [A-60], the disease is a cancer or an autoimmune disease.
[A-61a] In a preferred embodiment the cancer or autoimmune disease of [A-61] is a cancer or autoimmune disease that is susceptible of being improved or prevented by an increase or enhancement in IL-2R signalling.
[A-62] The present invention is also concerned with the pharmaceutical composition of [A-59] for use in therapy.
[A-63] In a preferred embodiment of the pharmaceutical composition for use of [A-62], the therapy is cancer immunotherapy or autoimmune disease immunotherapy.
[A-63a] In a preferred embodiment the cancer or autoimmune disease of [A-63] is a cancer or autoimmune disease that is susceptible of being improved or prevented by an increase or enhancement in IL-2R signalling.
[A-64] The present invention is also concerned with the use of the pharmaceutical composition of [A-61] in the manufacture of a medicament for treating a disease.
[A-65] In a preferred embodiment of the use of [A-64], the disease is a cancer or an autoimmune disease.
[A-65a] In a preferred embodiment the cancer or autoimmune disease of [A-65] is a cancer or autoimmune disease that is susceptible of being improved or prevented by an increase or enhancement in IL-2R signalling.
[A-66] In a preferred embodiment of the method of any one of [A-60] or [A-61], the pharmaceutical composition for use of any one of [A-62] or [A-63], or the use of any one of [A-64], [A-65] or [A-65a], the scaffold protein is PD1, the first receptor protein is IL-2R beta, and the second receptor protein is IL-2R gamma.
[A-66a] In a preferred embodiment of the method of any one of [A-60] or [A-61], the pharmaceutical composition for use of any one of [A-62] or [A-63], or the use of any one of [A-64], [A-65] or [A-65a], the scaffold protein is IL2R alpha, the first receptor protein is IL-2R beta, and the second receptor protein is IL-2R gamma.
[A-67] In a preferred embodiment of the method of [A-61], the pharmaceutical composition for use of [A-63], or the use of [A-65] or [A65a], the cancer or autoimmune disease is characterized by PD1, IL2R alpha, IL-2R beta, or IL-2R gamma expression.
[A-68] The present invention is also concerned with a method for treating a disease in a subject, the method comprising administering to a subject in need thereof a therapeutically effective amount of a composition comprising:
   a first target-binding molecule, comprising a first binding domain that is capable of binding to a scaffold protein and a second binding domain that is capable of binding to a first receptor protein, and
   a second target-binding molecule, comprising a third binding domain that is capable of binding to the scaffold protein and a fourth binding domain that is capable of binding to a second receptor protein, wherein the first target-binding molecule and the second target-binding molecule are capable of non-competitively binding to the scaffold protein, wherein the first receptor protein and the second receptor protein are each receptor subunits that can associate to form a receptor complex,
   wherein the composition is capable of inducing receptor signaling of the receptor complex.
[A-69] The present invention is also concerned with a method for treating a disease in a subject, the method comprising administering to a subject in need thereof a therapeutically effective amount of a composition comprising:
   a protein complex comprising a first target-binding molecule, comprising a first binding domain that is capable of binding to a scaffold protein and a second binding domain that is capable of binding to a first receptor protein, and a second target-binding molecule, comprising a third binding domain that is capable of binding to the scaffold protein and a fourth binding domain that is capable of binding to a second receptor protein, wherein the first target-binding molecule and the second target-binding molecule are capable of non-competitively binding to the scaffold protein,
   wherein the first receptor protein and the second receptor protein are each receptor subunits that can associate to form a receptor complex,
   wherein the protein complex is capable of inducing receptor signaling of the receptor complex.
[A-70] The present invention is also concerned with a method of conditionally inducing receptor signaling, comprising:
   (i) identifying a subject in need thereof expressing a scaffold protein, a first receptor protein and a second receptor protein, and
   (ii) administering the combination of target-binding molecules of any one of [A-1] to [A-17] or the protein complex of any one of [A-38] to [A-54].
[A-71] The present invention is also concerned with a method of screening for a combination of target-binding molecules, comprising the steps of:
   (i) identifying a scaffold protein, and a first binding domain and a third binding domain that can each bind to the scaffold protein non-competitively,
   (ii) identifying a first receptor protein and a second receptor protein, and identifying a second binding domain that can bind to the first receptor protein and a fourth binding domain that can bind to the second receptor protein,
   (iii) producing a first target-binding molecule comprising the first binding domain and the second binding domain and a second target-binding molecule comprising the third binding domain and the fourth binding domain, optionally wherein the first and second target-binding molecule are of any one of [A-1] to [A-17],
   (iv) determining the receptor signaling activity of the first receptor protein and the second receptor protein in the presence of the first target-binding molecule, the second target-binding molecule and the scaffold protein,
   (v) determining the receptor signaling activity of the first receptor protein and the second receptor protein in the presence of the second target-binding molecule and the scaffold protein, in the absence of the scaffold protein, and
   (vi) selecting the combination of the first target-binding molecule and the second target-binding molecule if the receptor signaling activity of (iv) is higher than (v), optionally wherein the receptor signaling activity of (iv) is higher than (v) by at least 10%, 20%, 30%, 40%, 50%, 60%, 70%, 80% or 90%.
[A-72] The present invention is also concerned with a method of screening for a protein complex, comprising the steps of:
   (i) identifying a scaffold protein, and a first binding domain and a third binding domain that can each bind to the scaffold protein non-competitively,
   (ii) identifying a first receptor protein and a second receptor protein, and identifying a second binding domain that can bind to the first receptor protein and a fourth binding domain that can bind to the second receptor protein,
   (iii) producing a protein complex comprising the first binding domain, the second binding domain, the third binding domain and the fourth binding domain, optionally wherein the protein complex is of any one of [A-38] to [A-54],
   (iv) determining the receptor signaling activity of the protein complex in the presence of the scaffold protein,
   (v) determining the receptor signaling activity of the protein complex in the absence of the scaffold protein, and
   (vi) selecting the protein complex if the receptor signaling activity of (iv) is higher than (v), optionally wherein the receptor signaling activity of (iv) is higher than (v) by at least 10%, 20%, 30%, 40%, 50%, 60%, 70%, 80% or 90%.
[A-72a] The method of [A-71] or [A-72], wherein the identifying of a first binding domain and a third binding domain that can each bind to the scaffold protein non-competitively comprises the steps of:
   (a) identifying neutralizing binding domains that bind the scaffold protein,
   (b) identifying non-neutralizing binding domains that bind the scaffold protein, and
   (c) selecting a neutralizing binding domain as the first binding domain and a non-neutralizing binding domain as the third binding domain or vice versa.
[A-72b] The present invention is also concerned with a method for screening for non-competitive and/or biparatopic protein binding domains that bind a scaffold protein, comprising the steps of
   (i) identifying neutralizing binding domains that bind the scaffold protein,
   (ii) identifying non-neutralizing binding domains that bind the scaffold protein, and
   (iii) selecting a neutralizing binding domain and a non-neutralizing binding domain as the non-competitive and/or biparatopic protein binding domains.
[A-72c] The present invention is also concerned with a method of screening for the combination of target-binding molecules of any one of [A-1] to [A-17] which comprise non-competitive and/or biparatopic scaffold protein binding domains, comprising the steps of
   (i) identifying target-binding molecules that comprise neutralizing binding domains that bind the scaffold protein,
   (ii) identifying target-binding molecules that comprise non-neutralizing binding domains that bind the scaffold protein, and
   (iii) selecting a target-binding molecule comprising a neutralizing binding domain that bind the scaffold protein and a target-binding molecule comprising a non-neutralizing binding domain that bind the scaffold protein.
[A-72d] The present invention is also concerned with a method of screening for a protein complex of any one of [A-38] to [A-54] which comprise non-competitive and/or biparatopic scaffold protein binding domains, comprising the steps of
   (i) identifying protein complexes comprising neutralizing binding domains that bind the scaffold protein,
   (ii) identifying protein complexes comprising non-neutralizing binding domains that bind the scaffold protein, and
   (iii) selecting a protein complex that comprises a neutralizing binding domain as the first binding domain and a non-neutralizing binding domain as the third binding domain or vice versa.
[A-72e] The present invention is also concerned with a method for producing the combination of target-binding molecules of any one of [A-1] to [A-17], comprising the steps of
   (a) identifying neutralizing binding domains that bind the scaffold protein,
   (b) identifying non-neutralizing binding domains that bind the scaffold protein, and
   (c) selecting a neutralizing binding domain as the first binding domain and a non-neutralizing binding domain as the third binding domain or vice versa.
[A-72e1] In an embodiment of the method of [A-72e], the method further comprises the steps of
   (d) Obtaining a polynucleotide encoding the neutralizing binding domain and a polynucleotide encoding the non-neutralizing binding domain,
   (e) Connecting, optionally via a linker, the polynucleotide encoding the neutralizing binding domain with a polynucleotide encoding the second binding domain,
   (f) Connecting, optionally via a linker, the polynucleotide encoding the non-neutralizing binding domain with a polynucleotide encoding the fourth binding domain,
   (g) Expressing the polynucleotides of (e) and (f).
[A-72e2] In an embodiment of the method of [A-72e], the method further comprises the steps of
   (d) Producing a first target-binding molecule comprising the first binding domain and the second binding domain, and a second target-binding molecule comprising the third binding domain and the fourth binding domain.
[A-72f] The present invention is also concerned with a method for producing the protein complex of any one of [A-38] to [A-54], comprising the steps of
   (a) identifying neutralizing binding domains that bind the scaffold protein,
   (b) identifying non-neutralizing binding domains that bind the scaffold protein, and
   (c) selecting a neutralizing binding domain as the first binding domain and a non-neutralizing binding domain as the third binding domain or vice versa.
[A-72f1] In an embodiment of the method of [A-72f], the method further comprises the steps of
   (d) Obtaining a polynucleotide encoding the neutralizing binding domain and a polynucleotide encoding the non-neutralizing binding domain,
   (e) Connecting, optionally via a linker, the polynucleotide encoding the neutralizing binding domain with a polynucleotide encoding at least a fragment of the the second binding domain,
   (f) Connecting, optionally via a linker, the polynucleotide encoding the non-neutralizing binding domain with a polynucleotide encoding at least a fragment of the fourth binding domain,
   (g) Expressing the polynucleotides of (e) and (f) to produce the protein complex.
[A-72f2] In an embodiment of the method of [A-72f], the method further comprises the steps of
   (d) Producing the protein complex any one of [A-38] to [A-54] comprising the first binding domain, the second binding domain, the third binding domain and the fourth binding domain.
[A-73] In a preferred embodiment of the method of any one of [A-68] to [A-72f2], the first binding domain and the third binding domain are capable of binding the scaffold protein biparatopically.
[A-74] In a preferred embodiment of the method of any one of [A-68] to [A-73], the combination of target-binding molecules or protein complex induces receptor signaling in a first concentration or amount of the scaffold protein and does not induce receptor signaling in a second concentration or amount of the scaffold protein, wherein when the first concentration or amount of the scaffold protein is increased by at least 10%, 20%, 30%, 40%, 50%, 60%, 70%, 80%, 90%, 100%, 150%, 200%, 250%, 300%, 350%, 400%, 450% or 500% as compared to the second concentration or amount of the scaffold protein, receptor signaling is increased by at least 10%, 20%, 30%, 40%, 50%, 60%, 70%, 80%, 90%, 100%, 150%, 200%, 250%, 300%, 350%, 400%, 450% or 500%.
[A-75] In a preferred embodiment of the method of any one of [A-68] to [A-74], the first binding domain is connected to the second binding domain, optionally via a linker, and/or the third binding domain is connected to the fourth binding domain, optionally via a linker. In a preferred embodiment, the linker is 20, 15, 10 amino acids or less. In an even more preferred embodiment, the linker is 10 amino acids or less.
[A-76] In a preferred embodiment of the method of any one of [A-68] to [A-75], the first binding domain and the third binding domain are capable of binding the scaffold protein biparatopically within a domain of the scaffold protein.
[A-76a] In a preferred embodiment of the method of any one of [A-68] to [A-75], the first binding domain and the third binding domain are each selected from the group consisting of a neutralizing binding domain and a non-neutralizing binding domain. Preferably, the first binding domain is a neutralizing binding domain and the third binding domain is a non-neutralizing binding domain or vice versa.
[A-77] In a preferred embodiment of the method of any one of [A-68] to [A-76a], the scaffold protein comprises two, three, four, or more subunits.
[A-78] In a preferred embodiment of the method of any one of [A-68] to [A-77], the first binding domain and the third binding domain each are capable of binding subunits of the scaffold protein which can associate to form the scaffold protein.
[A-79] In a preferred embodiment of the method of any one of [A-68] to [A-78], the scaffold protein is a membrane-bound protein, a soluble protein, or an insoluble protein deposition.
[A-80] In a preferred embodiment of the method of any one of [A-68] to [A-79], the scaffold protein is a cell surface marker, an immune cell surface marker, a T cell exhaustion marker, tumor-specific marker, tumor-associated marker, or a protein whose expression is characteristic of a disease or disorder.
[A-81] In a preferred embodiment of the method of any one of [A-68] to [A-80], the scaffold protein is selected from the group consisting of PD1, PDL1, CSF2RB, CD4, CD5, CD6, CD7, CD8, CD9, CCR4, CD8, CD25, CD27, CD38, CD39, CD45, CD62L, CCR6, CD69, CD103, CD73, CD127, GITR, LRRC32, ICOS, TIGIT, RBD, MUC1, CEA, CTLA4, IL2R alpha, CXCR5, Neuropilin-1, TIM3, LAG3, TNF alpha, CD19, CD20, CD22, CD30, CD33, Glycoprotein NMB, CD56, CD70, CD79A, CD79B, CD138, PSCA, PSMA, BCMA, FcRH5, GPRC5D, FAP, LRRC15, E-selectin, EphB2, Melanotransferrin, HER2, TROP2, Nectine-4, EGFRvIII, IL13RA2, TMEFF2, Muc16, EpCAM, FcRH2, Amyloid beta, AFP, PSA, MBP and ASGPR. In an even more preferred embodiment, the scaffold protein is selected from the group consisting of PD1, PDL1, TNF alpha, CD25, MUC1, CEA and CD8.
[A-82] In a preferred embodiment of the method of any one of [A-68] to [A-81], wherein the first and second receptor proteins are each independently selected from the group consisting of IL2R beta, IL2R gamma, IL4R, IL13RA1, IL7R, IL9R, IL21R, TSLPR, IL3RA, CSF2RB, IL5RA, CSF2RA, IL6R, gp130, IL11RA, IL12RB1, IL12RB2, IL27RA, IL31RA, OSMR, CNTFR, LIFR, IL10RA, IL10RB, IL20RA, IL20RB, IL22RA1, IL28RA, IFNAR1, IFNAR2, IFNGR1, IFNGR2, IL1R1, IL1RAP, IL18R1, IL18RAP, ST2, IL17RA, IL17RC, TLR3, TLR4, TLR7, TLR9, CSF1R, TNFR1, TNFR2, LTBR, HVEM, FAS, CD28, cMET, DR3, DR4, DR5, NGFR, RANK, FN14, CD40, 4-1BB, OX40, GITR, TGFBR1, TGFBR2, ACVRL1, ACVR2A, BMPR2, ACVR2B, ACVR1B, ACVR1C, ACVR1, AMHR2, BMPR1A, BMPR1B, BMPR2, TRA, TRB, CD3E, CD16, TREM2, FGFR1, FGFR2, FGFR3, FGFR4, Fzd1, Fzd2, Fzd3, Fzd4, Fzd5, Fzd6, Fzd7, Fzd8 Fzd9, Fzd10, LRP5, LRP6 and LGR5. In an even more preferred embodiment, the first and second receptor proteins are each independently selected from the group consisting of IL2R beta, IL2R gamma, Fzd1, Fzd2, Fzd3, Fzd4, Fzd5, Fzd6, Fzd7, Fzd8 Fzd9, Fzd10, LRP5, LRP6, CSF2RA, CSF2RB and cMET.
[A-82a] In a preferred embodiment of the method of any one of [A-68] to [A-81], the scaffold protein is PD1, the first receptor protein is IL2R beta, and the second receptor protein is IL2R gamma.
[A-82b] In a preferred embodiment of the method of any one of [A-68] to [A-81], the scaffold protein is IL2R alpha, the first receptor protein is IL2R beta, and the second receptor protein is IL2R gamma.
[A-82c] In a preferred embodiment of the method of any one of [A-68] to [A-81], the combination of target-binding molecules or protein complex competes for binding with or binds to the same epitope as any one of the target-binding molecules or protein complex of any one of Tables 1-17.
[A-82d] In a preferred embodiment of the method of any one of [A-68] to [A-81], the scaffold protein, the first receptor protein, and the second receptor protein is a combination chosen from any of (i) to (vi) below:
   (i) the scaffold protein is PDL1, the first receptor protein is IL2R beta, and the second receptor protein is IL2R gamma;
   (ii) the scaffold protein is TNFa, the first receptor protein is IL2R beta, and the second receptor protein is IL2R gamma;
   (iii) the scaffold protein is CD25, the first receptor protein is IL2R beta, and the second receptor protein is IL2R gamma;
   (iv) the scaffold protein is PDL1, the first receptor protein is CSF2RA, and the second receptor protein is CSF2RB;
   (v) the scaffold protein is MUC1 or CEA, the first receptor protein is a Fzd receptor, and the second receptor protein is a Lrp; or
   (vi) the scaffold protein is CD8, the first receptor protein is IL2R beta, and the second receptor protein is IL2R gamma.
[A-82e] In a preferred embodiment of the method of any one of [A-68] to [A-81], the receptor complex is IL-2R. In a more preferred embodiment, inducing IL-2 receptor complex signaling comprises evaluating IL-2 receptor complex activation using a colorimetric enzyme assay that determines alkaline phosphatase activity. In a even more preferred embodiment, alkaline phosphatase activity is determined by measuring optical density optionally at 620 nm. In a most preferred embodiment, receptor signaling of the receptor complex is induced when there is an increase in optical density by at least 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500% in the presence of the combination of target-binding molecules or protein complex as compared to in the absence of the combination of target-binding molecules or protein complex.
[A-83] In a preferred embodiment of the method of any one of [A-68] to [A-82e], each binding domain is an antigen binding domain comprising VH and VL, sdAb, VHH, scFv, Fab, scFab, Fab′, Fab′-SH, F(ab′)2, diabody, triabody, Fv, aptamer, Affibody, a cytokine, a ligand, or a split cytokine.
[A-84] In a preferred embodiment of the method of any one of [A-68] to [A-83], the combination of target-binding molecules or the protein complex further comprises a half-life extending domain.
[A-85] In a preferred embodiment of the method of any one of [A-68], [A-70], or [A-72] to [A-84], the first target-binding molecule is connected to the half-life extending domain at the first binding domain and the second target-binding molecule is connected to the half-life extending domain at the third binding domain.
[A-86] In a preferred embodiment of the method of [A-84] or [A-85], the half-life extending domain comprises an Fc domain, FcRn-binding domain, an albumin-binding domain, an albumin or variant thereof, or polyethylene glycol.
[A-87] In a preferred embodiment of the method of any one of [A-84] to [A-86], the half-life extending domain comprises an Fc domain, optionally wherein the Fc domain comprises modifications that diminish antibody-dependent cellular cytotoxicity (ADCC), diminish complement-dependent cytotoxicity (CDC), increase FcRn binding, increase pI, or promote heterodimerization.
[A-88] The invention is also concerned with a combination of target-binding molecules or a protein complex comprising the target-binding molecules or the protein complex of any one of Tables 1-17. As show in this application, the target-binding molecules or the protein complex of any one of Tables 1-17 are capable of inducing receptor signaling of the respective receptor complex.
[B-1] An antigen-binding molecule, comprising any one of (a1) to (a4) below:
   (a1) complementarity determing region (CDR) 1 having the amino acid sequence SYTMG, CDR2 having the amino acid sequence AIRWSGSITYYADSVKG, and CDR 3 having the amino acid sequence SPVAGWGTSPAWYDY;
   (a2) CDR 1 having the amino acid sequence SYGMG, CDR 2 having the amino acid sequence TISWNSGSIYYTDSVKG, and CDR 3 having the amino acid sequence GPRDWGNMRKFEEYEY;
   (a3) CDR 1 having the amino acid sequence EYGMG, CDR 2 having the amino acid sequence TISWDSDSIYYTDSVKG, and CDR 3 having the amino acid sequence RPRDWGNMRRFEAYEY; or
   (a4) CDR 1 having the amino acid sequence DYAGS, CDR 2 having the amino acid sequence SINWRGDTTYYADSVKG, and CDR 3 having the amino acid sequence KATDWSSTLYEYDY.
[B-2] The antigen-binding molecule of [B-1], wherein at least one of the CDR 1, CDR 2 or CDR 3 amino acid sequences further comprise one or more conservative amino acid substitutions.
[B-3] The antigen-binding molecule of any one of [B-1] or [B-2], wherein the antigen-binding molecule is a VHH or a single-domain antibody.
[B-4] The antigen-binding molecule of any one of [B-1] to [B-3], wherein the antigen-binding molecule comprises any one of (a1) to (a4) below:
   (a1) an amino acid sequence having at least 90%, 91%, 92%, 93%, 94%, 95%, 96%, 97%, 98%, 99%, or 100% sequence identity to the amino acid sequence

QLQLVESGGGLVQAGGSLRLSCAASGRGFSSYTMGWFRQAPGKEREFVSA
IRWSGSITYYADSVKGRFTISRDNAKNTMYLQMNSLKPEDTAVYYCAASP
VAGWGTSPAWYDYWGQGTQVTVSS,

(a2) an amino acid sequence having at least 90%, 91%, 92%, 93%, 94%, 95%, 96%, 97% 98%, 99%, or 100% sequence identity to the amino acid sequence

QLQLVESGGGLVQPGGSLTLSCAASGRSFSSYGMGWFRQAPGKEREFVAT
ISWNSGSIYYTDSVKGRFTISRDDAKNTMSLQMNSLRPEDTAVYSCAAGP
RDWGNMRKFEEYEYRGQGTQVTVSS,

(a3) an amino acid sequence having at least 90%, 91%, 92%, 93%, 94%, 95%, 96%, 97%, 98%, 99%, or 100% sequence identity to the amino acid sequence QVQLVESGGGLVQPGGSLTLSCAASGRSFSEYGMGWFRQAPGKEREFVATISW DSDSIYYTDSVKGRFTISRDNAKNTMSLQMNSLKPEDTAVYSCAARPRDWGN MRRFEAYEYRGQGTQVTVSS, or
(a4) an amino acid sequence having at least 90%, 91%, 92%, 93%, 94%, 95%, 96%, 97%, 98%, 99%, or 100% sequence identity to the amino acid sequence

QVQLVESGGGLVQTGGSLRLSCAASEPTFSDYAGSWFRQAPGKEREFLAS
INWRGDTTYYADSVKGRFTISRDNAKNMMYLEMNNLEPEDTAVYRCAAKA
TDWSSTLYEYDYWGQGTQVTVSS.

[B-5] An antigen-binding molecule comprising any one of (a1) to (a4) below:
   (a1) an amino acid sequence having at least 90%, 91%, 92%, 93%, 94%, 95%, 96%, 97%, 98%, 99%, or 100% sequence identity to the amino acid sequence

QLQLVESGGGLVQAGGSLRLSCAASGRGFSSYTMGWFRQAPGKEREFVSA
IRWSGSITYYADSVKGRFTISRDNAKNTMYLQMNSLKPEDTAVYYCAASP
VAGWGTSPAWYDYWGQGTQVTVSS,

(a2) an amino acid sequence having at least 90%, 91%, 92%, 93%, 94%, 95%, 96%, 97%, 98%, 99%, or 100% sequence identity to the amino acid sequence

QLQLVESGGGLVQPGGSLTLSCAASGRSFSSYGMGWFRQAPGKEREFVAT
ISWNSGSIYYTDSVKGRFTISRDDAKNTMSLQMNSLRPEDTAVYSCAAGP
RDWGNMRKFEEYEYRGQGTQVTVSS,

(a3) an amino acid sequence having at least 90%, 91%, 92%, 93%, 94%, 95%, 96%, 97%, 98%, 99%, or 100% sequence identity to the amino acid sequence QVQLVESGGGLVQPGGSLTLSCAASGRSFSEYGMGWFRQAPGKEREFVATISW DSDSIYYTDSVKGRFTISRDNAKNTMSLQMNSLKPEDTAVYSCAARPRDWGN MRRFEAYEYRGQGTQVTVSS, or
(a4) an amino acid sequence having at least 90%, 91%, 92%, 93%, 94%, 95%, 96%, 97%, 98%, 99%, or 100% sequence identity to the amino acid sequence

QVQLVESGGGLVQTGGSLRLSCAASEPTFSDYAGSWFRQAPGKEREFLAS
INWRGDTTYYADSVKGRFTISRDNAKNMMYLEMNNLEPEDTAVYRCAAKA
TDWSSTLYEYDYWGQGTQVTVSS.

[B-6] The antigen-binding molecule of any one of [B-1] to [B-5], wherein the antigen-binding molecule binds CD8.
[B-7] An antigen-binding molecule that binds to the same epitope in CD8 as the antigen-binding molecule of any one of [B-1] to [B-5]
[B-8]A multispecific antigen-binding molecule comprising the antigen-binding molecule of any one of [B-1] to [B-7].
[B-9] The combination of target-binding molecules of any one of [A-1] to [A-17] or protein complex of any one of [A-38] to [A-54], wherein the first binding domain and/or the third binding domain is any one of (a1) to (a4) as defined in any one of [B-1] to [B-7]
[B-10]A pharmaceutical composition comprising the antigen-binding molecule of any one of [B-1] to [B-9].
[B-11] The pharmaceutical composition of [B-10] for use in therapy.
[B-12] The pharmaceutical composition of [B-10] for use in therapy of a medical condition, wherein the medical condition is characterized by CD8 expression or an increase in CD8 expression.
[B-13]A method of treating an individual having a medical condition, comprising administering to the individual an effective amount of the pharmaceutical composition of [B-10], wherein the medical condition is characterized by CD8 expression or an increase in CD8 expression.

### BRIEF DESCRIPTION OF DRAWINGS

FIG. 1A show the schematic diagrams of the protein complex in the present disclosure. FIG. 1A shows two different VHH-VHH fusions, each with a scaffold protein-binding domain and a receptor protein-binding domain. Receptor activation only occurs in the presence of scaffold protein.

FIG. 1B show the schematic diagrams of the protein complex in the present disclosure. FIG. 1B shows the possible single-molecular format that can induce receptor activation in the presence of a scaffold protein.

FIG. 2A shows the conditional IL-2R activation by expressing the mixtures of VHH-VHH fusions that bind to SARS-CoV2 receptor binding domain (RBD) in a biparatopic manner and IL-2 receptors on either RBD- or PDL1-expressing HEK-Blue IL-2 Cells.

FIG. 2B shows the schematic diagram of experimental system of this example.

FIGS. 3A and 3B show the comparison between the mixture of recombinant VHH-VHH fusions that bind to IL-2 receptors and RBD in biparatopic manner and the mixtures of recombinant VHH-VHH fusions that bind to IL-2 receptors and RBD in mono-paratopic manner by treating these recombinant VHH-VHH fusions to RBD- (FIG. 3A) or PDL1-expressing HEK-Blue IL-2 Cells (FIG. 3B).

FIG. 4 shows the conditional IL-2R activation by expressing the mixtures of VHH-VHH fusions that bind to PDL1 and IL-2 receptors on either PDL1- or RBD-expressing HEK-Blue IL-2 Cells.

FIGS. 5A and 5B show the comparison between the fusion proteins of Split Neo-2/15 and VHH, which binds to RBD (FIG. 5A), and the mixture of Vhh2b-Nb36 and Nb21-Vhh2g that bind to RBD and IL-2 receptors (FIG. 5B) by transfecting expression plasmids to HEK-Blue IL-2 Cells with RBD-IL2RA or PDL1.

FIG. 6A shows the conditional IL-2R activation by expressing the mixtures of VHH-VHH fusions that bind to TNF alpha and IL-2 receptors on either TNF alpha- or PDL1-expressing HEK-Blue IL-2 Cells.

FIG. 6B shows schematic diagram of TNF alpha dependent IL-2R activation by mixture of VHH-VHH fusions.

FIGS. 7A and 7B show the conditional Wnt receptor activation by the mixture of recombinant VHH-VHH and VHH-scFv fusions that can bind to RBD and Wnt receptors, which are FZDs and LRPs, on either RBD- (FIG. 7A) or PDL1-expressing HEK293 STF cells (FIG. 7B).

FIG. 8A shows the conditional IL-2R signal induction by Vhh2b-Nb36/Nb21-Vhh2g Fc fusion protein on either RBD- or PDL1-expressing HEK-Blue IL-2 Cells. FIG. 8B shows IL-2R activation by recombinant IL-2 on either RBD- or PDL1-expressing HEK-Blue IL-2 Cells.

FIG. 9A shows the schematic diagram of IL-2R alpha dependent IL-2R activation by the mixture of IL2_RETR and Vhh2g-BT942 scFv.

FIG. 9B shows the IL-2R signal induction by the mixture of IL2_RETR and Vhh2g-BT942 scFv, which can bind to IL-2R alpha as a scaffold protein and IL-2R beta or IL-2R gamma as target receptor proteins, on HEK-Blue IL-2 Cells with or without Daclizumab (anti-IL-2R alpha neutralizing Ab) treatment.

FIG. 9C shows the IL-2R activation by recombinant IL-2 on HEK-Blue IL-2 Cells with or without Daclizumab treatment.

FIG. 10 shows the comparison between biparatopic binding and mono-paratopic binding to IL-2R alpha in the IL-2R activation by the mixtures of IL-2 mutant and/or VHH-scFV that can bind IL-2R alpha as a scaffold protein and IL-2R beta or IL-2R gamma as target receptor proteins.

FIGS. 11A and 11B show the conditional IL-2R signal induction by mixtures of VHH-scFv fusions that bind to PD1 and IL-2 receptors on either PD1- (FIG. 11A) or PDL1-expressing HEK-Blue IL-2 Cells (FIG. 11B).

FIG. 12 shows the comparison between biparatopic binding and mono-paratopic binding to PD1 in the IL-2R activation by the mixtures of VHH-scFv that bind to PD1 as a scaffold protein and IL-2 receptors as target receptor proteins.

FIG. 13 shows the binding activity of anti-PD1 Abs on PD1-expressing cell line that was evaluated by flow cytometry.

FIG. 14 shows the neutralizing activity of anti-PD1 Abs that was evaluated by using PD1+Jurkat reporter cell line.

FIG. 15 shows the conditional IL-2 receptor activation by the mixtures of VHH-IgG fusion proteins, which bind to IL-2R beta or IL-2R gamma as target receptor proteins and PD1 as a target scaffold protein, on either PD1- (FIG. 15A) or PDL1-expressing HEK-Blue IL-2 Cells (FIG. 15B). All mixtures are a combination of neutralizing and non-neutralizing anti-PD1 Abs.

FIG. 16 shows STAT5 activation by the mixtures of VHH-IgG or VHH-scFv fusion proteins, which bind to IL-2R beta or IL-2R gamma as target receptor proteins and PD1 as a target scaffold protein, on either activated PD1-expressing CD4+T cells (FIG. 16A) or PD1 negative NK92 cells (FIG. 16B).

FIG. 17 shows the conditional IL-2 receptor activation by the mixtures of VHH-IgG fusion proteins, which bind to IL-2R beta or IL-2R gamma as target receptor proteins and PD1 as a target scaffold protein, on either PD1- (FIG. 17A) or PDL1-expressing HEK-Blue IL-2 Cells (FIG. 17B).

FIG. 18 shows the conditional IL-2 receptor activation by the mixtures of VHH-IgG or scFv-IgG fusion proteins, which bind to IL-2R beta or IL-2R gamma as target receptor proteins and PD1 as a target scaffold protein, on either PD1- (FIG. 18A) or PDL1-expressing HEK-Blue IL-2 Cells (FIG. 18B).

FIG. 19 shows the activation of IL-2 receptor by the mixtures of VHH-scFv fusion proteins, which bind to IL-2R beta or IL-2R gamma as target receptor proteins and CD25 as a target scaffold protein, on CD25-expressing activated CD4+T cells (FIG. 19).

FIG. 20 shows the activation of GM-CSF receptors by the mixtures of VHH-scFv fusion proteins, which bind to CSF2RA or CSF2RB as target receptor proteins and PDL1 as a target scaffold protein, on either PDL1- (FIG. 20A) or RBD-expressing HEK-Blue GM-CSF Cells

FIG. 21 shows the activation of cMET receptor by VHH-Fab fusion proteins, which bind to cMET as a target receptor protein and RBD as a target scaffold protein in the presence or absence of recombinant RBD proteins. FIG. 21A shows the activation of cMET receptor by the mixture of anti-RBD biparatopic Abs that fused with cMET binding domain. FIGS. 21B and 21C show the activation of cMET receptor by anti-RBD monoparatopic antibodies that fused with cMET binding domain.

FIG. 22 shows conditional Wnt receptor activation by the mixture of bispecific Abs, which bind to Wnt receptors as target receptor proteins and MUC1 as a target scaffold protein, on either MUC1- or RBD-expressing HEK293 STF cells.

FIG. 23 shows conditional Wnt receptor activation by the mixture of bispecific Abs, which bind to Wnt receptors as target receptor proteins and CEA as a target scaffold protein, on either CEA- or RBD-expressing HEK293 STF cells. FIG. 23A shows the activation of Wnt receptor by the mixtures of anti-CEA biparatopic Abs and FIG. 23B shows the activation of Wnt receptor by the mixtures of anti-CEA monoparatopic Abs.

FIG. 24 shows STAT5 activation in CD8+ or CD4+ T cells by the mixtures of VHH-VHH fusion proteins, which bind to CD8 as a target scaffold protein and IL-2R beta or IL-2R gamma as a target receptor proteins.

FIG. 25 shows STAT5 activation in CD8+ T cells by the mixture of VHH-VHH fusion proteins, which bind to CD8 as a target scaffold protein and IL-2R beta or IL-2R gamma as target receptor proteins.

FIG. 26 shows proliferation of CD8+ or CD4+ T cells by the mixture of VHH-VHH fusion proteins, which bind to CD8 as a target scaffold protein and IL-2R beta or IL-2R gamma as target receptor proteins.

### DESCRIPTION OF EMBODIMENTS

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds 1987, and periodic updates); PCR: The Polymerase Chain Reaction, (Mullis et al., ed., 1994); A Practical Guide to Molecular Cloning (Perbal Bernard V., 1988); Phage Display: A Laboratory Manual (Barbas et al., 2001).

The definitions and detailed description below are provided to facilitate understanding of the present disclosure illustrated herein. All references mentioned herein are specifically incorporated by reference.

### I. Definitions

### Protein/Polypeptide

As used herein, the term “polypeptide” refers to a molecule composed of monomers (amino acids) linearly linked by amide bonds (also known as peptide bonds). The term “polypeptide” usually refers to a peptide having a length on the order of 4 amino acids or longer, and does not refer to a specific length of the product. As used herein, the term also includes fragments of polypeptides. Thus, peptides, dipeptides, tripeptides, oligopeptides, “protein”, “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded.

### Fusion Protein

As used herein, the term “fusion protein”, sometimes abbreviated to “fusion/fusions” refers to a single-chain protein, i.e. a protein formed by a single chain of amino acids, formed by the combination of at least two (poly)peptides. In embodiments, the combination occurs via peptide bonds. In embodiments, the combination occurs via peptide bonds provided by a peptide linker. An example for a fusion protein of the present invention is the first target-binding molecule of [A-1] or [A-4], wherein a first binding domain is connected or linked to a second binding domain, optionally via a linker. A further example for a fusion protein of the present invention is the second target-binding molecule of [A-1] or [A-4], wherein a third binding domain is linked to a fourth binding domain, optionally via a linker. Another example is comprised in the protein complex of [A-39] or [A-42], wherein the first binding domain and the second binding domain are linked, or wherein the third binding domain and the fourth binding domain are linked. In some embodiments, all four binding domains are linked and therefore part of one single fusion protein.

### Protein Complex

As used herein, the term “protein complex” refers to molecular construct that is formed by association of at least two proteins or polypeptides. The association of the polypeptides in the complex does not occur via peptide bonds and thus allows the separation of the polypeptides without destroying the primary structure of the involved proteins. For example, the polypeptides in the protein complex can be associated via one or more disulfide bonds or non-covalently. Exemplary embodiments of a protein complex of the present invention comprise the protein complex of [A-38], comprising a first target-binding molecule, comprising a first binding domain that is capable of binding to a scaffold protein and a second binding domain that is capable of binding to a first receptor protein, and a second target-binding molecule, comprising a third binding domain that is capable of binding to the scaffold protein and a fourth binding domain that is capable of binding to a second receptor protein, wherein the first target-binding molecule and the second target-binding molecule are capable of non-competitively binding to the scaffold protein, wherein the first receptor protein and the second receptor protein are each receptor subunits that can associate to form a receptor complex, wherein the protein complex is capable of inducing receptor signaling of the receptor complex. In one embodiment of the protein complex of [A-38], the first target-binding molecule and the second target-binding molecule are linked to each other.

Another embodiment of a protein complex of the present invention comprises the protein complex of [A-39], comprising a first binding domain, a second binding domain, a third binding domain and a fourth binding domain, wherein the first binding domain is capable of binding to a scaffold protein, the second binding domain is capable of binding to a first receptor protein, the third binding domain is capable of binding to the scaffold protein, and the fourth binding domain is capable of binding to a second receptor protein, wherein the first binding domain and the third binding domain are capable of non-competitively binding to the scaffold protein, wherein the first receptor protein and the second receptor protein are each receptor subunits that can associate to form a receptor complex, wherein the protein complex is capable of inducing receptor signaling of the receptor complex. In one embodiment of the protein complex of [A-39], the first, the second, the third, and the fourth binding domain are linked to each other to form one single polypeptide chain.

### Target-Binding Molecule

The term “target-binding molecule” refers to a molecule such as a protein or polypeptide or fusion protein or protein complex, which is capable of eliciting measurable and reproducible interactions between the protein or polypeptide or fusion protein or protein complex, with its target, which is determinative of the presence of its target, such as an antigen, e.g. IL-2R or a scaffold protein, in the presence of a heterogenous population of molecules including biological molecules. The target-binding molecule binds to its target with greater affinity, avidity, more readily, and/or with greater duration than it binds to other targets. In one embodiment, the extent of binding of the target-binding molecule to an unrelated target is less than about 10% of the binding of the target-binding molecule to the target as measured, e.g., by a radioimmunoassay (RIA). In certain embodiments, a target-binding molecule that specifically binds to a target has a dissociation constant (Kd) of 1 micromolar (micro M) or less, 100 nM or less, 10 nM or less, 1 nM or less, 0.1 nM or less, 0.01 nM or less, or 0.001 nM or less (e.g. 10-8 M or less, e.g. from 10-8 M to 10-13 M, e.g., from 10-9 M to 10-13 M). The “target-binding molecule” of the present invention can comprise an antibody, or an antibody-like construct, a VHH, or a protein complex comprising a heavy chain and a light chain associated with each other.

### Binding Domain

The target-binding molecule of the present invention comprises two binding domains, wherein each binding domain has a different target. Therefore, the target-binding molecule of the present invention is capable of binding two targets at the same time. The term “binding domain”, as used herein, refers to the part of a protein, which exhibits target/antigen binding activity. The binding domain can be an antigen binding domain comprising VH and VL, sdAb, VHH, scFv, Fab, scFab, Fab′, Fab′-SH, F(ab′)2, diabody, triabody, Fv, aptamer or Affibody. It can be derived from a cytokine (including cytokine mutants), or from a native ligand (including mutants). It can also be a split cytokine etc.

### Neutralizing and Non-Neutralizing Binding Domains

The term “neutralizing binding domain” refers to a binding domain as described above that binds to its target/antigen and results in the prevention, inhibition, reduction, delay, or interference of the activity of the target/antigen. This may be because the binding domain binds to an epitope that is involved in the activity of the target/antigen. In a non-limiting example, a neutralizing binding domain may bind to an epitope that is near or at the ligand-binding site of the target/antigen, thereby preventing, inhibiting, reducing, delaying or interfering with the activity of the target/antigen. The term “neutralizing binding domain” may also refer to an antagonistic binding domain, such as an antagonistic antibody. The term “non-neutralizing binding domain” refers to a binding domain as described above that binds to its target/antigen and does not cause the prevention, inhibition, reduction, delay, or interference of the activity of the target/antigen. The term “non-neutralizing binding domain” may also refer to an non-antagonistic binding domain, such as a non-antagonistic antibody.

### Multispecific Target-Binding Molecules

In the present invention, the target-binding molecule provided herein is a multispecific target-binding molecule, e.g. a bispecific target-binding molecule. Multispecific target-binding molecule are target-binding molecules that have binding specificities for at least two different sites. In the present invention, one of the binding specificities is for a scaffold protein, and the other is for a receptor protein. Bispecific target-binding molecules can be prepared as full-length antibodies or comprise antibody fragments.

Techniques for making multispecific target-binding molecules include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific target-binding molecules may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bispecific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

### Competitive Binding and Non-Competitive Binding

The first target-binding molecule and the second target-binding molecule of the present invention, or the first binding domain and the third binding domain of the protein complex of the present invention are capable of binding non-competitively to the scaffold protein. “Competitive binding” as used herein, refers to the process of two binding domains competing for the same epitope or paratope, resulting in only one binding domain being capable of binding to the scaffold protein. The epitope or paratope for both binding domains can be the same, or it can be overlapping. In any case, both binding domains cannot bind to their epitope paratope at the same time. “Non-competitive binding”, as used herein, conversely refers to the process of two binding domains, which can bind to their epitope or paratope without interfering with the binding mechanism of the respective other binding domain. In one embodiment, the epitopes or paratopes do not overlap, and there is no steric hinderance preventing simultaneous binding of both binding domains simultaneously. One example of non-competitive binding is a biparatopic binding. In another embodiment, the epitope can be the same or overlapping, but there exists more than one epitope on the target. This can be the case if the target is a multimer. For example, the scaffold protein of the present invention can be composed of several subunits, each comprising the epitope. In this case, the target-binding molecules bind to two distinct but identical epitopes, which have the same sequence, but are on different subunits of the multimer. It is essential that both target-binding molecules can bind simultaneously without interfering with the binding mechanism of the respective other target-binding molecule.

### Biparatopic Binding

“Biparatopic binding”, as used herein, refers to a binding mechanism that targets two epitopes on a molecular target. In the present invention, the two binding domains targeting the scaffold protein each target a distinct epitope on the protein, allowing simultaneous binding of both binding domains to the scaffold protein.

In an embodiment, the two binding domains targeting the scaffold protein are capable of binding the scaffold protein biparatopically, wherein the epitopes bound by each binding domain are close to one another. In an embodiment, the two binding domains targeting the scaffold protein are capable of binding the scaffold protein biparatopically within a domain of the scaffold protein. The epitope may be determined from the crystal structure of the complex of scaffold protein binding domain and scaffold protein. Specifically, the interatomic distance between a non-hydrogen atom constituting a side chain or main chain of an amino acid forming a scaffold protein binding domain and a non-hydrogen atom constituting a side chain or main chain of an amino acid forming the scaffold protein is calculated. Amino acid residues containing non-hydrogen atoms with interatomic distances below the threshold set at 3.5 angstrom, 4.0 angstrom, 4.2 angstrom, 4.5 angstrom or 5.0 angstrom were taken as residues comprised within an epitope.

The closeness of the epitopes, or the distance between the epitopes bound by each binding domain in the present specification can then be determined. In an embodiment, the interatomic distance between any alpha-carbon of the first epitope on the scaffold protein and any alpha-carbon of the second epitope on the scaffold protein is at least 2.0 angstrom, 2.5 angstrom, 3.0 angstrom, 3.5 angstrom, 4.0 angstrom, 4.5 angstrom, 5.0 angstrom, 5.5 angstrom, 6.0 angstrom, 6.5 angstrom, 7.0 angstrom, 7.5 angstrom, 8.0 angstrom, 8.5 angstrom, 9.0 angstrom, 9.5 angstrom, 10.0 angstrom, 10.5 angstrom, 11.0 angstrom, 11.5 angstrom, 12.0 angstrom, 12.5 angstrom, 13.0 angstrom, 13.5 angstrom, 14.0 angstrom, 14.5 angstrom, 15.0 angstrom, 15.5 angstrom, 16.0 angstrom, 16.5 angstrom, 17.0 angstrom, 17.5 angstrom, 18.0 angstrom, 18.5 angstrom, 19.0 angstrom, 19.5 angstrom, 20.0 angstrom, 20.5 angstrom, 21.0 angstrom, 21.5 angstrom, 22.0 angstrom, 22.5 angstrom, 23.0 angstrom, 23.5 angstrom, 24.0 angstrom, 24.5 angstrom, or 25.0 angstrom. In an embodiment, the interatomic distance between any alpha-carbon of the first epitope on the scaffold protein and any alpha-carbon of the second epitope on the scaffold protein is at most 30.0 angstrom, 30.5 angstrom, 31.0 angstrom, 31.5 angstrom, 32.0 angstrom, 32.5 angstrom, 33.0 angstrom, 33.5 angstrom, 34.0 angstrom, 34.5 angstrom, 35.0 angstrom, 35.5 angstrom, 36.0 angstrom, 36.5 angstrom, 37.0 angstrom, 37.5 angstrom, 38.0 angstrom, 38.5 angstrom, 39.0 angstrom, 39.5 angstrom, 40.0 angstrom, 40.5 angstrom, 41.0 angstrom, 41.5 angstrom, 42.0 angstrom, 42.5 angstrom, 43.0 angstrom, 43.5 angstrom, 44.0 angstrom, 44.5 angstrom, 45.0 angstrom, 45.5 angstrom, 46.0 angstrom, 46.5 angstrom, 47.0 angstrom, 47.5 angstrom, 48.0 angstrom, 48.5 angstrom, 49.0 angstrom, 49.5 angstrom, 50.0 angstrom, 50.5 angstrom, 51.0 angstrom, 51.5 angstrom, 52.0 angstrom, 52.5 angstrom, 53.0 angstrom, 53.5 angstrom, 54.0 angstrom, 54.5 angstrom, 55.0 angstrom, 55.5 angstrom, 56.0 angstrom, 56.5 angstrom, 57.0 angstrom, 57.5 angstrom, 58.0 angstrom, 58.5 angstrom, 59.0 angstrom, 59.5 angstrom, 60.0 angstrom, 60.5 angstrom, 61.0 angstrom, 61.5 angstrom, 62.0 angstrom, 62.5 angstrom, 63.0 angstrom, 63.5 angstrom, 64.0 angstrom, 64.5 angstrom, 65.0 angstrom, 65.5 angstrom, 66.0 angstrom, 66.5 angstrom, 67.0 angstrom, 67.5 angstrom, 68.0 angstrom, 68.5 angstrom, 69.0 angstrom, 69.5 angstrom, or 70.0 angstrom.

### Combination of Target-Binding Molecules

The term “combination of target-binding molecules”, as used herein, refers in one embodiment to a composition comprising both the first target-binding molecule and the second target-binding molecule. In this embodiment, the first target-binding molecule and the second target-binding molecule can be connected or linked with each other, leading to a single protein or protein complex combining both the first target-binding molecule and the second target-binding molecule.

In a further embodiment, the combination refers to two compositions, wherein the first comprises the first target-binding molecule, and the second comprises the second target-binding molecule. As the first target-binding molecule and the second target-binding molecule need to both be present at the biological site of action, both compositions need to be part of the combination. However, it is not necessary to have both compositions combined into one single composition. Having separated compositions, wherein the first comprises the first target-binding molecule, and the second comprises the second target-binding molecule, allows separate administration of the compositions, which in turn allows better fine tuning of the administration method. The term “combining”, as used herein, therefore encompasses adding both target-binding molecules to a single composition prior to administration, as well as administering both target-binding molecules separately, be it simultaneously, concurrently, or consecutively, which leads to a combination of the separately administered target-binding molecules at the biological site of action as defined in [A-33] and/or [A-34].

### Scaffold Protein

Each target-binding molecule of the present invention comprises a binding domain capable of binding a scaffold protein. The term “scaffold protein”, as used herein, refers, inter alia to cell surface proteins, soluble proteins or insoluble proteins that are expressed in the target cells, target tissues or target organs. As used herein, scaffold proteins are not limited by structure or function. Rather, scaffold proteins serve as an anchor for the first target-binding molecule and the second target-binding molecule, or the first binding domain and third binding domain of the protein complex, to bind non-competitively and/or biparatopically. Scaffold proteins may be cell surface markers, specific cell surface markers, immune cell surface markers, T cell exhaustion markers, tumor-associated marker, tumor-associated antigens or tumor-specific antigens, tissue-specific marker, disease tissue-specific marker, organ-specific marker, soluble protein, insoluble protein deposition, such as PD1, PDL1, CSF2RB, CD4, CD5, CD6, CD7, CD8, CD9, CD25, CCR4, CD8, CD27, CD38, CD39, CD45, CD62L, CCR6, CD69, CD103, CD73, CD127, GITR, LRRC32, ICOS, TIGIT, RBD, MUC1, CEA, CTLA4, IL2R alpha, CXCR5, Neuropilin-1, TIM3, LAG3, TNF alpha, CD19, CD20, CD22, CD30, CD33, Glycoprotein NMB, CD56, CD70, CD79A, CD79B, CD138, PSCA, PSMA, BCMA, FcRH5, GPRC5D, FAP, LRRC15, E-selectin, EphB2, Melanotransferrin, HER2, TROP2, Nectine-4, EGFRvIII, IL13RA2, TMEFF2, Muc16, EpCAM, FcRH2, AFP, PSA, Amyloid beta, MBP or ASGPR etc. In a preferred embodiment, the scaffold protein is PD1.

The scaffold protein of the present invention can be a membrane-bound protein, a soluble protein, or an insoluble protein deposition. It is preferably membrane-bound, which facilitates localization to the intended biological site of action. In the case of a soluble scaffold protein, localization to the intended biological site can be ensured by targeting a soluble scaffold protein, which is known to be present in high concentration at the intended biological site. For example, when the intended biological site of action is a tumoral tissue, it can be useful to target a soluble scaffold protein that is secreted by the tumoral tissue. Thereby, the localization is achieved, and a systemic activation of the signaling pathway can be avoided.

### Tumor-Specific Marker or Tumor-Associated Marker

As used herein, a “tumor-specific marker” or a “tumor-associated marker” as a non-limiting example of a scaffold protein means an antigen expressed by a cancer cell, which allows distinguishing between a cancer cell and a healthy cell. For example, a “tumor-specific marker” or a “tumor-associated marker” includes antigens that are expressed as cells become malignant and abnormal sugar chains that appear on the cell surface or on protein molecules when the cells become cancerous. Specific examples thereof include ALK, pleiotrophin (PTN), EpCAM, CA125, Prostatic acid phosphatase (PAP), Prostate-specific antigen (PSA), TYRP1, HMW-MAA, Prostate-specific membrane antigen (PSMA), CEA, MUC1, HMFG1, TAG-72, GICA (CA19-9), NY-ESO-1, LEA, CD15, CD17, CD19, CD20, CD22, CD30, CD33, CD38, CD77, CD79b, CD147, CD228, GD2, GD3, GM2, GM3, TSTA, virus-induced tumor antigens (e.g. envelope antigens of DNA and RNA tumor virus), EBV-specific antigen, alpha-fetoprotein (AFP), 5T4, differentiation antigens (e.g. L6 and L20 antigens), CD165, EGFR, ANKRD17, ErbB2, APO-1, SSEA-1, SCP-1, LeY, oligosaccharide antigens, SSEA-3, SSEA-4, CTAGE1, MART-1, sialyl Tn (STn), NY-CO-45, NY-LU-12, ART1, MA2, NOVA2, TSPAN8, MAGE-C1, MAGE-B1, MAGE-B2, MAGE-4A, MAGE-X2, YKL-40, EREG, CA15-3, CLEC12A, Nectin4, Trop2, BCMA, Tissue factor, FR alpha (FOLRT), ErbB3, Claudin18 (Claudin18.2), B7-H3 (CD276), MET, PSCA, PTK7, MSLN (Mesothelin), CCR4, CDH6, ILT3RA2, DLL3, GPC3, Claudin 6, fibroblast associated protein (FAP), FLT3, GD2, GD3 and any fragment of these polypeptides, and modified structures thereof.

### Multimeric Scaffold Protein

The scaffold protein of the present invention can be composed of several subunits. The subunits can be the same or different. Therefore, the scaffold protein can be a homomultimer, or a heteromultimer. In some embodiments, wherein the first binding domain and the third binding domain are directed at the same or overlapping epitope on the homomultimeric scaffold protein, the homomultimeric scaffold protein comprises more than one of the same epitope, thus allowing non-competitive binding of the first and third binding domain.

### Receptor

Each target-binding molecule of the present invention comprises a binding domain capable of binding a receptor protein. The term “receptor”, as used herein, refers to protein structures in or on the surface of cells that receive and transduce signals by binding to specific molecules and/or multimerizing. The receptor may be a cytokine receptor, a Wnt receptor, a receptor that induces signal by hetero-dimerization, homo-dimerization, or oligomerization activated only after the monomers of the homodimer, homotrimer, homomultimer, heterodimer, heterotrimer, or heteromultimer associate. According to the present invention, the first receptor protein and the second receptor protein are each receptor subunits that can associate to form a receptor complex. The receptor complex is activated upon association of the receptor subunits. Association of the receptor subunits happens upon binding of the first target-binding molecule to both its targets, and upon binding of the second target-binding molecule to both its targets. Examples for receptor subunits are IL2R beta, IL2R gamma, IL4R, IL13RAT, IL7R, IL9R, IL21R, TSLPR, IL3RA, CSF2RB, IL5RA, CSF2RA, IL6R, gp130, IL11RA, IL12RB1, IL12RB2, IL27RA, IL31RA, OSMR, CNTFR, LIFR, IL10RA, IL10RB, IL20RA, IL20RB, IL22RA1, IL28RA, IFNAR1, IFNAR2, IFNGR1, IFNGR2, IL1R1, IL1RAP, IL18R1, IL18RAP, ST2, IL17RA, IL17RC, TLR3, TLR4, TLR7, TLR9, CSF1R, TNFR1, TNFR2, LTBR, HVEM, FAS, CD25, CD28, DR3, DR4, DR5, NGFR, RANK, FN14, CD40, 4-1BB, OX40, GITR, TGFBR1, TGFBR2, ACVRL1, ACVR2A, BMPR2, ACVR2B, ACVR1B, ACVR1C, ACVR1, AMHR2, BMPR1A, BMPR1B, BMPR2, TRA, TRB, CD3E, CD16, TREM2, FGFR1, FGFR2, FGFR3, FGFR4, Fzd1, Fzd2, Fzd3, Fzd4, Fzd5, Fzd6, Fzd7, Fzd8 Fzd9, Fzd10, LRP5, LRP6 or LGR5. In a preferred embodiment, the first receptor protein is IL-2R beta, and the second receptor protein is IL-2R gamma.

### Inducing Receptor Signaling

The term “inducing receptor signaling”, as used herein, refers to the biological mechanism, which proceeds upon formation of the activated receptor complex. Activation of a receptor complex requires the association of the receptor protein subunits of the receptor complex. A receptor protein, as is known in the art, comprises an extracellular part. This extracellular part can interact with the target-binding molecule of the present invention. The receptor protein can also comprise a cytoplasmic part, which can interact with downstream members of the signaling pathway activated by the receptor complex. Upon association of the receptor subunits, the receptor subunits are in close proximity to one another, which, either by itself, or by facilitating a conformational change of a member of the receptor complex, induces receptor signaling, as is well known in the art. For example, the cytoplasmic part of the receptor protein subunit can recruit Janus kinases to start the intracellular signal cascade. Janus kinases will phosphorylate cytosolic STAT proteins. The phosphorylation status of STAT proteins can be easily monitored in assays established in the art. In certain embodiments, when the receptor protein is IL-2R, IL-2 receptor complex activation may be evaluated using Quanti-Blue solution (InvivoGen, #rep-qbs), a colorimetric enzyme assay that determines alkaline phosphatase activity, by measuring optical density at 620 nm, using Multiskan™ plate reader. In certain embodiments, receptor signaling of the receptor complex is induced when there is an increase in optical density by at least 5%, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490% or 500% in the presence of the combination of target-binding molecules or protein complex of the invention as compared to in the absence of the combination of target-binding molecules or protein complex of the invention.

### Proximity

Upon binding of the first, second, third and fourth binding domains of the combination of target-binding molecules or of the protein complex of the invention, the first and the second receptor proteins are brought into proximity with each other such that the receptor complex comprising the first and second receptor subunit is activated and signaling is induced. The term “proximity”, as used herein, refers to the closeness in distance between the two individual receptor proteins, for example, between IL-2R beta, and the second receptor protein is IL-2R gamma.

### Half-Life Extending Domain

In the present invention, the first binding domain and/or the third binding domain can be each connected to a half-life extending domain. One barrier for the application of protein therapeutics has been the short half-life in circulation of these small proteins. Persistence in the circulation is desirable because protein therapeutics generally cannot be administered orally and typically are administered by subcutaneous, intramuscular, or intravenous injection or infusion. Therefore, in a preferred embodiment, the present invention may encompass the use of one or more pharmaceutically acceptable half-life extending domain, such as polyethylene glycol (PEG), immunoglobulin Fc domain or a CH2 domain of Fc, albumin (e.g., Human Serum Albumin (HSA) or a variant thereof), an albumin-binding protein, FcRn-binding domain, transthyretin or thyroxine-binding globulin (TBG).

An exemplary way to improve the pharmacokinetics (PK) of a polypeptide is by expression of a domain in the polypeptide chain that binds to receptors that are recycled to the plasma membrane of cells rather than degraded in the lysosomes, such as the FcRn receptor on endothelial cells and transferrin receptor. Three types of proteins, e.g., human IgGs, HSA (or fragments), and transferrin, persist for much longer in human serum than would be predicted just by their size, which is a function of their ability to bind to receptors that are recycled rather than degraded in the lysosome. These proteins, or fragments of them that retain the FcRn binding are routinely linked to other polypeptides to extend their serum half-life. In one embodiment, the half-life extension domain is a human serum albumin (HSA) binding domain. HSA may also be directly bound to the pharmaceutical compositions or bound via a short linker. Fragments of HSA may also be used. HSA and fragments thereof can function as both a blocking moiety and a half-life extension domain. Human IgGs can also carry out a similar function.

The serum half-life extension domain can also be antigen-binding polypeptide that binds to a protein with a long serum half-life such as serum albumin, transferrin and the like. Examples of such polypeptides include antibodies and fragments thereof including, a polyclonal antibody, a recombinant antibody, a human antibody, a humanized antibody a single chain variable fragment (scFv), single-domain antibody such as a heavy chain variable domain (VH), a light chain variable domain (VL) and a variable domain of camelid-type nanobody (VHH), a dAb and the like. Other suitable antigen-binding domain include non-immunoglobulin proteins that mimic antibody binding and/or structure such as, anticalins, affilins, affibody molecules, affimers, affitins, alphabodies, avimers, DARPins, fynomers, kunitz domain peptides, monobodies, and binding domains based on other engineered scaffolds such as SpA, GroEL, fibronectin, lipocallin and CTLA4 scaffolds. Further examples of antigen-binding polypeptides include a ligand for a desired receptor, a ligand-binding portion of a receptor, a lectin, and peptides that binds to or associates with one or more target antigens.

In other embodiments, the serum half-life extension domain can be a water-soluble polymer or a peptide that is conjugated to a water-soluble polymer, such as PEG. “PEG”, “polyethylene glycol” and “poly(ethylene glycol)” as used herein, are interchangeable and encompass any nonpeptidic water-soluble poly(ethylene oxide). The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as “branched”, “linear”, “forked”, “multifunctional” and the like, to be described in greater detail below. The PEG is not limited to a particular structure and can be linear (e.g., an end capped, e.g., alkoxy PEG or a bifunctional PEG), branched or multi-armed (e.g., forked PEG or PEG attached to a polyol core), a dendritic (or star) architecture, each with or without one or more degradable linkages. Moreover, the internal structure of the PEG can be organized in any number of different repeat patterns and can be selected from the group consisting of homopolymer, alternating copolymer, random copolymer, block copolymer, alternating tripolymer, random tripolymer, and block tripolymer. PEGs can be conjugated to polypeptide and peptides through any suitable method. Typically a reactive PEG derivative, such as N-hydroxysuccinamidyl ester PEG, is reacted with a peptide or polypeptide that includes amino acids with a side chain that contains an amine, sulfhydryl, carboxylic acid or hydroxyl functional group, such as cysteine, lysine, asparagine, glutamine, theonine, tyrosine, serine, aspartic acid, and glutamic acid.

### Possible Fc Modifications

The target-binding molecules of the present invention or the protein complex of the present invention can further comprise other possible Fc modifications. These modification can be suitable to diminish antibody-dependent cellular cytotoxicity (ADCC), or complement-dependent cytotoxicity (CDC), increase the isoelectric point (pI), or to increase FcRn binding to improve the pharmacokinetic profile, or they can be Fc mutants for preparing bispecific molecules.

In one embodiment, the invention comprises Fc gamma RIIB-binding polypeptides comprising Fc modifications and methods of using the same. In some embodiments, a Fc modification with enhanced Fe gamma RIIb-binding activity of the present invention comprises at least one amino acid alteration in a parent Fc region. In further embodiments, the ratio of [KD value of the parent Fc region for monkey Fc gamma RIIb]/[KD value of the variant Fc region for monkey Fc gamma RIIb] is 2.0 or greater. In further embodiments, the ratio of [KD value of the parent Fc region for monkey Fc gamma RIIIa]/[KD value of the Fc modification for monkey Fc gamma RIIIa] is 0.5 or smaller. In further embodiments, the ratio of [KD value of the parent Fc region for human Fc gamma RIIb]/[KD value of the variant Fc region for human Fc gamma RIIb] is 2.0 or greater. In further embodiments, the ratio of [KD value of the parent Fc region for human Fc gamma RIIIa]/[KD value of the Fc modification for human Fc gamma RIIIa] is 0.5 or smaller. In further embodiments, the ratio of [KD value of the parent Fc region for human Fc gamma RIIa (type H)]/[KD value of the variant Fc region for human Fc gamma RIIa (type H)] is 5.0 or smaller. In further embodiments, the ratio of [KD value of the parent Fc region for human Fc gamma RIIa (type R)]/[KD value of the Fc modification for human Fc gamma RIIa (type R)] is 5.0 or smaller. In another embodiment, the KD value of the variant Fc region for monkey Fc gamma RIIb is 1.0×10⁻⁶ M or smaller. In another embodiment, the KD value of the Fc modification for monkey Fc gamma RIIIa is 5.0×10⁻⁷ M or greater. In another embodiment, the KD value of the variant Fc region for human Fc gamma RIIb is 2.0×10⁻⁶ M or smaller. In another embodiment, the KD value of the Fc modification for human Fc gamma RIIIa is 1.0×10⁻⁶ M or greater. In another embodiment, the KD value of the variant Fc region for human Fc gamma RIIa (type H) is 1.0×10⁻⁷ M or greater. In another embodiment, the KD value of the Fc modification for human Fc gamma RIIa (type R) is 2.0×10⁻⁷ M or greater.

In some embodiments, a Fc modification with enhanced Fc gamma RIIb-binding activity of the present invention comprises at least one amino acid alteration of at least one position selected from the group consisting of: 231, 232, 233, 234, 235, 236, 237, 238, 239, 264, 266, 267, 268, 271, 295, 298, 325, 326, 327, 328, 330, 331, 332, 334, and 396, according to EU numbering.

In further embodiments, the Fc modification with enhanced Fc gamma RIIb-binding activity comprises at least two amino acid alterations comprising: (a) one amino acid alteration at position 236, and (b) at least one amino acid alteration of at least one position selected from the group consisting of: (i) position 231, 232, 233, 234, 235, 237, 238, 239, 264, 266, 267, 268, 271, 295, 298, 325, 326, 327, 328, 330, 331, 332, 334, and 396; (ii) position: 231, 232, 235, 239, 268, 295, 298, 326, 330, and 396; or (iii) position 268, 295, 326, and 330; according to EU numbering.

In further embodiments, the Fe modification with enhanced Fc gamma RIIb-binding activity comprises at least two amino acid alterations comprising: (a) one amino acid alteration at position 236, and (b) at least one amino acid alteration of at least one position selected from the group consisting of: 231, 232, 233, 234, 235, 237, 238, 239, 264, 266, 267, 268, 271, 295, 298, 325, 326, 327, 328, 330, 331, 332, 334, and 396, according to EU numbering.

In further embodiments, the Fc modification with enhanced Fc gamma RIIb-binding activity comprises at least two amino acid alterations comprising: (a) one amino acid alteration at position 236, and (b) at least one amino acid alteration of at least one position selected from the group consisting of: 231, 232, 235, 239, 268, 295, 298, 326, 330, and 396, according to EU numbering.

In further embodiments, the Fc modification with enhanced Fc gamma RIIb-binding activity comprises at least two amino acid alterations comprising: (a) one amino acid alteration at position 236, and (b) at least one amino acid alteration of at least one position selected from the group consisting of: 268, 295, 326, and 330 according to EU numbering.

In some embodiments, a Fc modification with enhanced Fc gamma RIIb-binding activity of the present invention comprises at least one amino acid selected from the group consisting of: (a) Asp, Glu, Phe, Gly, His, Ile, Lys, Leu, Met, Asn, Pro, Gln, Arg, Ser, Thr, Val, Trp, Tyr at position 231; (b) Ala, Asp, Glu, Phe, Gly, His, Ile, Lys, Leu, Met, Asn, Gln, Arg, Ser, Thr, Val, Trp, Tyr at position 232; (c) Asp at position 233; (d) Trp, Tyr at position 234; (e) Trp at position 235; (f) Ala, Asp, Glu, His, Ile, Leu, Met, Asn, Gln, Ser, Thr, Val at position 236; (g) Asp, Tyr at position 237; (h) Glu, Ile, Met, Gln, Tyr at position 238; (i) Ile, Leu, Asn, Pro, Val at position 239; (j) Ile at position 264; (k) Phe at position 266; (1) Ala, His, Leu at position 267; (m) Asp, Glu at position 268; (n) Asp, Glu, Gly at position 271; (o) Leu at position 295; (p) Leu at position 298; (q) Glu, Phe, Ile, Leu at position 325; (r) Thr at position 326; (s) Ile, Asn at position 327; (t) Thr at position 328; (u) Lys, Arg at position 330; (v) Glu at position 331; (w) Asp at position 332; (x) Asp, Ile, Met, Val, Tyr at position 334; and (y) Ala, Asp, Glu, Phe, Gly, His, Ile, Lys, Leu, Met, Asn, Gln, Arg, Ser, Thr, Val, Trp, Tyr at position 396; according to EU numbering.

In further embodiments, the Fc modification with enhanced Fc gamma RIIb-binding activity comprises at least one amino acid selected from the group consisting of: (a) Gly, Thr at position 231; (b) Asp at position 232; (c) Trp at position 235; (d) Asn, Thr at position 236; (e) Val at position 239; (f) Asp, Glu at position 268; (g) Leu at position 295; (h) Leu at position 298; (i) Thr at position 326; (j) Lys, Arg at position 330, and (k) Lys, Met at position 396; according to EU numbering.

In another embodiment, the invention provides a polypeptide comprising an isoelectric point (pI)-increased Fe modification and a method of using the same. In some embodiments, a polypeptide comprising a Fc modification with increased pI comprises at least two amino acid alterations in a parent Fc region. In further embodiments, each of the amino acid alterations increases the isoelectric point (pI) of the Fc modification compared with that of the parent Fc region. In further embodiments, the amino acid can be exposed on the surface of the modified Fc region. In further embodiments, a polypeptide comprises the Fc modification and an antigen-binding domain. In further embodiments, antigen-binding activity of the antigen-binding domain changes according to ion concentration conditions. In further embodiments, the modified Fc region with increased pI of the present invention comprises at least two amino acid alterations of at least two positions selected from the group consisting of: 285, 311, 312, 315, 318, 333, 335, 337, 341, 342, 343, 384, 385, 388, 390, 399, 400, 401, 402, 413, 420, 422, and 431 according to EU numbering. In further embodiments, the Fc modification with increased pI comprises Arg or Lys at each of the positions selected.

In another embodiment, the invention provides isolated polypeptides comprising Fc modifications with increased isoelectric point (pI). In certain embodiments, a Fc modification described herein comprises at least two amino acid alterations in a parent Fc region. In certain embodiments, each of the amino acid alterations increases the isoelectric point (pI) of the variant Fc region compared with that of the parent Fc region. They are based on the findings that antigen elimination from plasma can be promoted with an antibody whose pI has been increased by modification of at least two amino acid residues, for example when the antibody is administered in vivo.

In an embodiment, pI may be either a theoretical or an experimentally determined pI. The value of pI can be determined, for example, by isoelectric focusing known to those skilled in the art. The value of a theoretical pI can be calculated, for example, using gene and amino acid sequence analysis software (Genetyx, etc.).

In an embodiment, the pI value may be increased, for example, at least by 0.01, 0.03, 0.05, 0.1, 0.2, 0.3, 0.4, 0.5, or more, at least by 0.6, 0.7, 0.8, 0.9, or more, at least by 1.0, 1.1, 1.2, 1.3, 1.4, 1.5, or more, or at least by 1.6, 1.7, 1.8, 1.9, 2.0, 2.1, 2.2, 2.3, 2.4, 2.5, 3.0 or more, as compared to before modification.

In certain embodiments, the amino acid for increased pI can be exposed on the surface of the Fc modification. In the present invention, an amino acid that can be exposed on the surface generally refers to an amino acid residue located on the surface of a polypeptide constituting a Fc modification. An amino acid residue located on the surface of a polypeptide refers to an amino acid residue whose side chain can be in contact with solvent molecules (which in general are mostly water molecules). However, the side chain does not necessarily have to be wholly in contact with solvent molecules, and when even a portion of the side chain is in contact with the solvent molecules, the amino acid is defined as an “amino acid residue located on the surface”. The amino acid residues located on the surface of a polypeptide also include amino acid residues located close to the surface and thereby can have an electric charge influence from another amino acid residue whose side chain, even partly, is in contact with the solvent molecules. Those skilled in the art can prepare a homology model of a polypeptide for example, using commercially available softwares. Alternatively, it is possible to use methods known to those skilled in the art, such as X-ray crystallography. The amino acid residues that can be exposed on the surface are determined, for example, using coordinates from a three-dimensional model using a computer program such as InsightII program (Accelrys). Surface-exposable sites may be determined using algorithms known in the technical field (for example, Lee and Richards (J. Mol. Biol. 55:379-400 (1971)); Connolly (J. Appl. Cryst. 16:548-558 (1983)). Surface-exposable sites can be determined using software suitable for protein modeling and three-dimensional structure information. Software available for such purposes includes, for example, the SYBYL Biopolymer Module software (Tripos Associates). When an algorithm requires a user input size parameter, the “size” of a probe which is used in the calculation may be set to about 1.4 Angstrom (A) or less in radius. Furthermore, methods for determining surface-exposable regions using software for personal computers have been described by Pacios (Comput. Chem. 18(4):377-386 (1994); J. Mol. Model. 1:46-53 (1995)). Based on such information as described above, appropriate amino acid residues located on the surface of a polypeptide that constitutes a variant Fc region can be selected.

In certain embodiments, a polypeptide comprises both the Fc modification and an antigen-binding domain. In further embodiments, the antigen is a soluble antigen. In one embodiment, the antigen is present in biological fluids (for example, plasma, interstitial fluid, lymphatic fluid, ascitic fluid, and pleural fluid) of subjects. The antigen may also be a membrane antigen.

In further embodiments, antigen-binding activity of the antigen-binding domain changes according to ion concentration conditions. In one embodiment, ion concentration is not particularly limited and refers to hydrogen ion concentration (pH) or metal ion concentration. Herein, metal ions refer to ions of group I elements except hydrogen, such as alkaline metals and the copper group elements, group II elements such as alkaline earth metals and zinc group elements, group III elements except boron, group IV elements except carbon and silicon, group VIII elements such as iron group and platinum group elements, elements belonging to subgroup A of groups V, VI, and VII, and metal elements such as antimony, bismuth, and polonium. In the present invention, metal ions include, for example, calcium ion, as described in WO 2012/073992 and WO 2013/125667. In one embodiment, “ion concentration condition” may be a condition that focuses on differences in the biological behavior of an antigen-binding domain between a low ion concentration and a high ion concentration. Furthermore, “antigen-binding activity of an antigen-binding domain changes according to ion concentration conditions” means that the antigen-binding activity of an antigen-binding domain changes between a low ion concentration and a high ion concentration (such an antigen-binding domain is referred to herein as “ion concentration-dependent antigen-binding domain”). The antigen-binding activity of an antigen-binding domain under a high ion concentration condition may be higher (stronger) or lower (weaker) than that under a low ion concentration condition. In one embodiment, ion concentration-dependent antigen-binding domains (such as pH-dependent antigen-binding domains or calcium ion concentration-dependent antigen-binding domains) can be obtained by known methods, for example, described in WO 2009/125825, WO 2012/073992, and WO 2013/046722.

In an embodiment, the antigen-binding activity of an antigen-binding domain under a high calcium ion concentration condition may be higher than under a low calcium ion concentration condition. The high calcium ion concentration is not particularly limited to but may be a concentration selected between 100 micro M and 10 mM, between 200 micro M and 5 mM, between 400 micro M and 3 mM, between 200 micro M and 2 mM, between 400 micro M and 1 mM, or between 500 micro M and 2.5 mM, which is preferable to be close to the plasma (blood) concentration of calcium ion in vivo. Meanwhile, the low calcium ion concentration is not particularly limited to but may be a concentration selected between 0.1 micro M and 30 micro M, between 0.2 micro M and 20 micro M, between 0.5 micro M and 10 micro M, between 1 micro M and 5 micro M, or between 2 micro M and 4 micro M, which is preferable to be close to the concentration of calcium ion in early endosomes in vivo.

In one embodiment, the ratio between the antigen-binding activities under a low calcium ion concentration condition and a high calcium ion concentration condition is not limited but the ratio of the dissociation constant (KD) under a low calcium ion concentration condition to the KD under a high calcium ion concentration condition, i.e., KD (low calcium ion concentration condition)/KD (high calcium ion concentration condition), is 2 or more, 10 or more, or 40 or more. The upper limit of the ratio may be 400, 1000, or 10000, as long as such an antigen-binding domain can be produced by techniques known to those skilled in the art. Alternatively, for example, the dissociation rate constant (kd) can be used instead of the KD. In this case, the ratio of the kd under a low calcium ion concentration condition to the kd under a high calcium ion concentration condition, i.e., kd (low calcium ion concentration condition)/kd (high calcium ion concentration condition), is 2 or more, 5 or more, 10 or more, or 30 or more. The upper limit of the ratio may be 50, 100, or 200, as long as the antigen-binding domain can be produced based on the common technical knowledge of those skilled in the art.

In an embodiment, the antigen-binding activity of an antigen-binding domain under a low hydrogen ion concentration (neutral pH) may be higher than under a high hydrogen ion concentration (acidic pH). The acidic pH may be, for example, a pH selected from pH4.0 to pH6.5, selected from pH4.5 to pH6.5, selected from pH5.0 to pH6.5, or selected from pH5.5 to pH6.5, which is preferable to be close to the in vivo pH in early endosomes. The acidic pH may also be, for example, pH5.8 or pH6.0. In particular embodiments, the acidic pH is pH5.8. Meanwhile, the neutral pH may be, for example, a pH selected from pH6.7 to pH10.0, selected from pH6.7 to pH9.5, selected from pH7.0 to pH9.0, or selected from pH7.0 to pH8.0, which is preferable to be close to the in vivo pH in plasma (blood). The neutral pH may also be, for example, pH7.4 or pH7.0. In particular embodiments, the neutral pH is pH7.4.

In one embodiment, the ratio between the antigen-binding activities under an acidic pH condition and a neutral pH condition is not limited but the ratio of the dissociation constant (KD) under an acidic pH condition to the KD under a neutral pH condition, i.e., KD (acidic pH condition)/KD (neutral pH condition), is 2 or more, 10 or more, or 40 or more. The upper limit of the ratio may be 400, 1000, or 10000, as long as such an antigen-binding domain can be produced by techniques known to those skilled in the art. Alternatively, for example, the dissociation rate constant (kd) can be used instead of the KD. In this case, the ratio of the kd under an acidic pH condition to the kd under a neutral pH condition, i.e., kd (acidic pH condition)/kd (neutral pH condition) is 2 or more, 5 or more, 10 or more, or 30 or more. The upper limit of the ratio may be 50, 100, or 200, as long as the antigen-binding domain can be produced based on the common technical knowledge of those skilled in the art.

In one embodiment, for example, at least one amino acid residue is substituted with an amino acid residue with a side-chain pKa of 4.0-8.0, and/or at least one amino acid with a side-chain pKa of 4.0-8.0 is inserted in the antigen-binding domain, as described in WO 2009/125825. The amino acid may be substituted and/or inserted at any site as long as the antigen-binding activity of the antigen-binding domain becomes weaker under an acidic pH condition than under a neutral pH condition as compared to before the substitution or insertion. When the antigen-binding domain has a variable region or CDR, the site may be within the variable region or CDR. The number of amino acids that are substituted or inserted can be appropriately determined by those skilled in the art; and the number may be one or more. Amino acids with a side-chain pKa of 4.0-8.0 can be used to change the antigen-binding activity of the antigen-binding domain according to the hydrogen ion concentration condition. Such amino acids include, for example, natural amino acids such as His (H) and Glu (E), and unnatural amino acids such as histidine analogs (US2009/0035836), m-NO2-Tyr (pKa 7.45), 3,5-Br2-Tyr (pKa 7.21), and 3,5-I2-Tyr (pKa 7.38) (Heyl et al., Bioorg. Med. Chem. 11(17):3761-3768 (2003)). Amino acids with a side-chain pKa of 6.0-7.0 can also be used, which include, e.g., His (H).

In another embodiment, preferable antigen-binding domains for the Fc modification with increased pI are described and can be obtained by methods described in Japanese patent applications JP2015-021371 and JP2015-185254.

In certain embodiments, the Fc modification with increased pI comprises at least two amino acid alterations of at least two positions selected from the group consisting of: 285, 311, 312, 315, 318, 333, 335, 337, 341, 342, 343, 384, 385, 388, 390, 399, 400, 401, 402, 413, 420, 422, and 431, according to EU numbering.

In further embodiments, the Fc modification with increased pI comprises at least two amino acid alterations of at least two positions selected from the group consisting of: 311, 341, 343, 384, 399, 400, 401, 402, and 413, according to EU numbering.

In another aspect, the invention provides polypeptides comprising Fc modifications with increased pI comprising amino acid alterations of any one of the following (1)-(10): (1) positions 311 and 341; (2) positions 311 and 343; (3) positions 311, 343 and 413; (4) positions 311, 384 and 413; (5) positions 311 and 399; (6) positions 311 and 401; (7) positions 311 and 413; (8) positions 400 and 413; (9) positions 401 and 413; and (10) positions 402 and 413; according to EU numbering.

A method for increasing the pI of a protein is, for example, to reduce the number of amino acids with a negatively charged side chain (for example, aspartic acid and glutamic acid) and/or to increase the number of amino acids with a positively charged side chain (for example, arginine, lysine and histidine) at a neutral pH condition. Amino acids with a negatively charged side chain have a negative charge represented as −1 at a pH condition that is sufficiently higher than their side chain pKa, which is a theory well known to those skilled in the art. For example, the theoretical pKa for the side chain of aspartic acid is 3.9, and the side chain has a negative charge represented as −1 at a neutral pH condition (for example, in a solution of pH7.0). Conversely, amino acids with a positively charged side chain have a positive charge represented as +1 at a pH condition that is sufficiently lower than their side chain pKa. For example, the theoretical pKa for the side chain of arginine is 12.5, and the side chain has a positive charge represented as +1 at a neutral pH condition (for example, in a solution of pH7.0). Meanwhile, amino acids whose side chain has no charge at a neutral pH condition (for example, in a solution of pH7.0) are known to include 15 types of natural amino acids, i.e., alanine, cysteine, phenylalanine, glycine, isoleucine, leucine, methionine, asparagine, proline, glutamine, serine, threonine, valine, tryptophan, and tyrosine. As a matter of course, it is understood that amino acids for increasing the pI may be unnatural amino acids.

From the above, a method for increasing the pI of a protein at a neutral pH condition (for example, in a solution of pH7.0) can confer a charge alteration of +1 to a protein of interest, for example, by substituting amino acids with non-charged side chains for aspartic acid or glutamic acid (whose side chain has a negative charge of −1) in the amino acid sequence of the protein. Furthermore, a charge alteration of +1 can be conferred to the protein, for example, by substituting arginine or lysine (whose side chain has a positive charge of +1) for amino acids whose side chain has no charge. Moreover, a charge alteration of +2 can be conferred at a time to the protein by substituting arginine or lysine (whose side chain has a positive charge of +1) for aspartic acid or glutamic acid (whose side chain has a negative charge of −1). Alternatively, to increase the pI of a protein, amino acids with a side chain having no charge and/or preferably amino acids having a positively charged side chain can be added or inserted into the amino acid sequence of the protein, or amino acids with a side chain having no charge and/or preferably amino acids with a negatively charged side chain present in the amino acid sequence of the protein can be deleted. It is understood that, for example, the N-terminal and C-terminal amino acid residues of a protein have a main chain-derived charge (NH3⁺ of the amino group at the N-terminus and COO⁻ of the carbonyl group at the C-terminus) in addition to their side chain-derived charges. Thus, the pI of a protein can also be increased by performing to the main chain-derived functional groups some addition, deletion, substitution, or insertion.

The substitution of an amino acid to increase the pI includes, for example, substitution of an amino acid whose side chain has no charge for an amino acid having a negatively charged side chain, substitution of an amino acid having a positively charged side chain for an amino acid whose side chain has no charge, and substitution of an amino acid having a positively charged side chain for an amino acid having a negatively charged side chain in the amino acid sequence of a parent Fc region, which are performed alone or in appropriate combinations.

The insertion or addition of an amino acid to increase the pI includes, for example, insertion or addition of an amino acid whose side chain has no charge, and/or insertion or addition of an amino acid having a positively charged side chain in the amino acid sequence of a parent Fc region, which are performed alone or in appropriate combinations.

The deletion of an amino acid to increase the pI includes, for example, deletion of an amino acid whose side chain has no charge, and/or deletion of an amino acid having a negatively charged side chain in the amino acid sequence of a parent Fc region, which are performed alone or in appropriate combinations.

In one embodiment, natural amino acids used for increasing pI can be classified as follows: (a) an amino acid with a negatively charged side chain can be Glu (E) or Asp (D); (b) an amino acid whose side chain has no charge can be Ala (A), Asn (N), Cys (C), Gln (Q), Gly (G), His (H), Ile (I), Leu (L), Met (M), Phe (F), Pro (P), Ser (S), Thr (T), Trp (W), Tyr (Y), or Val (V); and (c) an amino acid with a positively charged side chain can be His (H), Lys (K), or Arg (R). In one embodiment, the amino acid insertion or substitution after modification is Lys (K) or Arg (R).

### Affinity

The term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

In certain embodiments, a target-binding molecule provided herein has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M).

In one embodiment, Kd is measured by a radiolabeled antigen binding assay (RIA). In one embodiment, an RIA is performed with the Fab version of an target-binding molecule of interest and its target. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, NJ) is performed at 25 degrees C. with immobilized antigen CM5 chips at ~10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (~0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### And/or

The term “and/or” herein is used to indicate any one of the subjects shown before and after “and/or”, or any combination thereof. For example, “A, B, and/or C” includes the individual subjects “A”, “B”, and “C”, and also combinations “A and B”, “A and C”, “B and C”, and “A and B and C”.

### Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); single domain antibodies or VHH; and multispecific antibodies formed from antibody fragments.

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

If the term “variable fragment (Fv)” is used in the instant application, it may refer to the minimum unit of an antibody-derived portion binding to an antigen that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

If the terms “scFv”, “single-chain antibody”, and “sc(Fv)2” are used in the instant application, those refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

If the term “scFv” is used in the instant application, it may mean a single chain polypeptide in which VH and VL forming Fv are linked together by a peptide linker (Proc. Natl. Acad. Sci. U.S.A. (1988) 85(16), 5879-5883). VH and VL can be retained in close proximity by the peptide linker.

If the term “sc(Fv)2” is used in the instant application, it may mean a single-chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)2 preferably includes, for example, a bispecific sc(Fv)2 that recognizes two epitopes present in a single antigen as disclosed in the Journal of Immunology (1994) 152(11), 5368-5374. sc(Fv)2 can be produced by methods known to those skilled in the art. For example, sc(Fv)2 can be produced by linking scFv by a linker such as a peptide linker.

Herein, the sc(Fv)2 takes a form in which the two VH units and two VL units of an antibody are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Example order of the form is listed below.
[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

If the term “Fab”, “F(ab′)2”, and “Fab′” are used in the instant application, those may mean as below.

“Fab” consists of a single light chain, and a CH1 region and variable region from a single heavy chain. The heavy chain of a wild-type Fab molecule cannot form disulfide bonds with another heavy chain molecule. Depending on any purpose, Fab variants in which amino acid residue(s) in a wild-type Fab molecule may be altered by substitution, addition, or deletion are also included. In a specific embodiment, mutated amino acid residue(s) comprised in Fab variants (e.g., cysteine residue(s) or lysine residue(s) after substitution, addition, or insertion) can form disulfide bond(s) with another heavy chain molecule or a portion thereof (e.g., Fab molecule).

scFab is an antigen-binding domain in which a single light chain, and a CH1 region and variable region from a single heavy chain which form Fab are linked together by a peptide linker. The light chain, and the CH1 region and variable region from the heavy chain can be retained in close proximity by the peptide linker.

“F(ab′)2” or “Fab” is produced by treating an immunoglobulin (monoclonal antibody) with a protease such as pepsin and papain, and refers to an antibody fragment generated by digesting an immunoglobulin (monoclonal antibody) at near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)2” consists of two light chains and two heavy chains comprising the constant region of a CH1 domain and a portion of CH2 domains so that disulfide bonds are formed between the two heavy chains. For example, the F(ab′)2 disclosed herein can be produced as follows. A whole monoclonal antibody or such comprising a desired antigen-binding domain is partially digested with a protease such as pepsin; and Fc fragments are removed by adsorption onto a Protein A column. The protease is not particularly limited, as long as it can cleave the whole antibody in a selective manner to produce F(ab′)2 under an appropriate setup enzyme reaction condition such as pH. Such proteases include, for example, pepsin and ficin.

If the term “single domain antibodies” (sdAb) is used in the instant application, those are not particularly limited in their structure, as long as the domain can exert antigen-binding activity by itself. Ordinary antibodies exemplified by IgG antibodies exert antigen-binding activity in a state where a variable region is formed by the pairing of VH and VL. In contrast, a single domain antibody is known to be able to exert antigen-binding activity by its own domain structure alone without pairing with another domain. Single domain antibodies usually have a relatively low molecular weight and exist in the form of a monomer.

Examples of a single domain antibody include, but are not limited to, antigen binding molecules which naturally lack light chains, such as VHH of Camelidae animals and VNAR of sharks, and antibody fragments comprising the whole or a portion of an antibody VH domain or the whole or a portion of an antibody VL domain. Examples of a single domain antibody which is an antibody fragment comprising the whole or a portion of an antibody VH/VL domain include, but are not limited to, artificially prepared single domain antibodies originating from a human antibody VH or a human antibody VL as described, e.g., in U.S. Pat. No. 6,248,516 B1. In some embodiments of the present invention, one single domain antibody has three CDRs (CDR1, CDR2, and CDR3).

Single domain antibodies can be obtained from animals capable of producing single domain antibodies or by immunizing animals capable of producing single domain antibodies. Examples of animals capable of producing single domain antibodies include, but are not limited to, camelids and transgenic animals into which gene(s) for the capability of producing a single domain antibody has been introduced. Camelids include camel, llama, alpaca, dromedary, guanaco, and such. Examples of a transgenic animal into which gene(s) for the capability of producing a single domain antibody has been introduced include, but are not limited to, the transgenic animals described in International Publication No. WO2015/143414 or US Patent Publication No. US2011/0123527 A1. Humanized single chain antibodies can also be obtained, by replacing framework sequences of a single domain antibody obtained from an animal with human germline sequences or sequences similar thereto. A humanized single domain antibody (e.g., humanized VHH) is one embodiment of the single domain antibody of the present invention.

Alternatively, single domain antibodies can be obtained from polypeptide libraries containing single domain antibodies by ELISA, panning, and such. Examples of polypeptide libraries containing single domain antibodies include, but are not limited to, naive antibody libraries obtained from various animals or humans (e.g., Methods in Molecular Biology 2012 911 (65-78) and Biochimica et Biophysica Acta—Proteins and Proteomics 2006 1764:8 (1307-1319)), antibody libraries obtained by immunizing various animals (e.g., Journal of Applied Microbiology 2014 117:2 (528-536)), and synthetic antibody libraries prepared from antibody genes of various animals or humans (e.g., Journal of Biomolecular Screening 2016 21:1 (35-43), Journal of Biological Chemistry 2016 291:24 (12641-12657), and AIDS 2016 30:11 (1691-1701))

Antibody that Binds to the Same Epitope

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

### Chimeric

The term “chimeric” antibody refers to an antibody in which a portion of the heavy and/or light chain is derived from a particular source or species, while the remainder of the heavy and/or light chain is derived from a different source or species.

### Class

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

Combination with Existing Technologies

The combination of target-binding molecules of the present invention or protein complex of the present invention can be combined with a variety of existing technologies. As a non-limiting embodiment of such a combination of technologies, the generation of cells that express a chimeric antigen receptor (CAR) utilizing the combination of target-binding molecules of the present invention or protein complex of the present invention is exemplified. Cells herein include, for example, T cells, gamma delta T cells, NK cells, NKT cells, cytokine-induced killer (CIK) cells, and macrophages (Int J Mol Sci. (2019) 20(11), 2839, Nat Rev Drug Discov. (2020) 19(5), 308). One of the non-limiting methods for the generation of a T cell expressing a CAR (CAR-T) involves, for example, a method in which a CAR comprising the combination of target-binding molecules of the present invention or protein complex of the present invention that specifically bind to scaffold proteins, tumor-associated antigens, the transmembrane domain of the TCR, and the intracellular signal domain of a costimulatory molecule such as CD28 to enhance T-cell activation is introduced into an effector cell such as a T cell by genetic modification techniques. Another non-limiting method for the combination with CAR-T cell technology involves, for example, a method in which a scaffold protein is expressed on a CAR-T cell, and the combination of target-binding molecules of the present invention or protein complex of the present invention is used to deliver cytokine or costimulatory signals to the CAR-T cell in a specific manner.

### Effector Functions

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

### Antibody-Dependent Cell-Mediated Cytotoxicity

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc gamma RIII only, whereas monocytes express Fc gamma RI, Fc gamma RII, and Fc gamma RIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

### Complement Dependent Cytotoxicity

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a Fc modification) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

### Effective Amount

An “effective amount” of an agent, e.g., a pharmaceutical formulation, refers to an amount effective, at dosages and for periods of time necessary, to achieve the desired therapeutic or prophylactic result.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half life of human FcRn high affinity binding polypeptides can be assayed, e.g., in transgenic mice or transfected human cell lines expressing human FcRn, or in primates to which the polypeptides with a Fc modification are administered. WO 2000/42072 (Presta) describes antibody variants with increased or decreased binding to FcRs. See also, e.g., Shields et al. J. Biol. Chem. 9(2):6591-6604 (2001).

### Fc Region

The term “Fe region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

### Variant Fc Region

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

### Fc Region-Comprising Antibody

The term “Fc region-comprising antibody” refers to an antibody that comprises an Fc region. The C-terminal lysine (residue 447 according to the EU numbering system) or C-terminal glycine-lysine (residues 446-447) of the Fc region may be removed, for example, during purification of the antibody or by recombinant engineering of the nucleic acid encoding the antibody. Accordingly, a composition comprising an antibody having an Fc region according to this invention can comprise an antibody with G446-K447, with G446 and without K447, with all G446-K447 removed, or a mixture of three types of antibodies described above.

### Functional Fc Region

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fe region to be combined with a binding domain (e.g., an antibody variable domain) and can be assessed using various assays as disclosed, for example, in definitions herein.

### Native Sequence Fc Region

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

### Full Length Antibody, Intact Antibody, and Whole Antibody

The terms “full length antibody,” “intact antibody,” and “whole antibody” are used herein interchangeably to refer to an antibody having a structure substantially similar to a native antibody structure or having heavy chains that contain an Fc region as defined herein.

### Host Cell, Host Cell Line, and Host Cell Culture

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

### Individual or Subject

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

### Isolated Nucleic Acid

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

### Isolated Nucleic Acid Encoding the Target-Binding Molecule or the Protein Complex or the Combination

“Isolated nucleic acid encoding the target-binding molecule or the protein complex or the combination” refers to one or more nucleic acid molecules encoding the binding domains, for example antibody heavy and light chains (or fragments thereof), including such nucleic acid molecule(s) in a single vector or separate vectors, and such nucleic acid molecule(s) present at one or more locations in a host cell.

### Percent (%) Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Pharmaceutical Formulation/Pharmaceutical Composition

The terms “pharmaceutical formulation” or “pharmaceutical composition” refer to a preparation which is in such form as to permit the biological activity of an active ingredient contained therein to be effective, and which contains no additional components which are unacceptably toxic to a subject to which the formulation would be administered.

### Pharmaceutically Acceptable Carrier

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” The vector can be introduced into a host cell by a method using a virus, an electroporation method, or the like, but the introduction of a vector is not limited to in vitro introduction, and it is also possible to directly introduce a vector into a body.

### Cancer

The combination of target-binding molecules or the protein complex of the present invention can be used for treating or preventing cancer. The terms “cancer” and “cancerous” as used herein, refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

### Cell Proliferative Disorder

The terms “cell proliferative disorder” and “proliferative disorder” as used herein, refer to disorders that are associated with some degree of abnormal cell proliferation. In one embodiment, the cell proliferative disorder is cancer.

### In Vitro and In Vivo Assays for Inhibition of Cell Proliferation and Tumor Growth

In certain embodiments, a combination of target-binding molecules of the present invention or a protein complex of the present invention, or a target-binding molecule of the present invention as defined in [A-1] in combination with the other target-binding molecule as defined in [A-1] of the invention of the invention is tested for its ability to inhibit cell growth or proliferation in vitro. Assays for inhibition of cell growth or proliferation are well known in the art. Certain assays for cell proliferation, exemplified by the “cell killing” assays described herein, measure cell viability. One such assay is the CellTiter-Glo™ Luminescent Cell Viability Assay, which is commercially available from Promega (Madison, WI). That assay determines the number of viable cells in culture based on quantitation of ATP present, which is an indication of metabolically active cells. See Crouch et al (1993) J. Immunol. Meth. 160:81-88, U.S. Pat. No. 6,602,677. The assay may be conducted in 96- or 384-well format, making it amenable to automated high-throughput screening (HTS). See Cree et al (1995) AntiCancer Drugs 6:398-404. The assay procedure involves adding a single reagent (CellTiter-Glo (registered trademark) Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

Another assay for cell proliferation is the “MTT” assay, a colorimetric assay that measures the oxidation of 3-(4,5-dimethylthiazol-2-yl)-2,5-diphenyltetrazolium bromide to formazan by mitochondrial reductase. Like the CellTiter-Glo™ assay, this assay indicates the number of metabolically active cells present in a cell culture. See, e.g., Mosmann (1983) J. Immunol. Meth. 65:55-63, and Zhang et al. (2005) Cancer Res. 65:3877-3882.

In one aspect, a combination of target-binding molecules of the present invention or a protein complex of the present invention, or a target-binding molecule of the present invention as defined in [A-1] in combination with the other target-binding molecule as defined in [A-1] of the invention is tested for its ability to induce cell death in vitro. Assays for induction of cell death are well known in the art. In some embodiments, such assays measure, e.g., loss of membrane integrity as indicated by uptake of propidium iodide (PI), trypan blue (see Moore et al. (1995) Cytotechnology, 17:1-11), or 7AAD. In an exemplary PI uptake assay, cells are cultured in Dulbecco's Modified Eagle Medium (D-MEM): Ham's F-12 (50:50) supplemented with 10% heat-inactivated FBS (Hyclone) and 2 mM L-glutamine. Thus, the assay is performed in the absence of complement and immune effector cells. Cells are seeded at a density of 3×10⁶ per dish in 100×20 mm dishes and allowed to attach overnight. The medium is removed and replaced with fresh medium alone or medium containing various concentrations of the antibody or immunoconjugate. The cells are incubated for a 3-day time period. Following treatment, monolayers are washed with PBS and detached by trypsinization. Cells are then centrifuged at 1200 rpm for 5 minutes at 4 degrees C., the pellet resuspended in 3 ml cold Ca²⁺ binding buffer (10 mM Hepes, pH 7.4, 140 mM NaCl, 2.5 mM CaCl₂)) and aliquoted into 35 mm strainer-capped 12×75 mm tubes (1 ml per tube, 3 tubes per treatment group) for removal of cell clumps. Tubes then receive PI (10 micro g/ml). Samples are analyzed using a FACSCAN™ flow cytometer and FACSCONVERT™ CellQuest software (Becton Dickinson). Combinations of target-binding molecules of the present invention or protein complexes of the present invention, or target-binding molecules of the present invention as defined in [A-1] in combination with the other target-binding molecule as defined in [A-1] of the invention which induce statistically significant levels of cell death as determined by PI uptake are thus identified.

In one aspect, a combination of target-binding molecules of the present invention or a protein complex of the present invention, or a target-binding molecule of the present invention as defined in [A-1] in combination with the other target-binding molecule as defined in [A-1] of the invention is tested for its ability to induce apoptosis (programmed cell death) in vitro. An exemplary assay for combinations of target-binding molecules of the present invention or protein complexes of the present invention, or target-binding molecules of the present invention as defined in [A-1] in combination with the other target-binding molecule as defined in [A-1] of the invention or immunoconjugates that induce apoptosis is an annexin binding assay. In an exemplary annexin binding assay, cells are cultured and seeded in dishes as discussed in the preceding paragraph. The medium is removed and replaced with fresh medium alone or medium containing 0.001 to 10 micro g/ml of the antibody or immunoconjugate. Following a three-day incubation period, monolayers are washed with PBS and detached by trypsinization. Cells are then centrifuged, resuspended in Ca²⁺ binding buffer, and aliquoted into tubes as discussed in the preceding paragraph. Tubes then receive labeled annexin (e.g. annexin V-FITC) (1 micro g/ml). Samples are analyzed using a FACSCAN™ flow cytometer and FACSCONVERT™ CellQuest software (BD Biosciences). Antibodies that induce statistically significant levels of annexin binding relative to control are thus identified. Another exemplary assay for antibodies or immunoconjugates that induce apoptosis is a histone DNA ELISA colorimetric assay for detecting internucleosomal degradation of genomic DNA. Such an assay can be performed using, e.g., the Cell Death Detection ELISA kit (Roche, Palo Alto, CA).

Cells for use in any of the above in vitro assays include cells or cell lines that naturally express the scaffold protein or the receptor proteins as defined in [A-1] or that have been engineered to express the scaffold protein or comprise all components of the receptor complex comprising the receptor proteins as defined in [A-1]. Such cells include tumor cells that overexpress the scaffold protein relative to normal cells of the same tissue origin. Such cells also include cell lines (including tumor cell lines) that express the scaffold protein and cell lines that do not normally express the scaffold protein but have been transfected with nucleic acid encoding the scaffold protein.

In one aspect, an combination of target-binding molecules of the present invention or a protein complex of the present invention, or a target-binding molecule of the present invention as defined in [A-1] in combination with the other target-binding molecule as defined in [A-1] of the invention is tested for its ability to inhibit cell growth or proliferation in vivo. In certain embodiments, an combination of target-binding molecules of the present invention or a protein complex of the present invention, or a target-binding molecule of the present invention as defined in [A-1] in combination with the other target-binding molecule as defined in [A-1] of the invention is tested for its ability to inhibit tumor growth in vivo. In vivo model systems, such as syngeneic tumor cell graft models, can be used for such testing. In exemplary syngeneic tumor cell graft models, mouse tumor cells are introduced into a suitable strain of mice, e.g., C57BL/6 mouse. Additionally, if the target-binding molecules have no-cross reactivity to the animals or cell lines that used, introducing human gene expression by genetic engineering for a scaffold protein or a receptor protein is possible option. A combination of target-binding molecules of the present invention or a protein complex of the present invention, or a target-binding molecule of the present invention as defined in [A-1] in combination with the other target-binding molecule as defined in [A-1] of the invention is administered to the animal. The ability of the combination of target-binding molecules of the present invention or the protein complex of the present invention, or the target-binding molecule of the present invention as defined in [A-1] in combination with the other target-binding molecule as defined in [A-1] of the invention to inhibit or decrease tumor growth is measured. In certain embodiments, the mouse tumor cells are cells from a mouse tumor, such as MC38, CT26, A20, B16-F10 or Pan02. In certain embodiments, the mouse tumor cells are introduced into a suitable strain of mice by subcutaneous injection or by transplantation into a suitable site, such as a mammary fat pad.

### B-Cell Neoplasms/Hodgkin's Disease

“B cell neoplasms” include Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); and Hairy cell leukemia. The non-Hodgkins lymphoma include low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma and Waldenstrom's macroglobulinemia. Treatment of relapses of these cancers are also contemplated. LPHD is a type of Hodgkin's disease that tends to relapse frequently despite radiation or chemotherapy treatment. CLL is one of four major types of leukemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

### Breast Tumor

The term “breast tumor” or “breast cancer” refers to any tumor or cancer of the breast, including, e.g., adenocarcinomas, such as invasive or in situ ductal carcinoma, invasive or in situ lobular carcinoma, medullary carcinoma, colloid carcinoma, and papillary carcinoma; and less prevalent forms, such as cystosarcoma phylloides, sarcomas, squamous cell carcinomas, and carcinosarcomas.

### Colon Tumor

The term “colon tumor” or “colon cancer” refers to any tumor or cancer of the colon (the large intestine from the cecum to the rectum).

### Colorectal Tumor

The term “colorectal tumor” or “colorectal cancer” refers to any tumor or cancer of the large bowel, which includes the colon (the large intestine from the cecum to the rectum) and the rectum, including, e.g., adenocarcinomas and less prevalent forms, such as lymphomas and squamous cell carcinomas.

### Non-Hodgkin's Lymphoma

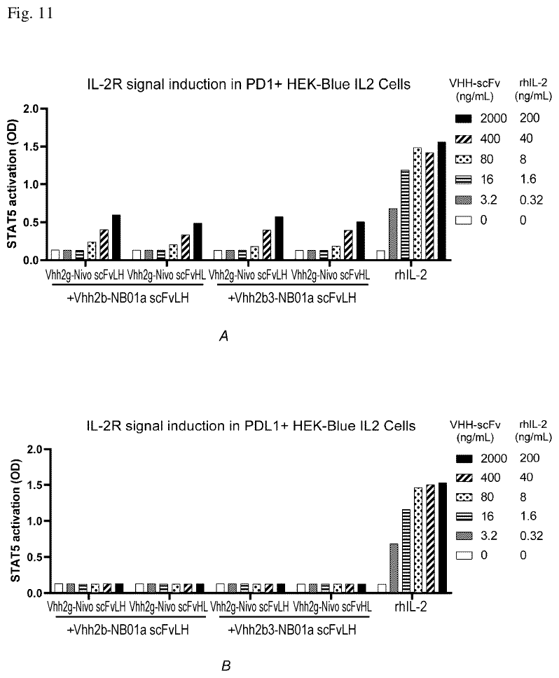

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Colour Atlas of Clinical Haematology, Third Edition; A. Victor Hoffbrand and John E. Pettit (eds.) (Harcourt Publishers Limited 2000) (see, in particular FIG. 11.57, 11.58 and/or 11.59). More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphocytic leukemia and/or prolymphocytic leukemia and/or small lymphocytic lymphoma, B-cell prolymphocytic lymphoma, immunocytoma and/or lymphoplasmacytic lymphoma, marginal zone B cell lymphoma, splenic marginal zone lymphoma, extranodal marginal zone—MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) T-cell lymphoblastic leukemia and/or lymphoma, adult T-cell lymphoma and/or leukemia, T cell chronic lymphocytic leukemia and/or prolymphacytic leukemia, large granular lymphocytic leukemia, mycosis fungoides and/or Sezary syndrome, extranodal natural killer/T-cell (nasal type) lymphoma, enteropathy type T-cell lymphoma, hepatosplenic T-cell lymphoma, subcutaneous panniculitis like T-cell lymphoma, skin (cutaneous) lymphomas, anaplastic large cell lymphoma, angiocentric lymphoma, intestinal T cell lymphoma, peripheral T-cell (not otherwise specified) lymphoma and angioimmunoblastic T-cell lymphoma.

### Ovarian Cancer

“Ovarian cancer” refers to a heterogeneous group of malignant tumors derived from the ovary. Approximately 90% of malignant ovarian tumors are epithelial in origin; the remainder are germ cell and stromal tumors. Epithelial ovarian tumors are classified into the following histological subtypes: serous adenocarcinomas (constituting about 50% of epithelial ovarian tumors); endometrioid adenocarcinomas (about 20%); mucinous adenocarcinomas (about 10%); clear cell carcinomas (about 5-10%); Brenner (transitional cell) tumors (relatively uncommon). The prognosis for ovarian cancer, which is the sixth most common cancer in women, is usually poor, with five-year survival rates ranging from 5-30%. For reviews of ovarian cancer, see Fox et al. (2002) “Pathology of epithelial ovarian cancer,” in Ovarian Cancer ch. 9 (Jacobs et al., eds., Oxford University Press, New York); Morin et al. (2001) “Ovarian Cancer,” in Encyclopedic Reference of Cancer, pp. 654-656 (Schwab, ed., Springer-Verlag, New York). The present invention contemplates methods of diagnosing or treating any of the epithelial ovarian tumor subtypes described above, and in particular, the serous adenocarcinoma subtype.

### Relapsed

“Relapsed” refers to the regression of the patient's illness back to its former diseased state, especially the return of symptoms following an apparent recovery or partial recovery. Unless otherwise indicated, relapsed state refers to the process of returning to or the return to illness before the previous treatment including, but not limited to, chemotherapies and stem cell transplantation treatments.

### Refractory

“Refractory” refers to the resistance or non-responsiveness of a disease or condition to a treatment (e.g., the number of neoplastic plasma cells increases even though treatment is given). Unless otherwise indicated, the term “refractory” refers to a resistance or non-responsiveness to any previous treatment including, but not limited to, chemotherapies and stem cell transplantation treatments.

### Stomach Tumor

The term “stomach tumor” or “stomach cancer” as used herein, refers to any tumor or cancer of the stomach, including, e.g., adenocarcinomas (such as diffuse type and intestinal type), and less prevalent forms such as lymphomas, leiomyosarcomas, and squamous cell carcinomas.

### Tumor

The term “tumor” (or “tumor”) as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

### Inhibiting Cell Growth or Proliferation/Suppressing Cell Growth

“Inhibiting cell growth or proliferation” or “suppressing cell growth” means decreasing a cell's growth or proliferation by at least 10%, 20%, 30%, 40%, 50%, 60%, 70%, 80%, 90%, 95%, or 100%, and includes inducing cell death.

### Oligonucleotide

“Oligonucleotide,” as used herein, refers to generally single-stranded, synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

### Substantially Reduced

The phrase “substantially reduced” or “substantially different,” as used herein, refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values).

### Substantially Similar

The term “substantially similar” or “substantially the same,” as used herein, refers to a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values).

### Autoimmune Disease

The combination of target-binding molecules or the protein complex of the present invention can be used for treating or preventing an autoimmune disease. “Autoimmune disease” refers to a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobulinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

### Immunosuppressive Agents/Anti-Inflammatories

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL (registered trademark) methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies including anti-interferon-alpha, -beta, or -gamma antibodies, anti-tumor necrosis factor (TNF)-alpha antibodies (infliximab (REMICADE (registered trademark)) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol., 23:113-5 (2002) and see also definition below); biologic agents that interfere with T cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate.

### II. Compositions and Methods

In one aspect, the present invention is concerned with a combination of target-binding molecules, comprising
a first target-binding molecule, comprising a first binding domain that is capable of binding to a scaffold protein and a second binding domain that is capable of binding to a first receptor protein, and
a second target-binding molecule, comprising a third binding domain that is capable of binding to the scaffold protein and a fourth binding domain that is capable of binding to a second receptor protein,
wherein the first target-binding molecule and the second target-binding molecule are capable of non-competitively binding to the scaffold protein, and
wherein the first receptor protein and the second receptor protein are each receptor subunits that can associate to form a receptor complex. The combination of target-binding molecules is capable of inducing receptor signaling of the receptor complex.

In another aspect, the present invention is concerned with a protein complex comprising a first target-binding molecule, comprising a first binding domain that is capable of binding to a scaffold protein and a second binding domain that is capable of binding to a first receptor protein, and a second target-binding molecule, comprising a third binding domain that is capable of binding to the scaffold protein and a fourth binding domain that is capable of binding to a second receptor protein,
wherein the first target-binding molecule and the second target-binding molecule are capable of non-competitively binding to the scaffold protein,
wherein the first receptor protein and the second receptor protein are each receptor subunits that can associate to form a receptor complex.
   The protein complex is capable of inducing receptor signaling of the receptor complex.

The present invention is also concerned with a protein complex comprising a first binding domain, a second binding domain, a third binding domain and a fourth binding domain, wherein the first binding domain is capable of binding to a scaffold protein, the second binding domain is capable of binding to a first receptor protein, the third binding domain is capable of binding to the scaffold protein, and the fourth binding domain is capable of binding to a second receptor protein,
wherein the first binding domain and the third binding domain are capable of non-competitively binding to the scaffold protein,
wherein the first receptor protein and the second receptor protein are each receptor subunits that can associate to form a receptor complex.
   The protein complex is capable of inducing receptor signaling of the receptor complex.

The present invention is also concerned with nucleic acids encoding the combination of target-binding molecules of the present invention or the protein complex of the present invention.

The present invention is also concerned with methods of producing the combination of target-binding molecules of the present invention or the protein complex of the present invention.

The present invention is also concerned with the medical use of the combination of target-binding molecules of the present invention or the protein complex of the present invention, or of a pharmaceutical composition comprising the combination of target-binding molecules of the present invention or the protein complex of the present invention.

The present invention is also concerned with a combination of target-binding molecules or a protein complex comprising the target-binding molecules or the protein complex of any one of Tables 1-17. As show in this application, the target-binding molecules or the protein complex of any one of Tables 1-17 are capable of inducing receptor signaling of the respective receptor complex.

### Recombinant Methods and Compositions

The combination of target-binding molecules of the present invention or the protein complex of the present invention may be produced using recombinant methods and compositions, e.g., as described in U.S. Pat. No. 4,816,567. In one embodiment, isolated nucleic acid encoding a combination of target-binding molecules of the present invention or the protein complex of the present invention described herein is provided. Such nucleic acid may encode an amino acid sequence comprising the VL and/or an amino acid sequence comprising the VH of the target-binding molecules (e.g., the light and/or heavy chains of an antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making a combination of target-binding molecules of the present invention or the protein complex of the present invention is provided, wherein the method comprises culturing a host cell comprising a nucleic acid encoding the combination of target-binding molecules of the present invention or the protein complex of the present invention, as provided above, under conditions suitable for expression of the combination of target-binding molecules of the present invention or the protein complex of the present invention, and optionally recovering the combination of target-binding molecules of the present invention or the protein complex of the present invention from the host cell (or host cell culture medium).

For recombinant production of the combination of target-binding molecules of the present invention or the protein complex of the present invention described herein, nucleic acid encoding a target-binding molecule, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, target-binding molecules may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the target-binding molecules may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

In certain embodiments, the invention provides a combination of target-binding molecules of the present invention or the protein complex of the present invention obtainable by a method set out above.

### Assays

A combination of target-binding molecules of the present invention or the protein complex of the present invention provided herein may be identified, screened for, or characterized for their physical/chemical properties and/or biological activities by various assays known in the art.

### Binding Assays and Other Assays

In one aspect, a target-binding molecule of the invention is tested for its target binding activity, e.g., by known methods such as ELISA, Western blot, etc.

In another aspect, competition assays may be used to identify a target-binding molecule binding to a scaffold protein that does not compete with a further target-binding molecule of the present invention binding to the scaffold protein. In certain embodiments, such a non-competing first target-binding molecule does not bind to the same epitope (e.g., a linear or a conformational epitope) that is bound by the second target-binding molecule. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, the immobilized scaffold protein is incubated in a solution comprising a first labeled target-binding molecule that binds to the scaffold protein and a second unlabeled target-binding molecule that is being tested for its ability to compete with the first target-binding molecule for binding to the scaffold protein. The second target-binding molecule may be present in a hybridoma supernatant. As a control, immobilized target-binding molecule is incubated in a solution comprising the first labeled target-binding molecule but not the second unlabeled target-binding molecule. After incubation under conditions permissive for binding of the first target-binding molecule to the scaffold protein, excess unbound target-binding molecule is removed, and the amount of label associated with immobilized the scaffold protein is measured. If the amount of label associated with immobilized the scaffold protein is substantially reduced in the test sample relative to the control sample, then that indicates that the second target-binding molecule is competing with the first target-binding molecule for binding to the scaffold protein. If the amount of label associated with immobilized the scaffold protein is not substantially reduced in the test sample relative to the control sample, then that indicates that the second target-binding molecule is not competing with the first target-binding molecule for binding to the scaffold protein (See Harlow and Lane (1988) Antibodies: A Laboratory Manual ch. 14 (Cold Spring Harbor Laboratory, Cold Spring Harbor, NY).

### Pharmaceutical Formulations/Pharmaceutical Compositions

Pharmaceutical formulations of the combination of target-binding molecules of the present invention or the protein complex of the present invention as described herein are prepared by mixing the combination of target-binding molecules of the present invention or the protein complex of the present invention having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Therapeutic Methods and Compositions

The combination of target-binding molecules of the present invention or the protein complex of the present invention provided herein may be used in therapeutic methods.

In one aspect, the combination of target-binding molecules of the present invention or the protein complex of the present invention is provided for use as a medicament. In further aspects, the combination of target-binding molecules of the present invention or the protein complex of the present invention is provided for use in treating cancer or an autoimmune disease. In certain embodiments, a target-binding molecule as defined in [A-1] is provided for medical use in combination with the other target-binding molecule as defined in [A-1]. In certain embodiments, the combination of target-binding molecules of the present invention or the protein complex of the present invention, or the target-binding molecule of the present invention as defined in [A-1] in combination with the other target-binding molecule as defined in [A-1] is provided for use in a method of treatment. In certain embodiments, the invention provides the combination of target-binding molecules of the present invention or the protein complex of the present invention, or the target-binding molecule of the present invention as defined in [A-1] in combination with the other target-binding molecule as defined in [A-1] for use in a method of treating an individual having cancer or an autoimmune disease, the method comprising administering to the individual an effective amount of the combination of target-binding molecules of the present invention or the protein complex of the present invention, or the target-binding molecule of the present invention as defined in [A-1] in combination with the other target-binding molecule as defined in [A-1].

The treatment mechanism of the combination of target-binding molecules of the present invention or the protein complex of the present invention, or the target-binding molecule of the present invention as defined in [A-1] in combination with the other target-binding molecule as defined in [A-1] is the conditional activation of the receptor complex upon binding of the four binding domains to their respective targets. The signal induced by the activated receptor complex leads to the therapeutic effect.

In a further aspect, the invention provides the use of the combination of target-binding molecules of the present invention or the protein complex of the present invention, or the target-binding molecule of the present invention as defined in [A-1] in combination with the other target-binding molecule as defined in [A-1] in the manufacture or preparation of a medicament. In one embodiment, the medicament is for treatment of cancer or an autoimmune disease. In a further embodiment, the medicament is for use in a method of treating cancer or an autoimmune disease, wherein treating comprises administering to an individual having cancer or an autoimmune disease an effective amount of the medicament.

In one embodiment, the method further comprises administering to the individual an effective amount of at least one additional therapeutic agent, e.g., as described below.

In a further aspect, the invention provides pharmaceutical formulations comprising the combination of target-binding molecules of the present invention or the protein complex of the present invention, or the target-binding molecule of the present invention as defined in [A-1] in combination with the other target-binding molecule as defined in [A-1] provided herein, e.g., for use in any of the above therapeutic methods. In one embodiment, a pharmaceutical formulation comprises any of the combination of target-binding molecules of the present invention or the protein complex of the present invention, or the target-binding molecule of the present invention as defined in [A-1] in combination with the other target-binding molecule as defined in [A-1] provided herein and a pharmaceutically acceptable carrier. In another embodiment, a pharmaceutical formulation comprises any of the combination of target-binding molecules of the present invention or the protein complex of the present invention, or the target-binding molecule of the present invention as defined in [A-1] in combination with the other target-binding molecule as defined in [A-1] provided herein and at least one additional therapeutic agent, e.g., as described below.

The combination of target-binding molecules of the present invention or the protein complex of the present invention, or the target-binding molecule of the present invention as defined in [A-1] in combination with the other target-binding molecule as defined in [A-1]can be used either alone or in combination with other agents in a therapy. For instance, a combination of target-binding molecules of the present invention or a protein complex of the present invention, or a target-binding molecule of the present invention as defined in [A-1] in combination with the other target-binding molecule as defined in [A-1] of the invention may be co-administered with at least one additional therapeutic agent.

Such combination therapies noted above encompass combined administration (where two or more therapeutic agents are included in the same or separate formulations), and separate administration, in which case, administration of the combination of target-binding molecules of the present invention or the protein complex of the present invention, or the target-binding molecule of the present invention as defined in [A-1] in combination with the other target-binding molecule as defined in [A-1] of the invention can occur prior to, simultaneously, and/or following, administration of the additional therapeutic agent or agents. In one embodiment, administration of the combination of target-binding molecules of the present invention or the protein complex of the present invention, or the target-binding molecule of the present invention as defined in [A-1] in combination with the other target-binding molecule as defined in [A-1] of the invention and administration of an additional therapeutic agent occur within about one month, or within about one, two or three weeks, or within about one, two, three, four, five, or six days, of each other. The combination of target-binding molecules of the present invention or the protein complex of the present invention, or the target-binding molecule of the present invention as defined in [A-1] in combination with the other target-binding molecule as defined in [A-1] of the invention can also be used in combination with radiation therapy.

An combination of target-binding molecules of the present invention or the protein complex of the present invention, or the target-binding molecule of the present invention as defined in [A-1] in combination with the other target-binding molecule as defined in [A-1] of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The combination of target-binding molecules of the present invention or the protein complex of the present invention, or the target-binding molecule of the present invention as defined in [A-1] in combination with the other target-binding molecule as defined in [A-1] of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The combination of target-binding molecules of the present invention or the protein complex of the present invention, or the target-binding molecule of the present invention as defined in [A-1] in combination with the other target-binding molecule as defined in [A-1] of the invention need not be, but is optionally formulated with one or more agents currently used to prevent or treat the disorder in question. The effective amount of such other agents depends on the amount of the combination of target-binding molecules of the present invention or the protein complex of the present invention, or the target-binding molecule of the present invention as defined in [A-1] in combination with the other target-binding molecule as defined in [A-1] of the invention present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a combination of target-binding molecules of the present invention or a protein complex of the present invention, or a target-binding molecule of the present invention as defined in [A-1] in combination with the other target-binding molecule as defined in [A-1] of the invention (when used alone or in combination with one or more other additional therapeutic agents) will depend on the type of disease to be treated, the type of target-binding molecules, the severity and course of the disease, whether the combination of target-binding molecules of the present invention or the protein complex of the present invention, or the target-binding molecule of the present invention as defined in [A-1] in combination with the other target-binding molecule as defined in [A-1] of the invention is administered for preventive or therapeutic purposes, previous therapy, the patient's clinical history and response to the target-binding molecules, and the discretion of the attending physician. The combination of target-binding molecules of the present invention or the protein complex of the present invention, or the target-binding molecule of the present invention as defined in [A-1] in combination with the other target-binding molecule as defined in [A-1] of the invention is suitably administered to the patient at one time or over a series of treatments. Depending on the type and severity of the disease, about 1 micro g/kg to 15 mg/kg (e.g. 0.1 mg/kg-10 mg/kg) of the combination of target-binding molecules of the present invention or the protein complex of the present invention, or the target-binding molecule of the present invention as defined in [A-1] in combination with the other target-binding molecule as defined in [A-1] of the invention can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the combination of target-binding molecules of the present invention or the protein complex of the present invention, or the target-binding molecule of the present invention as defined in [A-1] in combination with the other target-binding molecule as defined in [A-1] of the invention would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the combination of target-binding molecules of the present invention or the protein complex of the present invention, or the target-binding molecule of the present invention as defined in [A-1] in combination with the other target-binding molecule as defined in [A-1] of the invention). The progress of this therapy is easily monitored by conventional techniques and assays.

### Articles of Manufacture

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is a combination of target-binding molecules of the present invention or a protein complex of the present invention, or a target-binding molecule of the present invention as defined in [A-1] in combination with the other target-binding molecule as defined in [A-1] of the invention. The label or package insert indicates that the composition is used for treating the condition of choice. Moreover, the article of manufacture may comprise (a) a first container with a composition contained therein, wherein the composition comprises a combination of target-binding molecules of the present invention or the protein complex of the present invention, or the target-binding molecule of the present invention as defined in [A-1] in combination with the other target-binding molecule as defined in [A-1] of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### III. Examples

The following are examples of methods and compositions of the invention. It is understood that various other embodiments may be practiced, given the general description provided above.

### Example 1: The Concept of the Present Disclosure

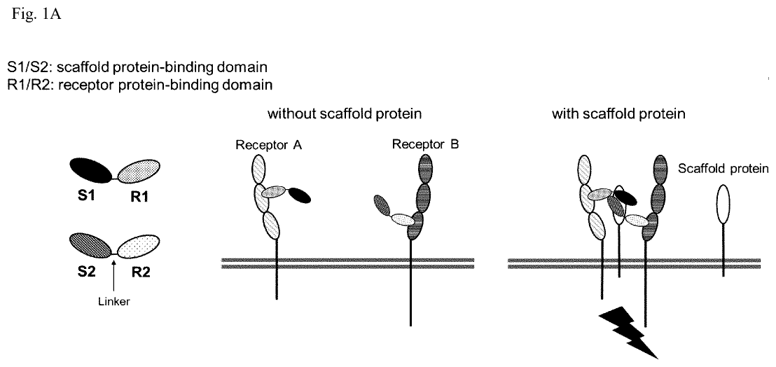

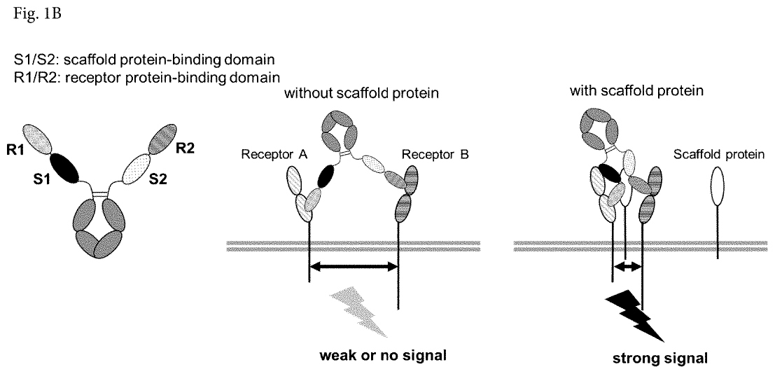

The schematic diagram of the protein complex in the present disclosure is illustrated in FIG. 1A. Conditional receptor signaling is achieved using a first target-binding molecule comprising a first binding domain that binds to a scaffold protein and a second binding domain that binds to a first receptor protein, and a second target-binding molecule, comprising a third binding domain that binds to the scaffold protein and a fourth binding domain that binds to a second receptor protein. The first and third binding domains of the target-binding molecules (S1 and S2) are capable of binding a scaffold protein simultaneously, non-competitively, in a biparatopic manner. The second binding domain (R1) binds the first receptor protein and fourth binding domain (R2) bind the second receptor protein (R1 binds Receptor A and R2 binds Receptor B), wherein Receptor A and Receptor B, when in proximity with one another or when associated with one another, can associate to form a receptor complex and induce receptor activation and/or signaling activity. Adding linker sequence between each binding domain is an option to optimize agonistic activity.

In the presence of receptor proteins and in the absence of scaffold protein, the two target-binding molecules only bind to their respective receptor proteins (Receptor A and Receptor B) through the first and third binding domain (R1 and R2), but since the receptor proteins (Receptor A and Receptor B) are not in proximity of one another or associated to one another, no signaling activity is induced. In the presence of both receptor receptor proteins and scaffold protein, the first and third binding domains of the target-binding molecules (S1 and S2) bind to the scaffold protein non-competitively and/or biparatopically, while the second binding domain binds the first receptor protein and fourth binding domain bind the second receptor protein. As a result, the receptor proteins are brought into proximity of one another or become associated with one another, thereby inducing receptor activation and/or signaling activity. Adding a linker sequence between each binding domain and a half-life extending domain, such as a Fc domain, is an option for improving functionality.

As a non-limiting example, this concept of conditional activation is illustrated using two VHH-VHH fusions. Each VHH-VHH fusion comprises a scaffold protein-binding domain and a receptor protein-binding domain. As a non-limiting example, SARS-CoV2 receptor binding protein (RBD) or human PDL1 may be the scaffold protein and IL-2R beta, IL-2R gamma or Wnt receptors may be the receptor proteins.

VHHs that are able to bind to RBD simultaneously in a biparatopic manner or human PDL1 simultaneously in a biparatopic manner, as well as VHHs that are able to bind IL-2R beta or IL-2R gamma were identified. Nb21 and Nb36 are VHHs that each recognize different epitopes of RBD and can therefore bind to RBD simultaneously in a biparatopic manner (Nature Communications 2021; 12: 4676).

FIG. 1B shows the possible single-molecular format that can induce conditional receptor activation by controlling receptor distance through binding to a scaffold protein. In the presence of receptor proteins and in the absence of scaffold protein, the target-binding molecule only bind to their respective receptor proteins (Receptor A and Receptor B) through the first and third binding domain (R1 and R2), but since the receptor proteins (Receptor A and Receptor B) are not in proximity of one another or associated to one another, only weak or no signaling activity is induced. In the presence of both receptor proteins and scaffold protein, the first and third binding domains of the target-binding molecule (S1 and S2) bind to the scaffold protein biparatopically, while the second binding domain binds the first receptor protein and fourth binding domain bind the second receptor protein. As a result, the receptor proteins are brought into proximity of one another or become associated with one another to form a receptor complex, thereby inducing receptor activation and/or signaling activity. Adding a linker sequence between each binding domain and a half-life extending domain, such as a Fc domain, is an option for improving functionality.

### Example 2: Preparation of Plasmids for VHH-VHH Fusions that Bind to RBD or PDL1 as the Scaffold Protein and Bind to IL-2R Beta or IL-2R Gamma as the Receptor Proteins

Each of the scaffold-binding proteins (VHHs that are able to bind to RBD or human PDL1) were connected to a VHH that is able to bind to human IL-2R beta or human IL-2R gamma with signal sequence to construct VHH-VHH fusions (SEQ ID NO: 1-10). RBD was fused with transmembrane and intracellular domain of human IL-2R alpha (RBD-IL2RA) with signal sequence (SEQ ID NO: 11) to be used as the scaffold protein. The DNA encoding VHH-VHH fusions, RBD-IL2RA or human PDL1 (SEQ ID NO: 12) were cloned into a mammalian expression vector.

Native SARS-CoV2 spike protein, which includes RBD, is a homo-trimer protein but RBD-IL2RA lacks the domain for trimer-formation and can be expressed as a monomer. Additionally, although IL2RA (IL-2R alpha) is the part of high affinity IL-2 receptor complex, it is not involved in IL-2R signal transduction (Cancer Communications 2018; 38: 62).

TABLE 1
SEQ ID NOS: 1 to 12

| SEQ ID NO | Abbreviation | Sequences |
|---|---|---|
| SEQ ID NO: 1 | Nb21-Vbh2g | MYRMQLLSCIALSLALVTNSAPTQVQLVESGGGLV |
| | | QAGGSLRLSCAVSGLGAHRVGWFRRAPGKEREEV |
| | | AAIGANGGNTNYLDSVKGRFTISRDNAKNTIYLQM |
| | | NSLKPQDTAVYYCAARDIETAEYTYWGQGTQVTV |
| | | SSGGGSQVQLQESGGGSVQAGGSLRLSCAASGYTY |
| | | RDYYMGWFRQAPGREREGVASIYTRGSREGSTRYS |
| | | SSVEGRFTITLDTAKNTLYLQMNSLKPEDTAMYYC |
| | | AADDRTWLPRVQLGGPRENEYNYWGQGTQVTVSS |
| | | GAPGSGLNDIFEAQKIEWHE |
| SEQ ID NO: 2 | Vhh2g-Nb21 | MYRMQLLSCIALSLALVTNSAPTQVQLQESGGGSV |
| | | QAGGSLRLSCAASGYTYRDYYMGWFRQAPGRERE |
| | | GVASIYTRGSREGSTRYSSSVEGRFTITLDTAKNTLY |
| | | LQMNSLKPEDTAMYYCAADDRTWLPRVQLGGPRE |
| | | NEYNYWGQGTQVTVSSGAPGSQVQLVESGGGLVQ |
| | | AGGSLRLSCAVSGLGAHRVGWFRRAPGKEREFVA |
| | | AIGANGGNTNYLDSVKGRFTISRDNAKNTIYLQMN |
| | | SLKPQDTAVYYCAARDIETAEYTYWGQGTQVTVSS |
| SEQ ID NO: 3 | Nb36-Vhh2b | MYRMQLLSCIALSLALVTNSAPVQLVESGGGLVQA |
| | | GGSLTLTCAASGRTFSSETMDMGWFRQAPGKEREF |
| | | VAADSWNDGSTYYADSVKGRFTISRDSAKNTLYLQ |
| | | MNSLKPEDTAVYYCAAETYSIYEKDDSWGYWGQG |
| | | TQVTVSGGGGSQVQLQESGGGSVQAGGSLRLSCA |
| | | ASSYTISSVCMGWFRQAPGKEREGVAGIAPDGSTG |
| | | YGDSVKGRFTISMLYDNAKNTLYLQMNSLKPEDTA |
| | | MYYCAAASPGRCFLPRTALEPALYYNWGQGTQVT |
| | | VSSGAPGSGLNDIFEAQKIEWE |
| SEQ ID NO: 4 | Vhh2b-Nb36 | MYRMQLLSCIALSLALVTNSAPQVQLQESGGGSVQ |
| | | AGGSLRLSCAASSYTISSVCMGWFRQAPGKEREGV |
| | | AGIAPDGSTGYGDSVKGRFTISMLYDNAKNTLYLQ |
| | | MNSLKPEDTAMYYCAAASPGRCFLPRTALEPALYY |
| | | NWGQGTQVTVSSGAPVQLVESGGGLVQAGGSLTL |
| | | TCAASGRTFSSETMDMGWFRQAPGKEREFVAADS |
| | | WNDGSTYYADSVKGRFTISRDSAKNTLYLQMNSL |
| | | KPEDTAVYYCAAETYSIYEKDDSWGYWGQGTQVT |
| | | VS |
| SEQ ID NO: 5 | Vhh2b-Nb21 | MYRMQLLSCIALSLALVTNSAPTQVQLQESGGGSV |
| | | QAGGSLRLSCAASSYTISSVCMGWFRQAPGKEREG |
| | | VAGIAPDGSTGYGDSVKGRFTISMLYDNAKNTLYL |
| | | QMNSLKPEDTAMYYCAAASPGRCFLPRTALEPALY |
| | | YNWGQGTQVTVSSGAPGSQVQLVESGGGLVQAGG |
| | | SLRLSCAVSGLGAHRVGWFRRAPGKEREFVAAIGA |
| | | NGGNTNYLDSVKGRFTISRDNAKNTIYLQMNSLKP |
| | | QDTAVYYCAARDIETAEYTYWGQGTQVTVSS |
| SEQ ID NO: 6 | Nb36-Vhh2g | MYRMQLLSCIALSLALVTNSAPVQLVESGGGLVQA |
| | | GGSLTLTCAASGRTFSSETMDMGWFRQAPGKEREF |
| | | VAADSWNDGSTYYADSVKGRFTISRDSAKNTLYLQ |
| | | MNSLKPEDTAVYYCAAETYSIYEKDDSWGYWGQG |
| | | TQVTVSGGGSQVQLQESGGGSVQAGGSLRLSCAAS |
| | | GYTYRDYYMGWFRQAPGREREGVASIYTRGSREG |
| | | STRYSSSVEGRFTITLDTAKNTLYLQMNSLKPEDTA |
| | | MYYCAADDRTWLPRVQLGGPRENEYNYWGQGTQ |
| | | VTVSSGAPGSGLNDIFEAQKIEWHE |
| SEQ ID NO: 7 | Vhh2g- | MYRMQLLSCIALSLALVTNSAPTQVQLQESGGGSV |
| | VhhPLI | QAGGSLRLSCAASGYTYRDYYMGWFRQAPGRERE |
| | | GVASIYTRGSREGSTRYSSSVEGRFTITLDTAKNTLY |
| | | LQMNSLKPEDTAMYYCAADDRTWLPRVQLGGPRE |
| | | NEYNYWGQGTQVTVSSQVQLQESGGGLVQPGGSL |
| | | RLSCAASGKMSSRRCMAWFRQAPGKERERVAKLL |
| | | TTSGSTYLADSVKGRFTISQNNAKSTVYLQMNSLK |
| | | PEDTAMYYCAADSEEDPTCTLVTSSGAFQYWGQG |
| | | TQVTVSS |
| SEQ ID NO: 8 | Vhh2b- | MYRMQLLSCIALSLALVTNSAPTQVQLQESGGGSV |
| | VlhPL1-12 | QAGGSLRLSCAASSYTISSVCMGWFRQAPGKEREG |
| | | VAGIAPDGSTGYGDSVKGRFTISMLYDNAKNTLYL |
| | | QMNSLKPEDTAMYYCAAASPGRCFLPRTALEPALY |
| | | YNWGQGTQVTVSSQVQLQESGGGSVQAGGSLRLS |
| | | CAASGYTRSSHCMVWFRQAPGKEREGVALIYTGSG |
| | | STYYADSVKGRFTISQDNAKKTLYLQMNSLKPEDT |
| | | AMYYCAAGTSSSSCPGLLGPPRYYNWGQGTQVTV |
| | | SS |
| SEQ ID NO: 9 | Vhh2b- | MYRMQLLSCIALSLALVTNSAPTQVQLQESGGGSV |
| | VhhPL1-50 | QAGGSLRLSCAASSYTISSVCMGWFRQAPGKEREG |
| | | VAGIAPDGSTGYGDSVKGRFTISMLYDNAKNTLYL |
| | | QMNSLKPEDTAMYYCAAASPGRCELPRTALEPALY |
| | | YNWGQGTQVTVSSQVQLQESGGGLVQPGGSLRLS |
| | | CIASGFTFSIMAMSWVRQAPGKGLEWVSTINSDGG |
| | | KTYYADSVKGRFTISRDNAKNTLYLQLNSLRTEDM |
| | | AMYYCRRCADIYCSGSGGWTGQGTQVTVSS |
| SEQ ID NO: 10 | Vhh2b- | MYRMQLLSCIALSLALVTNSAPTQVQLQESGGGSV |
| | VhhPL1 | QAGGSLRLSCAASSYTISSVCMGWFRQAPGKEREG |
| | | VAGIAPDGSTGYGDSVKGRFTISMLYDNAKNTLYL |
| | | QMNSLKPEDTAMYYCAAASPGRCFLPRTALEPALY |
| | | YNWGQGTQVTVSSQVQLQESGGGLVQPGGSLRLS |
| | | CAASGKMSSRRCMAWFRQAPGKERERVAKLLTTS |
| | | GSTYLADSVKGRFTISQNNAKSTVYLQMNSLKPED |
| | | TAMYYCAADSFEDPTCTLVISSGAFQYWGQGTQV |
| | | TVSS |
| SEQ ID NO: 11 | RBD-IL2RA | MDSYLLMWGLLTFIMVPGCQAGGGSTNLCPFGEVF |
| | | NATRFASVYAWNRKRISNCVADYSVLYNSASFSTF |
| | | KCYGVSPTKLNDLCFTNVYADSFVIRGDEVRQIAPG |
| | | QTGKIADYNYKLPDDFTGCVIAWNSNNLDSKVGG |
| | | NYNYLYRLFRKSNLKPFERDISTEIYQAGSTPCNGV |
| | | EGFNCYFPLQSYGFQPTNGVGYQPYRVVVLSFELL |
| | | HAPATVCGGGSYQVAVAGCVFLLISVLLLSGLTWQ |
| | | RRQRKSRRTI |
| SEQ ID NO: 12 | PDL1 | MRIFAVFIFMTYWHLLNAFTVTVPKDLYVVEYGSN |
| | | MTIECKFPVEKQLDLAALIVYWEMEDKNIIQFVHGE |
| | | EDLKVQHSSYRQRARLLKDQLSLGNAALQITDVKL |
| | | QDAGVYRCMISYGGADYKRITYKVNAPYNKINQRI |
| | | LVVDPVTSEHELTCQAEGYPKAEVIWTSSDHQVLS |
| | | GKTTTTNSKREEKLFNVTSTLRINTTTNEIFYCTFRR |
| | | LDPEENHTAELVIPELPLAHPPNERTHLVILGAILLC |
| | | LGVALTFIFRLRKGRMMDVKKCGIQDTNSKKQSDT |
| | | HLEET |

SEQ ID NO: 1 and 2 represent the fusion proteins of RBD binding domain (Nb21) and IL-2R gamma binding domain (Vhh2g). The binding of Nb21 to RBD does not compete with Nb36. The sequence of Vhh2g is underlined and the sequence of Nb21 is double underlined in Table 1.

SEQ ID NO: 3 and 4 represent the fusion proteins of RBD binding domain (Nb36) and IL-2R beta binding domain (Vhh2b). The binding of Nb36 to RBD does not compete with Nb21. The sequence of Vhh2b is underlined and the sequence of Nb36 is double underlined in Table 1.

SEQ ID NO: 5 represents the fusion protein of RBD binding domain (Nb21) and IL-2R beta binding domain (Vhh2b). The sequence of Vhh2b is underlined and the sequence of Nb21 is double underlined in Table 1.

SEQ ID NO: 7 represents the fusion protein of IL-2R gamma binding domain (Vhh2g) and PDL1 binding domain (VhhPL1). The sequence of Vhh2g is underlined and the sequence of VhhPL1 is double underlined in Table 1.

SEQ ID NO: 8 represents the fusion protein of IL-2R beta binding domain (Vhh2b) and PDL1 binding domain (VhhPL1-12). The sequence of Vhh2b is underlined and the sequence of VhhPL1-12 is double underlined in Table 1.

SEQ ID NO: 9 represents the fusion protein of IL-2R beta binding domain (Vhh2b) and PDL1 binding domain (VhhPL1-50). The sequence of Vhh2b is underlined and the sequence of VhhPL1-50 is double underlined in Table 1.

SEQ ID NO: 10 represents the fusion protein of IL-2R beta binding domain (Vhh2b) and PDL1 binding domain (VhhPL1). The sequence of Vhh2b is underlined and the sequence of VhhPL1 is double underlined in Table 1.

SEQ ID NO: 11 represents the fusion protein of SARS-CoV2 receptor binding domain (RBD) and transmembrane and intracellular domain of IL-2R alpha (IL-2RA).

SEQ ID NO: 12 represents human PDL1.

### Example 3: The Induction of IL-2 Receptor Signal by Mixtures of VHH-VHH Fusions, which Bind to RBD in Biparatopic Manner and IL-2 Receptor Proteins, is Dependent on RBD Expression

The conditional receptor activation in the presence of scaffold protein was demonstrated in this example using RBD as the scaffold protein and IL-2R beta and IL-2R gamma as conditionally activated receptor proteins.

As described in Example 2, VHH-VHH fusions were constructed by connecting a VHH that binds RBD (either Nb21 or Nb36) to a either a VHH that binds human IL-2R beta (Vhh2b) or a VHH that binds to human IL-2R gamma (Vhh2g). Nb21 and Nb36 are biparatopic antibodies that recognize different epitopes of RBD and can bind to RBD simultaneously (Nature Communications 2021; 12: 4676). Vhh2b (PDB: 7S2S) and Vhh2g (PDB: 7S2R) have been described (Cell 2022; 185: 1414-1430.e19) and their structure and binding epitope is shown in RCSB Protein Data Bank (7S2S and 7S2R).

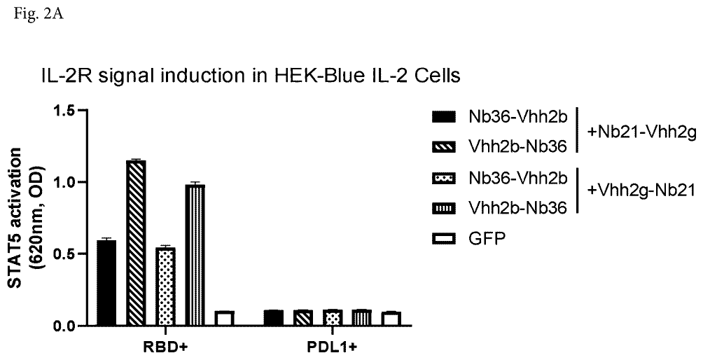

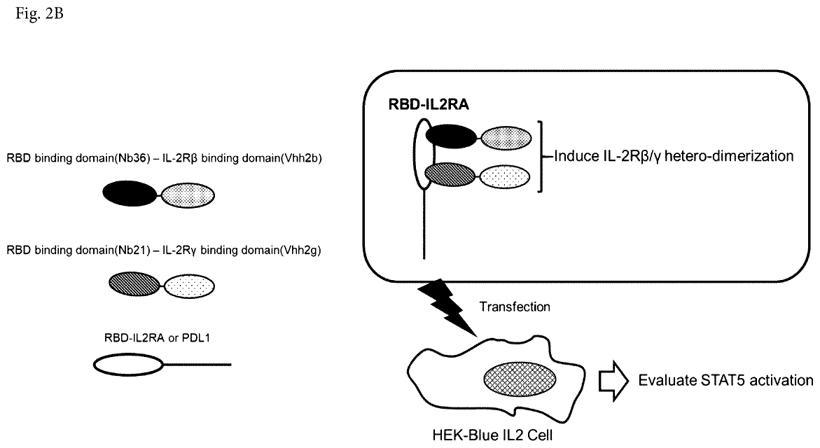

Plasmids for VHH-VHH fusions (SEQ ID NO: 1-4) were expressed in HEK-Blue IL-2 Cells (InvivoGen, #hkb-il2) by SF Cell Line 4D-Nucleofector X Kit L (Lonza, #V4XC-2024) with RBD-IL2RA (SEQ ID NO: 11) or PDL1 plasmid (SEQ ID NO: 12). pmaxGFP™ Vector was used as a negative control. Cells were cultured in Dulbecco's Modified Eagle Medium (DMEM) supplemented with 10% fetal bovine serum and 1% Penicillin-Streptomycin. These transfectants were seeded in 96-well plate for 3 days at 37 degrees C. in the presence of 5% CO₂. After 3 days culture, IL-2 receptor complex activation was evaluated using Quanti-Blue solution (InvivoGen, #rep-qbs) by measuring optical density at 620 nm, using Multiskan™ plate reader (FIG. 2A).

FIG. 2A shows the result of IL-2R signal induction. The VHH-VHH fusions were capable of activating IL-2 receptor complex in HEK-Blue IL-2 Cells in the presence of RBD-IL2RA, but not PDL1. This shows that IL-2R signal induction by the mixture of VHH-VHH fusions is dependent on the expression of the scaffold protein which is RBD. FIG. 2B shows the schematic diagram of experimental system for this example.

### Example 4: Expression and Purification of VHH-VHH Fusions that Bind to RBD and IL-2 Receptor Proteins

VHH-VHH fusions (SEQ ID NO: 1, 4, 5 and 6) were expressed transiently using Expi293 cell line (Thermo Fisher, Carlsbad, CA, USA). Antibody purification was carried out using Protein A affinity chromatography. For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4: 2411-2423).

### Example 5: The Activation of IL-2 Receptor Complex by Mixture of VHH-VHH Fusions, which Target RBD as the Scaffold Protein, is Dependent on Biparatopic Binding to RBD

To show that biparatopic binding to scaffold protein is a condition for IL-2R activation, the effect of combinations of VHH-VHH fusions which can bind the scaffold protein biparatopically was compared against combinations of VHH-VHH fusions which are unable to bind the scaffold protein biparatopically.

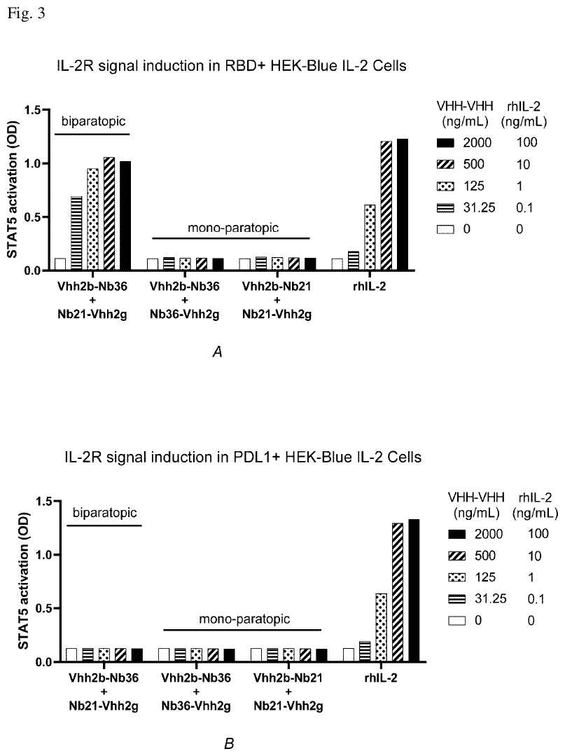

RBD-IL2RA (SEQ ID NO: 11) or PDL1 (SEQ ID NO: 12) was first expressed in HEK-Blue IL-2 Cells (InvivoGen, #hkb-il2) by electroporation using SF Cell Line 4D-Nucleofector X Kit L (Lonza, #V4XC-2024). After electroporation, these transfectants were seeded in 96-well plate and incubated overnight at 37 degrees C. in the presence of 5% CO₂. These cells were cultured in Dulbecco's Modified Eagle Medium (DMEM) supplemented with 10% fetal bovine serum and 1% Penicillin-Streptomycin. Day 1 post electroporation, cells were treated with different mixtures of recombinant VHH-VHH fusions (SEQ ID NO: 1, 4, 5 and 6) or recombinant human IL-2 (Peprotech, #200-02) and further incubated at 37 degrees C. in the presence of 5% CO₂ for another day. Day 2 post electroporation, IL-2 receptor complex activation was evaluated using Quanti-Blue solution (InvivoGen, #rep-qbs) by measuring optical density at 620 nm, using Multiskan™ plate reader (FIG. 3A and FIG. 3B).

As shown in FIGS. 3A and 3B, the combination of VHH-VHH fusions comprising biparatopic RBD antibodies, which is Vhh2b-Nb36 and Nb21-Vhh2g, could induce IL-2 receptor signaling in the presence of RBD expression. However, the combinations of mono-paratopic RBD antibodies, which are Vhh2b-Nb36 and Nb36-Vhh2g or Vhh2b-Nb21 and Nb21-Vhh2g, could not induce IL-2 receptor signal even in the presence of RBD. This result confirms that the activation of IL-2 receptor complex by mixture of VHH-VHH fusions is dependent on biparatopic binding to the scaffold protein that was RBD.

### Example 6: The Induction of IL-2 Receptor Complex Signal by Mixtures of VHH-VHH Fusions, which Bind to PDL1 and IL-2 Receptor Proteins, is Dependent on PDL1 Expression

The conditional activation of IL-2 receptor complex by mixtures of VHH-VHH fusions, which bind PDL1 as a target scaffold protein and IL-2 receptors proteins as target receptor proteins, was demonstrated in HEK-Blue IL-2 Cells. VHHs that bind PDL1 biparatopically were identified. VhhPL1 is a VHH that is able to bind PDL1 simultaneously with any one or VhhPL1-12 or VHHPL1-50 to achieve biparatopic binding.

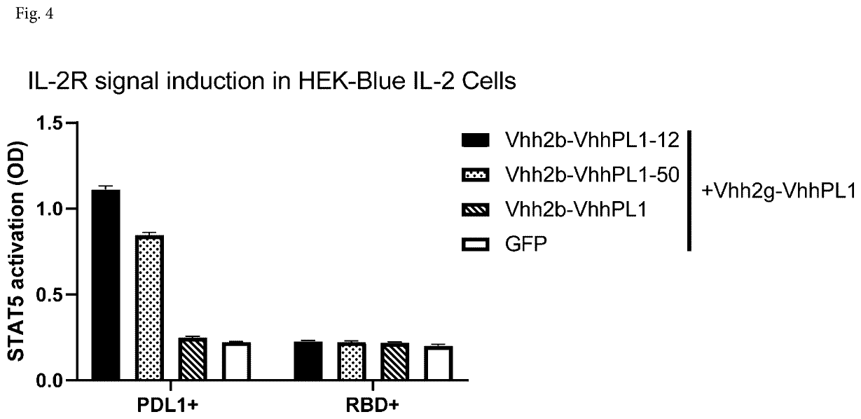

Plasmids for VHH-VHH fusions, which bind to PDL1 and human IL-2R beta or human IL-2R gamma (SEQ ID NO: 7-10), were expressed in HEK-Blue IL-2 Cells (InvivoGen, #hkb-il2) by SF Cell Line 4D-Nucleofector X Kit L (Lonza #V4XC-2024) with PDL1 (SEQ ID NO: 12) or RBD-IL2RA plasmid (SEQ ID NO: 11). The VHH-VHH fusion combinations to be tested were Vhh2g-VhhPL1 in combination with any one of Vhh2b-VhhPL1-12, Vhh2b-VhhPL1-50 or Vhh2b-VhhPL1. pmaxGFP™ Vector was used as a negative control. Cells were cultured in Dulbecco's Modified Eagle Medium (DMEM) supplemented with 10% fetal bovine serum and 1% Penicillin-Streptomycin. These transfectants were seeded in 96-well plate for 2 days at 37 degrees C. in the presence of 5% CO₂. After 2 days culture, IL-2 receptor complex activation was evaluated using Quanti-Blue solution (InvivoGen, #rep-qbs) by measuring optical density at 620 nm, using Multiskan™ plate reader (FIG. 4).

As shown in FIG. 4, the mixtures of VHH-VHH fusions that bind to PDL1 in a biparatopic manner and IL-2 receptor proteins can induce IL-2R signal on the condition of PDL1 expression. On the other hand, the mono-paratopic VHH-VHH fusion mixture, which is Vhh2b-VhhPL1 and Vhh2g-VhhPL1, could not induce clear IL-2R signal even in the presence of PDL1. This shows that IL-2R signal induction by mixtures of VHH-VHH fusions that target PDL1 and IL-2 receptor proteins is dependent on the expression of the scaffold protein that is PDL1 and biparatopic binding to this scaffold protein.

### Example 7: Preparation of Plasmids for Split Neo-2/15 Fused with VHHs which Bind to RBD

The present concept of conditional receptor signaling is compared with Split Neo-2/15, split versions of known IL-2/IL-15 mimetics that are active only upon colocalization of the two disjointed fragments at the site of the target cell (WO2020/106708A1).

Split Neo-2/15, which is Neo2A or Neo2B (WO2020/106708 A1), was fused to VHH that can bind to RBD with signal sequence and GS linker (SEQ ID NO: 13-15). The DNA encoding these fusion proteins were cloned into a mammalian expression vector.

TABLE 2
SEQ ID NOS: 13 to 15

| SEQ ID NO | Abbreviation | SEQUENCES |
|---|---|---|
| SEQ ID NO: 13 | Nb21-Neo2A | MYRMQLLSCIALSLALVTNSAPTQVQLVESGGGLVQAG |
| | | GSLRLSCAVSGLGAHRVGWFRRAPGKEREFVAAIGANG |
| | | GNTNYLDSVKGRFTISRDNAKNTIYLQMNSLKPQDTAV |
| | | YYCAARDIETAEYTYWGQGTQVTVSSGGGGSPKKKIQL |
| | | HAEHALYDALMILNIVKTNSGGGGSHHHHHHHH |
| SEQ ID NO: 14 | Nb21-Neo2B | MYRMQLLSCIALSLALVTNSAPTQVQLVESGGGLVQAG |
| | | GSLRISCAVSGLGAHRVGWERRAPGKEREFVAAIGANG |
| | | GNTNYLDSVKGRFTISRDNAKNTIYLQMNSLKPQDTAV |
| | | YYCAARDIETAEYTYWGQGTQVTVSSGGGSGGGSTNSP |
| | | PAEEKLEDYAFNFELILEEIARLFESGDQKDEAEKAKRM |
| | | KEWMKRIKTTASEDEQEEMANAIITILQSWIFSGGGGSH |
| | | HHHHHHH |
| SEQ ID NO: 15 | Nb36-Neo2A | MYRMQLLSCIALSLALVTNSAPVQLVESGGGLVQAGGS |
| | | LTLTCAASGRTFSSETMDMGWFRQAPGKEREFVAADS |
| | | WNDGSTYYADSVKGRFTISRDSAKNTLYLQMNSLKPED |
| | | TAVYYCAAETYSIYEKDDSWGYWGQGTQVTVSGGGSG |
| | | GGSPKKKIQLHAEHALYDALMILNIVKTNSGGGGSHHH |
| | | HHHHH |

SEQ ID NO: 13 represents the fusion protein of RBD binding domain (Nb21) and Neo2A of Split Noe-2/15. The sequence of Nb21 is underlined and the sequence of Neo2A is double underlined in Table 2.

SEQ ID NO: 14 represents the fusion protein of RBD binding domain (Nb21) and Neo2B of Split Neo-2/15. The sequence of Nb21 is underlined and the sequence of Neo2B is double underlined in Table 2.

SEQ ID NO: 15 represents the fusion protein of RBD binding domain (Nb36) and Neo2A of Split Neo-2/15. The sequence of Nb36 is underlined and the sequence of Neo2A is double underlined in Table 2.

### Example 8: The Combination of Vhh2b-Nb36 and Nb21-Vhh2g Demonstrated Superior Selectivity Compared to Split Neo-2/15

The activation of IL-2 receptor complex by the mixture of Vhh2b-Nb36 and Nb21-Vhh2g showed superior selectivity on RBD expression than the fusion proteins of Split Neo-2/15 and RBD binding domain.

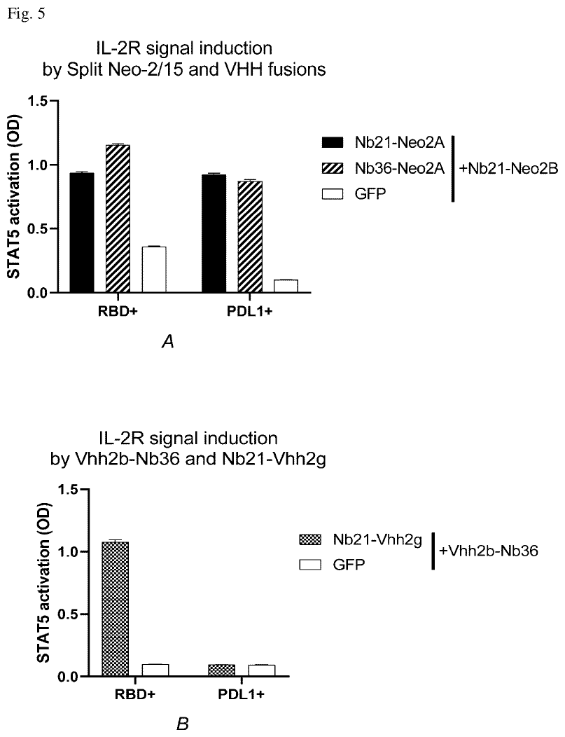

To prevent unintended heterodimer formation of Split Neo-2/15 within endosome of expressing cells, plasmids for Split Neo-2/15, which were conjugation of VHH that can bind to RBD and Neo2A or Neo2B (SEQ ID NO: 13-15), were expressed individually in HEK-Blue IL-2 Cells (InvivoGen, #hkb-il2) either with RBD-IL2RA (SEQ ID NO: 11) or PDL1 plasmid (SEQ ID NO: 12) by SF Cell Line 4D-Nucleofector X Kit L (Lonza, #V4XC-2024). To match this condition, plasmids of Vhh2b-Nb36 or Nb21-Vhh2g (SEQ ID NO:1 and 4) were expressed individually in HEK-Blue IL-2 Cells either with RBD-IL2RA or PDL1 plasmid. pmaxGFP™ Vector was used as a negative control. After electroporation, these transfectants were mixed in equal volume with different combinations and seeded in 96-well plate for 2 days at 37 degrees C. in the presence of 5% CO₂. These mixture of transfectants were cultured in Dulbecco's Modified Eagle Medium (DMEM) supplemented with 10% fetal bovine serum and 1% Penicillin-Streptomycin. After 2 days culture, IL-2 receptor complex activation was evaluated using Quanti-Blue solution (InvivoGen, #rep-qbs) by measuring optical density at 620 nm, using Multiskan™ plate reader (FIGS. 5A and 5B).

As shown in FIG. 5A, the conjugations of Split-Neo2/15 and RBD binding domain showed limited dependency on RBD expression. On the other hand, the mixture of Vhh2b-Nb36 and Nb21-Vhh2g, which bind to RBD in a biparatopic manner, demonstrated complete dependency on RBD expression (FIG. 5B). Therefore, the present concept of conditional receptor signaling has superior selectivity over Split Neo-2/15, a known method in the art.

### Example 9: Preparation of Plasmids for VHH-VHH Fusions that Bind to TNF Alpha as a Target Scaffold Protein and Bind to IL-2R Beta or IL-2R Gamma as Target Receptor Proteins

The present concept of conditional receptor signaling is further exemplified using TNF alpha as the scaffold protein and IL-2R beta or IL-2R gamma as target receptor proteins.

The scaffold-binding protein, which is Vhhtnfa3, was connected to another VHH that is able to bind to human IL-2R beta or human IL-2R gamma with signal sequence to construct VHH-VHH fusions (SEQ ID NO: 16-19). The DNA encoding these VHH-VHH fusions or human TNF alpha (SEQ ID NO: 20) was cloned into a mammalian expression vector.

TABLE 3
SEQ ID NOS: 16 to 20

| SEQ ID NO | Abbreviation | SEQUENCES |
|---|---|---|
| SEQ ID NO: 16 | Vhhtnfa3-Vhh2b | MYRMQLLSCIALSLALVTNSAPTQVQLQESGGGLVQ |
| | | PGGSLRLSCAASGRTFSDHSGYTYTIGWFRQAPGKER |
| | | EFVARIYWSSGNTYYADSVKGREAISRDIAKNTVDLT |
| | | MNNLEPEDTAVYYCAARDGIPTSRSVESYNYWGQG |
| | | TQVTVSSGGSQVQLQESGGGSVQAGGSLRLSCAASS |
| | | YTISSVCMGWFRQAPGKEREGVAGIAPDGSTGYGDS |
| | | VKGRFTISMLYDNAKNTLYLQMNSLKPEDTAMYYC |
| | | AAASPGRCELPRTALEPALYYNWGQGTQVTVSS |
| SEQ ID NO: 17 | Vhh2b-Vhhtnfa3 | MYRMQLLSCIALSLALVTNSAPTQVQLQESGGGSVQ |
| | | AGGSLRLSCAASSYTISSVCMGWFRQAPGKEREGVA |
| | | GIAPDGSTGYGDSVKGRFTISMLYDNAKNTLYLQMN |
| | | SLKPEDTAMYYCAAASPGRCFLPRTALEPALYYNWG |
| | | QGTQVTVSSGGSQVQLQESGGGLVQPGGSLRLSCAA |
| | | SGRTFSDHSGYTYTIGWFRQAPGKEREFVARIYWSSG |
| | | NTYYADSVKGRFAISRDIAKNTVDLTMNNLEPEDTA |
| | | VYYCAARDGIPTSRSVESYNYWGQGTQVTVSS |
| SEQ ID NO: 18 | Vhhtnfa3-Vhh2g | MYRMQLLSCIALSLALVTNSAPTQVQLQESGGGLVQ |
| | | PGGSLRLSCAASGRTFSDHSGYTYTIGWFRQAPGKER |
| | | EFVARIYWSSGNTYYADSVKGREAISRDIAKNTVDLT |
| | | MNNLEPEDTAVYYCAARDGIPTSRSVESYNYWGQG |
| | | TQVTVSSGGSQVQLQESGGGSVQAGGSLRLSCAASG |
| | | YTYRDYYMGWFRQAPGREREGVASIYTRGSREGSTR |
| | | YSSSVEGRFTITLDTAKNTLYLQMNSLKPEDTAMYY |
| | | CAADDRTWLPRVQLGGPRENEYNYWGQGTQVTVSS |
| SEQ ID NO: 19 | Vhh2g-Vhhtnfa3 | MYRMQLLSCIALSLALVTNSAPTQVQLQESGGGSVQ |
| | | AGGSLRLSCAASGYTYRDYYMGWFRQAPGREREGV |
| | | ASIYTRGSREGSTRYSSSVEGRFTITLDTAKNTLYLQ |
| | | MNSLKPEDTAMYYCAADDRTWLPRVQLGGPRENEY |
| | | NYWGQGTQVTVSSGGSQVQLQESGGGLVQPGGSLR |
| | | LSCAASGRTFSDHSGYTYTIGWFRQAPGKEREFVARI |
| | | YWSSGNTYYADSVKGREAISRDIAKNTVDLTMNNLE |
| | | PEDTAVYYCAARDGIPTSRSVESYNYWGQGTQVTVS |
| | | S |
| SEQ ID NO: 20 | TNFα | MSTESMIRDVELAEEALPKKTGGPQGSRRCLFLSLFS |
| | | FLIVAGATTLFCLLHFGVIGPQREEFPRDLSLISPLAQA |
| | | VRSSSRTPSDKPVAHVVANPQAEGQLQWLNRRANA |
| | | LLANGVELRDNQLVVPSEGLYLIYSQVLFKGQGCPST |
| | | HVLLTHTISRIAVSYQTKVNLLSAIKSPCQRETPEGAE |
| | | AKPWYEPIYLGGVFQLEKGDRLSAEINRPDYLDFAES |
| | | GQVYEGIIAL |

SEQ ID NO: 16 and 17 represent the fusion proteins of TNF alpha binding domain (Vhhtnfa3) and IL-2R beta binding domain (Vhh2b). The sequence of Vhh2b is underlined and the sequence of Vhhtnfa3 is double underlined in Table 3.

SEQ ID NO: 18 and 19 represent the fusion proteins of TNF alpha binding domain (Vhhtnfa3) and IL-2R gamma binding domain (Vhh2g). The sequence of Vhh2g is underlined and the sequence of Vhhtnfa3 is double underlined in Table 3.

SEQ ID NO: 20 represents human TNF alpha.

### Example 10: IL-2R Signal Induction by Mixtures of VHH-VHH Fusions that can Bind to TNF Alpha and IL-2 Receptor Complex could be Induced by Mono-Paratopic Binding to TNF Alpha, which is a Trimeric Antigen

Because TNF alpha is a homo-trimeric antigen, the VHH-VHH fusions may each bind to the same epitope on each TNF alpha monomer (i.e., mono-paratopic binding). When TNF alpha monomers form a trimer, the scaffold protein-binding domains each bind to a TNF alpha monomer and be in close proximity to one another. The receptor protein-binding domains connected to each scaffold protein-binding domain will therefore also be able to bring the receptor proteins into proximity of one another, allowing the induction of receptor activation and/or signaling activity. Exemplary VHH-VHH fusions of Vhh2b connected to Vhhtnfa3 and Vhh2g connected to Vhhtnfa3 were constructed.

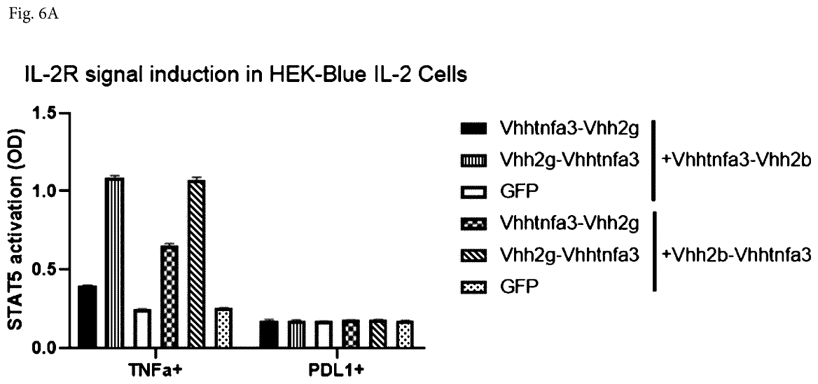

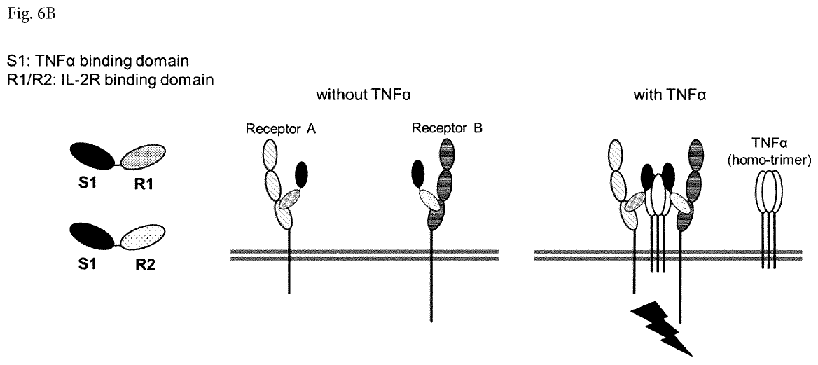

Plasmids with VHH-VHH fusions, which bind to TNF alpha as a target scaffold protein and human IL-2R beta or human IL-2R gamma as target receptor proteins (SEQ ID NO: 16-19), were expressed in HEK-Blue IL-2 Cells (InvivoGen, #hkb-il2) by SF Cell Line 4D-Nucleofector X Kit L (Lonza #V4XC-2024) with TNFa (SEQ ID NO: 20) or PDL1 (SEQ ID NO: 12). pmaxGFP™ Vector was used as a negative control. Cells were cultured in Dulbecco's Modified Eagle Medium (DMEM) supplemented with 10% fetal bovine serum and 1% Penicillin-Streptomycin. These transfectants were seeded in 96-well plate for 2 days at 37 degrees C. in the presence of 5% CO₂. After 2 days culture, IL-2 receptor complex activation was evaluated using Quanti-Blue solution (InvivoGen, #rep-qbs) by measuring optical density at 620 nm, using Multiskan™ plate reader (FIG. 6A).

As shown in FIG. 6A, the mono-paratopic TNF alpha-binding domains targeting the same epitope on monomers of a TNF alpha trimer could induce IL-2 receptor signal in the presence of TNF alpha. The schematic diagram for the mechanism of conditional IL-2R signal induction by the mixture VHH-VHH fusions that can bind to IL-2R beta or IL-2R gamma and TNF alpha in mono-paratopic manner was shown in FIG. 6B.

### Example 11: Expression and Purification of VHH-VHH or VHH-scFv Fusions that Bind to RBD and Wnt Receptors that are LRPs or FZDs

VHH that can bind to RBD (Nb21 or Nb36) was fused to another receptor protein-binding domain, which may comprise one or more VHH, or scFv that is able to bind to human lipoprotein receptor-related proteins (LRPs) or human Frizzled receptors (FZDs) (Vhhlrp36 and R2M3 respectively) with signal sequence (SEQ ID NO: 21-23). The DNA encoding these fusion proteins were cloned into a mammalian expression vector. These plasmids were expressed transiently using Expi293 cell line (Thermo Fisher, Carlsbad, CA, USA). Antibody purification was carried out using Protein A affinity chromatography. For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 4
SEQ ID NOS: 21 to 23

| SEQ ID NO | Abbreviation | SEQUENCES |
|---|---|---|
| SEQ ID NO: 21 | Vhhlrp36- | MYRMQLLSCIALSLALVTNSAPTQVKLEESGGGLVQ |
| | Vhhlrp36-Nb21 | AGGSLRLSCAASGRIFSIYDMGWFRQAPQKEREFVSG |
| | | IRWSGGTSYADSVKGRFTISKDNAKNTIYLQMNNLK |
| | | AEDTAVYYCGSRGYWGQGTLVTVSSGSGSQVKLEE |
| | | SGGQLVQAGGSLRLSCAASGRIFSIYDMGWFRQAPG |
| | | KEREFVSGIRWSGGTSYADSVKGRFTISKDNAKNTIY |
| | | LQMNNLKAEDTAVYYCGSRGYWGQGTLVTVSSGGS |
| | | GGSQVQLVESGGGLVQAGGSLRLSCAVSGLGAHRV |
| | | GWERRAPGKEREFVAAIGANGGNTNYLDSVKGRFTI |
| | | SRDNAKNTIYLQMNSLKPQDTAVYYCAARDIETAEY |
| | | TYWGQGTQVTVSSGGSHHHHHHHH |
| SEQ ID NO: 22 | Vhhlrp36- | MYRMQLLSCIALSLALVTNSAPTQVKLEESGGGLVQ |
| | Vhhlrp36-Nb36 | AGGSLRLSCAASGRIFSIYDMGWFRQAPGKEREFVSG |
| | | IRWSGGTSYADSVKGRFTISKDNAKNTIYLQMNNLK |
| | | AEDTAVYYCGSRGYWGQGTLVTVSSGSGSQVKLEE |
| | | SGGGLVQAGGSLRLSCAASGRIFSIYDMGWFRQAPG |
| | | KEREFVSGIRWSGGTSYADSVKGRFTISKDNAKNTIY |
| | | LQMNNLKAEDTAVYYCGSRGYWGQGTLVTVSSGGS |
| | | VQLVESGGGLVQAGGSLTLICAASGRTESSETMDMG |
| | | WFRQAPGKEREFVAADSWNDGSTYYADSVKGRFTIS |
| | | RDSAKNTLYLQMNSLKPEDTAVYYCAAETYSIYEKD |
| | | DSWGYWGQGTQVTVSGGSHHHHHHHH |
| SEQ ID NO: 23 | R2M3-Nb21 | MYRMQLLSCIALSLALVTNSAPTGQAVVLQEPSLSVS |
| | | PGGTVTLTCGLSSGSVSTNYYPSWYQQTPGQAPRTLI |
| | | YYTNTRSSDVPERFSGSIVGNKAALTITGAQPDDESV |
| | | YFCLLYLGRGIWVFGGGTKLTVLGQPKGGGGSGGG |
| | | GSGGGGSEVQLVQSGAEVKKPGASVKVSCKASGYT |
| | | FTSYGISWVRQAPGQGLEWMGWISAYNGNTNYAQK |
| | | LQGRVTMTTDTSTSTAYMELRSLRSDDTAVYYCASS |
| | | KEKATYYYGMDVWGQGTTVTVSSGGSGGSQVQLV |
| | | ESGGGLVQAGGSLRLSCAVSGLGAHRVGWFRRAPG |
| | | KEREFVAAIGANGGNTNYLDSVKGRFTISRDNAKNTI |
| | | YLQMNSLKPQDTAVYYCAARDIETARYTYWGQGTQ |
| | | VTVSSGGSHHHHHHHH |

SEQ ID NO: 21 represents the fusion protein of two LRP binding domain (Vhhlrp36) and RBD binding domain (Nb21). The sequence of Vhhlrp36 is underlined and the sequence of Nb21 is double underlined in Table 4.

SEQ ID NO: 22 represents the fusion protein of two LRP binding domains (Vhhlrp36) and RBD binding domain (Nb36). The sequence of Vhhlrp36 is underlined and the sequence of Nb36 is double underlined in Table 4.

SEQ ID NO: 23 represents the fusion protein of FZD binding domain (R2M3) and RBD binding domain (Nb21). The sequence of R2M3 is underlined and the sequence of Nb21 is double underlined in Table 4.

### Example 12: The Activation of Wnt Receptor Complex by Mixture of VHH-VHH and VHH-scFv Fusions is Dependent on Biparatopic Binding to RBD

The present concept of conditional receptor signaling is further exemplified using RBD as the scaffold protein and Wnt receptors, which comprise LRP and FZD, as conditionally activated receptor proteins. This example also shows that other binding proteins, such as scFv, can also be used as the scaffold protein-binding domain or receptor protein-binding domain.

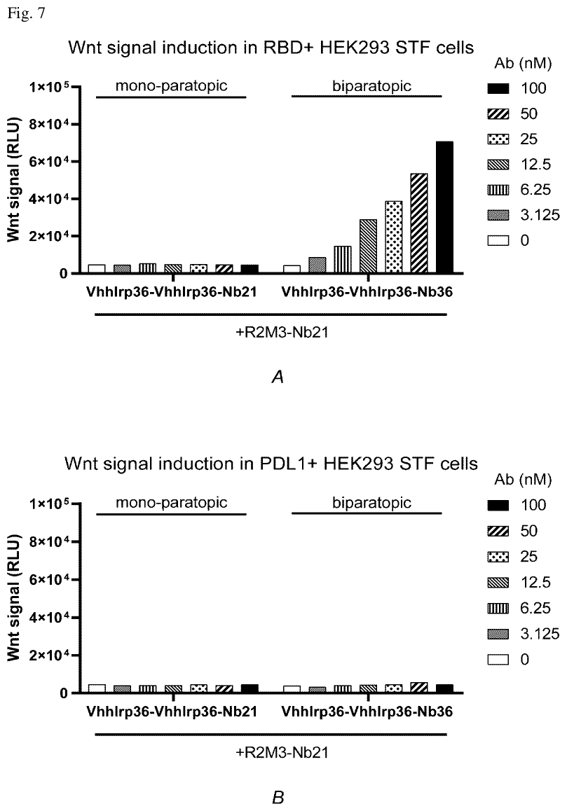

RBD-IL2RA (SEQ ID NO: 11) or PDL1 (SEQ ID NO: 12) was first expressed in HEK293 STF cells (ATCC, CRL-3249) by electroporation using SF Cell Line 4D-Nucleofector X Kit L (Lonza, #V4XC-2024). These transfectants were seeded in white 96-well plate and incubated overnight at 37 degrees C./5% CO₂ after addition of 200 nM RSPO1 protein (in-house, #PPU5200) and 50 nM LGK974 (Cayman chemical, #14072). These cells were cultured in Dulbecco's Modified Eagle Medium (DMEM) F-12, HEPES supplemented with 20% fetal bovine serum and 200 micro g/mL Geneticin. Day 1 post electroporation, these cells were treated with different mixtures of VHH-VHH or VHH-scFv fusions (SEQ ID NO: 21-23) that bind to RBD and Wnt receptor proteins (FZDs and LRPs) and further incubated at 37 degrees C./5% CO₂ for another day. Day 2 post electroporation, Wnt signal activation was evaluated using ONE-Glo Luciferase Assay System (Promega, #E6120) by measuring luminescence using Glomax Explorer (FIGS. 7A and 7B).

As shown in FIGS. 7A and 7B, only the combination of R2M3-Nb21 and Vhhlrp36-Vhhlrp36-Nb36 could induce Wnt signal in the presence of RBD expression. Consistent with the present concept of conditional receptor signaling, only when both Nb21 and Nb36 bind RBD in a biparatopic manner, the LRP-binding domain (Vhhlrp36) and FZD-binding domain (R2M3) could bind their respective antigens, and bring them into close proximity to induce Wnt receptor signaling. In contrast, Wnt receptor signaling was not observed for mono-paratopic VHH-VHH combinations where both VHH-VHH fusions comprise Nb21 (FIG. 7A), and in the absence of the scaffold protein that is RBD (FIG. 7B). The activation of Wnt receptor signaling by the mixture of VHH-VHH and VHH-scFv fusion, which bind to RBD and Wnt receptor proteins, is therefore dependent on RBD expression and biparatopic binding to RBD.

### Example 13: Expression and Purification of Vhh2b-Nb36/Nb21-Vhh2g Fc Fusion Protein

The present concept of conditional receptor activation may also be achieved in a single-molecule format that was described in FIG. 1B. The two target-binding molecules may be linked to form a single-molecule, or be associated to one another as a protein complex, though one or more domains that may extend the half-life of the molecule. In an example, the VHH-VHH fusions may be connected to a Fc domain through knobs-into-holes engineering (Protein Engineering 1996; 9: 617).

The VHH-VHH fusions Vhh2b-Nb36 (SEQ ID: 4) or Nb21-Vhh2g (SEQ ID: 1) were fused with Fc that carried mutations for facilitating the hetero-dimerization of Vhh2b-Nb36 and Nb21-Vhh2g. The DNA encoding these Fc fusion proteins were cloned into a mammalian expression vector (SEQ ID: 24 and 25). These plasmids were expressed transiently using Expi293 cell line (Thermo Fisher, Carlsbad, CA, USA). Antibody purification was carried out using Protein A affinity chromatography. For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 5
SEQ ID NOS: 24 and 25

| SEQ ID NO | Abbreviation | SEQUENCES |
|---|---|---|
| SEQ ID NO: 24 | Vhh2b-Nb36 Fc | MGWSCIILFLVATATGVHSQVQLQESGGGSVQAGGS |
| | fusion | LRLSCAASSYTISSVCMGWFRQAPGKEREGVAGIAP |
| | | DGSTGYGDSVKGRFTISMLYDNAKNTLYLQMNSLKP |
| | | EDTAMYYCAAASPGRCFLPRTALEPALYYNWGQGT |
| | | QVTVSSGAPVQLVESGGGLVQAGGSLTLTCAASGRT |
| | | FSSETMDMGWFRQAPGKEREFVAADSWNDGSTYYA |
| | | DSVKGRFTISRDSAKNTLYLQMNSLKPEDTAVYYCA |
| | | AETYSIYEKDDSWGYWGQGTQVTVSDKTHTCPPCP |
| | | APELRRGPSVFLFPPKPKDTLMISRTPEVTCVVVDVS |
| | | HEDPEVKFNWYVDGVEVHNAKTKPREEQYNSTYRV |
| | | VSVLTVLHQDWLNGKEYKCKVSNKALPAPIEKTISK |
| | | AKGQPREPQVYTLPPSREEMTCNQVSLTCLVKGWYP |
| | | SDIAVEWESNGQPENNYKTTPPVLDSDGSFELYSKLT |
| | | VDKSRWQQGNVESCSVMHEALHNHYTQKSLSLSP |
| SEQ ID NO: 25 | Nb21-Vhh2g Fc | MGWSCIILFLVATATGVHSQVQLVESGGGLVQAGGS |
| | fusion | LRLSCAVSGLGAHRVGWFRRAPGKEREFVAAIGANG |
| | | GNTNYLDSVKGRFTISRDNAKNTIYLQMNSLKPQDT |
| | | AVYYCAARDIETAEYTYWGQGTQVTVSSGGGSQVQ |
| | | LQESGGGSVQAGGSLRLSCAASGYTYRDYYMGWFR |
| | | QAPGREREGVASIYTRGSREGSTRYSSSVEGRFTITLD |
| | | TAKNTLYLQMNSLKPEDTAMYYCAADDRTWLPRV |
| | | QLGGPRENEYNYWGQGTQVTVSSGAPGSGLNDIFEA |
| | | QKIEWHEDKTHTCPPCPAPELRRGPSVFLFPPKPKDT |
| | | LMISRTPEVTCVVVDVSHEDPEVKFNWYVDGVEVH |
| | | NAKTKPREEQYNSTYRVVSVLTVLHQDWLNGKEYK |
| | | CKVSNKALPAPIEKTISKAKGQPREPQVYTLPPSCEE |
| | | MIKNQVSLTCLVKGSYASDIAVEWESNGQPENNYK |
| | | TTPPVLDSDGSFFLYSKLTVVKSRWQQGNVESCSVM |
| | | HEALHNHYTQKSLSLSP |

SEQ ID NO: 24 represents the Fe fusion of Vhh2b-Nb36 for preparing Vhh2b-Nb36/Nb21-Vhh2g Fe hetero-dimer. The sequence of Vhh2b-Nb36 is underlined and the sequence of Fe is double underlined in Table 5.

SEQ ID NO: 25 represents the Fe fusion of Nb21-Vhh2g for preparing Vhh2b-Nb36/Nb21-Vhh2g Fe hetero-dimer. The sequence of Nb21-Vhh2g is underlined and the sequence of Fe is double underlined in Table 5.

### Example 14: Vhh2b-Nb36/Nb21-Vhh2g Fe Fusion Protein can Induce Stronger IL-2 Receptor Signaling in the Presence of RBD Compared to in the Absence of RBD

The activation of IL-2 receptor complex by hetero-dimeric Fe fusion of Vhh2b-Nb36 and Nb21-Vhh2g was stronger in the presence of RBD compared to PDL1 expression in HEK-Blue IL-2 Cells.

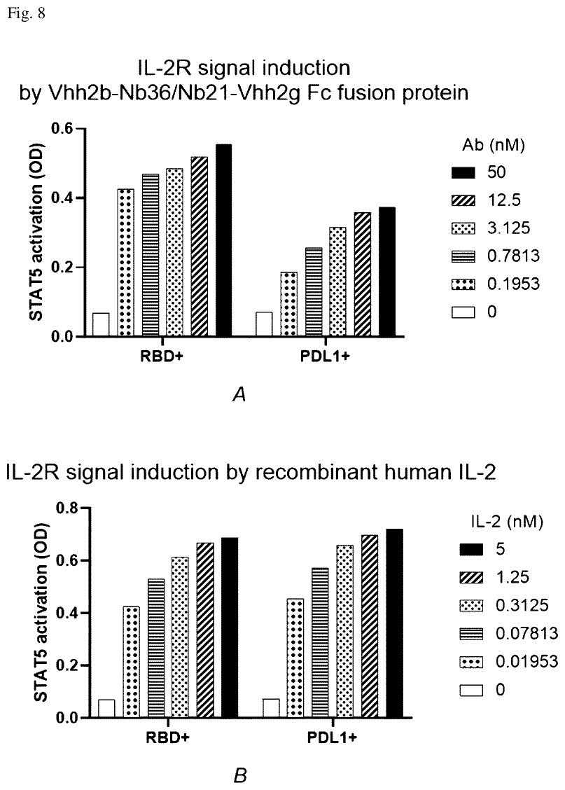

RBD-IL2RA (SEQ ID NO: 11) or PDL1 (SEQ ID NO: 12) was first expressed in HEK-Blue IL-2 Cells (InvivoGen, #hkb-il2) by electroporation using SF Cell Line 4D-Nucleofector X Kit L (Lonza, #V4XC-2024). After electroporation, these transfectants were seeded in 96-well plate and incubated overnight at 37 degrees C. in the presence of 5% CO₂. These cells were cultured in Dulbecco's Modified Eagle Medium (DMEM) supplemented with 10% fetal bovine serum and 1% Penicillin-Streptomycin. Day 1 post electroporation, cells were treated with Vhh2b-Nb36/Nb21-Vhh2g Fc fusion protein (the hetero-dimer of SEQ ID: 24 and 25) or recombinant human IL-2 (Peprotech, #200-02) and further incubated at 37 degrees C. in the presence of 5% CO₂ for another day. Day 2 post electroporation, IL-2 receptor complex activation was evaluated using Quanti-Blue solution (InvivoGen, #rep-qbs) by measuring optical density at 620 nm, using Multiskan™ plate reader (FIGS. 8A and 8B).

Vhh2b-Nb36/Nb21-Vhh2g Fc fusion protein induced stronger IL-2 receptor complex signal in the presence of RBD compared to in the absence of it (FIG. 8A). Unlike conventional conjugation of recombinant IL-2 and targeting antibody (Nature 2022; 610: 161-172), Vhh2b-Nb36/Nb21-Vhh2g Fc fusion showed not only stronger IL-2R signal from lower concentration but also it induced higher maximum STAT5 activation when it compared in the presence and absence of RBD. This shows that the expression of the scaffold protein RBD is a condition for strong IL-2 receptor complex signal induction using the Vhh2b-Nb36/Nb21-Vhh2g Fc fusion protein. On the other hand, the direct administration of the IL-2R ligand, recombinant human IL-2, did not show any selectivity between RBD- and PDL1-expressing HEK-Blue IL-2 Cells (FIG. 8B).

### Example 15: Expression and Purification of VHH-scFv Fusions or IL-2 Mutant that Bind to IL2R Alpha as the Scaffold Protein and IL-2R Beta and IL-2R Gamma as the Receptor Proteins

IL2_RETR, which is IL-2 mutant, was fused with His tag sequence (SEQ ID NO: 26). VHH that can bind to human IL-2R gamma (Vhh2g) was fused with BT942 scFv (SEQ ID NO: 27) or Dac scFv (SEQ ID NO:28), which are IL-2R alpha binding domains. VHH that can bind to human IL-2R beta (Vhh2b) was connected with BT942 scFv (SEQ ID NO: 29). The DNA encoding these proteins were cloned into a mammalian expression vector. These plasmids were expressed transiently using Expi293 cell line (Thermo Fisher, Carlsbad, CA, USA). Protein purification was carried out using Protein A or immobilized metal ion affinity chromatography. For the concentration of the purified proteins, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 6
SEQ ID NOS: 26 to 29

| SEQ ID NO | Abbreviation | Sequences |
|---|---|---|
| SEQ ID NO: 26 | IL2_RETR | MYRMQLLSCIALSLALVTNSAPTSSSTKKTQLQLEH |
| | | LRLDLEMILNGINNYKNPKLTRMLTFKFYMPKKAT |
| | | ELKHLQCLEEELKPLEEVLNLAQSKNFHLRPRDLIS |
| | | NINVIVLELKGSETTFMCEYADETATIVEFLNRWITF |
| | | CTSIIRTLTGGSHHHHHHHH |
| SEQ ID NO: 27 | Vhh2g-BT942 scFv | MGWSCIILFLVATATGVHSQVQLQESGGGSVQAGG |
| | | SLRLSCAASGYTYRDYYMGWFRQAPGREREGVASI |
| | | YTRGSREGSTRYSSSVEGRFTITLDTAKNTLYLQMN |
| | | SLKPEDTAMYYCAADDRTWLPRVQLGGPRENEYN |
| | | YWGQGTQVTVSSDIQMTQSPDSLAVSLGERATINC |
| | | KSSQSVLYSSNNKNYLAWYQQKPGQPPKLLIYWAS |
| | | TRESGVPDRFSGSGSGTDFTLTISSLQAEDVAVYYC |
| | | QQYYSTPYTFGQGTKVEIKRTVGGGGSGGGGSGGG |
| | | GSQVQLVQSGAEVKKPGSSVKVSCKASGGTFSSDA |
| | | INWVRQAPGQGLEWMGRIIPIFGVADYAQKFQGRV |
| | | TLTADKSTSTAYMDLSSLRSEDTAVFYCARERGDY |
| | | SNFWYFDLWGRGTLVTVSSGGSHHHHHHHH |
| SEQ ID NO: 28 | Vhh2g-Dac scFv | MGWSCIILFLVATATGVHSQVQLQESGGGSVQAGG |
| | | SLRLSCAASGYTYRDYYMGWFRQAPGREREGVASI |
| | | YTRGSREGSTRYSSSVEGRFTITLDTAKNTLYLQMN |
| | | SLKPEDTAMYYCAADDRTWLPRVQLGGPRENEYN |
| | | YWGQGTQVTVSSDIQMTQSPSTLSASVGDRVTITCS |
| | | ASSSISYMHWYQQKPGKAPKLLIYTTSNLASGVPA |
| | | RFSGSGSGTEFTLTISSLQPDDFATYYCHQRSTYPLT |
| | | FGQGTKVEVKRTVGGGGGGGGSGGGGSQVQLVQ |
| | | SGAEVKKPGSSVKVSCKASGYTFTSYRMHWVRQA |
| | | PGQGLEWIGYINPSTGYTEYNQKFKDKATITADEST |
| | | NTAYMELSSLRSEDTAVYYCARGGGVEDYWGQGT |
| | | LVTVSSGGSHHHHHHHH |
| SEQ ID NO: 29 | Vhh2b-BT942 scFv | MGWSCIILFLVATATGVHSQVQLQESGGGSVQAGG |
| | | SLRLSCAASSYTISSVCMGWFRQAPGKEREGVAGIA |
| | | PDGSTGYGDSVKGRFTISKDNAKNTLYLQMNSLKP |
| | | EDTAMYYCAAASPGRCFLPRTALEPALYYNWGQG |
| | | TQVTVSSDIQMTQSPDSLAVSLGERATINCKSSQSV |
| | | LYSSNNKNYLAWYQQKPGQPPKLLIYWASTRESGV |
| | | PDRFSGSGSGTDFTLTISSLQAEDVAVYYCQQYYST |
| | | PYTFGQGTKVEIKRTVGGGGSGGGGSGGGGSQVQL |
| | | VQSGAEVKKPGSSVKVSCKASGGTFSSDAINWVRQ |
| | | APGQGLEWMGRIIPIFGVADYAQKFQGRVTLTADK |
| | | STSTAYMDLSSLRSEDTAVFYCARERGDYSNFWYF |
| | | DLWGRGTLVTVSSGGSHHHHHHHH |

SEQ ID NO: 26 represents a IL-2 mutant that lacks binding to IL-2R gamma but remained binding to IL-2R alpha and IL-2R beta.

SEQ ID NO: 27 represents the fusion protein of IL-2R gamma binding domain (Vhh2g) and IL-2R alpha binding domain (BT942 scFv). The binding of BT942 scFv to IL-2R alpha does not compete with the binding of IL2_RETR to IL-2R alpha. The sequence of BT942 scFv is underlined and the sequence of Vhh2g is double underlined in Table 6.

SEQ ID NO: 28 represents the fusion protein of IL-2R gamma binding domain (Vhh2g) and IL-2R alpha binding domain (Dac scFv). The binding of Dac scFv to IL-2R alpha competes with the binding of IL2_RETR to IL-2R alpha. The sequence of Dac scFv is underlined and the sequence of Vhh2g is double underlined in Table 6.

SEQ ID NO: 29 represents the fusion protein of IL-2R beta binding domain (Vhh2b) and IL-2R alpha binding domain (BT942 scFv). The binding of BT942 scFv to IL-2R alpha does not compete with the binding of IL2_RETR to IL-2R alpha. The sequence of BT942 scFv is underlined and the sequence of Vhh2b is double underlined in Table 6.

### Example 16: The Activation of IL-2R Beta and IL-2R Gamma by Mixture of IL-2 Mutant and VHH-scFv Fusion, which Bind to IL-2R Alpha as a Target Scaffold Protein in Biparatopic Manner and IL-2R Beta/Gamma as Target Receptor Proteins, is Dependent on IL-2R Alpha Binding

The present concept of conditional receptor signaling is further exemplified using IL-2R alpha as the scaffold protein to bring IL-2R beta and IL-2R gamma into proximity for conditional receptor signaling. This example also shows that other non-antibody binding proteins can also be used as the scaffold protein-binding domain or receptor protein-binding domain. The structure of the quaternary complex of IL-2 with IL-2R alpha, IL-2R beta, and IL-2R gamma is described (Science 2005; 310: 1159-1163) and their structure and binding epitope is shown in RCSB Protein Data Bank (2B5I).

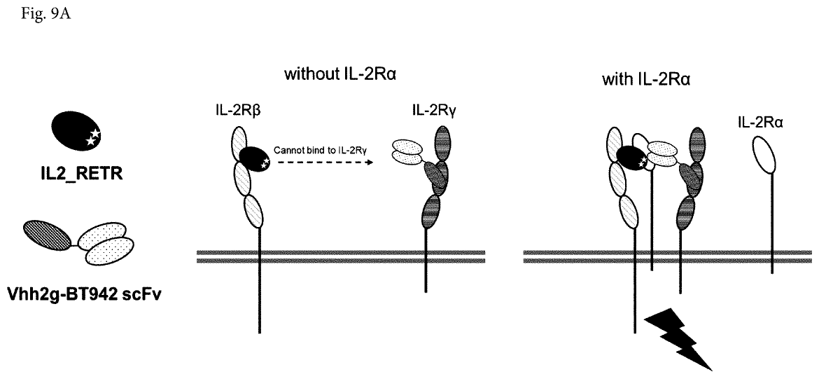

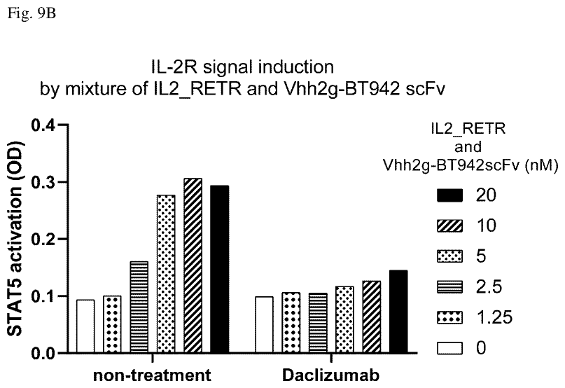

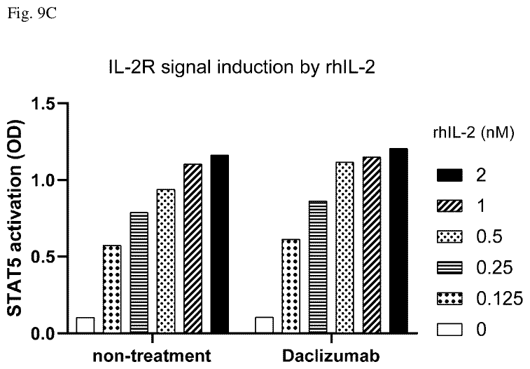

As an example, IL2_RETR is a non-agonistic IL-2 mutant and that binds to IL-2R alpha as a scaffold protein and IL-2R beta as a target receptor, but lacks binding activity to IL-2R gamma (Immunity 2015; 42: 826-838). Vhh2g-BT942 scFv is a fusion protein of VHH, which binds to IL-2R gamma, and BT942 scFv, which binds to IL-2R alpha as a scaffold protein. The binding epitope of BT942 on IL2-R alpha has been described (Scientific Reports 2021; 11: 22966) and its structure and binding epitope is shown in RCSB Protein Data Bank (7F9W). Because the binding of BT942 does not effect on IL-2 and IL-2R alpha binding, IL2_RETR and BT942 scFv do not compete with one another for the binding to IL-2R alpha. Together, the mixture of IL2_RETR and Vhh2g-BT942 scFv may allow the colocalization of IL-2R alpha, IL-2R beta and IL-2R gamma to induce IL-2R signaling dependent on IL-2R alpha binding. The schematic diagram for the mechanism of IL-2R alpha dependent IL-2R activation by the mixture of IL2_RETR and Vhh2g-BT942 scFv was shown in FIG. 9A.

HEK-Blue IL-2 Cells (InvivoGen, #hkb-il2) were seeded in 96-well plate overnight at 37 degrees C. in the presence of 5% CO₂. The cells were cultured in Dulbecco's Modified Eagle Medium (DMEM) supplemented with 10% fetal bovine serum and 1% Penicillin-Streptomycin. Day 1 post seeding, cells were treated with or without 5 micro g/mL of Daclizumab (RnD Systems, #MAB9927) for half-hour at 37 degrees C. in the presence of 5% CO₂. Daclizumab, which is anti-IL-2R alpha antibody and can suppress binding of IL2_RETR to IL-2R alpha, was used to evaluate IL-2R alpha-binding dependent IL-2R activation. Mixtures of IL2_RETR (SEQ ID: 26) and Vhh2g-BT942 scFv (SEQ ID: 27) or recombinant human IL-2 (Peprotech, #200-02) were added and further incubated at 37 degrees C./5% CO2 for another day. Day 2 post seeding, IL-2 receptor complex activation was evaluated using Quanti-Blue solution (InvivoGen, #rep-qbs) by measuring optical density at 620 nm, using Multiskan™ plate reader (FIGS. 9B and 9C).

As shown in FIGS. 9B and 9C, the combination of IL2_RETR and Vhh2g-BT942 scFv, which targets IL2R alpha as a scaffold protein and targets IL-2R beta and IL-2R gamma as receptor proteins, induced IL-2R signaling in HEK-Blue IL-2 Cells that constitutively express IL2R alpha. Additionally, this IL-2R signal induction was suppressed by treatment of Daclizumab (5 micro g/mL), an anti-IL-2R alpha neutralizing antibody (FIG. 9B). On the other hand, Daclizumab treatment did not show significant effect on IL-2R signaling that induced by recombinant IL-2 (FIG. 9C). The significant suppressive effect of Daclizumab on IL-2R signal suggested the IL-2R activation by IL2_RETR and Vhh2g-BT942 scFv is dependent on IL-2R alpha binding.

### Example 17: The Activation of IL-2 Receptor Complex by the Mixtures of IL-2 Mutant or VHH-VHH Fusions, which Target IL-2R Alpha as the Scaffold Protein, is Dependent on Biparatopic Binding to IL-2R Alpha

To show that biparatopic binding to scaffold protein, which is IL-2R alpha in this case, is a condition for IL-2R beta/gamma activation, the effect of combinations of proteins which can bind to IL-2R alpha biparatopically was compared against combinations which are unable to bind IL-2R alpha biparatopically.

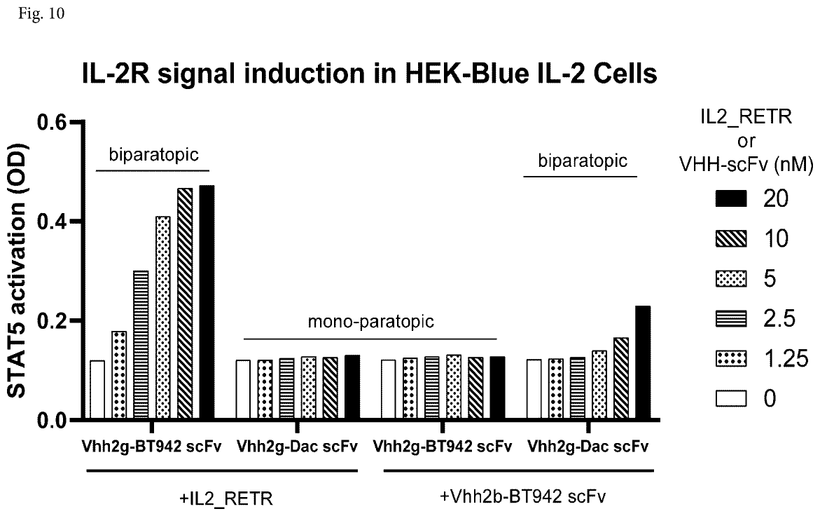

HEK-Blue IL-2 Cells (InvivoGen, #hkb-il2) were seeded in 96-well plate overnight at 37 degrees C. in the presence of 5% CO₂. The cells were cultured in Dulbecco's Modified Eagle Medium (DMEM) supplemented with 10% fetal bovine serum and 1% Penicillin-Streptomycin. After seeding, these cells were treated with different mixtures of recombinant IL2_RETR and/or VHH-scFv fusions (SEQ ID NO: 26-29) and incubated at 37 degrees C./5% CO₂ for 3 days. IL-2 receptor complex activation was evaluated using Quanti-Blue solution (InvivoGen, #rep-qbs) by measuring optical density at 620 nm, using Multiskan™ plate reader (FIG. 10).

FIG. 10 shows the IL-2R signal induction by the mixture of IL-2 mutant or VHH-ScFV, which target IL-2R alpha as the scaffold protein and IL-2R beta/gamma as the receptor proteins was dependent on biparatopic binding to IL-2R alpha. The combination of biparatopic binding, which are IL2_RETR and Vhh2g-BT942 scFv or Vhh2b-BT942 scFv and Vhh2g-Dac scFv, induced IL-2R signal, but the mono-paratopic binding combination, which are IL2_RETR and Vhh2g-Dac scFv or Vhh2b-BT942 scFv and Vhh2g-BT942 scFv, did not induce IL-2R signal in HEK-Blue IL-2 Cells that constitutively express IL-2R alpha. Dac scFv is a single-chain variable fragment version of Daclizumab, which competitively binds to IL-2R alpha with IL2_RETR but not with BT942 scFv.

### Example 18: Expression and Purification of VHH-scFv Fusions that Bind to PD1 as a Target Scaffold Protein and IL-2 Receptors as Target Receptor Proteins

VHH that can bind to human IL-2R beta (Vhh2b or Vhh2b3) or human IL-2R gamma (Vhh2g or Vhh2g6) was connected with human PD1 binding domain, which is Nivo scFvLH, Nivo scFvHL, NB01a scFvLH or NB01a scFvHL (SEQ ID NO: 30-36). The DNA encoding these proteins were cloned into a mammalian expression vector. These plasmids were expressed transiently using Expi293 cell line (Thermo Fisher, Carlsbad, CA, USA). Antibody purification was carried out using Protein A. For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 7
SEQ ID NOS: 30 to 37

| SEQ ID NO | Abbreviation | Sequences |
|---|---|---|
| SEQ ID NO: 30 | Vhh2g-Nivo | MGWSCIILFLVATATGVHSQVQLQESGGGSVQ |
| | scFvLH | AGGSLRLSCAASGYTYRDYYMGWFRQAPGRE |
| | | REGVASIYTRGSREGSTRYSSSVEGRFTITLDTA |
| | | KNTLYLQMNSLKPEDTAMYYCAADDRTWLPR |
| | | VQLGGPRENEYNYWGQGTQVTVSSEIVLTQSP |
| | | ATLSLSPGERATLSCRASQSVSSYLAWYQQKP |
| | | GQAPRLLIYDASNRATGIPARFSGSGSGTDFTLT |
| | | ISSLEPEDFAVYYCQQSSNWPRTFGQGTKVEIK |
| | | RTVGGGGSGGGGSGGGGSQVQLVESGGGVVQ |
| | | PGRSLRLDCKASGITESNSGMHWVRQAPGKGL |
| | | EWVAVIWYDGSKRYYADSVKGRFTISRDNSKN |
| | | TLFLQMNSLRAEDTAVYYCATNDDYWGQGTL |
| | | VTVSSGGSHHHHHHHH |
| SEQ ID NO: 31 | Vhh2g-Nivo | MGWSCIILFLVATATGVHSQVQLQESGGGSVQ |
| | scFvHL | AGGSLRLSCAASGYTYRDYYMGWFRQAPGRE |
| | | REGVASIYTRGSREGSTRYSSSVEGRFTITLDTA |
| | | KNTLYLQMNSLKPEDTAMYYCAADDRTWLPR |
| | | VQLGGPRENEYNYWGQGTQVTVSSQVQLVES |
| | | GGGVVQPGRSLRLDCKASGITESNSGMHWVRQ |
| | | APGKGLEWVAVIWYDGSKRYYADSVKGRFTIS |
| | | RDNSKNTLFLQMNSLRAEDTAVYYCATNDDY |
| | | WGQGTLVTVSSGGGGSGGGGSGGGGSEIVLTQ |
| | | SPATLSLSPGERATLSCRASQSVSSYLAWYQQK |
| | | PGQAPRLLIYDASNRATGIPARESGSGSGTDETL |
| | | TISSLEPEDFAVYYCQQSSNWPRTFGQGTKVEI |
| | | KRTVGGSHHHHHHHH |
| SEQ ID NO: 32 | Vhh2b-NB01a | MGWSCIILFLVATATGVHSQVQLQESGGGSVQ |
| | scFvLH | AGGSLRLSCAASSYTISSVCMGWFRQAPGKER |
| | | EGVAGIAPDGSTGYGDSVKGRFTISKDNAKNT |
| | | LYLQMNSLKPEDTAMYYCAAASPGRCELPRTA |
| | | LEPALYYNWGQGTQVTVSSDIQMTQSPSSVSA |
| | | SVGDRVTITCSASQGISGDLNWYQQKPGKAPK |
| | | LLIYHTSSLHSGVPSRFSGSGSGTDFTLTISSLQP |
| | | EDFATYYCQYYSKDLLTFGGGTKLEIKRTVGG |
| | | GGSGGGGSGGGGSEVQLVQSGAEVKKPGESLK |
| | | ISCKGSGYSFTNFYIHWVRQAPGQRLEWMGSI |
| | | YPNYGDTAYNQKFKDRFVFSLDTSVSTAYLQIS |
| | | SLKAEDTAVYYCARGYSYAMDYWGQGTTVT |
| | | VSSGGSHHHHHHHH |
| SEQ ID NO: 33 | Vhh2b3-NB01a | MGWSCIILFLVATATGVHSQVQLQESGGGLVQ |
| | scFvLH | PGGSLKLSCAASGFRFSNYGMSWVRQAPGEGL |
| | | EWVSYINGDGSRTHYADSVKGRFTISRDNAKN |
| | | TLYLQLNSLKTEDTAMYYCEKGLSRDGWSLSA |
| | | ASRGQGTQVTVSSDIQMTQSPSSVSASVGDRVT |
| | | ITCSASQGISGDLNWYQQKPGKAPKLLIYHTSS |
| | | LHSGVPSRFSGSGSGTDFTLTISSLQPEDFATYY |
| | | CQYYSKDLLTEGGGTKLEIKRTVGGGGGGGG |
| | | SGGGGSEVQLVQSGAEVKKPGESLKISCKGSG |
| | | YSFTNFYIHWVRQAPGQRLEWMGSIYPNYGDT |
| | | AYNQKFKDRFVFSLDTSVSTAYLQISSLKAEDT |
| | | AVYYCARGYSYAMDYWGQGTTVTVSSGGSH |
| | | HHHHHHH |
| SEQ ID NO: 34 | Vhh2g6-Nivo | MYRMQLLSCIALSLALVTNSAPTQVQLQESGG |
| | scFvHL | GAVQAGGSLRLSCAASGFTFSNAHMSWVRQA |
| | | PGKGREWISSIYSGGSTWYADSVKGRFTISRDN |
| | | SKNTLYLQLNSLKTEDTAMYYCAENRLHYYSD |
| | | DDSLRGQGTQVTVSSQVQLVESGGGVVQPGRS |
| | | LRLDCKASGITFSNSGMHWVRQAPGKGLEWV |
| | | AVIWYDGSKRYYADSVKGRFTISRDNSKNTLF |
| | | LQMNSLRAEDTAVYYCATNDDYWGQGTLVT |
| | | VSSGGGGSGGGGSGGGGSEIVLTQSPATLSLSP |
| | | GERATLSCRASQSVSSYLAWYQQKPGQAPRLL |
| | | IYDASNRATGIPARFSGSGSGTDFTLTISSLEPED |
| | | FAVYYCQQSSNWPRTEGQGTKVEIKRTVGGSH |
| SEQ ID NO: 35 | Vhh2g-NB01a | MYRMQLLSCIALSLALVTNSAPTQVQLQESGG |
| | scFVLH | GSVQAGGSLRISCAASGYTYRDYYMGWFRQA |
| | | PGREREGVASIYTRGSREGSTRYSSSVEGRFTIT |
| | | LDTAKNTLYLQMNSLKPEDTAMYYCAADDRT |
| | | WLPRVQLGGPRENEYNYWGQGTQVTVSSGGS |
| | | DIQMTQSPSSVSASVGDRVTITCSASQGISGDLN |
| | | WYQQKPGKAPKLLIYHTSSLHSGVPSRFSGSGS |
| | | GTDFTLTISSLQPEDFATYYCQYYSKDLLTFGG |
| | | GTKLEIKRTVGGGGSGGGGSGGGGSEVQLVQS |
| | | GAEVKKPGESLKISCKGSGYSFTNFYIHWVRQA |
| | | PGQRLEWMGSIYPNYGDTAYNQKFKDRFVFSL |
| | | DTSVSTAYLQISSLKAEDTAVYYCARGYSYAM |
| | | DYWGQGTTVTVSSGGSHHHHHHHH |
| SEQ ID NO: 36 | Vhh2g-NB01a | MYRMQLLSCIALSLALVTNSAPTQVQLQESGG |
| | scFvHL | GSVQAGGSLRLSCAASGYTYRDYYMGWFRQA |
| | | PGREREGVASIYTRGSREGSTRYSSSVEGRFTIT |
| | | LDTAKNTLYLQMNSLKPEDTAMYYCAADDRT |
| | | WLPRVQLGGPRENEYNYWGQGTQVTVSSGGS |
| | | EVQLVQSGAEVKKPGESLKISCKGSGYSFTNFY |
| | | IHWVRQAPGQRLEWMGSIYPNYGDTAYNQKF |
| | | KDRFVFSLDTSVSTAYLQISSLKAEDTAVYYCA |
| | | RGYSYAMDYWGQGTTVTVSSGGGGSGGGGSG |
| | | GGGSDIQMTQSPSSVSASVGDRVTITCSASQGIS |
| | | GDLNWYQQKPGKAPKLLIYHTSSLHSGVPSRFS |
| | | GSGSGTDFTLTISSLQPEDFATYYCQYYSKDLL |
| | | TFGGGTKLEIKRTVGGSHHHHHHHH |
| SEQ ID NO: 37 | PD1 | MQIPQAPWPVVWAVLQLGWRPGWFLDSPDRP |
| | | WNPPTFSPALLVVTEGDNATFTCSFSNTSESFV |
| | | LNWYRMSPSNQTDKLAAFPEDRSQPGQDCRFR |
| | | VTQLPNGRDFHMSVVRARRNDSGTYLCGAISL |
| | | APKAQIKESLRAELRVTERRAEVPTAHPSPSPRP |
| | | AGQFQTLVVGVVGGLLGSLVLLVWVLAVICSR |
| | | AARGTIGARRTGQPLKEDPSAVPVFSVDYGEL |
| | | DFQWREKTPEPPVPCVPEQTEYATIVFPSGMGT |
| | | SSPARRGSADGPRSAQPLRPEDGHCSWPL |

SEQ ID NO: 30 represents the fusion protein of IL-2R gamma binding domain (Vhh2g) and PD1 binding domain (Nivo scFvLH). The sequence of Nivo scFvLH is underlined and the sequence of Vhh2g is double underlined in Table 7.

SEQ ID NO: 31 represents the fusion protein of IL-2R gamma binding domain (Vhh2g) and PD1 binding domain (Nivo scFvHL). The sequence of Nivo scFvHL is underlined and the sequence of Vhh2g is double underlined in Table 7.

SEQ ID NO: 32 represents the fusion protein of IL-2R beta binding domain (Vhh2b) and PD1 binding domain (NB01a scFvLH). The sequence of NB01a scFvLH is underlined and the sequence of Vhh2b is double underlined in Table 7.

SEQ ID NO: 33 represents of IL-2R beta binding domain (Vhh2b3) and PD1 binding domain (NB01a scFvLH). The sequence of NB01a scFvLH is underlined and the sequence of Vhh2b3 is double underlined in Table 7.

SEQ ID NO: 34 represents the fusion protein of IL-2R gamma binding domain (Vhh2g6) and PD1 binding domain (Nivo scFvHL). The sequence of Nivo scFvHL is underlined and the sequence of Vhh2g6 is double underlined in Table 7.

SEQ ID NO: 35 represents the fusion protein of IL-2R gamma binding domain (Vhh2g) and PD1 binding domain (NB01a scFvLH). The sequence of NB01a scFvLH is underlined and the sequence of Vhh2g is double underlined in Table 7.

SEQ ID NO: 36 represents the fusion protein of IL-2R gamma binding domain (Vhh2g) and PD1 binding domain (NB01a scFvHL). The sequence of NB01a scFvHL is underlined and the sequence of Vhh2g is double underlined in Table 7.

SEQ ID NO: 37 represents human PD1.

### Example 19: The Induction of IL-2R Signal by Mixtures of Recombinant VHH-scFv Fusions, which Bind to PD1 in Biparatopic Manner and IL-2 Receptor Proteins, is Dependent on PD1 Expression

The present concept of conditional receptor signaling is further exemplified using PD1 as the scaffold protein and IL-2R beta and IL-2R gamma as the receptor proteins for conditional receptor signaling.

As an example, Nivo scFvLH or Nivo scFvHL and NB01a scFvLH are biparatopic antibodies that recognize different epitope of PD1 and can bind to PD1 simultaneously, and the epitope on PD1 bound by NB01a has also been described (J. Exp. Med 2019; 216: 1525-1541), and its structure and binding epitope is shown in RCSB Protein Data Bank (6HIG). Because the binding of Nivo scFvLH or Nivo scFvHL does not compete on the binding of NB01a scFvLH to PD1, the mixture of these protein may allow the colocalization of PD1, IL-2R beta and IL-2R gamma to induce IL-2R signaling dependent on PD1 binding. The epitope that Nivo binds on PD1 is described (Nature Communications 2017; 08: 14369) and its structure and binding epitope is shown in RCSB Protein Data Bank (5WT9).

Human PD1 (SEQ ID NO: 37) or PDL1 (SEQ ID NO: 12) was first expressed in HEK-Blue IL-2 Cells (InvivoGen, #hkb-il2) by electroporation using SF Cell Line 4D-Nucleofector X Kit L (Lonza, #V4XC-2024). After electroporation, these transfectants were seeded in 96-well plate and incubated overnight at 37 degrees C./5% CO₂. These cells were cultured in Dulbecco's Modified Eagle Medium (DMEM) supplemented with 10% fetal bovine serum and 1% Penicillin-Streptomycin. Day 1 post electroporation, these cells were treated with different mixtures of recombinant VHH-scFv fusions (SEQ ID NO: 30-33) or recombinant human IL-2 (Peprotech, #200-02) and further incubated at 37 degrees C./5% CO₂ for another day. Day 2 post electroporation, IL-2 receptor complex activation was evaluated using Quanti-Blue solution (InvivoGen, #rep-qbs) by measuring optical density at 620 nm, using Multiskan™ plate reader (FIGS. 11A and 11B).

As shown in FIGS. 11A and 11B, the mixtures of VHH-scFv fusions that bind to PD1 as a scaffold protein in biparatopic manner and IL-2R beta or IL-2R gamma as target receptor proteins can induce IL-2R signal on the condition of PD1 scaffold protein expression. In the absence of the scaffold protein that is PD1, IL-2R signaling was not induced.

### Example 20: The Activation of IL-2 Receptor Complex by Mixtures of VHH-VHH Fusions, which Target PD1 as a Scaffold Protein, is Dependent on Biparatopic Binding to PD1

To show that biparatopic binding to scaffold protein, which is PD1 in this case, is a condition for IL-2R beta/gamma activation, the effect of combinations of VHH-VHH fusions which can bind to PD1 biparatopically was compared against combinations of VHH-VHH fusions which are unable to bind to PD1 biparatopically.

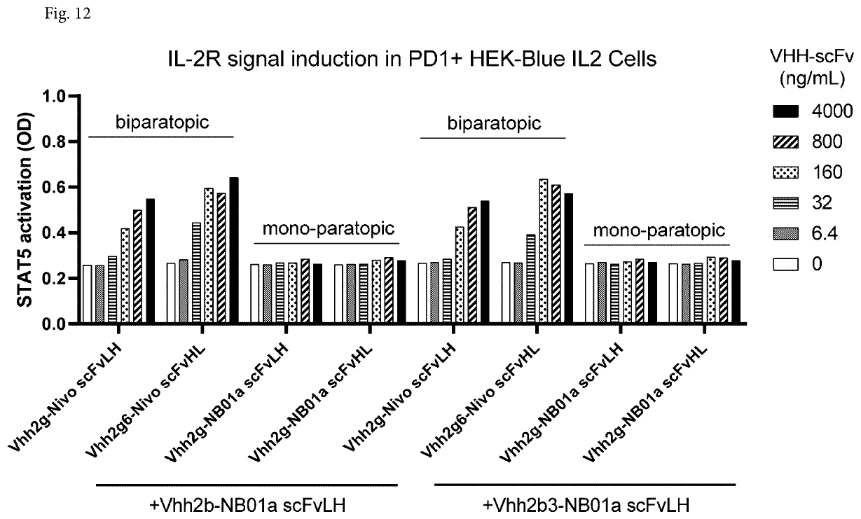

Human PD1 (SEQ ID NO: 37) was first expressed in HEK-Blue IL-2 Cells (InvivoGen, #hkb-il2) by electroporation using SF Cell Line 4D-Nucleofector X Kit L (Lonza, #V4XC-2024). After electroporation, these transfectants were seeded in 96-well plate and incubated overnight at 37 degrees C./5% CO₂. These cells were cultured in Dulbecco's Modified Eagle Medium (DMEM) supplemented with 10% fetal bovine serum and 1% Penicillin-Streptomycin. Day 1 post electroporation, these cells were treated with different mixtures of recombinant VHH-scFv fusions (SEQ ID NO: 30, 32-36) and further incubated at 37 degrees C./5% CO₂ for another day. Day 2 post electroporation, IL-2 receptor complex activation was evaluated using Quanti-Blue solution (InvivoGen, #rep-qbs) by measuring optical density at 620 nm, using Multiskan™ plate reader (FIG. 12).

As shown in FIG. 12, the IL-2R signal induction by the mixtures of VHH-scFv fusions, which bind to PD1 as a scaffold protein and IL-2R beta or IL-2R gamma as the target receptor proteins, is dependent on biparatopic binding to PD1. The biparatopic PD1 binding combinations, which used Nivo scFvHL or Nivo scFvLH and NB01a scFvHL and NB01a scFvLH as PD1 binding domain, induced IL-2R signal, but the mono-paratopic binding combination, which used only NB01a scFvHL or NB01a scFvLH as PD1 binding domain, did not induce IL-2R signal in PD1-expressing HEK-Blue IL-2 Cells.

### Example 21: Expression and Purification of Anti-PD1 Antibodies (Abs)

The DNA encoding heavy chain (Hch) and light chain (Lch) of anti-PD1 antibodies (SEQ ID NO: 38-59) were cloned into a mammalian expression vector. To prepare purified anti-PD1 Abs, corresponding Hch and Lch expressing plasmids were expressed transiently using Expi293 cell line (Thermo Fisher, Carlsbad, CA, USA). The Hch and Lch pairs of each antibody are described in Table 8. Protein purification was carried out using Protein A. For the concentration of the purified proteins, their absorbance at 280 nm was measured using a spectrophotometer. From the obtained value, the extinction coefficient calculated by the methods such as PACE was used to calculate the antibody concentration (Protein Science 1995; 4: 2411-2423).

| | SEQ | | SEQ | | |
|---|---|---|---|---|---|
| abbre- | ID | | ID | | |
| viation | NO | Hch Sequences | NO | Lch Sequences | Description |
| PDA0044 | SEQ | MGWSCIILFLVATATGVHSQSV | SEQ | MGWSCIILFLVATATGVHSAFELTQTPSSVEAAVG | The |
| | ID | EESGGRLVTPGTPLTLTCTASG | ID | GTVTIKCQASQSISSYLAWYQQKPGQPPKLLIYGA | sequences |
| | NO: | FSLSGYYMSWVRQAPGKGLEWI | NO: | SNLKSGVPSRFKGSGSGTQFTLTISSDEQLKSGTA | of heavy |
| | 38 | GSISYGQSTYYASWAKGRFTIS | 49 | SVVCLINNEYPREAKVQWKVQNALQSQNSQESVTE | chain |
| | | KTSTTYDLKITSPTTEDTATYF | | DLEAADAATYYCQSYYYSSSSSYSFQGGTEVVVKR | and light |
| | | CAAGYTINTAFDPWGPGTLVTV | | TVAAPSVFIFPPQDSKDSTYSISSTLTLSKADYEK | chain |
| | | SSASTKGPSVEPLAPSSKSTSG | | HKVYACEVTHQGLSSPVTKSFNRGEC | of anti- |
| | | QTAALGQLVKDYFPEPVTVSWN | | | human |
| | | SGALISQVHTFPAVLQSSGLYS | | | PD1 |
| | | LSSVVTVPSSSIGTQTYICNVN | | | antibodies |
| | | HKPSNTKVDKRVEPKSCQKTRT | | | |
| | | CPPCPAPBLRRGPSVELFPPKP | | | |
| | | KDILMISRTPEVTCVVVQVSHE | | | |
| | | DPEVKFNWYVDGVEVHNAKTKP | | | |
| | | REEQYNSTYRVVSVLIVLHQDW | | | |
| | | LNGKEYKCKVSNKALPAPIEKT | | | |
| | | ISKAKGQPREPQVYTLPPSREE | | | |
| | | MTKNQVSLTCLVKGFYPSDIAV | | | |
| | | EWESNGQPENNYKTTPPVLDSD | | | |
| | | GSFFLYSKLTVDKSRWQQGNVF | | | |
| | | SCSVMHEALHNHYTQKSLSLSP | | | |
| PDA0107 | SEQ | MGWSCIILFLVATATGVHSQSV | SEQ | MGWSCIILFLVATATGVHSAAVLTQTPSPVSAAVGG | |
| | ID | EESGGRLVTPGTPLTLTCTVSG | ID | TVSISCQSSQSVYGKNELSWYQQKPGQPPKLLIYY | |
| | NO: | ESLSRYYMSWVRQAPGKGLEWI | NO: | ASTLASGVPSRESGSGSGTQFALTISGVQADDAAT | |
| | 39 | GYIYADSGSTWYASWAKGRETI | 50 | YYCAGGYSSSVDTTFGGGTEVVVKRIVAAPSVFIF | |
| | | SKTSTTVDLKMTSITTEDTATY | | PPSDEQLKSGTASVVCLLNNEYPREAKVQWKVDNA | |
| | | PCARRADHDGAFDLWGPGTLVE | | LQSGNSQESVTEQDSKDSTYSLSSTLTLSKADYEK | |
| | | VSSASTKGPSVFPLAPSSKSTS | | HKVYACEVTHQGLSSPVIKSFNRGEC | |
| | | GGTAALGCLVKDYFPEPVTVSW | | | |
| | | NSGALTSQVHTFPAVLQSSQLY | | | |
| | | SLSSVVTVPSSSLGTQTYICNV | | | |
| | | NHKPSNTKVDKRVEPKSCDKTH | | | |
| | | TCPPCPAPELRRGPSVFLFPPK | | | |
| | | PKDTLMISRIPEVTCVVVDVSH | | | |
| | | EDPEVKFNWYVDGVEVHNAKTK | | | |
| | | PREEQYNSTYRVVSVLIVIHQD | | | |
| | | WINGKEYKCKVSNKALPAPIEK | | | |
| | | TISKAKQQPREPQVYILPPSRE | | | |
| | | EMTKNQVSLTCLVKGFYPSDIA | | | |
| | | VEWESNGQPENNYKTTPPVLDS | | | |
| | | DGSFFLYSKITVDKSRWQQGNV | | | |
| | | FSCSVMHEALHNRYTQKSLSLS | | | |
| | | P | | | |
| PDA0129 | SEQ | MGWSCIILFLVATATGVHSQSV | SEQ | MQWSCIILFLVATATGVHSIEMTQTPESVSAAVGG | |
| | ID | EESGGRLVTPGTPLTLTCTVSG | ID | TVTIKCQASEDIYSLLAWYQQKPGQPPKLLIYSAS | |
| | NO: | ESLSSYDMSWVRQAPGKGLEYI | NO: | TLASGVPSRFKGSGYGTEFTLTISGVQAADAATYY | |
| | 40 | GIISSSGSTYYASWAKGRFTIS | 51 | CQGGYYSSSADNTFGGGJEVVVKRTVAAPSVFIEP | |
| | | KISTTVDLKITSPTTEDTATYF | | PSDEQLKSGTASVVCLLNNPYPREAKVQWKVDNAL | |
| | | CARLGYVGYGYADIWGPGTLVI | | QSGNSQESVTEQDSKDSTYSLSSTLTLSKADYEKH | |
| | | VSSASTKGPSVFPLAPSSKSTS | | KVYACEVTRQGLSSPYTKSFNRGEC | |
| | | GGTAALGCLVKDYFPEPVTVSW | | | |
| | | NSGALTSGVHTFPAVLQSSGLY | | | |
| | | SISSVVIVPSSSLGTQTYICNV | | | |
| | | NHKPSNTKVDKRVEPKSCDKTH | | | |
| | | TCPPCPAPELRRGPSVFLFPPK | | | |
| | | PKDTLMISRTPEVTCVVVDVSH | | | |
| | | EDPEVKFNWYVDGVEVHNAKTK | | | |
| | | PREEQYNSTYRVVSVLTVLHQD | | | |
| | | WINGKEYKCKVSNKALPAPIEK | | | |
| | | TISKAKGQPREPQVYTLPPSRE | | | |
| | | EMTKNQVSLTCLVKGFYPSDIA | | | |
| | | VEWESNGQPENNYKTTPPVLDS | | | |
| | | DGSFFLYSKLTVDKSRWQQGNV | | | |
| | | FSCSVMHEALBNBYIQKSLSLS | | | |
| | | P | | | |
| PDA0138 | SEQ | MGWSCIILFLVATATGVHSQSV | SEQ | MGWSCIIFLVATATGVHSAAVLTQTPSPVSAAVGA | |
| | ID | EESGGRLVTPGTPLTLTCTVSG | ID | TVSISCQSSQSVYGNSELAWYQQKPGQRPKLLIFE | |
| | NO: | ESLSSYHMCWVRQPPQKGLEYI | NO: | ASKLASGVPSRESGSGSGTQFSLAISDVQADDAAT | |
| | 41 | GHISGGAITYYATWAKQRFTIS | 52 | YYCAGGYSSTSDITFQGQTEVVVKRTVAAPSVEIF | |
| | | ITSSTIVDLKITSPTTEDTATY | | PPSDEQLKSGTASVVCLLNNFYPREAKVQWKVDNA | |
| | | FCARGGGYGGGDYNEDLWGQGT | | LQSGNSQESVTEQDSKDSTYSLSSTLTLSKADYEK | |
| | | LVTVSSASTKGPSVFPLAPSSK | | HKVYACEVTHQGLSSPVIKSFNRGEC | |
| | | STSGGTAALGCLVKDYFPEPVT | | | |
| | | VSWNSGALTSGVIITFPAVLQS | | | |
| | | SGLYSLSSVVIVPSSSLGTQTY | | | |
| | | ICNVNHKPSNTKVDKRVEPKSC | | | |
| | | DKTHTCPPCPAPELRRGPSVFL | | | |
| | | FPPKPKDTLMISRTPEVTCVVV | | | |
| | | DVSHEDPEVKFNWYVDGVEVEN | | | |
| | | AKIKPREEQYNSTYRVVSVLIV | | | |
| | | LHQDWINGKEYKCKVSNKALPA | | | |
| | | PIEKIISKAKGQPREPQVYTLP | | | |
| | | PSREEMTKNQVSLTCLVKGFYP | | | |
| | | SDIAVEWESNGQPENNYKITPP | | | |
| | | VEDSDGSPFLYSKLTVDKSRWQ | | | |
| | | QGNVPSCSVMHEALHNBYTQKS | | | |
| | | LSLSP | | | |
| PDC0037 | SEQ | MGWSCIILFLVATATGVHSQSV | SEQ | MGWSCIILFLVATATGVHSAQVLTQTPSPVSAAVG | The |
| | ID | EESGGRLVTPGTPLTLTCTVSG | ID | GTVTISCQSSESVYNNNWLAWYQQKPGQPPKFLIY | sequences |
| | NO: | FSLSYYAMIWVRQAPQKGLEWI | NO: | DASTLASGVPSRESGSGSGTQFTLTISGVQADDAA | of heavy |
| | 42 | GFINSYGKTYYANWAKGRFTIS | 53 | TYYCQGTYYSSDWYFVEGGGTEVVVKRIVAAPSVF | chain |
| | | KTSITVDLKMTSLTSFDTATYF | | IFPPSDEQLKSGTASVVQLLNNFYPREAKVQWKVD | and light |
| | | CARFRDNYHENLWGQGTLVTVS | | NALQSGNSQESVTEQDSKQSTYSLSSTEILSKADY | chain |
| | | SASTKGPSVFPLAPSSKSTSGG | | EKHKVYACEVTHQGLSSPVTKSFNRQEC | of anti- |
| | | TAALGCLVKDYFPEPVTVSWNS | | | human |
| | | GALTSGVHTFPAVLQSSGLYSL | | | PD1 |
| | | SSVVTVPSSSLGTQTYICNVNH | | | antibodies |
| | | KPSNTKVDKRVEPKSCDKTHTC | | | |
| | | PPCPAPELRRGPSVFLFPPKPK | | | |
| | | DTLMISRTPEVTCVVVDVSHED | | | |
| | | PFVKFNWYVDGVEVHNAKTKPR | | | |
| | | EEQYNSTYRVVSVLTVLHQDWL | | | |
| | | NGKEYKCKVSNKALPAPIEKTI | | | |
| | | SKAKGQPREPQVYTLPPSREEM | | | |
| | | IKNQVSLTCLVKGFYPSDIAVE | | | |
| | | WESNGQPENNYKTTPPVLDSDG | | | |
| | | SFFLYSKLTVDKSRWQQGNVFS | | | |
| | | CSVMHEALENHYTQKSLSLSP | | | |
| PDC0053 | SEQ | MGWSCIILFLVATATGVHSQQHL | SEQ | MGWSCIILFLVATATGVHSADIVMTQTPASVEAAV | |
| | ID | VESGGGLVKPGASLTLTCKASG | ID | GGIVTIKCQASQSISSYLAWYQQKPGQPPNLLIYR | |
| | NO: | FYESGYYYMSWVRQAPGKGLEW | NO: | ASTLASGVPSRFKGSGSGTQFTLTISDLEAADAAT | |
| | 43 | IASEYSGGSQNTYYASWAKGRE | 54 | YYCQGYHESSTSSNYGNAFGQGTEVVVKRTVAAPS | |
| | | TISKTSSTTVTLQMTSLTAADT | | VFIFPPSDEQLKSGTASVVQLLNNFYPREAKVQWK | |
| | | ATYFCARGADYTEDYPADAAAW | | VDNALQSGNSQESVIEQDSKQSTYSLSSILILSKA | |
| | | SEDPWGPGTLVTVSSASTKGPS | | DYEKHKVYACEVTHQGLSSPVTKSFNRGEC | |
| | | VEPLAPSSKSTSGGTAALGCLV | | | |
| | | KDYPPEPYTVSWNSGALISGVH | | | |
| | | TFPAVLQSSGLYSLSSVVTVPS | | | |
| | | SSLGTQTYICNVNHKPSNTKVD | | | |
| | | KRVEPKSCDKTHTCPPCPAPEL | | | |
| | | RRGPSVELFPPKPKDILMISRT | | | |
| | | PEVICVVVDVSHEDPEVKFNWY | | | |
| | | VDGVEVHNAKTKPREEQYNSTY | | | |
| | | RVVSVLTVLHQDWLNGKEYKCK | | | |
| | | VSNKALPAPIEKTISKAKGQPR | | | |
| | | EPQVYILPPSREEMTKNQVSLI | | | |
| | | CLVKGFYPSDIAVEWESNGQPE | | | |
| | | NNYKTTPPVLDSDGSFFLYSKL | | | |
| | | TVDKSRWQQGNVFSCSYMHEAL | | | |
| | | ENHYTQKSISLSP | | | |
| PDE0171 | SEQ | MGWSCIILFLVATATGVIISQS | SEQ | MGWSCIILFLVATATGVHSDIVMTQTPASVSAAVG | |
| | ID | LEESGGGLVKPGASLTLTCTAS | ID | GTVSINCQASEDIENFLAWYQQKPGQPPSLLIYET | |
| | NO: | GFSFSGSYYMCWVRQAPGKGLE | NO: | SKLASGVPSRFSGSGSGTEFILTISDLEAADAATY | |
| | 44 | CIACIYAGSSGATYYASWAKGR | 55 | YCQSYDWSDSSTYDNTFQGQTEVVVKRTVAAPSVF | |
| | | FTVSKTSSTTVTLQMTSLTAAD | | IFPPSDEQLKSGTASYVCLLNNFYPREAKVQWKVD | |
| | | TATYFCARHTNGIGLGLWGPGT | | NALQSGNSQESVTEQDSKDSTYSLSSTLTLSKADY | |
| | | LVTVSSASTKGPSVFPLAPSSK | | EKHKVYACEVTHQGLSSPVIKSFNRGEC | |
| | | STSGGTAALGCLVKDYFPEPVT | | | |
| | | VSWNSGALTSGVHTFPAVLQSS | | | |
| | | GLYSLSSVVTVPSSSLGTQTYI | | | |
| | | CNVNHKPSNIKVDKRVEPKSCD | | | |
| | | KTHTCPPCPAPELRRGPSVFLF | | | |
| | | PPKPKDTLMISRTPEVTCVVVD | | | |
| | | VSHEDPEVKFNWYVDGVEVHNA | | | |
| | | KTKPREEQYNSTYRVVSVLIVL | | | |
| | | HQDWINGKEYKCKVSNKALPAP | | | |
| | | IEKTISKAKGQPREPQVYTIPP | | | |
| | | SREEMTKNQVSLTCLVKGFYPS | | | |
| | | DIAVEWESNGQPENNYKTTPPV | | | |
| | | LDSDQSFFLYSKLTVDKSRWQQ | | | |
| | | GNVFSCSVMHEALHNHYTQKSL | | | |
| | | SLSP | | | |
| GY5 | SEQ | MGWSCIILFLVATATGVHSQVQL | SEQ | MGWSCIILFLVATATGVHSDIMLTQSPLSLPVSLG | |
| | ID | QQSGPELVRPGVSVKISCKGSG | ID | DQASISCRSSQGIYHSDGNTYLEWYLQKPGQSPKL | |
| | NO: | HTFIDYALHWVRQSHVKSLEWI | NO: | LIYKVSNRESGVPDRESGSGSGTDFILKISRVEAE | |
| | 45 | GVISSYNGIINYNQRFKGKATM | 56 | DLGVYYCFQGSHVPYTFGGGTKLEIKRIVAAPSVF | |
| | | TVDKSSSTAYLELARETSEDSA | | IFPPSDEQLKSGTASVVCLLNNFYPREAKVQWKVD | |
| | | IYYCSREEWDVFYYFDYWGQGI | | NALQSGNSQESVTEQDSKDSTYSLSSTLTLSKADY | |
| | | TLTVSSASTKQPSVFPLAPSSK | | EKHIKVYACEVTHQGLSSPVTKSFNRGEC | |
| | | STSGGTAALGQLVKDYFPEPVI | | | |
| | | VSWNSGALTSGVHTEPAVLQSS | | | |
| | | QLYSLSSVVTVPSSSLQTQTYI | | | |
| | | CNVNHKPSNTKVDKRVEPKSCD | | | |
| | | KTHTCPPCPAPELRRGPSVFLF | | | |
| | | PPKPKDTLMISRTPEVTCVVVD | | | |
| | | VSHEDPEVKFNWYVDGVEVHNA | | | |
| | | KIKPREEQYNSTYRVVSVITVL | | | |
| | | HQDWINGKEYKCKVSNKALPAP | | | |
| | | IEKTISKAKGQPREPQVYTIPP | | | |
| | | SREEMTKNQVSLTCLVKGFYPS | | | |
| | | DIAVEWESNGQPENNYKTTPPV | | | |
| | | LDSDGSFFLYSKLTVDKSRWQQ | | | |
| | | GNVFSCSVMHEALHNHYTQKSL | | | |
| | | SLSP | | | |
| GY14 | SEQ | MGWSCIILFLVATATGVHSQVQ | SEQ | MGWSCIILFLVATATGVHASILMTQDETSLPVSLG | The |
| | ID | LQQSGAELVRPGASVKLSCKAL | ID | DQASISCRSSQTIVHTNGNTYLEWYLQKPGQSPKL | sequences |
| | NO: | GDTFTDYEIHWVKQTPVHGLEW | NO: | LIYKVSNRFSGVPDRFSGSGSGTYFTLKISRLEAE | of heavy |
| | 46 | IGVIHPGSGGTVYNQKPKGKAT | 57 | DLGVYYCFQGSHVPYTFGGGTKLEMKRTVAAPSVF | chain |
| | | LTADKYSSTAYMELSSLTSEDS | | IFPPSDEQLKSGTASVVCLLNNFYPREAKVQWKVD | and light |
| | | AVYYCTREGMNTDWYFDVWGAG | | NALQSGNSQESVTEQDSKDSTYSLSSTLTLSKADY | chain |
| | | TTVTVSSASTKGPSVFPLAPSK | | EKHKVYACEVTHQGESSPVTKSFNRGEC | of anti- |
| | | STSGGTAALGCLVKDYFPEPVT | | | human |
| | | VSWNSGALTSGVHTFPAVLQSS | | | PD1 |
| | | GLYSLSSVVYVPSSSLGTQTYI | | | antibodies |
| | | CNVNHKPSNTKVDKRVEPKSCD | | | |
| | | KTHTCPPCPAPELRRGPSVFLF | | | |
| | | PPKPKDTLMISRTPEVTCVVDV | | | |
| | | SHEDPEVKFNWYVDGVEVHNAK | | | |
| | | TKPREEQYNSTYRVVSVLTVLH | | | |
| | | QDWLNGKEYKCKVSNKALPAPI | | | |
| | | EKTISKAKGQPREPQVYTLPPS | | | |
| | | REEMTKNQVSLTCLVKGFYPSD | | | |
| | | IAVEWESNGQPENNYKTTPPVL | | | |
| | | DSDGSFFLYSKLTVDKSRWQQG | | | |
| | | NVFSCSVMHEALHNHYTQKSLS | | | |
| | | LSP | | | |
| PDA0041 | SEQ | MGWSCIILFLVATATGVHSQEQ | SEQ | MGWSCIILFLVATATGVHSAFELTQTPSSVEAAVG | |
| | ID | LEESGGDLVKPGASLTLTCTAS | ID | GTVSINCQASEDIERYLAWYQQRPGQPPKLLIYTA | |
| | NO: | GFSFNSYWMSWVRQAPGKGLEW | NO: | SSLPSGVPSRFKGSGSGTQFTLTISDLEAADAATY | |
| | 47 | IADIGAGGSGSTFYASWAKGRF | 58 | YCQQGWSDANYDNTFGGGTEVVVKRTVAAPSVFIF | |
| | | TISKTSSTTVTLQMTSLTAADT | | PPSDEQLKSGTASVVCLLNNFYPREAKVQWKVDNA | |
| | | ATYFCARYVDGGHSDLWGPGTL | | LQSGNSQESVTEQDSKDSTYSLSSTLTLSKADYEK | |
| | | VTVSSASTKGPSVFPLAPSSKS | | HKVYACEVTHQGLSSPVTKFNRGEC | |
| | | TSGGTAALGCLVKDYFPEPVTV | | | |
| | | SWNSGALTSGVHTFPAVLQSSG | | | |
| | | LYSLSSVVTVPSSSLGTQTYIC | | | |
| | | NVNHKPSNTKVDKRVEPKSCDK | | | |
| | | THTCPPCPAPELRRGPSVFLFP | | | |
| | | PKPKDTLMISRTPEVTCVVVDV | | | |
| | | SHEDPEVKFNWYVDGVEVHNAK | | | |
| | | TKPREEQYNSTYRVVSVLTLHQ | | | |
| | | DWLNGKEYKCKVSNKALPAPIE | | | |
| | | KTISKAKGQPREPQVYTLPPSR | | | |
| | | EEMTKNQVSLTCLVKGFYPSDI | | | |
| | | AVEWESNGQPENNYKITPPVLD | | | |
| | | SDGSFFLYSKLTVDKSRWQQGN | | | |
| | | VFSCSVMHEALHNHYTQKSLSL | | | |
| | | SP | | | |
| PDA0070 | SEQ | MGWSCIILFLVATATGVHSQEQ | SEQ | MGWSCIILFLVATATGVHSAFELTQTPSSVFAAVGG | |
| | ID | LEESGGDLVKPGASLTLTCTAS | ID | TVSINCQASEDIERYLAWYQQRPGQPPKLLIYTAS | |
| | NO: | GFSFNSYWMSWVRQAPGKGLEW | NO: | SLPSGVPSRFKGSGSGTQFTLTISDLEAADAATYY | |
| | 48 | IADIGAGGSGSTFYASWAKGRF | 59 | CQQGWSDANVDNTFGGGTEVVVKRIVAAPSVFIFP | |
| | | TISKTSSTTVTLQMTSLTAADT | | PSDEQLKSGTASVVCLLNNEYPREAKVQWKVDNAL | |
| | | ATYFCARYVDGGJSDLWGPGTL | | QSQNSQESVTEQDSKDSTYSLSSTLTLSKADYEKH | |
| | | VTVSSASTKGPSVFPLAPSSKS | | KVYACEVTHQGLSSPYTKSFNRGEC | |
| | | TSGGTAALGCLVKDYFPEPVTV | | | |
| | | SWNSGALTSGVHTFPAVLQSSG | | | |
| | | LYSLSSVVTVPSSSLGTQTYIC | | | |
| | | NVNHKPSNTKVDKRVEPKSCDK | | | |
| | | THTCPPCPAPELRRGPSVFLFP | | | |
| | | PKPKDRLMISRTPEVTCVVVDV | | | |
| | | SHEDPEVKFNWYVDGVEVHNAK | | | |
| | | TKPREEQYNTYRVVSVLTVLHQ | | | |
| | | DWLNGKEYKCKVSNKALPAPIE | | | |
| | | KTISKAKGQPREPQVYTLPPSR | | | |
| | | EEMTKNQVSLTCLVKGFYPSDI | | | |
| | | AVEWESNGQPENNYKTTPPVLD | | | |
| | | SGSFFLYSKLTVDKSRWQQGNV | | | |
| | | FSCSVMHEALHNHYTQKSLSIS | | | |
| | | P | | | |

### Example 22: The Binding Activity of Anti-PD1 Abs

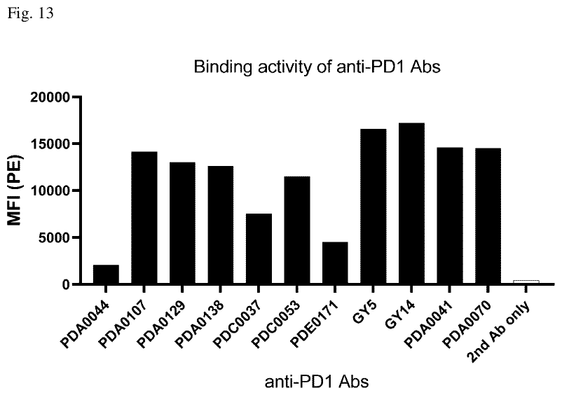

The binding activity of anti-PD1 Abs were evaluated by flow cytometry. Anti-PD1 Abs (10 micro g/mL) and human PD1-expressing cell line (NFAT-luc2/PD1 Jurkat Cell line, Promega, #CS187102) were incubated for 20 minutes in staining buffer (Biolegend, #420201). Unbound Abs were removed by washing twice with staining buffer. PD1 bounded human IgG Abs were detected by Goat F(ab′)2 anti-Human IgG, Mouse ads-PE (Southern Biotech, #2043-09). Data acquisition was performed on an LSRFortessa X-20 (Becton Dickinson) (FIG. 13).

As shown in FIG. 13, all anti-PD1 Abs showed clear binding to human PD1-expressing cell line.

### Example 23: The Neutralizing Activity of Anti-PD1 Abs

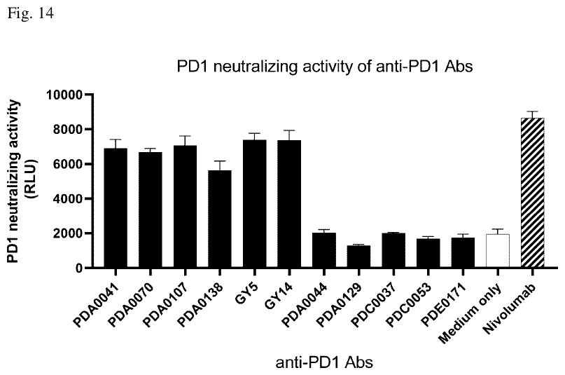

The neutralizing activity of anti-PD1 Abs were evaluated by PD1/PD-L1 blockade assay system (Promega, #CS187109). PD-L1+CHOK1 cells (Promega, #CS187108) were seeded in 384-well white plate overnight at 37 degrees C. in the presence of 5% CO₂. The cells were cultured in RPMI1640 with 5% fetal bovine serum and 1% Penicillin-Streptomycin. Day 1 post seeding, anti-PD1 Abs (5 micro g/mL as a final concentration) and PD1+Jurkat cells (NFAT-luc2/PD1 Jurkat Cell line, Promega, #CS187102) were added to PD-L1+CHOK1 seeded wells and further incubated for 6 hours. Nivolumab (Selleck, #A2002, 50 micro g/mL as a final concentration) was used as a positive control. After 6 hours, TCR activation was detected by Bio-Glo Luciferase Assay System (Promega, #G7940) and GloMax multi plate reader (Promega, #GM3500) (FIG. 14).

As shown in FIG. 14, PDA0041, PDA0070, PDA0107, PDA0138, GY5 and GY14 demonstrated PD1 blocking activity, but not for PDA0044, PDA0129, PDC0037, PDC0053 and PDE0171.

### Example 24: Expression and Purification of VHH-IgG Fusion Proteins that Bind to PD1 as a Target Scaffold Protein and IL-2R Beta and IL-2R Gamma as Target Receptor Proteins

Anti-IL-2R gamma VHH or anti-IL-2R beta VHH were fused to Lch of neutralizing anti-PD1 IgG Abs (SEQ ID NO: 60-65, 71-76) or Hch of non-neutralizing anti-PD1 IgG Abs (SEQ ID NO: 66-70, 77-81) by linker. Also, anti-IL-2R beta VHH or IL-2R gamma VHH were fused to anti-PD1 scFv (SEQ ID NO: 82, 83) by linker. The DNA encoding these proteins were cloned into a mammalian expression vector. To prepare purified Abs, corresponding Hch and Lch expressing plasmids were expressed transiently using Expi293 cell line (Thermo Fisher, Carlsbad, CA, USA). The Hch and Lch pairs of each antibody are described in Table 9. Protein purification was carried out using Protein A. For the concentration of the purified proteins, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 9

| | SEQ | | SEQ | | |
|---|---|---|---|---|---|
| abbreviation | ID NO | Hch Sequences | ID NO | Lch Sequences | Description |
| Vhh2g- | SEQ ID | MGWSCIILFLVATATGVHSQEQLEESGGDLVKPGASLTLTCT | SEQ ID | MGWSCIILFLVATATGVHSQVQLQESGGGSVQAGGSLRLSC | The fusion proteins |
| PDA0041 | NO: 47 | ASGFSFNSYWMSWVRQAPGKGLEWIADIGAGGSGSTFYASW | NO: 60 | AASGYTYRDYYMGWFRQAPGREREGVASIYTRGSREGST | of PD1 binding |
| | | AKGRFTISKTSSTTVTLQMTSLTAADTATYFCARYVDGGHSD | | RYSSSVEGRFTITLDTAKNTLYLQMNSLKPEDTAMYYCAA | domain and IL-2Rγ |
| | | LWGPGTLVTVSSASTKGPSVFPLAPSSKSTSGGTAALGCLVKD | | DDRTWLPRVQLGGPRENEYNYWGQGTQVTVSSGGSAFEL | binding domain. |
| | | YFPEPVTVSWNSGALTSGVHTFPAVLQSSGLYSLSSVVTVPSS | | TQTPSSVEAAVGGTVSINCQASEDIERYLAWYQQRPGQPPKL | The sequence of |
| | | SLGTQTYICNVNHKPSNTKVDKRVEPKSCDKTHICPPCPAPEL | | LIYTASSLPSGVPSRFKGSGSGTQFTLTISDLEAADAATYYCQQ | PD1 binding |
| | | RRGPSVFLFPPKPKDILMISRTPEVTCVVVDVSHEDPEVKENW | | GWSDANVDNTFGGGTEVVVKRTVAAPSVFIFPPSDEQLKSGT | domain is |
| | | YVDGVEVHNAKTKPREEQYNSTYRVVSVLTVLHQDWLNGK | | ASVVCLLNNFYPREAKVQWKVDNALQSGNSQESVTEQDSKD | underlined and the |
| | | EYKCKVSNKALPAPIEKTISKAKGQPREPQVYTLPPSREEMTK | | STYSLSSILTLSKADYEKHKVYACEVTHQGLSSPVTKSFNRGE | sequence of IL-2Rγ |
| | | NQVSLTCLVKGFYPSDIAVEWESNGQPENNYKTTPPVLDSDG | | C | binding domain is |
| | | SFFLYSKLTVDKSRWQQGNVFSCSVMHEALHNHYTQKSLSLS | | | shown in bold. |
| | | P | | | |
| Vhb2g- | SEQ ID | MGWSCIILFLVATATGVHSQEQLEESGGDLVKPGASLTLTCT | SEQ ID | MGWSCIILFLVATATGVHSQVQLQESGGGSVQAGGSLRLSC | |
| PDA0070 | NO: 48 | ASGFSFNSYWMSWVRQAPGKGLEWIADIGAGGSGSTFYASW | NO: 61 | AASGYTYRDYYMGWFRQAPGREREGVASIYTRGSREGST | |
| | | AKGRFTISKTSSTTVTLQMTSLTAADTATYFCARYVDGGHSD | | RYSSSVEGRFTITLDTAKNTLYLQMNSLKPEDTAMYYCAA | |
| | | LWGPGTLVTVSSASTKGPSVFPLAPSSKSTSGGTAALGCLVKD | | DDRIWLPRVQLGGPRENEYNYWGQGTQVTVSSGGSAFEL | |
| | | YFPEPVTVSWNSGALTSGVHTFPAVLQSSGLYSLSSVVTVPSS | | TQTPSSVEAAVGGTVSINCQASEDIERYLAWYQQRPGQPPKL | |
| | | SLGTQTYICNVNHKPSNTKVDKRVEPKSCDKTHTCPPCPAPEL | | LIYTASSLPSGVPSRFKGSGSGTQFTLTISDLEAADAATYYCQQ | |
| | | RRGPSVFLFPPKPKDTLMISRTPEVTCVVVDVSHEDPEVKFNW | | GWSDANVDNTFGGGTEVVVKRTVAAPSVFIFPPSDEQLKSGT | |
| | | YVDGVEVHNAKTKPREEQYNSTYRVVSVLTVLHQDWLNGK | | ASVVCLLNNFYPREAKVQWKVDNALQSGNSQESVTEQDSKD | |
| | | EYKCKVSNKALPAPIEKTISKAKGQPREPQVYTLPPSREEMTK | | STYSLSSTLTLSKADYEKHKVYACEVTHQGLSSPVTKSFNRGE | |
| | | NQVSLICLVKGFYPSDIAVEWESNGQPENNYKTTPPVLDSDG | | C | |
| | | SFFLYSKLTVDKSRWQQGNVFSCSVMHEALANHYTQKSLSLS | | | |
| | | P | | | |
| Vhh2g- | SEQ ID | MGWSCIILFLVATATGVHSQSVEESGGRLVTPGTPLTLTCTVS | SEQ ID | MGWSCIILFLVATATGVHSQVQLQESGGGSVQAGGSERLSC | |
| PDA0107 | NO: 39 | GFSLSRYYMSWVRQAPGKGLEWIGYIYADSGSTWYASWAK | NO: 62 | AASGYTYRDYYMGWFRQAPGREREGVASIYTRGSREGST | |
| | | GRFTISKTSTTYDLKMTSLTTEDTATYFCARRADHDGAFDLW | | RYSSSVEGRFTITLDTAKNTLYLQMNSLKPEDTAMYYCAA | |
| | | GPGTLVTVSSASTKGPSVFPLAPSSKSTSGGTAALGCLVKDYF | | DDRIWLPRVQLGGPRENEYNYWGQGTQVTVSSGGSAAVL | |
| | | PEPVTVSWNSGALTSGVHTFPAVLQSSGLYSLSSVVTVPSSSL | | TQTPSPVSAAVGGTVSISCQSSQSVYGKNELSWYQQKPGQPP | |
| | | GTQTYICNVNHKPSNTKVDKRVEPKSCDKTHTCPPCPAPELR | | KLLIYYASTLASGVPSRESGSGSGTQFALTISGVQADDAATYY | |
| | | RGPSVFLFPPKPKDTLMISRTPEVTCVVVDVSHEDPEVKFNW | | CAGGYSSSVDTTFGGGTEVVVKRTVAAPSVFIFPPSDEQLKSG | |
| | | YVDGVEVHNAKTKPREEQYNSTYRVVSVLTVLHQDWLNGK | | TASVVCLLNNEYPREAKVQWKVDNALQSGNSQESVTEQDSK | |
| | | EYKCKVSNKALPAPIEKTISKAKGQPREPQVYTLPPSREEMTK | | DSTYSLSSTLTLSKADYEKHKVYACEVTHQGLSSPVTKSFNR | |
| | | NQVSLTCLVKGFYPSDIAVEWESNGQPENNYKTTPPVLDSDG | | GEC | |
| | | SFFLYSKLIVDKSRWQQGNVFSCSVMHEALHNHYTQKSLSLS | | | |
| | | P | | | |
| Vhh2g- | SEQ ID | MGWSCIILFLVATATGVHSQSVEESGGRLVTPGTPLTLTCTVS | SEQ ID | MGWSCIILFLVATATGVHSQVQLQESGGGSVQAGGSLRLSC | The fusion proteins |
| PDA0138 | NO: 41 | GFSLSSYHMCWVRQPPGKGLEYIGIISGGAITYYATWAKGRFT | NO: 63 | AASGYTYRDYYMGWFRQAPGREREGVASIYTRGSREGST | of PD1 binding |
| | | ISITSSTTVDLKITSPTTEDTATYFCARGGGYGGGDYNEDLWG | | RYSSSVEGRFTITLDTAKNTLYLQMNSLKPEDTAMYYCAA | domain and IL-2Rγ |
| | | QGTLVTVSSASTKGPSVFPLAPSSKSTSGGTAALGQLVKDYFP | | DDRTWIPRVQLGGPRENEYNYWGQGTQVIVSSGGSAAVL | binding domain. |
| | | EPVTVSWNSGALTSGVHTFPAVLQSSGLYSLSSVVTVPSSSLG | | TQTPSPVSAAVGATVSISCQSSQSVYGNSELAWYQQKPGQRP | The sequence of |
| | | TQTYICNVNHKPSNTKVDKRVEPKSCDKTHTCPPCPAPELRR | | KLLIFEASKLASGVPSRFSGSGSGTQFSLAISDVQADDAATYY | PD1 binding |
| | | GPSVFLFPPKPKDTLMISRTPEVTCVVVDVSHEDPEVKENWY | | CAGGYSSTSDTTFGGQTEVVVKRTVAAPSVFIFPPSDEQLKSG | domain is |
| | | VDGVEVHNAKTKPREEQYNSTYRVVSVLTVLHQDWLNGKE | | TASVVCLLNNFYPREAKVQWKVDNALQSGNSQESVTEQDSK | underlined and the |
| | | YKCKVSNKALPAPIEKTISKAKGQPREPQVYTLPPSREEMTKN | | DSTYSLSSTLTLSKADYEKHKVYACEVTHQGLSSPVTKSENR | sequence of |
| | | QVSLTCLVKGFYPSDIAVEWESNGQPENNYKTTPPVLDSDGS | | GEC | IL-2Rγ |
| | | FFLYSKLTVDKSRWQQGNVFSCSVMHEALHNHYTQKSLSLSP | | | binding |
| | | | | | domain is |
| | | | | | shown in bold. |
| Vhh2g-GY5 | SEQ ID | MGWSCHLFLVATATGVHSQVQLQQSGPBLVRPGVSVKISCK | SEQ ID | MGWSCIILFLVATATGVHSQVQLQESGGGSVQAGGSLRLSC | |
| | NO: 45 | GSGHTFTDYALHWVRQSHIVKSLEWIGVISSYNGITNYNQRFK | NO: 64 | AASGYTYRDYYMGWFRQAPGREREGVASIYTRGSREGST | |
| | | GKATMTVDKSSSTAYLELARLTSEDSAIYYCSREEWDVEYYF | | RYSSSVEGRFTITLDTAKNTLYLQMNSLKPEDTAMYYCAA | |
| | | DYWGQGTTLTVSSASTKGPSVFPLAPSSKSTSGGTAALGCLV | | DDRTWLPRVQLGGPRENEYNYWGQGTQVTVSSGGSDIML | |
| | | KDYFPEPVTVSWNSGALTSGVHTFPAVLQSSGLYSLSSVVTVP | | TQSPLSLPVSLGDQASISCRSSQGIVHSDGNTYLEWYLQKPGQ | |
| | | SSSLGTQTYICNVNHKPSNTKVDKRVEPKSCDKTHTCPPCPAP | | SPKLLIYKYSNRFSGVPDRFSGSGSGTDFILKISRVEAEDLGVY | |
| | | ELRRGPSVFLFPPKPKDTLMISRTPEVTCVVVDVSHEDPEVKF | | YCFQGSHVPYTEGGGTKLEIKRTVAAPSVFIFPPSDEQLKSGT | |
| | | NWYVDGVEVHNAKTKPREEQYNSTYRVVSVLTVLHQDWLN | | ASVVCLLNNFYPREAKVQWKVDNALQSGNSQESVTEQDSKD | |
| | | GKEYKCKVSNKALPAPIEKTISKAKGQPREPQVYTLPPSREEM | | STYSLSSTLTLSKADYEKHKVYACEVTHQGLSSPVTKSENRGE | |
| | | TKNQVSLTCLVKGFYPSDIAVEWESNGQPENNYKTTPPVLDS | | C | |
| | | DGSFFLYSKLTVDKSRWQQGNVFSCSVMHEALHNHYTQKSL | | | |
| | | SLSP | | | |
| Vhh2g-GY14 | SEQ ID | MGWSCIILFLVATAIGVHSQVQLQQSGAELVRPGASVKLSCK | SEQ ID | MGWSCIILFLVATATGVHSQVQLQESGGGSYQAGGSLRLSC | |
| | NO: 46 | ALGDTFTDYEIHWYKQTPVHGLEWIGVIHPGSGGTVYNQKFK | NO: 65 | AASGYTYRDYYMGWFRQAPGREREGVASIYTRGSREGST | |
| | | GKATLTADKYSSTAYMELSSLTSEDSAVYYCTREGMNTDWY | | RYSSSVEGRFTITLDTAKNTLYLQMNSLKPEDTAMYYCAA | |
| | | FDVWGAGTTVTVSSASTKGPSVFPLAPSSKSTSGGTAALGCL | | DDRTWLPRVQLGGPRENEYNYWGQGTQVTVSSGGSDILM | |
| | | VKDYFPEPVIVSWNSGALTSGVHTFPAVLQSSGLYSLSSVVI | | TQDELSLPVSLGDQASISCRSSQTIVHITNGNTYLEWYLQKPGQ | |
| | | VPSSSLGTQTYICNVNHKPSNTKVDKRVEPKSCDKTHTCPPCP | | SPKLLIYKVSNRFSGVPDRFSGSGSGTYFTLKISRLEAEDLGVY | |
| | | APELRRGPSVFLFPPKPKDTLMISRIPEVTCVVVDVSHEDPEV | | YCFQGSHVPYTFGGGTKLEMKRTVAAPSVFIFPPSDEQLKSGT | |
| | | KFNWYVDGVEVHNAKTKPREEQYNSTYRVVSVLTVLHQDW | | ASVVCLLNNFYPREAKVQWKVDNALQSGNSQESVTEQDSKD | |
| | | LNGKEYKCKVSNKALPAPIEKTISKAKGQPREPQVYTLPPSRE | | STYSLSSTLTLSKADYEKHKVYACEVTHQGLSSPVTKSENRGE | |
| | | EMTKNQVSLTCLVKGFYPSDIAVEWESNGQPENNYKTTPPVL | | C | |
| | | DSDGSFFLYSKLTVDKSRWQQGNVFSCSVMHEALHNHYTQK | | | |
| | | SLSLSP | | | |
| Vhh2b- | SEQ ID | MGWSCIILFLVATATGVHSQVQLQESGGGSVQAGGSLRESC | SEQ ID | MGWSCIILFLVATATGVHSAFELTQTPSSVEAAVGGTVTIKCQ | The fusion proteins |
| PDA0044 | NO: 66 | AASSYTISSVCMGWFRQAPGKEREGVAGIAPDGSTGYGDS | NO: 49 | ASQSISSYLAWYQQKPGQPPKLLIYGASNLKSGVPSRFKGSGS | of PD1 binding |
| | | VKGRFTISMLYDNAKNTLYLQMNSLKPEDTAMYYCAAAS | | GTQFTLTISDLEAADAATYYCQSYYYSSSSSYSFGGGTEVYVK | domain and IL-2Rβ |
| | | PGRCFLPRTALEPALYYNWGQGTQVTVSSGGSQSVEESGG | | RTVAAPSVFIFPPSDEQLKSGTASVVCLLNNFYPREAKVQWK | binding domain |
| | | RLVTPGTPLTLTCTASGFSLSGYYMSWVRQAPGKGLEWIGSIS | | VDNALQSGNSQESVTEQDSKDSTYSLSSTLTLSKADYEKHKV | The sequence of |
| | | YGGSTYYASWAKGRFTISKTSTTVDLKITSPTTEDTATYFCAA | | YACEVTHQGLSSPVIKSFNRGEC | PD1 binding |
| | | GYTTNTAFDPWGPGTLVTVSSASTKGPSVFPLAPSSKSTSGGT | | | domain is |
| | | AALGCLVKDYFPEPVTVSWNSGALTSGVHTFPAVLQSSGLYS | | | underlined and the |
| | | LSSVVTVPSSSLGTQTYICNVNHKPSNTKVDKRVEPKSCDKTH | | | sequence of IL-2Rβ |
| | | TCPPCPAPELRRGPSVFLFPPKPKDTLMISRTPEVTCVVVDVSH | | | binding domain is |
| | | EDPEVKFNWYVDGVEVHNAKTKPREEQYNSTYRVVSVLTVL | | | shown in bold. |
| | | HQDWLNGKEYKCKVSNKALPAPIEKTISKAKGQPREPQVYTL | | | |
| | | PPSREEMTKNQVSLTCLVKGFYPSDIAVEWESNGQPENNYKT | | | |
| | | TPPVLDSDGSFFLYSKLTVDKSRWQQGNVFSCSVMHEALHN | | | |
| | | HYTQKSLSLSP | | | |
| Vhb2b- | SEQ ID | MGWSCIILFLVATATGVHSQVQLQESGGGSVQAGGSLRLSC | SEQ ID | MGWSCHILELVATATGVHSIEMTQTPESVSAAVGGTVTIKCQA | |
| PDA0129 | NO: 67 | AASSYTISSVCMGWFRQAPGKEREGVAGIAPDGSTGYGDS | NO: 51 | SEDIYSLLAWYQQKPGQPPKLLIYSASTLASGVPSREKGSGYG | |
| | | VKGRFTISMLYDNAKNTLYLQMNSLKPEDTAMYYCAAAS | | TEFTLTISGVQAADAATYYCQGGYYSSSADNTFGGGTEVVVK | |
| | | PGRCFLPRTALEPALYYNWGQGTQVTVSSGGSQSVEESGG | | RTVAAPSVFIFPPSDEQLKSGTASVVCLLNNFYPREAKVQWK | |
| | | RLVTPGTPLTLTCTVSGESLSSYDMSWVRQAPGKGLEYIGIISS | | VDNALQSGNSQESVTEQDSKDSTYSLSSTLILSKADYEKHKV | |
| | | SGSTYYASWAKGRFTISKTSTTVDLKITSPTTEDTATYFCARL | | YACEVTHQGLSSPVTKSFNRGEC | |
| | | GYVGYGYADIWGPGTLVTVSSASTKGPSVFPLAPSSKSTSGGT | | | |
| | | AALGCLVKDYFPEPVTVSWNSGALTSGVHTFPAVLQSSGLYS | | | |
| | | LSSVVTVPSSSLGTQTYICNVNHKPSNTKVDKRVEPKSCDKTH | | | |
| | | TCPPCPAPELRRGPSVFLFPPKPKDTLMISRTPEVTCVVVDVSH | | | |
| | | EDPEVKFNWYVDGVEVHNAKTKPREEQYNSTYRVVSVLTVL | | | |
| | | HQDWLNGKEYKCKVSNKALPAPIEKTISKAKGQPREPQVYTL | | | |
| | | PPSREEMTKNQVSLTCLVKGFYPSDIAVEWESNGQPENNYKT | | | |
| | | TPPVLDSDGSFFLYSKLTVDKSRWQQGNVFSCSVMHEALHN | | | |
| | | HYTQKSLSLSP | | | |
| Vhh2b- | SEQ ID | MGWSCIILFLVATATGVHSQVQLQESGGGSVQAGGSLRLSC | SEQ ID | MGWSCIILFLVATATGVHSAQVLTQTPSPVSAAVGGTVTISCQ | The fusion proteins |
| PDC0037 | NO: 68 | AASSYTISSVCMGWFRQAPGKEREGVAGIAPDGSTGYGDS | NO: 53 | SSESVYNNNWLAWYQQKPGQPPKFLIYDASTLASGVPSRFSG | of PD1 binding |
| | | VKGRFTISMLYDNAKNTLYLQMNSLKPEDTAMYYCAAAS | | SGSGTQFTLTISGVQADDAATYYCQGTYYSSDWYFVFGGGTE | domain and IL-2Rβ |
| | | PGRCFLPRTALEPALYYNWGQGTQVTVSSGGSQSVEESGG | | VVVKRTVAAPSVFIFPPSDEQLKSGTASVVCLLNNFYPREAKY | binding domain. |
| | | RLVTPGTPLTLTCTVSGESLSYYAMIWVRQAPGKGLEWIGFIN | | QWKVDNALQSGNSQESVTEQDSKDSTYSLSSTLTLSKADYEK | The sequence of |
| | | SYGKTYYANWAKGRFTISKTSTTVDLKMTSLTSEDTATYFCA | | HKVYACEVTHQGLSSPVTKSFNRGEC | PD1 binding |
| | | RERDNYHENLWGQGILVTVSSASTKGPSVFPLAPSSKSTSGG | | | domain is |
| | | TAALGCLVKDYFPEPVTVSWNSGALTSGVHTFPAVLQSSGLY | | | underlined and the |
| | | SLSSVVTVPSSSLGTQTYICNVNHKPSNTKVDKRVEPKSCDKT | | | sequence of IL-2Rβ |
| | | HTCPPCPAPELRRGPSVFLFPPKPKDTLMISRTPEVTCVVVDVS | | | binding domain is |
| | | HEDPEVKFNWYVDGVEVHNAKTKPREEQYNSTYRVVSVLIV | | | shown in bold. |
| | | LHQDWLNGKEYKCKVSNKALPAPIEKTISKAKGQPREPQVYT | | | |
| | | LPPSREEMTKNQVSLTCLVKGFYPSDIAVEWESNGQPENNYK | | | |
| | | TTPPVLDSDGSFFLYSKLTVDKSRWQQGNVFSCSVMHEALHN | | | |
| | | HYTQKSLSLSP | | | |
| Vhh2b- | SEQ ID | MGWSCIILFLVATATGVHSQVQLQESGGGSVQAGGSLRISC | SEQ ID | MGWSCIILFLVATATGVHSADIVMTQTPASVEAAVGGIVTIKC | |
| PDC0053 | NO: 69 | AASSYTISSVCMGWERQAPGKEREGVAGIAPDGSTGYGDS | NO: 54 | QASQSISSYLAWYQQKPGQPPNLLIYRASTLASGVPSRFKGSG | |
| | | VKGRFTISMLYDNAKNILYLQMINSLKPEDTAMYYCAAAS | | SGTQFTLTISDLEAADAATYYCQGYHESSTSSNYGNAFGGGT | |
| | | PGRCFLPRTALEPALYYNWGQGTQVTVSSGGSQQHLVESG | | EVVVKRTVAAPSVFIFPPSDEQLKSGTASVVCLLNNFYPREAK | |
| | | GGLVKPGASLTLTCKASGFYFSGYYYMSWVRQAPGKGLEWI | | VQWKVDNALQSGNSQESVTEQDSKDSTYSLSSTLILSKADYE | |
| | | ASIYSGGSGNTYYASWAKGRFTISKTSSTTVTLQMTSLTAADT | | KHKVYACEVTHQGLSSPVTKSFNRGEC | |
| | | ATYFCARGADYTHDYPADAAAWSFDPWGPGTLVTVSSASTK | | | |
| | | GPSVFPLAPSSKSTSGGTAALGCLVKDYFPEPVTVSWNSGALT | | | |
| | | SGVHTFPAVLQSSGLYSLSSVVTVPSSSLGTQTYICNYNHKPS | | | |
| | | NTKVDKRVEPKSCDKTHTCPPCPAPELRRGPSVFLFPPKPKDI | | | |
| | | LMISRTPEVTCVVVDVSHEDPEVKFNWYVDGVEVHNAKTKP | | | |
| | | REEQYNSTYRVVSVLTVLHQDWLNGKEYKCKVSNKALPAPI | | | |
| | | EKTISKAKGQPREPQVYTLPPSREEMTKNQVSLTCLVKGFYPS | | | |
| | | DIAVEWESNGQPENNYKITPPVLDSDGSPFLYSKLTVDKSRW | | | |
| | | QQGNVFSCSVMHEALHNHYTQKSLSLSP | | | |
| Vhh2b- | SEQ ID | MGWSCIILFLVATATGVHSQVQLQESGGGSVQAGGSERLSC | SEQ ID | MGWSCIILFLVATATGVHSDIVMTQTPASVSAAVGGTVSINC | The fusion proteins |
| PDE0171 | NO: 70 | AASSYTISSVCMGWFRQAPGKEREGVAGIAPDGSTGYGDS | NO: 55 | QASEDIENFLAWYQQKPGQPPSLLIYETSKLASGVPSRESGSGS | of PD1 binding |
| | | VKGRFTISMLYDNAKNTLYLQMNSLKPEDTAMYYCAAAS | | GTEFTLTISDLEAADAATYYCQSYDWSDSSTYDNTFGGGTEV | domain and IL-2Rβ |
| | | PGRCFLPRTALEPALYYNWGQGTQVTVSSGGSQSLEESGG | | VVKRTVAAPSVFIFPPSDEQLKSGTASVVCLLNNFYPREAKVQ | binding domain. |
| | | GLVKPGASLILTCTASGESFSGSYYMCWVRQAPGKGLECIAC | | WKVDNALQSGNSQESVTEQDSKDSTYSLSSILTLSKADYEKH | The sequence of |
| | | IYAGSSGATYYASWAKGRETVSKTSSTTVTLQMTSLTAADTA | | KVYACEVTHQGLSSPVTKSFNRGEC | PD1 binding |
| | | TYFCARHTNGIGLGLWGPGTLVTVSSASTKGPSVFPLAPSSKS | | | domain is |
| | | TSGGTAALGCLVKDYFPEPVTVSWNSGALTSGVHIFPAVLQS | | | underlined and the |
| | | SGLYSLSSVVTVPSSSLGTQTYICNVNHKPSNTKVDKRVEPKS | | | sequence of IL-2Rβ |
| | | CDKTHTCPPCPAPELRRGPSVFLFPPKPKDTLMISRTPEVTCVV | | | binding domain is |
| | | VDVSHEDPEVKFNWYVDGVEVHNAKTKPREEQYNSTYRVVS | | | shown in bold. |
| | | VLTVLHQDWLNGKEYKCKVSNKALPAPIEKTISKAKGQPREP | | | |
| | | QVYTLPPSREEMTKNQVSLTCLVKGFYPSDIAVEWESNGQPE | | | |
| | | NNYKTTPPVLDSDGSFFLYSKLTVDKSRWQQGNVFSCSVMH | | | |
| | | EALHNHYTQKSLSLSP | | | |
| Vhh2b- | SEQ ID | MGWSCIILFLVATATGVHSQEQLEESGGDLVKPGASLTLTCT | SEQ ID | MGWSCIILFLVATATGVHSQVQLQESGGGSVQAGGSERLSC | |
| PDA0041 | NO: 47 | ASGFSFNSYWMSWVRQAPGKGLEWIADIGAGGSGSTFYASW | NO: 71 | AASSYTISSVCMGWFRQAPGKEREGVAGIAPDGSTGYGDS | |
| | | AKGRFTISKTSSTTYTLQMTSLTAADTATYFCARYVDGGHSD | | VKGRFTISMLYDNAKNTLYLQMNSLKPEDTAMYYCAAAS | |
| | | LWGPGTLVTVSSASTKGPSVFPLAPSSKSISGGTAALGCLVKD | | PGRCFLPRTALEPALYYNWGQGTQVTVSSGGSAFELTQTPS | |
| | | YFPEPVTVSWNSGALTSGVHTFPAVLQSSGLYSLSSVVTVPSS | | SVEAAVGGTVSINCQASEDIERYLAWYQQRPGQPPKLLIYTAS | |
| | | SLGTQTYICNVNHKPSNTKVDKRVEPKSCDKTHICPPCPAPEL | | SLPSGVPSRFKGSGSGTQFTLTISDLEAADAATYYCQQGWSD | |
| | | RRGPSVFLFPPKPKDTLMISRTPEVTCVYVDVSHEDPEVKENW | | ANVDNTFGGGTEVVVKRTVAAPSVFIFPPSDEQLKSGTASVV | |
| | | YVDGVEVHNAKTKPREEQYNSTYRVVSVLTVLHQDWLNGK | | CLLNNFYPREAKVQWKVDNALQSGNSQESVTEQDSKDSTYS | |
| | | EYKCKVSNKALPAPIEKTISKAKGQPREPQVYTLPPSREEMTK | | LSSTLTLSKADYEKHKVYACEVTHQGLSSPVTKSFNRGEC | |
| | | NQVSLTCLVKGFYPSDIAVEWESNGQPENNYKTTPPVLDSDG | | | |
| | | SFFLYSKLTVDKSRWQQGNVFSCSVMHEALHNHYTQKSLSLS | | | |
| | | P | | | |
| Vhh2b- | SEQ ID | MGWSCIILFLVATATGVHSQEQLEESGGDLVKPGASLTLTCT | SEQ ID | MGWSCIILFLVATATGVASQVQLQESGGGSVQAGGSLRLSC | |
| PDA0070 | NO: 48 | ASGFSFNSYWMSWVRQAPGKGLEWIADIGAGGSGSTFYASW | NO: 72 | AASSYTISSVCMGWFRQAPGKEREGVAGIAPDGSTGYGDS | |
| | | AKGRFTISKTSSTTVTLQMTSLTAADTATYFCARYVDGGHSD | | VKGRFTISMLYDNAKNTLYLQMNSLKPEDTAMYYCAAAS | |
| | | LWGPGTLVTVSSASTKGPSVFPLAPSSKSTSGGTAALGCLVKD | | PGRCFLPRTALEPALYYNWGQGTQVTVSSGGSAFELTQTPS | |
| | | YFPEPVTVSWNSGALISGVHTFPAVLQSSGLYSLSSVVTVPSS | | SVEAAVGGTVSINCQASEDIERYLAWYQQRPGQPPKLLIYTAS | |
| | | SLGTQTYICNVNHKPSNTKVDKRVEPKSCDKTHTCPPCPAPEL | | SLPSGVPSRFKGSGSGTQFTLTISDLEAADAATYYCQQGWSD | |
| | | RRGPSVFLFPPKPKDTLMISRTPEVTCVVVDVSHEDPEVKFNW | | ANVDNTEGGGTEVVVKRTVAAPSVFIFPPSDEQLKSGTASVV | |
| | | YVDGVEVHNAKTKPREEQYNSTYRVVSVLTVLHQDWLNGK | | CLLNNFYPREAKVQWKVDNALQSGNSQESVTEQDSKDSTYS | |
| | | EYKCKVSNKALPAPIEKTISKAKGQPREPQVYTLPPSREEMTK | | LSSTLTLSKADYEKHKVYACEVTHQGLSSPVTKSFNRGEC | |
| | | NQVSLTCLVKGFYPSDIAVEWESNGQPENNYKTTPPVLDSDG | | | |
| | | SFFLYSKLTVDKSRWQQGNVFSCSVMHEALHNHYTQKSLSLS | | | |
| | | P | | | |
| Vhh2b- | SEQ ID | MGWSCIILFLVATATGVHSQSVEESGGRLVTPGTPLTLTCTVS | SEQ ID | MGWSCIILFLVATATGVHSQVQLQESGGGSVQAGGSERLSC | The fusion proteins |
| PDA0107 | NO: 39 | GFSLSRYYMSWVRQAPGKGLEWIGYIYADSGSTWYASWAK | NO: 73 | AASSYTISSVCMGWFRQAPGKEREGVAGIAPDGSTGYGDS | of PD1 binding |
| | | GRFTISKTSTTVDLKMTSLTTEDTATYFCARRADHDGAFDLW | | VKGRFTISMLYDNAKNTLYLQMNSLKPEDTAMYYCAAAS | domain and IL-2Rβ |
| | | GPGTLVTVSSASTKGPSVFPLAPSSKSTSGGTAALGCLVKDYF | | PGRCFLPRTALEPALYYNWGQGTQVTVSSGGSAAVLTQTP | binding domain. |
| | | PEPVTVSWNSGALTSGVHTFPAVLQSSGLYSLSSVVTVPSSSL | | SPYSAAVGGTVSISCQSSQSVYGKNELSWYQQKPGQPPKLLIY | The sequence of |
| | | GTQTYICNVNHKPSNTKVDKRVEPKSCDKTATCPPCPAPELR | | YASTLASGVPSRESGSGSGTQFALTISGVQADDAATYYCAGG | PD1 binding |
| | | RGPSVFLFPPKPKDTLMISRTPEVTCVVVDYSHEDPEVKFNW | | YSSSVDTTFGGGTEVVVKRTVAAPSVFIFPPSDEQLKSGTASV | domain is |
| | | YVDGVEVHNAKTKPREEQYNSTYRVVSVLTVLHQDWLNGK | | VCLLNNFYPREAKVQWKVDNALQSGNSQESVTEQDSKDSTY | underlined and the |
| | | EYKCKVSNKALPAPIEKTISKAKGQPREPQVYTLPPSREEMTK | | SLSSILTLSKADYEKHKVYACEVTHQGLSSPVTKSFNRGEC | sequence of IL-2Rβ |
| | | NQVSLTCLVKGFYPSDIAVEWESNGQPENNYKTTPPVLDSDG | | | binding domain is |
| | | SFFLYSKLTVDKSRWQQGNVFSCSVMHEALHNHYTQKSLSLS | | | shown in bold. |
| | | P | | | |
| Vhh2b- | SEQ ID | MGWSCIILFLVATATGVHSQSVEESGGRLVTPGTPLTLICIVS | SEQ ID | MGWSCIILFLVATAGVHSQVQLQESGGGSVQAGGSERLSC | |
| PDA0138 | NO: 41 | GESLSSYHIMCWVRQPPGKGLEYIGIISGGAITYYATWAKGRET | NO: 74 | AASSYTISSVCMGWFRQAPGKEREGVAGIAPDGSTGYGDS | |
| | | ISITSSTTVDLKITSPTTEDTATYFCARGGGYGGGDYNFDLWG | | VKGRFTISMLYDNAKNTLYLQMNSLKPEDTAMYYCAAAS | |
| | | QGTLVTVSSASTKGPSVFPLAPSSKSTSGGTAALGCLVKDYFP | | PGRCFLPRTALEPALYYNWGQGTQVTVSSGGSAAVLTQTP | |
| | | EPVTVSWNSGALISGVHTFPAVLQSSGLYSLSSVVTVPSSSLG | | SPVSAAVGATYSISCQSSQSVYGNSELAWYQQKPGQRPKLLIE | |
| | | TQTYICNVNHKPSNTKVDKRVEPKSCDKTHTCPPCPAPELRR | | EASKLASGVPSRESGSGSGTQFSLAISDVQADDAATYYCAGG | |
| | | GPSVFLFPPKPKDTLMISRTPEVTCVVVDVSHEDPEVKENWY | | YSSTSDTTFGGGTEVVVKRTVAAPSVFIFPPSDEQLKSGTASV | |
| | | VDGVEVHNAKTKPREEQYNSTYRVVSVLTVLHQDWLNGKE | | VCLLNNFYPREAKVQWKVDNALQSGNSQESVTEQDSKDSTY | |
| | | YKCKVSNKALPAPIEKTISKAKGQPREPQVYTIPPSREEMTKN | | SLSSTLILSKADYEKHKVYACEVTHQGLSSPVTKSFNRGEC | |
| | | QVSLICLVKGFYPSDIAVEWESNGQPENNYKTIPPVLDSDGS | | | |
| | | FFLYSKLTVDKSRWQQGNVFSCSVMHEALHNHYTQKSLSLSP | | | |
| Vhh2b-GY5 | SEQ ID | MGWSCHLELVATATGVHSQVQLQQSGPELVRPGVSVKISCK | SEQ ID | MGWSCIILFLVATATGVHSQVQLQESGGGSVQAGGSLRLSC | |
| | NO: 45 | GSGHTFTDYALHWVRQSHVKSLEWIGVISSYNGITNYNQRFK | NO: 75 | AASSYTISSVCMGWFRQAPGKEREGVAGIAPDGSTGYGDS | |
| | | GKATMTVDKSSSTAYLELARLTSEDSAIYYCSREEWDYFYYE | | VKGRFTISMLYDNAKNTLYLQMNSLKPEDTAMYYCAAAS | |
| | | DYWGQGTTLTVSSASTKGPSVFPLAPSSKSTSGGIAALGCLV | | PGRCFLPRTALEPALYYNWGQGTQVTVSSGGSDIMLTQSP | |
| | | KDYFPEPVTVSWNSGALTSGVHTFPAVLQSSGLYSLSSVVTVP | | LSLPVSLGDQASISCRSSQGIVHSDGNTYLEWYLQKPGQSPKL | |
| | | SSSLGTQTYICNVNHKPSNTKVDKRVEPKSCDKTHTCPPCPAP | | LIYKVSNRFSGVPDRFSGSGSGTDFILKISRVEAEDLGVYYCFQ | |
| | | ELRRGPSVFLFPPKPKDTLMISRTPEVTCVVVDVSHEDPEVKF | | GSHVPYTEGGGTKLEIKRTVAAPSVFIFPPSDEQLKSGTASVV | |
| | | NWYVDGVEVHNAKTKPREEQYNSTYRVVSVLTVLHQDWLN | | CLLNNFYPREAKVQWKVDNALQSGNSQESVTEQDSKDSTYS | |
| | | GKEYKCKVSNKALPAPIEKTISKAKGQPREPQVYTLPPSREEM | | LSSTLTLSKADYEKFIKVYACEVTHQGLSSPVTKSFNRGEC | |
| | | TKNQVSLTCLVKGFYPSDIAVEWESNGQPENNYKTTPPVLDS | | | |
| | | DGSFFLYSKLIVDKSRWQQGNVFSCSVMHEALHNHYTQKSL | | | |
| | | SLSP | | | |
| Vhh2b-GY14 | SEQ ID | MGWSCIILFLVATATGVHSQVQLQQSGAELVRPGASYKLSCK | SEQ ID | MGWSCHLELVATATGVHSQVQLQESGGGSVQAGGSLRLSC | The fusion proteins |
| | NO: 46 | ALGDTFTDYEIHWVKQTPVHGLEWIGVIHPGSGGTVYNQKFK | NO: 76 | AASSYTISSVCMGWFRQAPGKEREGVAGIAPDGSTGYGDS | of PD1 binding |
| | | GKATLTADKYSSTAYMELSSLTSEDSAVYYCTREGMNTDWY | | VKGRFTISMLYDNAKNTLYLQMNSEKPEDTAMYYCAAAS | domain and IL-2Rβ |
| | | FDVWGAGTTYTVSSASTKGPSVFPLAPSSKSTSGGIAALGCL | | PGRCFLPRTALEPALYYNWGQGTQVTVSSGGSDILMTQDE | binding domain. |
| | | VKDYFPEPVIVSWNSGALTSGVHTFPAVLQSSGLYSLSSVVT | | LSLPVSLGDQASISCRSSQTIVHTNGNTYLEWYLQKPGQSPKL | The sequence of |
| | | VPSSSLGTQTYICNVNHKPSNTKVDKRVEPKSCDKTHTCPPCP | | LIYKVSNRESGVPDRFSGSGSGTYFTLKISRLEAFDLGVYYCE | PD1 binding |
| | | APELRRGPSVFLFPPKPKDTLMISRTPEVTCVVVDVSHEDPEV | | QGSHVPYTFGGGTKLEMKRTVAAPSVFIFPPSDEQLKSGTASV | domain is |
| | | KFNWYVDGVEVHNAKTKPREEQYNSTYRVVSVLTVLHQDW | | VCLLNNFYPREAKVQWKVDNALQSGNSQESVTEQDSKDSTY | underlined and the |
| | | LNGKEYKCKVSNKALPAPIEKTISKAKGQPREPQVYTLPPSRE | | SLSSTLTLSKADYEKHKVYACEVTHQGLSSPVTKSFNRGEC | sequence of IL-2Rβ |
| | | EMTKNQVSLTCLVKGFYPSDIAVEWESNGQPENNYKTTPPVL | | | binding domain is |
| | | DSDGSFFLYSKLTVDKSRWQQGNVFSCSVMHEALHNHYTQK | | | shown in bold. |
| | | SLSLSP | | | |
| Vhh2g- | SEQ ID | MGWSCIILFLVATATGVHSQVQLQESGGGSVQAGGSLRLSC | SEQ ID | MGWSCIILFLVAIATGVASAFELTQTPSSVEAAVGGTVTIKCQ | The fusion proteins |
| PDA0044 | NO: 77 | AASGYTYRDYYMGWERQAPGREREGVASIYIRGSREGST | NO: 49 | ASQSISSYLAWYQQKPGQPPKLLIYGASNLKSGVPSREKGSGS | of PD1 binding |
| | | RYSSSVEGRETTILDTAKNTLYLQMNSLKPEDTAMYYCAA | | GTQFTLTISDLEAADAATYYCQSYYYSSSSSYSFGGGTEVVVK | domain and IL-2Rγ |
| | | DDRTWLPRVQLGGPRENEYNYWGQGTQVTVSSGGSQSVE | | RTVAAPSVFIFPPSDEQLKSGTASVVCLLNNFYPREAKVQWK | binding domain. |
| | | ESGGRLVTPGTPLTLICTASGESLSGYYMSWVRQAPGKGLEW | | VDNALQSGNSQESVTEQDSKQSTYSLSSTLILSKADYEKHKV | The sequence of |
| | | IGSISYGGSTYYASWAKGRFTISKTSTTVDLKITSPTTEDTATY | | YACEVTHQGLSSPVTKSFNRGEC | PD1 binding |
| | | FCAAGYTINTAFDPWGPGTLVTVSSASTKGPSVFPLAPSSKST | | | domain is |
| | | SGGTAALGCLVKDYFPEPVTVSWNSGALTSGVHTFPAVLQSS | | | underlined and the |
| | | GLYSLSSVVTVPSSSLGTQTYICNVNHKPSNTKVDKRVEPKSC | | | sequence of IL-2Rγ |
| | | DKTHTCPPCPAPELRRGPSVFLFPPKPKDTLMISRTPEVTCVV | | | binding domain is |
| | | VDVSHEDPEVKFNWYVDGVEVHNAKTKPREEQYNSTYRVVS | | | shown in bold. |
| | | VLTVLHQDWLNGKEYKCKVSNKALPAPIEKTISKAKGQPREP | | | |
| | | QVYTIPPSREEMTKNQVSLTCLVKGFYPSDIAVEWESNGQPE | | | |
| | | NNYKTTPPVLDSDGSFFLYSKLIVDKSRWQQGNVFSCSVMH | | | |
| | | EALHNHYTQKSLSLSP | | | |
| Vh12g- | SEQ ID | MGWSCIILFLVATATGVHSQVQLQESGGGSVQAGGSLRLSC | SEQ ID | MGWSCIILFLVATATGVHSIEMTQTPESVSAAVGGTVTIKCQA | The fusion proteins |
| PDA0129 | NO: 78 | AASGYTYRDYYMGWERQAPGREREGVASTYTRGSREGST | NO: 51 | SEDIYSLLAWYQQKPGQPPKLLIYSASTLASGVPSREKGSGYG | of PD1 binding |
| | | RYSSSVEGRETITLDTAKNTLYLQMNSLKPEDTAMYYCAA | | TEFTLTISGVQAADAATYYCQGGYYSSSADNTFGGGTEVVVK | domain and IL-2Rγ |
| | | DDRTWLPRVQLGGPRENEYNYWGQGTQVTVSSGGSQSVE | | RTVAAPSVFIFPPSDEQLKSGTASVVCLINNFYPREAKVQWK | binding domain. |
| | | ESGGRLVTPGTPLTLTCTVSGFSLSSYDMSWVRQAPGKGLEYI | | VDNALQSGNSQESVTEQDSKDSTYSLSSTLILSKADYEKHKV | The sequence of |
| | | GIISSSGSTYYASWAKGRFTISKTSTTVDLKITSPTTEDTATYFC | | YACEVTHQGLSSPVTKSFNRGEC | PD1 binding |
| | | ARLGYVGYGYADIWGPGTLVTVSSASTKGPSVFPLAPSSKSTS | | | domain is |
| | | GGTAALGCLVKDYFPEPVTVSWNSGALTSGVHTFPAVLQSSG | | | underlined and the |
| | | LYSLSSVVTVPSSSLGTQTYICNVNHKPSNTKVDKRVEPKSCD | | | sequence of IL-2Rγ |
| | | KTHTCPPCPAPELRRGPSVFLFPPKPKDTLMISRTPEVTCVVV | | | binding domain is |
| | | DVSHEDPEVKFNWYVDGVEVHNAKIKPREEQYNSTYRVVSV | | | shown in bold. |
| | | LTVLHQDWLNGKEYKCKVSNKALPAPIEKTISKAKGQPREPQ | | | |
| | | VYTLPPSREEMTKNQVSLTCLVKGFYPSDIAVEWESNGQPEN | | | |
| | | NYKTTPPVLDSDGSFFLYSKLTVDKSRWQQGNVFSCSVMHE | | | |
| | | ALHNHYTQKSLSLSP | | | |
| Vhh2g- | SEQ ID | IMGWSCIILFLVATATGVHSQVQLQESGGGSVQAGGSLRLSC | SEQ ID | MGWSCIILFLVATATGVHSAQVLTQTPSPVSAAVGGTVTISCQ | |
| PDC0037 | NQ. 79 | AASGYTYRDYYMGWFRQAPGREREGVASIYTRGSREGST | NO: 53 | SSESVYNNNWLAWYQQKPGQPPKELIYDASTLASGVPSRESG | |
| | | RYSSSVEGRFTITLDTAKNTLYLQMNSLKPEDTAMYYCAA | | SGSGTQFTLTISGVQADDAATYYCQGTYYSSDWYFVFGGGTE | |
| | | DDRTWLPRVQLGGPRENEYNYWGQGTQVTVSSGGSQSVE | | VVVKRTVAAPSVFIFPPSDEQLKSGTASVVCLLNNFYPREAKV | |
| | | ESGGRLVTPGTPLTLTCTVSGESLSYYAMIWVRQAPGKGLEW | | QWKVDNALQSGNSQESVTEQDSKDSTYSLSSTLILSKADYEK | |
| | | IGFINSYGKTYYANWAKGRFTISKTSTTVDLKMTSLTSEDTAT | | HKVYACEVTHQGLSSPVIKSFNRGEC | |
| | | YFCARERDNYHENLWGQGTLVTVSSASTKGPSVFPLAPSSKS | | | |
| | | TSGGTAALGCLVKDYFPEPVTVSWNSGALTSGVHTFPAVLQS | | | |
| | | SGLYSLSSVVTVPSSSLGTQTYICNVNHKPSNTKVDKRVEPKS | | | |
| | | CDKTHTCPPCPAPELRRGPSVFLFPPKPKDTLMISRTPEVTCVV | | | |
| | | VDVSHEDPEVKFNWYVDGVEVHNAKTKPREEQYNSTYRVVS | | | |
| | | VLTVLHQDWLNGKEYKCKVSNKALPAPIEKTISKAKGQPREP | | | |
| | | QVYTLPPSREEMTKNQVSLTCLVKGFYPSDIAVEWESNGQPE | | | |
| | | NNYKITPPVLDSDGSFFLYSKLTVDKSRWQQGNVFSCSVMH | | | |
| | | EALHNHYTQKSLSLSP | | | |
| Vhh2g- | SEQ ID | MGWSCIILFLVATATGVHSQVQLQESGGGSVQAGGSERLSC | SEQ ID | MGWSCIILFLVATATGVHSADIVMTQTPASVEAAVGGIVTIKC | The fusion proteins |
| PDC0053 | NO: 80 | AASGYTYRDYYMGWFRQAPGREREGVASIYTRGSREGST | NO: 54 | QASQSISSYLAWYQQKPGQPPNLLIYRASTLASGVPSREKGSG | of PD1 binding |
| | | RYSSSVEGRFTITLDTAKNTLYLQMNSLKPEDTAMYYCAA | | SGTQFILTISDLEAADAATYYCQGYHESSTSSNYGNAFGGGI | domain and IL-2Rγ |
| | | DDRTWLPRVQLGGPRENEYNYWGQGTQVTVSSGGSQQHL | | EVVVKRTVAAPSVFIFPPSDEQLKSGTASVVCLLNNFYPREAK | binding domain. |
| | | VESGGGLVKPGASLTLTCKASGFYESGYYYMSWVRQAPGKG | | VQWKVDNALQSGNSQESVTEQDSKDSTYSLSSTLTLSKADYE | The sequence of |
| | | LEWIASIYSGGSGNTYYASWAKGRFTISKTSSTTVTLQMTSLT | | KHKVYACEVTHQGLSSPVTKSFNRGEC | PD1 binding |
| | | AADTATYFCARGADYTHDYPADAAAWSFDPWGPGILVTVSS | | | domain is |
| | | ASTKGPSVFPLAPSSKSTSGGTAALGCLVKDYFPEPVTVSWNS | | | underlined and the |
| | | GALTSGVHTFPAVLQSSGLYSLSSVVTVPSSSLGTQTYICNVN | | | sequence of IL-2Rγ |
| | | HKPSNTKVDKRVEPKSCDKTHTCPPCPAPELRRGPSVFLFPPK | | | binding domain is |
| | | PKDTLMISRTPEVTCVVVDVSHEDPEVKFNWYVDGVEVHNA | | | shown in bold. |
| | | KTKPREEQYNSTYRVVSVLIVLHQDWLNGKEYKCKVSNKAL | | | |
| | | PAPIEKTISKAKGQPREPQVYTLPPSREEMTKNQVSLTCLVKG | | | |
| | | FYPSDIAVEWESNGQPENNYKTTPPVLDSDGSFFLYSKLTVDK | | | |
| | | SRWQQGNVFSCSVMHEALHNHYTQKSLSLSP | | | |
| Vhh2g- | SEQ ID | MGWSCIILFLVATATGVHSQVQLQESGGGSVQAGGSERLSC | SEQ ID | MGWSCIILFLVATATGVHSDIVMTQTPASVSAAVGGTVSINC | |
| PDE0171 | NO: 81 | AASGYTYRDYYMGWERQAPGREREGVASIYTRGSREGST | NO: 55 | QASEDIENFLAWYQQKPGQPPSLLIYETSKLASGVPSRESGSGS | |
| | | RYSSSVEGRFTITLDTAKNTLYLQMNSLKPEDTAMYYCAA | | GTEFTLTISDLEAADAATYYCQSYDWSDSSTYDNTFGGGTEV | |
| | | DDRTWLPRVQLGGPRENEYNYWGQGTQVTVSSGGSQSLE | | VVKRTVAAPSVFIFPPSDEQLKSGTASVVCLLNNFYPREAKVQ | |
| | | ESGGGLVKPGASLTLTCTASGESESGSYYMCWVRQAPGKGLE | | WKVDNALQSGNSQESVTEQDSKDSTYSLSSILTLSKADYEKH | |
| | | CIACIYAGSSGATYYASWAKGRFTVSKTSSTTVTLQMTSLTA | | KVYACEVTHQGLSSPVTKSFNRGEC | |
| | | ADTATYFCARHTNGIGLGLWGPGTLVTVSSASTKGPSVFPLA | | | |
| | | PSSKSTSGGTAALGCLVKDYFPEPVTVSWNSGALTSGVHTPP | | | |
| | | AVLQSSGLYSLSSVVTVPSSSLGTQTYICNVNRKPSNTKVDKR | | | |
| | | VEPKSCDKTHTCPPCPAPELRRGPSVFLFPPKPKDTLMISRTPE | | | |
| | | VTCVVVDVSHEDPEVKFNWYVDGVEVHNAKTKPREEQYNS | | | |
| | | TYRVVSVLTVLAQDWLNGKEYKCKVSNKALPAPIEKTISKAK | | | |
| | | GQPREPQVYTLPPSREEMTKNQVSLTCLVKGFYPSDIAVEWE | | | |
| | | SNGQPENNYKTTPPVLDSDGSFFLYSKLIVDKSRWQQGNVFS | | | |
| | | CSVMHEALHNHYTQKSLSLSP | | | |
| | SEQ | | | | |
| abbreviation | ID NO | Sequences | | | Description |

| | | | |
|---|---|---|---|
| Vhh2b-Nivo | SEQ ID | MGWSCIILFLVATATGVHSQVQLQESGGGSVQAGGSLRLSCAASSYTISSVCMGWFRQAPGKEREGVAGIAPDGSTGYGDSVKGRFT | The fusion proteins |
| scFv | NO: 82 | ISKDNAKNTLYLQMNSLKPEDTAMYYCAAASPGRCFLPRTALEPALYYNWGQGTQVTVSSGGSEIVLTQSPARLSLSPGERATLSCRA | of PD1 binding |
| | | SQSVSSYLAWYQQKPGQAPRLLIYDASNRATGIPARFSGSGSGTDFTLTISSLEPEDFAVYYCQQSSNWPRTFGQGTKVEIKRTVGGGGSGG | domain and IL-2Rβ |
| | | GGSGGGGSQVQLVESGGGVVQPGRSLRLDCKASGITFSNSGMHWVRQAPGKGLEWVAVIWYDGSKRYYADSVKGRFTISRDNSKNTLFL | binding domain. |
| | | QMNSLRAEDTAYYDATNDDYWGQGTLVTVSS | The sequence of |
| | | | PD1 binding |
| | | | domain is |
| | | | underlined and the |
| | | | sequence of IL-2Rβ |
| | | | binding domain is |
| | | | shown in bold. |
| Vhh2g4- | SEQ ID | MGWSCIILFLVATATGVHSQVQLQESGGGSVQAGGSLRLSCTASGFTFDDSDMGWYRQAPGNECELVSTISSDGNTYYTDSVKGRFT | The fusion proteins |
| NB01a scFv | NO: 83 | ISQDNAKNTVYLQMNSLGPEDTAVYYCAAEPRGYYSNYGGRRECNYWGQGTQVTVSSGGSEVQLVQSGARVKKPGESLPGESLKISCKGSG | of PD1 binding |
| | | YSFTNTYIHWVRQAPGQRLEWMGSIYPNYGDTAYNQKFKDRFVFSLDTSVSTAYLQISSLKAEDTAVYYCARGYSYAMDYWGQGTTVTV | domain and IL-2Rγ |
| | | SSGGGGSGGGGSGGGGSGIQMTWSPSSVSASVGDRVTITCSASQGISGDLNWYQQKPGKAPKLLIYHTSSLHSGVPSRFSGSGSGTDFTLTIS | binding domain. |
| | | SLQPEDFATYYCQYYSKDLLTFGGGTKLEIK | The sequence of |
| | | | PD1 binding |
| | | | domain is |
| | | | underlined and the |
| | | | sequence of IL-2Rγ |
| | | | binding domain is |
| | | | shown in bold. |

### Example 25: The Combinations of Neutralizing and Non-Neutralizing Anti-PD1 Abs that Fused with Anti-IL-2R Beta or IL-2R Gamma VHH Efficiently Induced IL-2 Receptor Activation in PD1+ HEK-Blue IL2 Cells

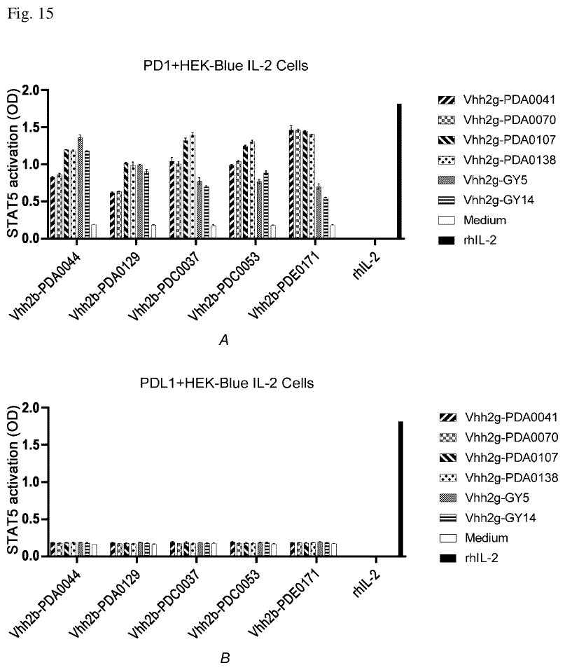

Human PD1 (SEQ ID NO: 37) or PDL1 (SEQ ID NO: 12) was first expressed in HEK-Blue IL-2 Cells (InvivoGen, #hkb-il2) by electroporation using SF Cell Line 4D-Nucleofector X Kit L (Lonza, #V4XC-2024). After electroporation, these transfectants were seeded in 384-well plate and incubated overnight at 37 degrees C./5% CO₂. These cells were cultured in Dulbecco's Modified Eagle Medium (DMEM) supplemented with 10% fetal bovine serum and 1% Penicillin-Streptomycin. Day 1 post electroporation, these cells were treated with different mixtures of neutralizing anti-PD1 Abs that fused with anti-IL-2R gamma VHH (12.5 nM as final concentration) and non-neutralizing anti-PD1 Abs that fused with anti-IL-2R beta VHH (12.5 nM as final concentration) for another day at 37 degrees C./5% CO₂. Recombinant human IL-2 (Peprotech, #200-02) was used as a positive control. Day 2 post electroporation, IL-2 receptor complex activation was evaluated using Quanti-Blue solution (InvivoGen, #rep-qbs) by measuring optical density at 620 nm (FIGS. 15A and B).

As shown in FIGS. 15A and B, the mixture of neutralizing anti-PD1 Abs and non-neutralizing anti-PD1 Abs with IL2R beta or IL2R gamma binding domain (Vhh2b or Vhh2g) induced IL-2 receptor activation efficiently in PD1+HEK-Blue IL-2 Cells, but not in the absence of PD1 expression. These data suggested it is possible to identify the combinations that can demonstrate PD1-dependent IL-2R agonistic activity by combining neutralizing and non-neutralizing anti-PD1 Abs. This is an effective way of screening for non-competitive or biparatopic binders to construct the combination of target-binding molecules or protein complex of the present application.

### Example 26: The Combinations of Neutralizing Anti-PD1 Ab and Non-Neutralizing Anti-PD1 Ab that Fused with Anti-IL-2R Beta or IL-2R Gamma VHH Induce IL-2 Receptor Activation in PD1+ Primary T Cells, but not in PD1 Negative NK92 Cells

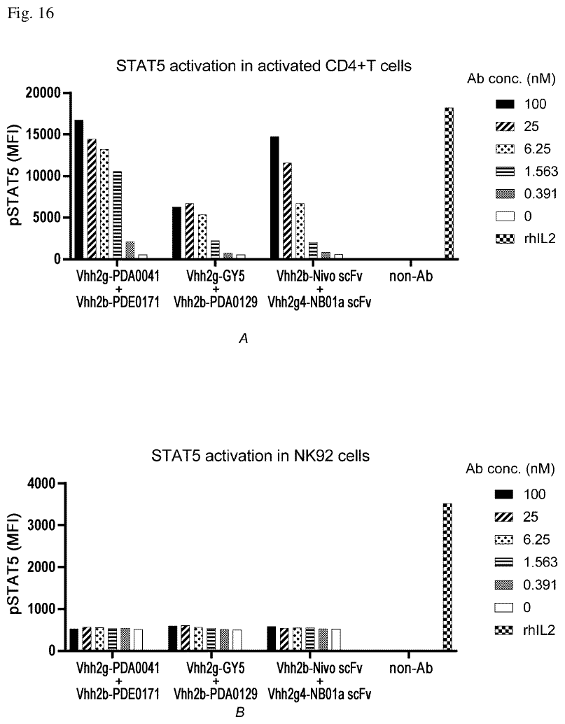

Human CD4+T cells were isolated from human PBMCs (STEMCELL, #70025.2) using EasySep™ Human CD4+T cell isolation kit (STEMCELL, #17952). After isolation, CD4+T cells were treated with plate coated anti-CD3 Ab (Biolegend, #317347, 5 micro g/mL) and anti-CD28 Ab (Biolegend, #302943, 5 micro g/mL) and recombinant human IL-2 (Peprotech, #200-02) for 3 days at 37 degrees C./5% CO₂. CTS Optimizer T cell expansion SFM (GIBCO, #A1048501) with penicillin/streptomycin (GIBCO, #15140122), GlutaMax (GIBCO, #35050061) and 10% fetal bovine serum was used for culture medium. After 3 days culture, CD4+ T cells were harvested and washed with culture medium twice and rested overnight with culture medium only. After resting, T cells were stimulated with the mixtures of Vhh2g-PDA0041 and Vhh2b-PDE0171, Vhh2g-GY5 and Vhh2b-PDA0129, or Vhh2b-Nivo scFv and Vhh2g4-NB01a scFv for 30 min at 37 degrees C. Recombinant human IL-2 (Peprotech, #200-02) was used as a positive control and NK92 cells (ATCC, #CRL-2407) was used as a PD1 negative IL-2 responsive cell. These cells were immediately fixed with CytoFix (BD Biosciences, #554655) for 20 minutes at room temperature. Cells were washed with staining buffer (Biolegend, #420201) once and chilled permeabilization buffer (BD Biosciences, #558050) were subsequently added to the samples and incubated on ice for 30 minutes. Cells were washed twice with staining buffer before staining with Alexa Fluor (registered trademark) 647 Mouse Anti-Stat5 (pY694) (BD Biosciences, #562076) for at least 60 minutes at room temperature. Cells were washed once with staining buffer before analysis. Data acquisition was performed on an LSRFortessa X-20 (Becton Dickinson) (FIGS. 16A and B).

As shown in FIGS. 16A and B, the mixtures of Vhh2g-PDA0041 and Vhh2b-PDE0171, Vhh2g-GY5 and Vhh2b-PDA0129, or Vhh2b-Nivo scFv and Vhh2g4-NB01a scFv induced significant STAT5 activation in activated human CD4+T cells, which highly express PD1, but not in the NK92 cells that are negative for PD1 expression. These data suggested the combinations of neutralizing anti-PD1 Ab and non-neutralizing anti-PD1 Ab that fused with anti-IL-2R beta or IL-2R gamma binding domain can induce PD1-dependent IL-2 receptor activation not only in reporter cells, but also in primary T cells.

### Example 27: Expression and Purification of VHH-IgG Fusion Proteins that Bind to PD1 as a Scaffold Protein and IL-2R Beta and IL-2R Gamma as Receptor Proteins

Anti-IL-2R beta VHH, which is Vhh2b1, Vhh2b3 or Vhh2b4, was fused to Hch of PDE0171 (SEQ ID NO: 84-86) and anti-IL-2R gamma VHH, which is Vhh2g1, Vhh2g2, Vhh2g4, Vhh2g6, Vhh2g8 or Vhh2g10, was fused to Lch of PDA0041 (SEQ ID NO: 87-92) by linker. These anti-IL-2R beta/gamma VHHs were described in WO2022/032006 A2, WO2022/031884 A2 or Cell (2022; 185: 1414-1430.e19). The DNA encoding these proteins were cloned into a mammalian expression vector. To prepare purified Abs, corresponding Hch and Lch expressing plasmids were expressed transiently using Expi293 cell line (Thermo Fisher, Carlsbad, CA, USA). The Hch and Lch pairs of each antibody are described in Table 10. Protein purification was carried out using Protein A. For the concentration of the purified proteins, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 10

| | SEQ | | SEQ | | |
|---|---|---|---|---|---|
| abbre- | ID | | ID | | Des- |
| viation | NO | Hch Sequences | NO | Lch Sequences | cription |
| Vhh2b1- | SEQ | MGWSCIILFLVATATGVHSQVQLQ | SEQ | MGWSCIILFLVATATGVHSDIVMTQTPASVSAAVGQTVSINCQASEDIENE | The fusion |
| PDE0171 | ID | ESGGGSYQAGGSLRLSCVGSGYTY | ID | LAWYQQRPGQPPSLLIYETSKLASGVPSRPSGSGSGTEFTLTISDLEAAD | proteins |
| | NO: | DTSDMSWYRQAPGKEREFVSDIDS | NO: | AATYYCQSYDWSDSSTYDNTFGGGTEVVVKRTVAAPSVFIFEPSDEQLKS | of PD1 |
| | 84 | GDWAAYADAVKGRFTISRDNAKK | 55 | GTASVVCLLNNFYPREAKVQWKVDNALQSGNSQESVTEQDSKDSTYSLSS | binding |
| | | TVYLQMNSLEPEDTAMYYCKASY | | TLTLSKADYEKHKVYACEVTHQGLSSPVTKSFNRGEC | domain |
| | | WKWGKLNNPWGPGTQVTVSSGG | | | and IL-2Rβ |
| | | SQSLEESGGGLVKPGASLTLTCYAS | | | binding |
| | | GFSFSGSGYYMCWVRQAPGKGLSCIA | | | domain. |
| | | CIYAGSSGATYYASWAKGRFTVSKT | | | The |
| | | SSITVTLQMTSLTAADTATYFCARH | | | sequence |
| | | TNGIGLGLWGPGTLVTVSSASTKGP | | | of PD1 |
| | | SVPPLAPSSKSTSGGTAALGCLVKDY | | | binding |
| | | FPEPVTVSWNSGALTSGVHTFPAVLQ | | | domain is |
| | | SSGLYSLSSVVTVPSSSLGTQTYTC | | | underlined |
| | | NVNHKPSNTKVDKRVEFKSCDKTHTC | | | and the |
| | | PPCPAPELRRGPSVTLEFPPKPKDTL | | | sequence |
| | | MISRTPEVTCVVVDVSREDPEVKFN | | | of IL-2Rβ |
| | | WYVDGVEVHNAKTKPREEQYNSTY | | | binding |
| | | RVVSVLTVLHQDWLNGKEYKCKVSN | | | domain is |
| | | KALPAPTEKHSKAKGQPREPQVYT | | | shown in |
| | | LPPSREEMTKNQVSLTCLVKGFYPS | | | bold. |
| | | DIAVEWESNGQPENNYKTTPPVLDS | | | |
| | | DGSEELYSKLTVDKSRWQQGNVF | | | |
| | | SCSVMHEALHNHYTQKSLSLSP | | | |
| Vhh2b3- | SEQ | MGWSCIILFLVATATGVHSQVQLQ | | | |
| PDE0171 | ID | ESGGGLVQPGGSLKLSCAASGFRF | | | |
| | NO: | SNYGMSWVRQAPGEGLEWVSYING | | | |
| | 85 | DGSRTHYADSVKGRFTISRDNAK | | | |
| | | NTLYLQLNSLKTEDTAMYYCEKG | | | |
| | | LSRDGWSLSAASRGQGTQVTVSSG | | | |
| | | GSQSLEESGGGLVKPGASLTLTCTA | | | |
| | | SGFSFSGSYYNCWVRQAPGKGLECI | | | |
| | | ACIYAGSSGATYYASWAKGRFTVSK | | | |
| | | TSSTTVTLQMTSLLTAADTATYFCAR | | | |
| | | HTNGIGLGLWGPGTLVTVSSASTKGP | | | |
| | | SVFFLAAPSSKSTSGGTAALGCLVKD | | | |
| | | YEPEPVTVSWNSGALTSGVHTFPAVL | | | |
| | | QSSGLYSLSSVVTVPSSSLGTQTYI | | | |
| | | CNVNHKPSNTKVDKRVEPKSCDKTHT | | | |
| | | CPPCPAPELRRGPSVRLFPPKPKDT | | | |
| | | LMISRTPEVTCVVVDVSHEDPECKF | | | |
| | | NWYVDGVEVHNAKTKPREEQYNST | | | |
| | | YRVVSVLTVLHQDWLNGKEYKCKVS | | | |
| | | NKALPAPIEKTISKAKGQPREPQV | | | |
| | | YTLPPSREEMTKNQVSLTCLVKGTY | | | |
| | | PSDIAVEWESNGQPENNYKTTPPVL | | | |
| | | DSDGSFFLYSKLTVDKSRWQQGNV | | | |
| | | ESCSVMHEALHNHYTQKSLSLSP | | | |
| | | DSDGSSFFLYSKLHVDKSRWQQGN | | | |
| | | VESCSVMHEALHNHYTQKSLSLSP | | | |
| Vhh2b4- | SEQ | MGWSCIILFLVATAGVHSQVQLQE | | | |
| PDE0171 | ID | SGGGSVQTGGSLRLSCAVSGYTT | | | |
| | NO: | YSFNMGWFRQAPGKEREFVAVIY | | | |
| | 86 | TGGGSTLYADSVKGRFTISQDNA | | | |
| | | KNTVYLQMNSLKPEDTAMYYCAA | | | |
| | | DDQRFASPLYAYFCYWGQGTQV | | | |
| | | TVSSGGSQSLEFSGGGLVKPGASLT | | | |
| | | LTCTASGFSFSGSYYMCWVRQAPGK | | | |
| | | GLECIACTYAGSSGATYYASWAKGR | | | |
| | | FTVSKTSSTTVTLQMTSLTAADTAT | | | |
| | | YFCARGTNGIGLGLWGPGTLVTVSS | | | |
| | | ASTKGPSVFPLAPSSKSTSGGTAALG | | | |
| | | CLVKDYFPEPVTVSWNSGALTSGVHT | | | |
| | | FPAVLQSSGLYSLSSVVTVPSSSLG | | | |
| | | TQTYICNVNHKPSNTKVDKRVEPKS | | | |
| | | CDKTHTCPPCPAPELRRGPSVFLFPP | | | |
| | | KPKDTLMISRTPEVTCVVVDVSHE | | | |
| | | DPEKFNWYVDGVEVHNAKTKPREP | | | |
| | | QYNSTYRVVSVLTVLHQDWLNGKE | | | |
| | | YKCKVSNKALPAPIEKTISKAKGQP | | | |
| | | REPQVYTLPPSREEMTKNQVSLTCL | | | |
| | | VKGPYPSDIAVEWESNGQPIENNYKT | | | |
| | | TPPVLDSDGSEFLYSKLTVDKSRWQ | | | |
| | | QGNVFSCSVMHEALHNHYTQKSLSL | | | |
| Vhh2g1- | SEQ | MGWSCIILFLVATAGVHSQEQLEES | SEQ | MGWSCIILFLVATAGVHSQVQLQFSGGGVSQAGGSLRLSCAASGFTF | The fusion |
| PDA0041 | ID | GGDLVKPGASLTLTCTASGPSFNSY | ID | DDSDMGWYRQAPGNECDLVSTISSDGSTYYADSVKGRFTISQDNAKN | proteins |
| | NO: | WMSWVRQAPGKGLEWLADIGAGGSG | NO: | TVYLQMDSVKPEDTAVYYCAADFMIATQAPGAGCTGQGTQVTVSSG | of PD1 |
| | 47 | STFYASWAKFRGTISKTSSTTVTL | 87 | GSAFELTTQTPSSVEAAVGGTVSINCQASFDIERYLAWYQQRPGQPPKLL | binding |
| | | QMTSLTAADTATYECARYVDGGHSD | | IYTASSLPSGVPSRFKGSGSGTQFTLTISDLEAADAATYYCQQGWSDANV | domain |
| | | LEGPGTLVTVSSASTKGPSVFPLAP | | DNTFGGGTEVVVKRTVAAPSVFIFPPSDEQLKSGTASVVCLLNNFYPREA | and IL-2Rγ |
| | | SSKSTSGGTAALGCLVKDYFPEPVT | | KVQWKVDNALQSGNSQESVTEQDSKDSTYSLSSTLTLSKADYFKHKVYAC | binding |
| | | VSWNSGALTSGVHTFPAVLQSSGLY | | EVTEQFLSSPVTKSFNRGEC | domain. |
| Vhh2g2- | | SLSSVVTVPSSSLGTQTYICNVNHK | SEQ | MGWSCIILFLVATATGVHSQVQLQESGGGSVPAGGSLKLSCAASGFSFS | The |
| PDA0041 | | PSNTKVDKRVEPKSCDKTHTCPPCPA | ID | SYPMTWARQAPGKGLEWVSTIASDGGSTAYAASVEGRFTISRDNAKS | sequence |
| | | PELRRGPSVFLFPPKPKDTLMISRT | NO: | TLYLQLNSLKTEDTAMYYCTKGYGDGTPAPGQGTQVTVSSGGSAFELT | of PD1 |
| | | PEVTCVVVDVSHEDPEVKFNWYVDG | 88 | QTPSSVEAAVGGTVSINCQASEDIERYLAWYQQRPGQPPKLLIYTASSLP | binding |
| | | VEVMNAKTKPREEQYNSTYRVVSV | | SGVPSREKGSGSGTQFTLTISDLEAADAATYYCQQGWSDAKVDNTEGGGT | domain is |
| | | LTVLHQDWLNGKEYKCKYSNKALP | | EVVYKRTVAAPSVFIFPPSDEQLKSGTASVVCLLNNFYPREAKVQWKVDN | underlined |
| | | APIEKTISKAKGQPREPQVYTLFPSR | | ALQSGNSQESVTEQDSKDSTYSLSSTLTLSKADYEKHKVYACEVTHQGES | and the |
| | | EEMTKNQVSLTCLVKGPYPSDIAVE | | SPVTKSFNRGEC | sequence |
| Vhh2g4- | | WESNGQPENNYKTTPPVLDSDGSFF | SEQ | MGWSCIILFLVATATGVHSQVQLQESGGGSVQAGGSLRLSCTASGFTF | of IL-2Rγ |
| PDA0041 | | LYSKLTVDKSRWQQGNVFSCSVMH | ID | DDSDMGWYRQAPGNECELVSTISSDGNTYYTDSVKGRFTISQDNAKN | binding |
| | | EALHNHYTQKSLSLSP | NO: | TVYLQMNSLGPEDTAVYYCAAEPRGYYSNYGGRRECNYWGQGTQVT | domain is |
| | | | 89 | VSSGQSAFELTQTPSSVEAAVGGTVSINCQASEDIERYLAWYQQRPGQPPK | shown in |
| | | | | LLIYTASSLPSGYPSREKGSGSGTQFTLTISDLEAADAATYYCQQGWSDAN | bold. |
| | | | | VDNTFGGGTEVVYKRTVAAPSVFIFPPSDEQLKSGTASVVCLENNFYPREA | |
| | | | | KYQWKVQNALQSGNSQESVTEQDSKQSTYSLSSTLTLSKADYEKHKVYA | |
| | | | | CEVTHQGLSSPVTKSFNRGEC | |
| Vhh2g6- | | | SEQ | MGWSCIILFLVATATQVHSQVQLQESGGGAVQAGGSLRLSCAASGFTF | |
| PDA0041 | | | ID | SNAHMSWVRQAPGKGREWISSIYSGGSTWYADSYKGRFTISRDNSKN | |
| | | | NO: | TLYLQLNSLKTEDTAMYYCAENRLHYYSDDDSLRGQGTQVTVSSGGS | |
| | | | 90 | AFELTQTPSSVEAAVGGTVSINCQASEDIERYLAWYQQRPGQPPKLLIYTA | |
| | | | | SSLPSGVPSREKGSGSGTQFTLTISDLEAADAATYYCQQGWSDANVDNTE | |
| | | | | GGGTEVVVKRTVAAPSVFIFPPSDEQLKSGTASVVCLLNNFYPREAKVQW | |
| | | | | KVQNALQSGNSQESVTEQDSKQSTYSLSSTLTLSKADYEKHKVYACEVTH | |
| | | | | QQLSSPVTKSFNRGEC | |
| Vhh2g8- | | | SEQ | MGWSCIILFLVATATGVHSQVQLQESGGGSVQAGGSLRLSCVASGYTF | |
| PDA0041 | | | ID | SSYCMGWFRQAPGKEREGVAALGGGSTYYADSVKGRFTISQDNAKN | |
| | | | NO: | TLYLQMNSLKPEDTAMYYCAAAWVACLEFGGSWYDLARYKHWGQ | |
| | | | 91 | GTQVTVSSGGSAFELTQTPSSVEAAVGGTVSINCQASEDIERYLAWYQQRP | |
| | | | | GQPPKLLIYTASSLPSGVPSRFKGSGSGTQFTLTISDLEAADAATYYCQQG | |
| | | | | WSDANVQNTFGGGTEVVVKRTVAAPSVFIFPPSDEQLKSGTASVVCLLNN | |
| | | | | PYPREAKVQWKVDNALQSQNSQESVTEQDSKDSTYSLSSTLTLSKADYEK | |
| | | | | HKVYACEVTHQGLSSPVTKSFNRGEC | |
| Vhh2g10- | | | SEQ | MGWSCIILFLVATATGVHSQVQLQESGGGSVQAGGSLRLSCAASGSTYS | |
| PDA0041 | | | ID | SAYIGWFRQAPGKKREGVAGIYTRDGSTAYADSVKGRFTISQDSAKK | |
| | | | NO: | TVYLQMNSLKPEDTAMYYCAAGRRTKSYVYIFRPEEYNYWGQGTQV | |
| | | | 92 | TVSSGGSAFELTQTESSVEAAVGGTVSINCQASEDIERYLAWYQQRPGQPP | |
| | | | | KLLTYTASSLPSGYPSREKQSGSGTQFTLTISDLEAADAATYYCQQGWSDA | |
| | | | | NYDNTFGGGTEVVYKRTVAAPSVFIFPPSDEQLKSGTASVVCLLNNFYPRE | |
| | | | | AKVQWKVQNALQSGNSQESVTEQDSKQSTYSLSSTETLSKADYEKHKVY | |
| | | | | ACEVTHQGLSSPVTKSFNRGEC | |

### Example 28: The Mixture of Anti-PD1 Ab (PDA0041) Fused with Various IL-2R Gamma Binding Domains and Anti-PD1 Ab (PDE00171) Fused with Various IL-2R Beta Binding Domains Induce IL-2 Receptor Activation in PD1+ HEK-Blue IL-2 Cells

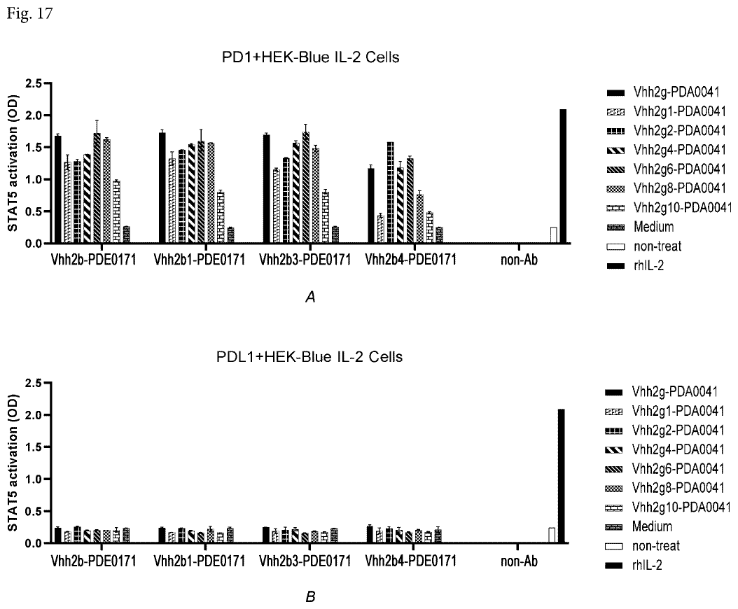

Human PD1 (SEQ ID NO: 37) or PDL1 (SEQ ID NO: 12) was first expressed in HEK-Blue IL-2 Cells (InvivoGen, #hkb-il2) by electroporation using SF Cell Line 4D-Nucleofector X Kit L (Lonza, #V4XC-2024). After electroporation, these transfectants were seeded in 384-well plate and incubated overnight at 37 degrees C./5% CO₂. These cells were cultured in Dulbecco's Modified Eagle Medium (DMEM) supplemented with 10% fetal bovine serum and 1% Penicillin-Streptomycin. Day 1 post electroporation, these cells were treated with different mixtures of anti-PD1 Ab (PDA0041) that fused with various IL-2R gamma binding domains (Vhh2g, Vhh2g1, Vhh2g2, Vhh2g4, Vhh2g6, Vhh2g8 or Vhh2g10) and anti-PD1 Ab (PDE0171) that fused with various IL-2R beta binding domain (Vhh2b, Vhh2b1, Vhh2b3 or Vhh2b4), and further incubated at 37 degrees C./5% CO₂ for another day. Each Ab concentration was 12.5 nM. Recombinant human IL-2 (Peprotech, #200-02) was used as a positive control. Day 3 post electroporation, IL-2 receptor complex activation was evaluated using Quanti-Blue solution (InvivoGen, #rep-qbs) by measuring optical density at 620 nm (FIGS. 17A and B).

As shown in FIGS. 17A and B, the mixture of the fusion proteins of anti-PD1 Ab (PDA0041) with various IL-2R gamma binding domains and anti-PD1 Ab (PDEA00171) with various IL-2R beta binding domain induced IL-2 receptor activation in PD1+ HEK-Blue IL-2 Cells, but not in the absence of PD1 expression. These data suggested various IL-2R beta/gamma binding domain can be used for inducing PD1-dependent IL-2 receptor activation.

### Example 29: Expression and Purification of scFv-IgG or VHH-IgG Fusion Proteins that Bind to PD1 as a Scaffold Protein and IL-2R Beta and IL-2R Gamma as Receptor Proteins

Anti-PD1 VHH (Vhhpc13) was fused to Hch of anti-IL2R gamma (AM3) (SEQ ID NO: 93) and anti-PD1 scFv (PDA0129 scFv) was fused to Hch of anti-IL2R beta (AL1, AL2, AL3, AL4, or AL5) (SEQ ID NO: 94) by linker. These Hch fusion proteins and corresponding Lch (SEQ ID NO: 95-100) were cloned into a mammalian expression vector. These anti-IL-2R beta or anti-IL-2R gamma antibodies were reported on WO2023/139293 A1. To prepare purified Abs, corresponding Hch and Lch expressing plasmids were expressed transiently using Expi293 cell line (Thermo Fisher, Carlsbad, CA, USA). The pairs of Hch and Lch of each antibody were described in Table 11. Protein purification was carried out using Protein A. For the concentration of the purified proteins, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 11

| | SEQ | | SEQ | | |
|---|---|---|---|---|---|
| abbre- | ID | | ID | | |
| viation | NO | Hch Sequences | NO | Lch Sequences | Description |
| Vhhpc13- | SEQ | MGWSCIILFLVATATGVHSDVQLVESGGGVVQPGGSLRLSCAASGSIASIH | SEQ | MQWSCIILFLVATATGVHSDIQMTQSPSSLSASVGDRVTITCRASHYITT | The fusion |
| AM3 | ID | AMGWFRQAPGKEREPVAVITVSGGITYYADSVKGRFTISRDPSKNTVYLQ | ID | WLAWYQQKPGKAPKLLIYDYSSLESGVPSRFSGSGSGTDFTFTISSLQP | proteins of PD1 |
| | NO: | MNSLRPEDTALYYCAGDKHQSSFYDYWGQGTLVTVSSGGSEVQLLESG | NO: | EDIATYYCQQYESYSPTFGQGTKVEIKRTVAAPSVFIFPPSDEQLKSGTA | binding domain |
| | 93 | GGLVQPGGSLRLSCAASGFTFSSYAMSWVRQAPGKGLEWVSAISGSG | 95 | SVVCLLNNFYPREAKVQWKVDNALQSGNSQESVTEQDSKQSTYSLSSTLT | and IL-2Rγ |
| | | GSTYYADSVKGRFTISRDNSKNTLYLQMNSLRAEDTAVYYCAKSYGA | | LSKADYEKHKVYACEVTHQGLSSPVTKSFNRGEC | binding domain. |
| | | FDYWGQGTLVTVSSASTKGPSVFPLAPSSKSTSGGTAALGCLVKDYFPEP | | | The sequence of |
| | | VTVSWNSGALTSGVHTFPAVLQSSGLYSLSSSVVTVPSSSLGTQTYICNVNH | | | PD1 binding |
| | | KPSNTKVDKRVKPKSCDKTHTCPPCPAPEAAGGPSVFLFPPKPKDTLMISR | | | domain is |
| | | TPEVTCVVVDVSHEDPRVKFNWYVDGVEVHNAKTKPRPPQYNSTYRVVS | | | underlined and the |
| | | VLRVLHQDWLNGKEYKCKVSNKALAAPIEKRISKAKGQPREPQVYTLPPS | | | sequence of IL-2Rγ |
| | | REEMTKNQVSLTCLVKGFYPSDIAVEWESNGQPRNNYKTTPPVLDSDGSF | | | binding domain |
| | | FLYSKLTVDKSRWQQGNVFSCSVMHEALHNHYTQKSLSLSPG | | | is shown in bold |
| PDA0129 | SEQ | MGWSCIILFLVATATGGVHSQSVEESGGRLVTPGTPGTPLTLTCTVSFPSLSSYD | SEQ | MGWSCIILFLVATATGVHSDIQMTQSPSSLSASVGDRVTITCQASQDISN | The fusion |
| scFv-AL1 | ID | MSWVRQAPGKGLFYIGHSSSGSTYYASWAKGRFTISKTSTTVDLKTTSPTT | ID | YLNWYQQKPGKAPKLLIYDASNLETGVPSRESGSGSGTDETFTISSLQP | proteins of PD1 |
| | NO: | EDTATYFCARLGYVGYGYADIWGPGTLVTVSSGGGGSGGGGSGGGGSIE | NO: | EDIATYYCQQDANDPRTEGQGTKVEIKRTVAAPSVHIPPPSDEQLKSGT | binding domain |
| | 94 | MTQTPFSVSAAVGGTVTIKCQASEDIYSLLAWYQQKPGQPPKLLIYSASTL | 96 | ASVVCLLNNFYPREAKVQWKVQNALQSGNSQESVTEQDSKDSTYSLSSTL | and IL-2Rβ |
| | | ASGVPSRFKGSGYGTEFTLTISGVQAADAATYYCQGGYYSSSADNTFGGG | | TLSKADYEKHKVYACEVTHQGLSSPVTKSFNRGEC | binding domain |
| PDA0129 | | TEVVVKRGGSEVQLLESGGGLVQPGGSLRLSCAASGFTFSSVAMSWV | SEQ | MGWSCIILFLVATATGVHSDIQMTQSPSSLSASVGDRVTITCQASQDIGT | The sequence of |
| scFv-AL2 | | RQAPGKGLEWVSAISGSGGSTYYADSVKGRFTISRDNSKNTLYLQMN | ID | YLNWYQQKPGKAPKLLIYEASTLETGVPSRFSGSGSGTDFTFTISSLQP | PD1 binding |
| | | SLRAEDTAVYYCAKSYGAFDYWGQGTLVTVSSASTKGPSVFPLAPSSKS | NO: | EDIATYYCQQDGARDDYATFGQGTKVEIKRTVAAPSVFIFPPSDEQLKS | domain is |
| | | TSGGTAALGCLVKDYFPEPVTVSWNSGALTSGVHTFPAVLQSSGLYSLSS | 97 | GTASVVCLLNNFYPREAKVQWKVDNALQSGNSQESVTEQDSKDSTYSLS | underlined and the |
| | | VVTVPSSSLGTQTYICNVNHKPSNTKVDKRVEPKSCDKTHTCPPCPAPEAA | | STITLSKADYEKHKVYACEVTHQGESSPVTKSFNRGEC | sequence of IL-2Rβ |
| PDA0129 | | GGPSVFLFPPKPKDTLMISRTPEVTCVVVDVSHEDPEVKFNWYVDGVEVH | SEQ | MGWSCIILFLVATATGVHSDIQMTQSPSSLSASVGDRVTITCQASQDIDD | binding domain |
| scFv-AL3 | | NAKTKPREEQYNSTYRVVSVLTVLHQDWLNGKEYKCKVSNKALAAPLEK | ID | YLNWYQQKPGKAPKLLIYDASNLQSGVPSRFSGSGSGTDFTFTISSLQP | is shown in bold. |
| | | TISKAKGQPREPQVYTLPPSREEMTKNQVSLTCLVKGFYPSDIAVEWESNG | NO: | EDIATYYCQQDSMDPRTFGQGTKVEIKRTVAAPSVFIFPPSDEQLKSGT | |
| | | QPENNYKTTPPVLDSDGSPPLYSKLTVDKSRWQQGNVFSCSVMHEALHN | 98 | ASVVCLLNNFYPREAKVQWKVQNALQSGNSQESVTEQDSKQSTYSLSSTL | |
| | | HYTQKSLSLSPG | | TLSKADYBKHKVYACEYTHQGLSSPVTKSFNRGEC | |
| PDA0129 | | | SEQ | MGWSCIILFLVATATGVHSDIQMTQSPSSLSASVGDRVTITCRASQSIDE | |
| scFv-AL4 | | | ID | YLNWYQQKPGKAPKLLIYEASKLQSGVPSRESGSGSGTDFTFTISSLQP | |
| | | | NO: | EDIATYYCQQDGAMDTYATFGQGTKVEIKRTVAAPSVFIFPPSDEQLKS | |
| | | | 99 | GTASVYCLLNNFYPREAKVQWKVQNALQSGNSQESVTEQDSKQSTYSLS | |
| | | | | STLTLSKADYEKHKVYACEVTHQGLSSPVTKSFNRGEC | |
| PDA0129 | | | SEQ | MGWSCIILFLVATATGVHSEIVLTQSPATLSLSPGERATLSCRASQSVDE | |
| scFv-AL5 | | | ID | YLAWYQQKPGQAPRLLIYDASERATGIPARFSGSGSGTDFTLTISSLEP | |
| | | | NO: | EDFAVYYCQQDATDPRTFGQGTKVEIKRTVAAPSVFIFPPSDEQLKSGT | |
| | | | 100 | ASVVCLLNNFYPREAKVQWKVDNALQSGNSQESVTEQDSKDSTYSLSSTL | |
| | | | | TLSKADYEKHKVYACEVTBQGLSSPVTKSFNRGEC | |

### Example 30: The Mixture of Vhhpc13-AM3 and PDA0129 scFv-AL1, 2, 3, 4 or 5 which Bind PD1 as a Target Scaffold Protein and IL-2R Beta or IL-2R Gamma as Target Receptor Proteins Induce IL-2R Activation in HEK-Blue IL-2 Cells

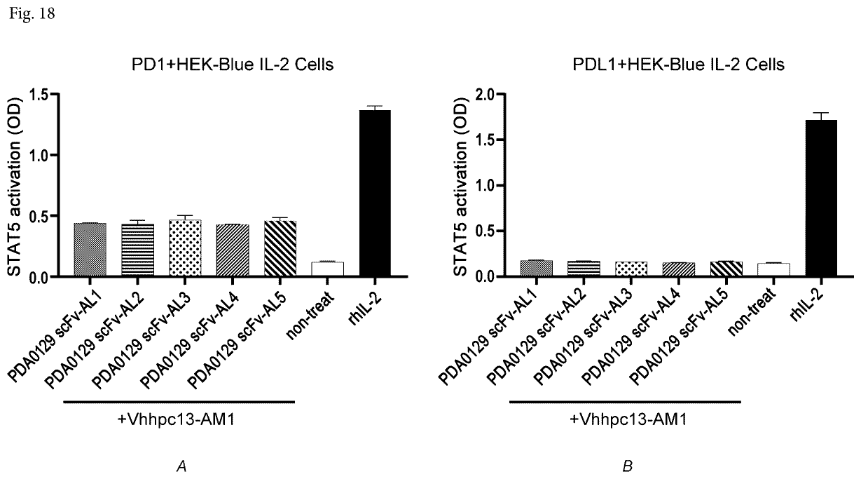

Human PD1 (SEQ ID NO: 37) or PDL1 (SEQ ID NO: 12) was first expressed in HEK-Blue IL-2 Cells (InvivoGen, #hkb-il2) by electroporation using SF Cell Line 4D-Nucleofector X Kit L (Lonza, #V4XC-2024). After electroporation, these transfectants were seeded in 384-well plate and incubated overnight at 37 degrees C./5% CO₂. These cells were cultured in Dulbecco's Modified Eagle Medium (DMEM) supplemented with 10% fetal bovine serum and 1% Penicillin-Streptomycin. Day 1 post electroporation, these cells were treated with different mixtures of fusion proteins that bind to PD1 as a scaffold protein and IL-2R beta or IL-2R gamma as receptor proteins (25 nM as a final concentration) and further incubated at 37 degrees C./5% CO₂ for another day. Recombinant human IL-2 (Peprotech, #200-02) was used as a positive control. Day 2 post electroporation, IL-2 receptor complex activation was evaluated using Quanti-Blue solution (InvivoGen, #rep-qbs) by measuring optical density at 620 nm (FIGS. 18A and B).

As shown in FIGS. 18A and B, the mixture of Vhhpc13-AM3 and PDA0129 scFv-AL1, PDA0129 scFv-AL2, PDA0129 scFv-AL3, PDA0129 scFv-AL4 or PDA0129 scFv-AL5 induced IL-2 receptor activation in PD1+HEK-Blue IL-2 Cells, but not in the absence of PD1 expression. These data suggested not only anti-IL-2R beta/gamma VHHs, but also other binding domain, like Fab, can be used for inducing PD1-dependent IL-2 receptor activation.

### Example 31: Expression and Purification of scFv-VHH Fusion Proteins that Bind to CD25 as a Target Scaffold Protein and IL-2R Beta and IL-2R Gamma as Targets Receptor Proteins

Anti-CD25 scFv were fused with VHH that can bind to human IL-2R gamma (Vhh2g) or IL-2R beta (Vhh2b) (SEQ ID NO: 101-109). RG6292 scFv (Front. Oncol. 2023; 13: 1150149), BT942 scFv (Scientific Reports 2021; 11: 22966) or 7G7/B6 scFv (Cancer Research 2000; 60: 6977-6984) was used as a non-neutralizing anti-CD25 antibody and Gen scFv (U.S. Pat. No. 8,961,968 B2) or Dac scFv (Biomedicines 2019; 7: 18) was used as a neutralizing anti-CD25 antibody. The DNA encoding these proteins were cloned into a mammalian expression vector. These plasmids were expressed transiently using Expi293 cell line (Thermo Fisher, Carlsbad, CA, USA). Protein purification was carried out using Protein A or immobilized metal ion affinity chromatography. For the concentration of the purified proteins, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 12

| SEQ ID NO | abbreviation | Sequences | Description |
|---|---|---|---|
| SEQ ID NO: | Vhh2g-Gen | MGWSCIILFLVATATGVHSQVQLQESGGGSVQAGGSLRLSCAASGYTYRDYYMGWFRQAPGREREGVASIYTRGSR | The fusion proteins of |
| 101 | scFvHL | EGSTRYSSSVEGRFTITLDTAKNTLYLQMNSLKPEDTAMYYCAADDRTWLPRVQLGGPRENEYNYWGQGTQVTVS | CD25 binding domain |
| | | SQVQLVQSGAEVKKPGSSVKVSCKASGGTFSRYPINWVRQAPGQGLEWMGRIIPILGIADYAQRFQGRVTITADKSTNTA | and IL-2Rγ binding |
| | | YMELSSLRSEDTAVYYCARRDWGDYWGQGTLVTVSSGGGGGGGGSGGGGSEIVLTQSPGTLSLSPGERATLSCRASQS | domain. The sequence of |
| | | VSSSFLAWYQQKPGQAPRLLIYGASSRATGIPDRFSGSGSGTDFTLTISRLEPEDFAVYYCQQYSSSPLTFGGGTKVEIKRTV | CD25 binding domain is |
| | | GGSHHHHHHHH | underlined and the |
| SEQ ID NO: | Vhh2g-Gen | MGWSCIILFLVATATGVHSQVQLQESGGGSVQAGGSLRLSCAASGYTYRDYYMGWFRQAPGREREGVASIYTRGSR | sequence of IL-2Rγ |
| 102 | scFvLH | EGSTRYSSSVEGRFTITLDTAKNTLYLQMNSLKPEDTAMYYCAADDRTWLPRVQLGGPRENEYNYWGQGTQVTVS | binding domain is shown |
| | | SEIVLTQSPGTLSLSPGERATLSCRASQSVSSSFLAWYQQKPGQAPRLLIYGASSRATGIPDRFSGSGSGTDFTLTISRLEPED | in bold. |
| | | FAVYYCQQYSSSPLTFGGGTKVEIKRTYGGGGSGGGGSGGGGSQVQLVQSGAEVKKPGSSVKVSCKASGGTFSRYPINW | |
| | | VRQAPGQGLEWMGRIIPILGIADYAQRFQGRVTITADKSTNTAYMELSSLRSEDTAVYYCARRDWGDYWGQGTLVTVSS | |
| | | GGSHHHHHHHH | |
| SEQ ID NO: | Vhh2g- | MGWSCIILFLVATATGVHSQVQLQESGGGSVQAGGSLRLSCAASGYTYRDYYMGWFRQAPGREREGVASIYTRGSR | |
| 103 | BT942 | EGSTRYSSSVEGRFTITLDTAKNTLYLQMNSLKPEDTAMYYCAADDRTWLPRVQLGGPRENEYNYWGQGTQVTVS | |
| | scFvHL | SQVQLVQSGAEVKKPGSSVKVSCKASGGTESSDAINWVRQAPGQGLEWMGRIIPIFGVADYAQKFQGRVTLTADKSTSTA | |
| | | YMDLSSLRSEDTAVFYCARERGDYSNFWYFDLWGRGTLVTVSSGGGGSGGGGSGGGGSDIQMTQSPDSLAVSLGERATI | |
| | | NCKSSQSVLYSSNNKNYLAWYQQKPGQPPKLLIYWASTRESGVPDRPSGSGSGTDFTLTISSLQAEDVAVYYCQQYYSTP | |
| | | YTFGQGTKVEIKRTVGGSAHHHHHHH | |
| SEQ ID NO: | Vhh2g- | MGWSCIILFLVATATGVHSQVQLQESGGGSVQAGGSLRLSCAASGYTYRDYYMGWFRQAPGREREGVASIYTRGSR | |
| 104 | BT942 | EGSTRYSSSVEGRFTITLDTAKNTLYLQMNSLKPEDTAMYYCAADDRTWLPRVQLGGPRENEYNYWGQGTQVTVS | |
| | scFvLH | SDIQMTQSPDSLAVSLGERATINCKSSQSVLYSSNNKNYLAWYQQKPGQPPKLLIYWASTRESGVPDRFSGSGSGTDFTLTI | |
| | | SSLQAEDVAVYYCQQYYSTPYTFGQGTKVEIKRTVGGGGSGGGGSGGGGSQVQLVQSGAEVKKPGSSVKVSCKASGGTF | |
| | | SSDAINWVRQAPGQGLEWMGRIIPIFGVADYAQKFQGRVTLTADKSTSTAYMDLSSLRSEDTAVFYCARERGDYSNFWY | |
| | | FDLWGRGTLVTVSSGGSAHHHHHHH | |
| SEQ ID NO: | Vhh2b- | MGWSCIILFLVATATGVHSQVQLQESGGGSVQAGGSLRLSCAASSYTISSVCMGWFRQAPGKEREGVAGIAPDGSTG | The fusion proteins of |
| 105 | RG6292 scFv | YGDSVKGRFTISKDNAKNTLYLQMNSLKPEDTAMYYCAAASPGRCFLPRTALEPALYYNWGQGTQVTVSSQVQLV | CD25 binding domain |
| | | QSGAEVKKPGSSVKVSCKASGGTFSSLAISWVRQAPGQGLEWMGGIIPIFGTANYAQKFQGRVTITADESTSTAYMELSSL | and IL-2Rγ binding |
| | | RSEDTAVYYCARGGSVSGTLVDFDIWGQGTMVTVSSGGGSGGGSGGGSDIQMTQSPSTLSASVGDRVTITCRASQSI | domain. The sequence of |
| | | SSWLAWYQQKPGKAPKLLIYKASSLESGVPSRFSGSGSGTEFTLTISSLQPDDFATYYCQQYNIYPITFGGGTKVEIKRTVG | CD25 binding domain is |
| | | GSHHHHHHHH | underlined and the |
| SEQ ID NO: | Vhh2b- | MGWSCIILFLVATATGVHSQVQLQESGGGSVQAGGSLRLSCAASSYTISSVCMGWFRQAPGKEREGVAGIAPDGSTG | sequence of IL-2Rγ |
| 106 | BT942 scFv | YGDSVKGRFTISKDNAKNTLYLQMNSLKPEDTAMYYCAAASPGRCFLPRTALEPALYYNWGQGTQVTVSSQVQLV | binding domain is shown |
| | | QSGAEVKKPGSSVKVSCKASGGTFSSDAINWVRQAPGQGLEWMGRIIPIFGVADYAQKFQGRVTLTADKSTSTAYMDLSS | in bold. |
| | | LRSFDTAVFYCARERGDYSNFWYFDLWGRGTLVTVSSGGGGSGGGGSGGGGSDIQMTQSPDSLAVSLGFRATINCKSSQ | |
| | | SVKYSSNNKNYLAWYQQKPGQPPKLLIYWASTRESGVPDRFSGSGSGTDFTLTISSLQAEDVAVYYCQQYYSTPYTFGQG | |
| | | TKVEIKRTVGGSHHHHHHHH | |
| SEQ ID NO: | Vhh2b- | MGWSCIILFLVATATGVHSQVQLQESGGGSVQAGGSLRLSCAASSYTISSVCMGWFRQAPGKEREGVAGIAPDGSTG | |
| 107 | 7G7/B6 scFv | VGDSVKGRFTISKDNAKNTLYLQMNSLKPEDTAMYYCAAASPGRCFLPRTALEPALYYNWGQGTQVTVSSQIVLSQ | |
| | | SPAILSASPGERVTMTCRASSSVSFMHWLQQKPGSSPKPWIYATSNLASGVSARFSGSGSGTSYSLTITRVEAEDAATYYC | |
| | | QQWSSNPPAFGGGTKLEIKGGGGSGGGGSGGGGSEVQLVESGGDLVQPRGSLKLSCAASGFTFSSYGMSWVRQTPDKRL | |
| | | ELVATINGYGDTTYYPDSVKGRFTISRDNAKNTLYLQMSSLKSEDTAMYFCARDRDYGNSYYYALDYWGQGTSVTVSSG | |
| | | GSHHHHHHHH | |
| SEQ ID NO: | Vhh2b-Gen | MGWSCIILFLVATATGVHSQVQLQESGGGSVQAGGSLRLSCAASSYTISSVCMGWFRQAPGKEREGVAGIAPDGSTG | |
| 108 | scFv | YGDSVKGRFTISKDNAKNTLYLQMNSLKPEDTAMYYCAAASPGRCFLPRTALEPALYYNWGQGTQVYVSSQVQLY | |
| | | QSGAEVKKPGSSVKVSCKASGGTESRYPINWVRQAPGQGLEWMGRIIPILGIADYAQRFQGRVTITADKSTNTAYMELSSL | |
| | | RSEDTAVYYCARRDWGDYWGQGTLVTVSSGGGGSGGGGSGGGGSEIVLTQSPFTLSLPGERATLSCRASQSVSSSFLA | |
| | | WYQQKPGQAPRLLIYGASSRATGIPDRFSGSGSGTDFTLTISRLEPEDFAVYYCQQYSSSPLRFGGGTKVEIKRTVGGSHHH | |
| | | HHHHH | |
| SEQ ID NO: | Vhh2b-Dac | MGSWCIILFLVATATGVHSQVQLQESGGGSVQAGGSLRLSCAASSYTISSVCMGWFRQAPGKEREGVAGIAPDGSTG | |
| 109 | scFv | YGDSVKGRFTISKDNAKNTLYLQMNSLKPEDTAMYYCAAASPGRCFLPRTALEPALYYNWGQGTQVTVSSQVQLV | |
| | | QSGAEVKKPGSSVKVSCKASGYTFTSYRMHWVRQAPGQGLEWIGYINPSTGYTEYNQKFKDKATITADESNTAYMELS | |
| | | SLRSEDTAVYYCARGGGVFDYWGQGTLVTVSSGGGGSGGGGSGGGGSDIQMTQSPSTLSASVGDRVTITCSASSSISYMH | |
| | | WYQQKPGKAPKLLIYTTSNLASGVPARFSGSGSGTEFTLTISSLQPDDFATYYCHQRSTYPLTFGQGTKVEVKRTVGGSHH | |
| | | HHHHHH | |

### Example 32: The Mixture of Neutralizing Anti-CD25 scFv with IL-2R Beta/Gamma Binding Domain and Non-Neutralizing Anti-CD25 scFv with IL-2R Beta/Gamma Binding Domain Induced IL-2 Receptor Activation in Activated Primary CD4+T Cells

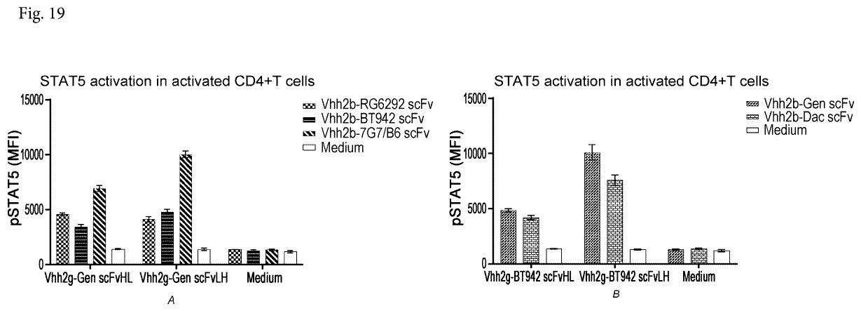

Human CD4+T cell were isolated from human PBMCs (STEMCELL, #70025.2) using EasySep™ Human CD4+T cell isolation kit (STEMCELL, #17952). After isolation, CD4+T cells were treated with plate coated anti-CD3 Ab (Biolegend, #317347, 5 micro g/mL) and anti-CD28 Ab (Biolegend, #302943, 5 micro g/mL) and recombinant human IL-2 (Peprotech, #200-02) for 3 days at 37 degrees C./5% CO₂. CTS Optimizer T cell expansion SFM (GIBCO, #A1048501) with penicillin/streptomycin (GIBCO, #15140122), GlutaMax (GIBCO, #35050061) and 10% fetal bovine serum was used as culture medium. After 3 days culture, CD4+ T cells were harvested and washed with culture medium twice and rested overnight under culture medium condition. After resting, T cells were stimulated with the mixtures of VHH-scFv fusion proteins (25 nM as a final concentration) that bind to CD25 as a target scaffold protein and IL-2R beta or IL-2R gamma as target receptor proteins at 37 degrees C. for 25 minutes. After stimulation, these cells were immediately fixed with CytoFix (BD Biosciences, #554655) for 20 minutes at room temperature. Cells were washed once with staining buffer (Biolegend, #420201) and chilled permeabilization buffer (BD Biosciences, #558050) were subsequently added to the cells and incubated on ice for 30 minutes. Cells were washed twice with staining buffer and Alexa Fluor (registered trademark) 647 Mouse Anti-Stat5 (pY694) (BD Biosciences, #562076) were added and incubated for at least 60 minutes at room temperature. Cells were washed once with staining buffer before analysis. Data acquisition was performed on an LSRFortessa X-20 (Becton Dickinson) (FIG. 19).

As shown in FIG. 19, the mixtures of Vhh2g-Gen scFvHL/LH and Vhh2b-RG6292 scFv, Vhh2b-BT942 scFv or Vhh2b-7G7/B6 scFv, which is the combination of neutralizing and non-neutralizing anti-CD25 scFv, induced significant STAT5 activation in activated CD4+T cells. Also, the mixtures of Vhh2g-BT942 scFvHL/LH and Vhh2b-Gen scFv or Vhh2b-Dac scFv, which is also combination of neutralizing and non-neutralizing anti-CD25 scFv, induced significant STAT5 activation in activated CD4+T cells. Again, this shows that identifying combinations of neutralizing Abs and non-neutralizing Abs to the scaffold protein is an effective way of screening for non-competitive or biparatopic binders to construct the combination of target-binding molecules or protein complex of the present application.

### Example 33: Expression and Purification of VHH-scFv Fusion Proteins that Bind to PDL1 as a Target Scaffold Protein and CSF2RA or CSF2RB as Target Receptor Proteins

VhhPL1, which is an anti-PDL1 VHH, was fused with anti-CSF2RA scFv (116.08 scFv, 116.18 scFv) (SEQ ID NO: 110-113) and VhhPL1-1, which is another anti-PDL1 VHH, was fused with anti-CSF2RB scFv (131.16 scFv, 131.B2 scFv) (SEQ ID NO: 114-117) by linker. These anti-CSF2RA or anti-CSF2RB Ab were reported in WO2023/027177 A1. The DNA encoding these proteins were cloned into a mammalian expression vector. These plasmids were expressed transiently using Expi293 cell line (Thermo Fisher, Carlsbad, CA, USA). Protein purification was carried out using Protein A or immobilized metal ion affinity chromatography. For the concentration of the purified proteins, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 13

| SEQ ID NO | abbreviation | Sequences | Description |
|---|---|---|---|
| SEQ ID NO: | VhhPL1- | MGWSCIILFLVATATGVHSQVQLQESGGGLVQPGGSLRLSCAASGKMSSRRCMAWFRQAPGKERERVAKLLTTSGSTYL | The fusion proteins of |
| 110 | 116.08 | ADSVKGRFTISQNNAKSTVYLQMNSLKPEDTAMYYCAADSFEDPTCTLVTSSGAFQYWGQGTQVTVSSGGSEVQLVESG | PDL1 binding domain |
| | scFvHL | GGLVKPGGSLRLSCVASELTFSNVWMTWVRQAPGKGLEWVGRIKSKADGGTTDYAAPVKGRFTISRDDSKNTLYL | and CSF2RA binding |
| | | QMNSLKTEDTAVYYCTTGGYSYGDNWFDPWGQGTLVTVSSGGGGSGGGGSGGGGSEIVLTQSPATLSLSPGERAT | domain. The sequence of |
| | | LSCRASQSVSSYLAWYQQKPGQAPRLLIYDASNRATGIPARFSGSGSGTDFTLTISSLEPEDFAVYYCQQRSNWPWT | PDL1 binding domain is |
| | | FGQGTKVEIKHHHHHHHH | underlined and the |
| SEQ ID NO: | VhhPL1- | MGWSCIILFLVATATGVHSQVQLQESGGGLVQPGGSLRLSCAASGKMSSRRCMAWFRQAPGKERERVAKLLTTSGSTYL | sequence of CSF2RA |
| 111 | 116.08 | ADSVKGRFTISQNNAKSTVYLQMNSLKPEDTAMYYCAADSFEDPTCTIVISSGAFQYWGQGTQVTVSSGGSEIVLTQSP | binding domain is shown |
| | scFvLH | ATLSLSPGERATLSCRASQSVSSYLAWYQQKPGQAPRLLIYDASNRATGIPARFSGSGSGTDFTLTISSLEPEDFAVY | in bold. |
| | | YCQQRSNWPWTFGQGTKVEIKGGGGSGGGGSGGGGSEVQLVESGGGLVKPGGSLRLSCVASELTFSNVWMTWV | |
| | | RQAPGKGLEWVGRIKSKADGGTTDYAAPVKGRFTISRDDSKNTLYLQMNSLKTEDTAVYYCTTGGYSYGDNWFD | |
| | | PWGQGTLVTVSSHHHHHHHH | |
| SEQ ID NO: | VhhPL1- | MGWSCIILFLVATATGVHSQVQLQESGGGLVQPGGSLRLSCAASGKMSSRRCMAWFRQAPGKERERVAKLLTTSGSTYL | |
| 112 | 116.18- | ADSVKGRFTISQNNAKSTVYLQMNSLKPEDTAMYYCAADSFEDPTCTLVTSSGAFQYWGQGTQVTVSSGGSAIQLTQSP | |
| | scFvLH | SSLSASVGDRVTITCRASQGISSALAWYQQKPGKAPKLLIYDASSLESGVPSRFSGSGSGTDFTLTISSLQPEDFATYY | |
| | | CQQFNSYPITFGQGTRLEIKGGGGSGGGGSGGGGSEVQLVESGGGLVQPGRSLRLSCAASGFTFGDYAMHWVRQA | |
| | | PGKGLEWVSGISWNSGIIGYADSVKGRFTISRDNAKNSLYLQMNSLRAEDTALYYCVKDEGSSGWYDFNYFGMDV | |
| | | WGQGTTVTVSSHHHHHHHH | |
| SEQ ID NO: | 116.08 | MGWSCIILFLVATATGVHSEIVLTQSPATLSLSPGERATLSCRASQSVSSYLAWYQQKPGQAPRLLIYDASNRATGIPA | |
| 113 | scFvLH- | RFSGSGSGTDFTLTISSLEPEDFAVYYCQQRSNWPWTFGQGTKVEIKGGGGSGGGGSGGGGSEVQLVESGGGLVK | |
| | VhhPL1 | PGGSLRLSCVASELTFSNVWMTWVRQAPGKGLEWVGRIKSKADGGTTDYAAPVKGRFTISRDDSKNTLYLQMNSL | |
| | | KTEDTAVYYCTTGGYSYGDNWEDPWGQGTLVTVSSGGSQVQLQESGGGLVQPGGSLRLSCAASGKMSSRRCMAWFR | |
| | | QAPGKERERVAKLLTTSGSTYLADSVKGRFTISQNNAKSTVYLQMNSLKPEDTAMYYCAADSFEDPTCTLVTSSGAFQY | |
| | | WGQGTQVTVSSHHHHHHHH | |
| SEQ ID NO: | 131.16 | MGWSCIILFLVATATGVHSQVQLVESGGGLVKPGGSLRLSCAASGFTFSDYYMSWIRQAPGKGLEWVSFISSSGIIIYY | The fusion proteins of |
| 114 | scFvHL- | ADSVKGRFTISRDNAKNSLYLQMNSLRAEDTAVYYCARRGYSGYDYYYYGLDVWGQGTTVTVSSGGGGSGGGGS | PDL1 binding domain |
| | VhhPL1-1 | GGGGSEIVLTQSPGTLSLSPGERATLSCRASQSVSSSYLAWYQQKPGQAPRLLIYGASSRATGIPDRFSGSGSGTDFT | and CSF2Rβ binding |
| | | LTISRLEPEDFAVYYCQQYGSTFGGGTKVEIKGSQVQLQESGGGSVQTGGSLRLSCTASTSIYSNNYMAWFSQSPGKGR | domain. The sequence of |
| | | EGVAAVYMDDGRPYYADSVKGRFTISLDSAKNTMYLQMNSLKPEDTAMYYCAAAPGPLSRNYWYTSANYDYWGQGT | PDL1 binding domain is |
| | | QVTVSSHHHHHHHH | underlined and the |
| SEQ ID NO: | 131.16 | MGWSCIILFLVATATGVHSEIVLTQSPGTLSLSPGERATLSCRASQSVSSSYLAWYQQKPGQAPRLLTYGASSRATGIP | sequence of CSF2Rβ |
| 115 | scFvLH- | DRFSGSGSGTDFTLTISRLEPEDFAVYYCQQYGSTFGGGTKVEIKGGGGSGGGGSGGGGSQVQLVESGGGLVKPG | binding domain is shown |
| | VhhPL1-1 | GSLRLSCAASGFTFSDYYMSWIRQAPGKGLEWVSFISSSGIDIYYADSVKGRFTISRDNAKNSLYLQMNSLRAEDTAV | in bold. |
| | | YYCARRGYSGYDYYYYGLDVWGQGTTVTVSSGSQVQLQESGGGSVQTGGSLRLSCTASTSIYSNNYMAWFSQSPGKG | |
| | | REGVAAVYMDDGRPYYADSVKGRFTISLDSAKNTMYLQMNSLKPEDTAMYYCAAAPGPLSRNYWYTSANYDYWGQG | |
| | | TQVTVSSHHHHHHHH | |
| SEQ ID NO: | 131.B2 | MGWSCIILFLVATATGVHSQVQLVQSGAEVKEPGASVKISCKASGYTFTNDYMHWVRQAPGQGLEWMGIINPTGG | |
| 116 | scFvHL- | GTSYAQKFRGRVTMTRDTSTSTVYMELSSLRSEDTAVFYCARERYYGSGTYFNIGLWYFDLWGRGTLVTVSSGGG | |
| | VhhPL1-1 | GSGGGGSGGGGSEIVLTQSPGTLSLSPGERATLSCRASQSVSSSYLAWYQQKPGQAPRLLIYGASSRATGIPDRFSGS | |
| | | GSGTDFTLTISRLEPEDFAVYYCQQYGSSPLTFGGGTKVEIKGSQVQLQESGGGSVQTGGSLRLSCTASTSIYSNNYMA | |
| | | WFSQSPGKGREGVAAVYMDDGRPYYADSVKGRFTISLDSAKNTMYLQMINSLKPEDIAMYYCAAAPGPLSRNYWYTSA | |
| | | NYDYWGQGTQVTVSSHHHHHHHH | |
| SEQ ID NO: | 131.B2 | MGWSCIILFLVATATGVHSEIVLTQSPGTLSLSPGERATLSCRASQSVSSSYLAWYQQKPGQAPRLLIYGASSRATGIP | |
| 117 | scFvLH- | DRFSGSGSGTDFTLTISRLEPEDFAVYYCQQYGSSPLTFGGGTKVEIKGGGGSGGGGSGGGGSQVQLVQSGAEVKE | |
| | VhhPL1-1 | PGASVKISCKASGYTFINDYMHWVRQAPGQGLEWMGIINPTGGGTSYAQKFRGRVTMTRDTSTSTVYMELSSLRS | |
| | | EDTAVFYCARERYYGSGTYENIGLWYFDLWGRGTLVTVSSGSQVQLQESGGGSVQTGGSLRLSCTASTSIYSNNYMA | |
| | | WFSQSPGKGREGVAAVYMDDGRPYYADSVKGRFTISIDSAKNTMYIQMNSIKPEDTAMYYCAAAPGPISRNYWYTSA | |
| | | NYDYWGQGTQVTVSSHHHHHHHH | |

### Example 34: The Mixture of the VHH-scFv Fusion Proteins, which Bind to PDL1 as a Scaffold Protein and CSF2RA or CSF2RB as Target Receptor Proteins, Induce CSF2RA/B Activation in HEK-Blue GM-CSF Cells

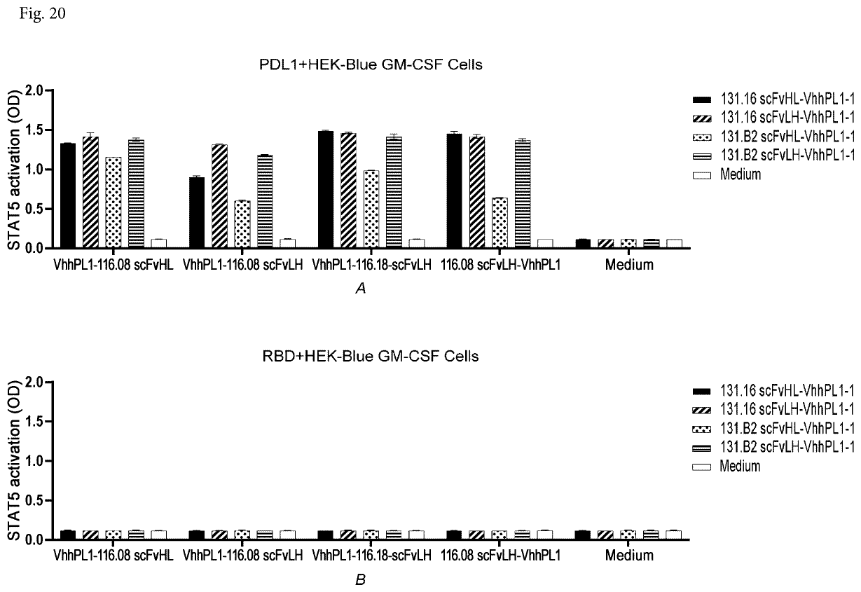

Human PDL1 (SEQ ID NO: 12) or RBD-IL2RA (SEQ ID NO: 11) was first expressed in HEK-Blue GM-CSF Cells (InvivoGen, #hkb-gmcsfr) by electroporation using SF Cell Line 4D-Nucleofector X Kit L (Lonza, #V4XC-2024). After electroporation, these transfectants were seeded in 96-well plate and incubated overnight at 37 degrees C./5% CO₂. These cells were cultured in Dulbecco's Modified Eagle Medium (DMEM) supplemented with 10% fetal bovine serum and 1% Penicillin-Streptomycin. Day 1 post electroporation, these cells were treated with different mixtures of VHH-scFv fusion proteins that can bind to PDL1 (VHH) and CSF2RA (scFv) (SEQ ID NO: 110-113) and VHH-scFv fusion proteins that can bind to PDL1 (VHH) and CSF2RB (scFv) (SEQ ID NO: 114-117) for another day at 37 degrees C./5% CO₂. The final concentration of each Ab was l0 nM. Day 2 post electroporation, the activation of GM-CSF receptors, which are CSF2RA and CSF2RB, was evaluated using Quanti-Blue solution (InvivoGen, #rep-qbs) by measuring optical density at 620 nm (FIGS. 20A and B).

As shown in FIGS. 20A and B, the mixtures of VHH-scFv fusions proteins that bind to PDL1 as a target scaffold protein and CSF2RA or CSF2RB as target receptor proteins induced significant GM-CSF receptor activation in the presence of PDL1 expression, but not in the absence of PDL1. These data suggested that not only IL-2 receptors, but also GM-CSF receptors or other cytokine receptors can be activated by the scaffold-dependent mechanism.

### Example 35: Expression and Purification of VHH-Fab Fusion Proteins that Bind to RBD as a Target Scaffold Protein and cMET as a Target Receptor Protein

VHH that can bind to SARS-CoV2 RBD (Nb21 or Nb36) was fused to Lch of anti-human cMET Fab (SEQ ID NO: 119, 120). The DNA encoding these VHH-Lch fusion proteins and Hch of anti-human cMET Fab (SEQ ID NO: 118) were cloned into a mammalian expression vector. Anti-cMET Fab was reported on Protein Data Bank (PDB: 6I04). To prepare purified Abs, the corresponding Hch and Lch expressing plasmids were expressed transiently using Expi293 cell line (Thermo Fisher, Carlsbad, CA, USA). The Hch and Lch pairs of each antibody were described in Table 14. Antibody purification was carried out using Protein A or immobilized metal ion affinity chromatography. For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 14

| | SEQ ID | | SEQ ID | | |
|---|---|---|---|---|---|
| abbreviation | NO | Hch Sequences | NO | Lch Sequences | Description |
| Nb21- | SEQ ID | MGWSCIILFLVATATGVHSQLQLQE | SEQ ID | MGWSCIILFLVATATGVHSQVQLVE | The fusion |
| Met6104 Fab | NO: 118 | SGPGLVKPSETLSLTCTVSGGSISS | NO: 119 | SGGGLVQAGGSLRLSCAVSGLGAH | proteins of |
| | | SVYYWSWIRQPPGKGLEWIGVIYP | | RVGWFRRAPGKEREFVAAIGANGG | SARS-CoV-2 |
| | | SGNTYYSPSLKSRVTISVDTSKNQF | | NTNYLDSVKGRFTISRDNAKNTIYL | RBD binding |
| | | SLKLSSVTAADTAVYYCARTIYDL | | QMNSLKPQDTAVYYCAARDIETAE | domain and |
| | | FDIWGQGTMVTVSSASTKGPSVFP | | YTYWGQGTQVTVSSGGSDIQMTQS | cMET binding |
| | | LAPSSKSTSGGTAALGCLVKDYFPEP | | PSSVSASVGDRVTITCRASQGISSW | domain. The |
| | | VTVSWNSGALTSGVHTFPAVLQSSG | | LAWYQQKPGKAPKLLIYAASSLQS | sequence of |
| | | LYSLSSVVTVPSSLGTQTYICNVNH | | GVPSRFSGSGSGTDFTLTISSLQPE | SARS-CoV-2 |
| | | KPSNTKVDKKVEPKSCAAAHHHHH | | DFATYYCLQANSFPPTFGGGTKVE | RBD binding |
| | | HHH | | IKRTVAAPSVFIFPPSDEQLKSGTAS | domain is |
| | | | | VVCLLNNFYPREAKVQWKVDNALQ | underlined and |
| | | | | SGNSQESVTEQDSKDSTYSLSSTLTL | the sequence of |
| | | | | SKADYEKHKVYACEVTHQGLSSPV | cMET binding |
| | | | | TKSFNRGEC | domain is |
| Nb36- | | | SEQ ID | MGWSCIILFLVATATGVHSVQLVES | shown in bold. |
| Met6104 Fab | | | NO: 120 | GGGLVQAGGSLTLTCAASGRIFSSE | |
| | | | | TMDMGWERQAPGKEREFVAADSW | |
| | | | | NDGSTYYADSVKGRFTISRDSAKNT | |
| | | | | LYLQMNSLKPEDTAVYYCAAETYSI | |
| | | | | YEKDDSWGYWGQGTQVTVSGGSDI | |
| | | | | QMTQSPSSVSASVGDRVTITCRAS | |
| | | | | QGISSWLAWYQQKPGKAPKLLIY | |
| | | | | AASSLQSGVPSRFSGSGSGTDFTLT | |
| | | | | ISSLQPEDFATYYCLQANSFPPTFG | |
| | | | | GGTKVEIKRTVAAPSVFIFPPSDEQL | |
| | | | | KSGTASVVCLLNNFYPREAKVQWK | |
| | | | | VDNALQSGNSQESVTEQDSKDSTYS | |
| | | | | LSSTLTLSKADYEKHIKVYACEVTHQ | |
| | | | | GLSSPVTKSFNRGEC | |

### Example 36: The Activation of cMET Receptor by Mixtures of VHH-Fab Fusions, which Bind to RBD and cMET, is Dependent on Biparatopic Binding to RBD

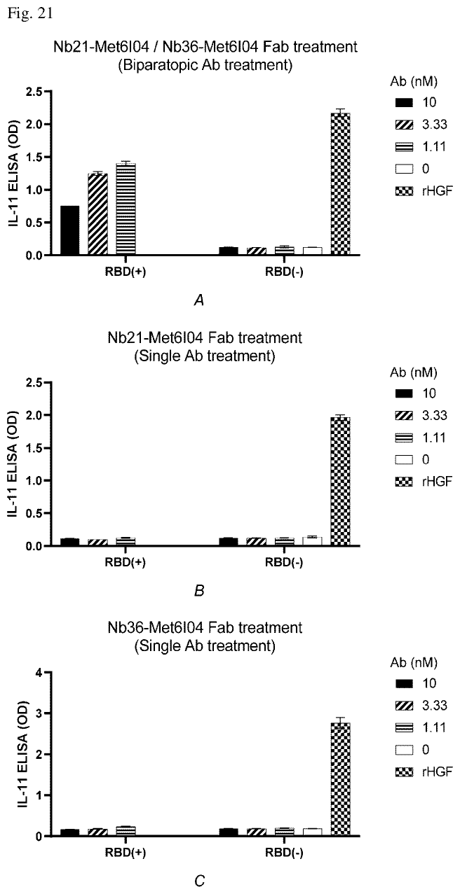

Saos-2 cells (ATCC, #HTB-85) were used and maintained in RPMI1640 supplemented with 10% fetal bovine serum and 1% Penicillin-Streptomycin. Saos-2 cells were seeded to 96-well plates and stimulated with VHH-Fab fusion proteins which bind to RBD as a target scaffold protein and cMET as a target receptor protein (Nb21-Met6I04 Fab, Nb36-Met6I04 Fab) in the presence of recombinant SARS-CoV-2 spike RBD protein (R&D systems, #10523-CV-100, 640 pM as a final concentration) for 2 days. Recombinant human HGF protein (R&D systems, #294-HGN/CF) was used as a positive control. 2 days after stimulation, culture supernatant was collected and detection of IL-11, which is induced by cMET activation, was performed using Human IL-11 DuoSet ELISA kit (R&D systems, #DY218) (FIGS. 21A, 21B and 21C).

As shown in FIGS. 21A, 21B and 21C, the mixture of VHH-Fab fusion proteins of anti-RBD and anti-cMET Fab, which bind to RBD in biparatopic manner, induced significant cMET activation in the presence of recombinant SARS-CoV-2 spike RBD protein (FIG. 21A), but not for monoparatopic Abs (FIGS. 21B and 21C). These data suggested that not only IL-2 receptors, but also other cytokine receptors can be activated by the scaffold-dependent mechanism.

### Example 37: Expression and Purification of Bispecific Antibodies that Bind to MUC1 or CEA as a Target Scaffold Protein and FZDs or LRPs as Target Receptor Proteins

The DNA encoding Hch and Lch of R2M3-R2M3 (anti-FZDs Ab, SEQ ID NO: 121, 123), AR20.5 (anti-MUC1 Abs, SEQ ID NO: 124, 127) and CEA.Mab3 (anti-CEA Abs, SEQ ID NO: 126, 128) were cloned into a mammalian expression vector. Also, the DNA encoding Vhhlrp36-Vhhlrp36 (anti-LRPs Ab, SEQ ID NO: 122), CEA.VHH (anti-CEA Ab, SEQ ID NO: 125) were cloned into a mammalian expression vector. To prepare purified Abs, corresponding Hch and Lch expressing plasmids were expressed transiently using Expi293 cell line (Thermo Fisher, Carlsbad, CA, USA). The pairs of Hch and Lch of each antibody were described in Table 15. From these purified Abs, bispecific antibodies against FZDs and MUC1 (R2M3-R2M3//AR20.5), or FZDs and CEA (R2M3-R2M3//CEA.VHH, R2M3-R2M3//CEA.MAb3) were prepared by using Fab arm exchanging technology (as described in WO2015/046467). Also, bispecific antibodies against LRPs and MUC1 (Vhhlrp36-Vhhlrp36//AR20.5), or LRPs and CEA (Vhhlrp36-Vhhlrp36//CEA.VHH, Vhhlrp36-Vhhlrp36//CEA.MAb3) were prepared by using Fab arm exchanging technology. For the concentration of the purified antibodies, their absorbance at 280 nm was measured using a spectrophotometer. From the obtained value, the extinction coefficient calculated by the methods such as PACE was used to calculate the antibody concentration (Protein Science 1995; 4: 2411-2423).

TABLE 15
Bispecific Ab Arm1

| abbreviation | | Hch Sequences | | Lch Sequences | Description |
|---|---|---|---|---|---|
| R2M3-R2M3 | SEQ I NO: | MDMRVPAQLLGLLLLWLRGARCEVQLVQSGAEVKKPG | SEQ ID NO: | MDMRVPAQLLGLLLLWLRGARCQAVVLQEPSLSVSPG | anti-Frizzled |
| | 121 | ASVKVSCKASGYTFTSYGISWVRQAPGQGLEWMGWIS | 123 | GTVTLTCGLSSGSVSTNYYPSWYQQTPGQAPRTLIYYIN | receptors (FZDs) |
| | | AYNGNTNYAQKLQGRVTMTTDTSTSTAYMELRSLRSD | | TRSSDVPERFSGSIVGNKAALTITGAQPDDESVYFCLLYL | antibody. This |
| | | DTAVYYCASSKEKATYYYGMDVWGQGTTVTVSSASTK | | GRGIWVFGGGTKLTVLGQPKAAPSVTLFPPSSEELQANK | antibody was used |
| | | GPSVFPLAPSSKSTSGGTAALGCLVKDYFPEPVTVSWNS | | ATI.VCLISDFYPGAVTVAWKADSSPVKAGVETTTPSKQ | for preparation of |
| | | GALTSGVHTFPAVLQSSGLYSLSSVVTVPSSSLGTQTYIC | | SNNKYAASSYLSLTPEQWKSHRSYSCQVTHEGSTVEKT | bispecific |
| | | NVNHKPSNTKVDKKVEPKSCGGGGSGGGGSEVQLVQS | | VAPTECS | antibodies. |
| | | GAEVKRPGASVKVSCKASGYTFTSYGISWVRQAPGQGL | | | |
| | | EWMGWISAYNGNTNYAQKLQGRVTMITDISTSTAYM | | | |
| | | ELRSLRSDDTAVYYCASSKEKATYYYGMDVWGQGTTV | | | |
| | | TVSSASTKGPSVFPLAPSSKSTSGGTAALGCLVKDYFPEP | | | |
| | | VTVSWNSGALTSGVHTFPAVLQSSGLYSLSSVVTVPSSS | | | |
| | | LGTQTYICNVNHKPSNTKVDKRVEPKSCDKTHTCPPCP | | | |
| | | APELRRGPSVFLFPPKPKDTLMISRTPEVTCVVVDVSHE | | | |
| | | DPEVKFNWYVDGVEVHNAKTKPREEQYNSTYRVVSVL | | | |
| | | TVLHQDWLNGKEYKCKYSNKALPAPIEKTISKAKGQPR | | | |
| | | EPQVYTLPPSRKEMTKNQVSLTCLVKGFYPSDIAVEWES | | | |
| | | NGQPENNYKTTPPYLDSDGSFFLYSKLTVDKSRWQQGN | | | |
| | | VFSCSVMHEALHNHYTQKSLSLSP | | | |
| Vhhlrp36- | SEQ ID NO: | MDMRVPAQLLGLLLLWLRGARCQVKLEESGGGLVQA | | none | anti-Low-density |
| Vhhlrp36 | 122 | GGSLRLSCAASGRIFSIYDMGWFRQAPGKEREFVSGIRW | | | lipoprotein |
| | | SGGTSYADSVKGRFTISKDNAKNTIYLQMNNLKAEDTA | | | receptor-related |
| | | VYYCGSRGYWGQGTLVTVSSGGGGSGGGGSQVKLEES | | | proteins (LRPs) |
| | | GGGLVQAGGSLRLSCAASGRIFSTYDMGWFRQAPGKER | | | antibody. This |
| | | EFVSGIRWSGGTSYADSVKGRFTISKDNAKNTIYLQMN | | | antibody was used |
| | | NLKAEDTAVYYCGSRGYWGQGTLVTVSSGGGGSEPKS | | | for preparation of |
| | | SDKTHTCPPCPAPELRRGPSVFLFPPKPKDTLMISRTPEV | | | bispecific |
| | | TCVVVDVSHEDPEVKFNWYVDGVEVHNAKTKPREEQY | | | antibodies. |
| | | NSTYRVVSVLTVLHQDWLNGKEYKCKVSNKALPAPIEK | | | |
| | | TISKAKGQPREPQVYTLPPSRKEMTKNQVSLTCLVKGFY | | | |
| | | PSDIAVEWESNGQPENNYKTTPPYLDSDGSFFLYSKLTV | | | |
| | | DKSRWQQGNVFSCSVMHEALHNHYTQKSLSLSP | | | |
| | | GVHSEVQLVESGGGFVQAGESLT | | | |
| AR20.5 | SEQ ID NO: | MGWSCIILFLVATATGVHSEVKLVESGGGLVAPGGSLK | SEQ ID NO: | MGWSCIILFLVATATGVHSDVLMTQTPLSLPVSLGDQAS | anti-MUC1 Ab for |
| | 124 | LSCAASGFTFSSYPMSWVRQTPEKRLEWVAYINNGGGN | 127 | ISCRSSQTIVHSNGKIYLEWYLQKPGQSPKLLIYRVSKRF | preparation |
| | | PYYPDTVKGRFTISRDNAKNTLYLQMSSLKSEDTAIYYC | | SGVPDRFSGSGSGTDFTLKISRVEAEDLGVYYCFQGSHV | of bispecific Abs |
| | | IRQYYGFDYWGQGTTLTVSSASTKGPSVFPLAPSSKSTS | | PWTPGGGTKLEIKRTVAAPSVFIFPPSDEQLKSGTASVV | |
| | | GGTAALGCLVKDYFPEPVTVSWNSGALTSGVHTFPAVL | | CLLNNFYPREAKVQWKVDNALQSGNSQESVTEQDSKD | |
| | | QSSGLYSLSSVVTVPSSSLGTQTYICNVNHKPSNTKVDK | | STYSLSSTLTLSKADYEKHKVYACEVTHQGLSSPVTKSF | |
| | | RVEPKSCDKTHTCPPCPAPELRRGPSVFLFPPKPKDTLMI | | NRGEC | |
| | | SRTPEVTCVVVDVSHEDPEVKFNWYVDGVEVHNAKTK | | | |
| | | PREEQYNSTYRVVSVLTVLHQDWLNGKEYKCKVSNKA | | | |
| | | LPAPIEKTISKAKGQPREPQVYTLPPSREEMTKNQVSLTC | | | |
| | | LVKGFYPSDIAVEWESNGQPENNYKTTPPYLDSDGSFFL | | | |
| | | YSKLTVDKSRWQQGNFSCSVMHEALHNHYTQESLSL | | | |
| | | SP | | | |
| CEA VHH | SEQ ID NO: | MGWSCIILFLVATATGVHSEVQLVESGGGFVQAGESLT | | none | anti-CEA Abs for |
| | 125 | LSCTSSTLTFTPYRMAWYRQAPGKQRDLVADISSGDGR | | | preparation |
| | | TTNYADFAKGRFTISRDNIKNTVFLRMTNLKPEDTAVY | | | of bispecific Abs |
| | | YCNTFVSFVGIARSWGQGTQVTVSSGGGGSEPKSSDKT | | | |
| | | HTCPPCPAPELRRGPSVFLFPPKPKDTLMISRTPEVTCVV | | | |
| | | VDVSHEDPEVKFNWYVDGVEVHNAKTKPREEQYNSTY | | | |
| | | RVVSVLTVLHQDWLNGKEYKCKVSNKALPAPIEKTISK | | | |
| | | AKGQPREQVYTLPPSREEMTKNQVSLTCLYKGFYPSDI | | | |
| | | AYEWESNGQPENNYKTTPPYLDSDGSFFLYSKLTVDKS | | | |
| | | RWQQGNVFSCSVMHEALHNHYTQESLSLSP | | | |
| CEA.MAb3 | SEQ ID NO: | MGWSCIILFLVATATGVHSEVKLVESGGGLVKPGGSLT | SEQ ID NO: | MGWSCIILFLVATATGVHSDIVMTQSQRFMSTLEGDRV | |
| | 126 | LPCAASGFTFSRYAMSWVRQTPEKRLEWVASISSGGQT | 128 | SVTCKASQNVGTNVAWYQQKPGQSPKALIYSASYRYS | |
| | | YYPDSVKGRFTVSRDNARNILFLQMSSLRSEDTGMYYC | | GVPDRFTGSGSGTDFTLTISNVQSEDLAEYFCQQYNNYP | |
| | | ARVNYYDSSFLDWWGQGTTLTVSSASTKGPSVFPLAPS | | LYTFGGGTKLEIKRTVAAPSVFIFPPSDEQLKSGTASVVC | |
| | | SKSTSGGTAALGCLVKDYFPEPVTVSWNSGALTSGVHT | | LLNNFYPREAKVQWKVDNALQSGNSQESVTEQDSKDS | |
| | | FPAYLQSSGLYSLSSVVTVPSSSLGTQTYICNVNHKPSNT | | TYSLSSTLTLSKADYEKHKVYACEVTHQGLSSPVTKSFN | |
| | | KVDKRVEPKSCDKHTCPPCPAPELRRGPSVFLFPPKPK | | RGEC | |
| | | DTLMISRTPEVTCVVVDVSHEDPEYKFNWYVDGVEVH | | | |
| | | NAKTKPREEQYNSTYRVVSVLIVLHQDWLNGKEYKCK | | | |
| | | VSNKALPAPIEKTISKAKGQPREPQVYTLPPSREEMTKN | | | |
| | | QVSLTCLVKGFYPSDIAVEWESNGQPENNYKTTPPYLDS | | | |
| | | DGSFFLYSKLTVDKSRWQQGNVFSCSVMHEALHNHYT | | | |
| | | QESLSLSP | | | |

### Example 38: The Mixture of Bispecific Abs that Bind to MUC1 and Wnt Receptor (FZDs or LRPs) Induced Wnt Receptor Activation in the Presence of MUC1 Expression

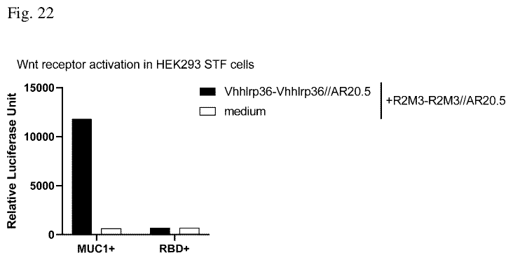

MUC1 (SEQ ID NO:129) or RBD-IL2RA (SEQ ID NO: 11) was first expressed in HEK293 STF cells (ATCC, #CRL-3249) by electroporation using SF Cell Line 4D-Nucleofector X Kit L (Lonza, #V4XC-2024). These transfectants were seeded in white 384-well plate and incubated overnight at 37 degrees C./5% CO₂ after addition of 200 nM RSPO1 protein (in-house, #PPU5200) and 50 nM LGK974 (Cayman chemical, #14072). These cells were cultured in Dulbecco's Modified Eagle Medium (DMEM) F-12, HEPES supplemented with 20% fetal bovine serum and 200 micro g/mL Geneticin. Day 1 post electroporation, these cells were treated with mixtures of bispecific Abs (R2M3-R2M3//AR20.5 and Vhhlrp36-Vhhlrp36//AR20.5, 25 nM each as a final concentration) that bind to MUC1 as a target scaffold protein and Wnt receptor proteins (FZDs or LRPs) as target receptor proteins, and further incubated at 37 degrees C./5% CO₂ for another day. Day 2 post electroporation, Wnt signal activation was evaluated using ONE-Glo Luciferase Assay System (Promega, #E6120) (FIG. 22). AR20.5 recognize the repeated sequence of MUC1 protein (Hybrid Hybridomics 2001; 20: 313-24), and therefore, this Ab can bind to multiple sites of MUC1.

As shown in FIG. 22, the mixture of bispecific Abs that bind to MUC1 and Wnt receptors can induce Wnt receptor activation in the presence of MUC1.

### Example 39: Bispecific Ab Mixture that Bind to CEA and Wnt Receptors Induce Wnt Receptor Activation in the Presence of CEA

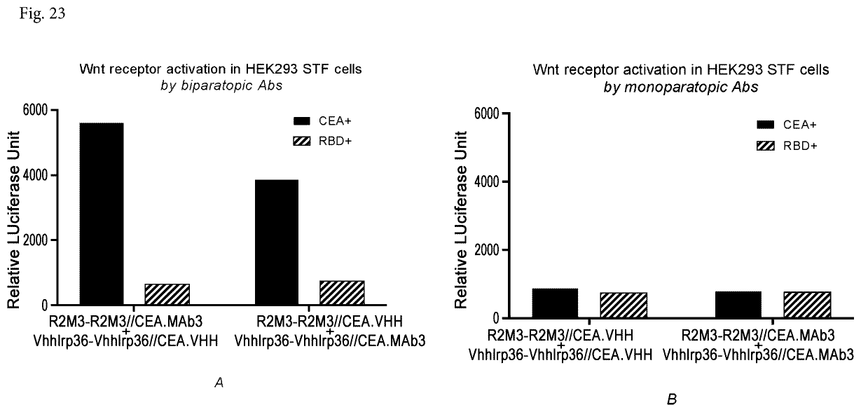

CEA (SEQ ID NO:130) or RBD-IL2RA (SEQ ID NO: 11) was first expressed in HEK293 STF cells (ATCC, #CRL-3249) by electroporation using SF Cell Line 4D-Nucleofector X Kit L (Lonza, #V4XC-2024). These transfectants were seeded in white 384-well plate and incubated overnight at 37 degrees C./5% CO₂ after addition of 200 nM RSPO1 protein (in-house, #PPU5200) and 50 nM LGK974 (Cayman chemical, #14072). These cells were cultured in Dulbecco's Modified Eagle Medium (DMEM) F-12, HEPES supplemented with 20% fetal bovine serum and 200 micro g/mL Geneticin. Day 1 post electroporation, these cells were treated with mixtures of bispecific Abs (25 nM, as a final concentration) that bind to CEA as a target scaffold protein and Wnt receptors (FZDs or LRPs) as target receptor proteins and further incubated at 37 degrees C./5% CO₂ for another day. Day 2 post electroporation, Wnt signal activation was evaluated using ONE-Glo Luciferase Assay System (Promega, #E6120) (FIGS. 23A and 23B).

As shown in FIGS. 23A and 23B, the mixture of biparatopic anti-CEA Abs that were fused with Wnt receptor binding domain induced activation of Wnt receptor in the presence of CEA.

TABLE 16

| SEQ ID NO | abbreviation | Sequences | Description |
|---|---|---|---|
| SEQ ID NO: | MUC1 | MTPGTQSPFFLLLLLTVLTVVTGSGHASSTPGGEKETSATQ | Human |
| 129 | | RSSVPSSTEKNAVSMTSSVLSSHSPGSGSSTTQG | MUC1 |
| | | QDVTLAPATEPASGSAATWGQDVTSVPVTRPALGSTT | |
| | | PPAHDVTSAPDNKPAPGSTAPPAHGVTSAPDTRPAPG | |
| | | STAPPAHGVTSAPDTRPAPGSTAPPAHGVTSAPDTRP | |
| | | APGSTAPPAHGVTSAPDTRPAPGSTAPPAHGVTSAPDT | |
| | | RPAPGSTAPPAHGVTSAPDTRPAPGSTAPPAHGVTSAP | |
| | | DTRPAPGSTAPPAHGVTSAPDTRPAPGSTAPPAHGVT | |
| | | SAPDTRPAPGSTAPPAHGVTSAPDTRPAPGSTAPPAHG | |
| | | VTSAPDTRPAPGSTAPPAHGVTSAPDTRPAPGSTAPP | |
| | | AHGVTSAPDTRPAPGSTAPPAHGVTSAPDTRPAPGSTA | |
| | | PPAHGVTSAPDTRPAPGSTAPPAHGVTSAPDTRPAP | |
| | | GSTAPPAHGVTSAPDTRPAPGSTAPPAHGVTSAPDTRP | |
| | | APGSTAPPAHGVTSAPDTRPAPGSTAPPAHGVTSAPD | |
| | | TRPAPGSTAPPAHGVTSAPDTRPAPGSTAPPAHGVTSA | |
| | | PDTRPAPGSTAPPAHGVTSAPDTRPAPGSTAPPAHGV | |
| | | TSAPDTRPAPGSTAPPAHGVTSAPDTRPAPGSTAPPAH | |
| | | GVTSAPDTRPAPGSTAPPAHGVTSAPDTRPAPGSTAP | |
| | | PAHGVTSAPDTRPAPGSTAPPAHGVTSAPDTRPAPGST | |
| | | APPAHGVTSAPDTRPAPGSTAPPAHGVTSAPDTRPAP | |
| | | GSTAPPAHGVTSAPDTRPAPGSTAPPAHGVTSAPDTRP | |
| | | APGSTAPPAHGVTSAPDTRPAPGSTAPPAHGVTSAPD | |
| | | TRPAPGSTAPPAHGVTSAPDTRPAPGSTAPPAHGVTSA | |
| | | PDTRPAPGSTAPPAHGVTSAPDTRPAPGSTAPPAHGV | |
| | | TSAPDTRPAPGSTAPPAHGVTSAPDTRPAPGSTAPPAH | |
| | | GVTSAPDNRPALGSTAPPVHNVTSASGSASGSASTLV | |
| | | HNGTSARATTTPASKSTPFSIPSHHSDTPTTLASHSTKT | |
| | | DASSTHASSVPPLTSSNHSTSPQLSTGVSFFFLSFHISN | |
| | | LQFNSSLEDPSTDYYQELQRDISEMFLQIYKQGGFLGLS | |
| | | NIKFRPGSVVVQLTLAFREGTINVHDVETQFNQYKT | |
| | | EAASRYNLTISDVSVSDVPFPFSAQSGAGVPGWGIALLV | |
| | | LVCVLVALAIVYLIALAVCQCRRKNYGQLDIFPAR | |
| | | DTYHPMSEYPTYHTHGRYVPPSSTDRSPYEKVSAGNGGS | |
| | | SLSYTNPAVAATSANL | |
| SEQ ID NO: | CEA | MESPSARPPHRWCIPWQRLLLTASLLTFWNPPTTAKL | Human |
| 130 | | TIESTPENVAEGKEVLLLVHNLPQHLFGYSWYKGERVD | CEA |
| | | GNRQIIGYVIGTQQATPGPAYSGREIIYPNASLLIQNII | (CECAM1) |
| | | QNDTGFYTLHVIKSDLVNEEATGQFRVYPELPKPSISSN | |
| | | NSKPVEDKDAVAFTCEPETQDATYLWWVNNQSLPVSPRL | |
| | | QLSNGNRTLTLFNVTRNDTASYKCETQNPVSAR | |
| | | RSDSVILNVLYGPDAPTISPLNTSYRSGENLNLSCHAAS | |
| | | NPPAQYSWFVNGTFQQSTQELFIPNITVNNSGSYTCQ | |
| | | AHNSDTGLNRTTVTTITVYAEPPKPFITSNNSNPVEDED | |
| | | AVALTCEPEIQNTTYLWWVNNQSLPVSPRLQLSND | |
| | | NRTLTLLSVTRNDVGPYECGIQNELSVDHSDPVILNVLY | |
| | | GPDDPTISPSYTYYRPGVNLSLSCHAASNPPAQYSW | |
| | | LIDGNIQQHTQELFISNITEKNSGLYTCQANNSASGHSRT | |
| | | TVKTITVSAELPKPSISSNNSKPVEDKDAVAFTCEPE | |
| | | AQNTTYLWWVNGQSLPVSPRLQLSNGNRTLTLFNVTRNDA | |
| | | RAYVCGIQNSVSANRSDPVTLDVLYGPDTPIISP | |
| | | PDSSYLSGANLNLSCHSASNPSPQYSWRINGIPQQHTQVL | |
| | | FIAKITPNNNGTYACFVSNLATGRNNSIVKSITVSA | |
| | | SGTSPGLSAGATVGIMIGVLVGVALI | |

### Example 40: Expression and Purification of VHH-VHH Fusion Proteins that Bind to CD8 as a Scaffold Protein and IL-2R Beta and IL-2R Gamma as Receptor Proteins

Anti-CD8a or anti-CD8b VHH were fused with another VHH that can bind to human IL-2R gamma or IL-2R beta (SEQ ID NO: 131-141) by linker. The DNA encoding these proteins were cloned into a mammalian expression vector. These plasmids were expressed transiently using Expi293 cell line. (thermo Fisher, Carlsbad, CA, USA). Protein purification was carried out using Protein A or immobilized metal ion affinity chromatography. For the concentration of the purified proteins, their absorbance at 280 nm coefficient calculated by the methods such as PACE was used to calculate the antibody concentration (Protein Science 1995; 4:2411-2423).

TABLE 17

| SEQ ID NO | abbreviation | Hch Sequences | Description |
|---|---|---|---|
| SEQ ID NO: | Vhh2g- | MGWSCIILFLVATATGVHSQVQLQESGGGSVQAGGSLRLSCAASGYTYR | The fusion proteins of |
| 131 | VhhC8.2 | DYYMGWFRQAPGREREGVASIYTRGSREGSTRYSSSVEGRFTITLDTA | CD8 binding domain and |
| | | KNTLYLQMNSLKPEDTAMYYCAADDRTWLPRVQLGGPRENEYNYW | IL-2Rγ binding domain. |
| | | GQGTQVTVSSGGSQLQLVESGGGLVQAGGSLRLSCAASGRGFSSYTMGW | The sequence of CD8 |
| | | FRQAPGKEREFVSAIRWSGSITYYADSVKGRFTISRDNAKNTMYLQMNSL | binding domain is |
| | | KPEDTAVYYCAASPVAGWGTSPAWYDYWGQGTQVTVSS | underlined and the |
| SEQ ID NO: | Vhh2g30- | MGWSCIILFLVATATGVHSQVQLQESGGGSVQAGGSLRLSCAASGYTYS | sequence of IL-2Rγ |
| 132 | VhhC8.2 | KNWYMGWFRQTPGKEREGVAVIAYDDWPTYADSVKGRFTISKDNTK | binding domain is shown |
| | | NTLYLQMNSLKPEDTAMYYCAARQLGGDYCYFPNLSRFCYNYWGQG | in bold. |
| | | TQVTVSSGGSQLQLVESGGGLVQAGGSLRLSCAASGRGESSYTMGWFRQ | |
| | | APGKEREFVSAIRWSGSITYYADSVKGRFTISRDNAKNTMYLQMNSLKPED | |
| | | TAVYYCAASPVAGWGTSPAWYDYWGQGTQVTVSS | |
| SEQ ID NO: | Vlih2g40- | MGWSCIILFLVATATGVHSQVQLQESGGGSVQAGGSLRLSCTASGFTFN | |
| 133 | VhhC8.2 | EANHMGWYRQAPGNECELVSTISSDGTTYYPDSVKGRFTISQDNAKKT | |
| | | AFLQMNSLKPEDTAVYYCAADQSRRGSLCLGQGTQVTVSSGGSQLQLV | |
| | | ESGGGLVQAGGSLRLSCAASGRGESSYTMGWFRQAPGKEREFVSAIRWSG | |
| | | SITYYADSVKGRFTISRDNAKNTMYLQMNSLKPEDTAVYYCAASPVAGW | |
| | | GTSPAWYDYWGQGTQVTVSS | |
| SEQ ID NO: | VhhC8.4- | MGWSCIILFLVATATGVHSQLQLVESGGGLVQPGGSLTLSCAASGRSFSSY | The fusion proteins of |
| 134 | Vhh2b | GMGWFRQAPGKEREFVATISWNSGSIYYTDSVKGREFTISRDDAKNTMSLQ | CD8 binding domain and |
| | | MNSLRPEDTAVYSCAAGPRDWGNMRKFEEYEYRGQGTQVTVSSGGSQV | IL-2Rβ binding domain. |
| | | QLQESGGGSVQAGGSLRLSCAASSYTISSVCMGWFRQAPGKEREGVA | The sequence of CD8 |
| | | GIAPDGSTGYGDSVKGRFTISKDNAKNTLYLQMNSLKPEDTAMYYCA | binding domain is |
| | | AASPGRCFLPRTALEPALYYNWGQGTQVTVSS | underlined and the |
| SEQ ID NO: | VhhC8.8- | MGWSCIILFLVATATGVHSQVQLVESGGGLVQPGGSLTLSCAASGRSFSEY | sequence of IL-2Rβ |
| 135 | Vhh2b | GMGWERQAPGKEREFVATISWDSDSIYYTDSVKGRFTISRDNAKNTMSLQ | binding domain is shown |
| | | MNSLKPEDTAVYSCAARPRDWGNMRRFEAYEYRGQGTQVTVSSGGSQV | in bold. |
| | | QLQESGGGSVQAGGSLRLSCAASSYTISSVCMGWFRQAPGKEREGVA | |
| | | GIAPDGSTGYGDSVKGRFTISKDNAKNTLYLQMNSLKPEDTAMYYCA | |
| | | AASPGRCFLPRTALEPALYYNWGQGTQVTVSS | |
| SEQ ID NO: | VhhC8.9- | MGWSCIILFLVATATGVHSQVQLVESGGGLVQTGGSLRLSCAASEPTFSDY | |
| 136 | Vhh2b | AGSWFRQAPGKEREFLASINWRGDTTYYADSVKGRFTISRDNAKNMMYL | |
| | | EMNNLEPEDTAVYRCAAKATDWSSTLYEYDYWGQGTQVTVSSGGSQVQ | |
| | | LQESGGGSVQAGGSLRLSCAASSYTISSVCMGWFRQAPGKEREGVAGI | |
| | | APDGSTGYGDSVKGRFTISKDNAKNTLYLQMNSLKPEDTAMYYCAAA | |
| | | SPGRCFLPRTALEPALYYNWGQGTQVTVSS | |
| SEQ ID NO: | VhhC8.4- | MGWSCIILFLVATATGVHSQLQLVESGGGLVQPGGSLTLSCAASGRSFSSY | |
| 137 | Vhh2b1 | GMGWFRQAPGKEREFVATISWNSGSIYYTDSVKGRFTISRDDAKNTMSLQ | |
| | | MNSLRPEDTAVYSCAAGPRDWGNMRKFEEYEYRGQGTQVTVSSGGSQV | |
| | | QLQESGGGSVQAGGSLRLSCVGSGYTYDTSDMSWYRQAPGKEREFVS | |
| | | GIAPDGSTGYGDSVKGRFTISKDNAKNTLYLQMNSLKPEDTAMYYCA | |
| | | ASYWKWGKLNNFWGPGTQVTVSSHHHHHH | |
| SEQ ID NO: | VhhC8.4- | MGWSCIIFLVATATGVHSQLQLVESGGGLVQPGGSLTLSCAASGRSFSSY | The fusion protein |
| 138 | Vhh2b3 | GMGWFRQAPGKEREFVATISWNSGSIYYTDSVKGRFTISRDDAKNTMSLQ | CD8 binding doma |
| | | MNSLRPEDTAVYSCAAGPRDWGNMRKFEEYEYRGQGTQVTVSSGGSQV | IL-2Rβ binding do |
| | | QLQESGGGLVQPGGSLKLSCAASGFRFSBYGMSWVRQAPGEGLEWV | The sequence of CD8 |
| | | SYINGDGSRTHYADSVKGRFTISRDNAKNTLYLQLNSLKTEDTAMYYC | binding domain |
| | | EKGLSRDGWSLSAASRGQGTQVTVSSHHHHHH | underlined and the |
| SEQ ID NO: | VhhC8.4- | MGWSCIILFLVATATGVHSQLQLVESGGGLVQPGGSLTLSCAASGRSFSSY | sequence of IL-2Rβ |
| 139 | Vhh2b5 | GMGWFRQAPGKEREFVATISWNSGSIYYTDSVKGRFTISRDDAKNTMSLQ | binding domain is shown |
| | | MNSLRPEDTAVYSCAAGPRDWGNMRKFEEYEYRGQGTQVTVSSGGSQV | in bold. |
| | | QLQESGGGSVQAGGSLRLSCVTSGYTYSSANMAWFRQAPGKEREGVA | |
| | | IITPSGRATTYADSVKGRFTISRDNAANTLYLQMNSLKPEDTAMYYCA | |
| | | ADTPPYSGLWYAERTYNYWGQGTQVTVSSHHHHHH | |
| SEQ ID NO: | VhhC8.4- | MGWSCIILFLVATATGVHSQLQLVESGGGLVQPGGSLTLSCAASGRSFSSY | |
| 140 | Vhh2b6 | GMGWFRQAPGKEREFVATISWNSGSIYYTDSVKGRFTISRDDAKNTMSLQ | |
| | | MNSLRPEDTAVYSCAAGPRDWGNMRKFEEYEYRGQGTQVTVSSGGSQV | |
| | | QLQESGGGSVQAGGSLRLSCTASGFTFDDEDMGWYRQAPGNECELVS | |
| | | SLGSLGRRYYADSVKDRFAISQDNAKNTVYLQMNSLKPEDTAVYYCA | |
| | | ATKGGSWLDSILASCQGAFGYWGQGTQVTVSSHHHHHH | |
| SEQ ID NO: | VhhC8.4- | MGWSCIILFLVATATGVHSQLQLVESGGGLVQPGGSLTLSCAASGRSFSSY | |
| 141 | Vhh2b7 | GMGWFRQAPGKEREFVATISWNSGSIYYTDSVKGRFTISRDDAKNTMSLQ | |
| | | MNSLRPEDTAVYSCAAGPRDWGNMRKFEEYEYRGQGTQVTVSSGGSQV | |
| | | QLQESGGGSVQAGGSLRLSCAASGSTSCSSVMRWYRQAPGKEREFVSS | |
| | | INSDRRTVYADSVKGRFTISQDNAKSTLYLQMNSLKAEDTATYYCQRE | |
| | | LYGDSWCQGNYWGQGTQVTVSSHHHHHH | |

### Example 41: The Combinations of VHH-VHH Fusion Proteins that Target CD8 as a Target Scaffold Protein and IL-2R Beta/Gamma as Target Receptor Proteins Induce IL-2 Receptor Activation in CD8+T Cells, but not in CD4+T Cells

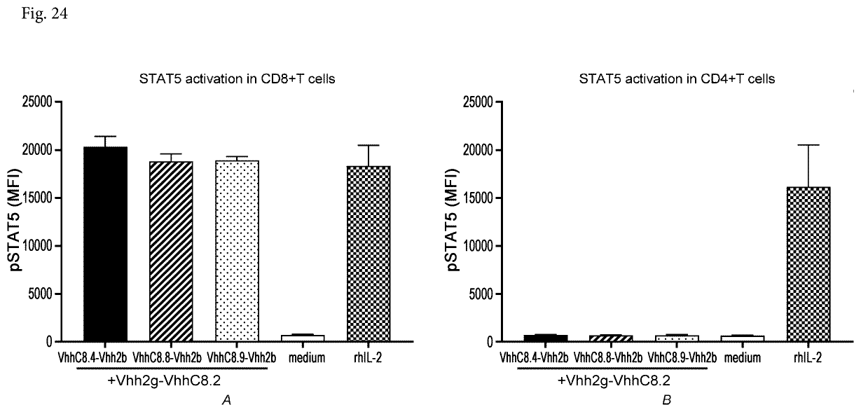

Human CD8+T cells or human CD4+T cells were isolated from human PBMCs (STEMCELL, #70025.2) using EasySep™ Human CD8+T cell isolation kit (STEMCELL, #19053) or EasySep™ Human CD4+T cell isolation kit (STEMCELL, #17952). After isolation, CD8+ or CD4+T cell were treated with plate coated anti-CD3/CD28 Abs (Biolegend, #317347, #302943, 5 micro g/mL each) and rhIL-2 (Peprotech, #200-02) for 3 days at 37 degrees C./5% CO₂. CTS Optimizer T cell expansion SFM (GIBCO, #A1048501) with penicillin/streptomycin (GIBCO, #15140122), GlutaMax (GIBCO, #35050061) and 10% fetal bovine serum was used for culture medium. After 3 days culture, CD8+ or CD4+ T cell were harvested and washed with culture medium twice and rested overnight with culture medium. After resting, T cells were stimulated with the mixtures of Vhh2g-VhhC8.2 (SEQ ID NO: 131) and VhhC8.4-Vhh2b, VhhC8.8-Vhh2b or VhhC8.9-Vhh2b (SEQ ID NO: 134-136) with 100 nM concentration for 30 minutes at 37 degrees C. rhIL-2 (Peprotech, #200-02) was used as a positive control. These cells were immediately fixed with CytoFix (BD Biosciences, #554655) for 20 minutes at room temperature. Cells were washed once with staining buffer (Biolegend, #420201) and chilled permeabilization buffer (BD Biosciences, #558050) were subsequently added to the cells and incubated on ice for 30 minutes. Cells were washed twice with staining buffer and Alexa Fluor (registered trademark) 647 Mouse Anti-Stat5 (pY694) (BD Biosciences, #562076) were added and incubated for at least 60 minutes at room temperature. Cells were washed once with staining buffer before analysis. Data acquisition was performed on an LSRFortessa X-20 (Becton Dickinson) (FIGS. 24A and 24B).

As shown in FIGS. 24A and B, the mixtures of Vhh2g-VhhC8.2 and VhhC8.4-Vhh2b, VhhC8.8-Vhh2b or VhhC8.9-Vhh2b induced significant STAT5 activation in activated human CD8+T cells but not in the CD4+T cells. These data indicated various CD8 binding domain can be used for CD8-dependent IL-2 receptor activation.

### Example 42: The Combinations of VHH-VHH Fusion Proteins that Target CD8 as a Target Scaffold Protein and IL-2R Beta/Gamma as Target Receptors Induce IL-2 Receptor Activation in CD8+T Cells

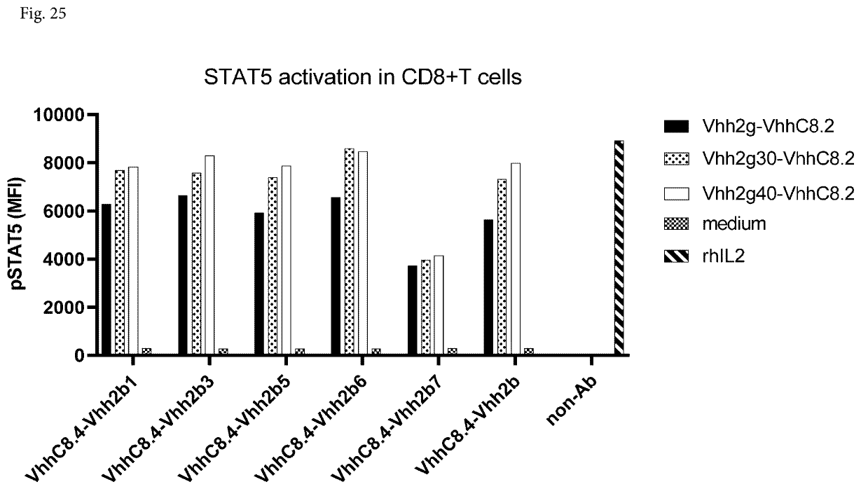

Human CD8+T cells were isolated from human PBMCs (STEMCELL, #70025.2) using EasySep™ Human CD8+T cell isolation kit (STEMCELL, #19053). After isolation, CD8+T cell were treated with plate coated anti-CD3/CD28 Abs (Biolegend, #317347, #302943, 5 micro g/mL each) and rhIL-2 (Peprotech, #200-02) for 3 days at 37 degrees C./5% CO₂. CTS Optimizer T cell expansion SFM (GIBCO, #A1048501) with penicillin/streptomycin (GIBCO, #15140122), GlutaMax (GIBCO, #35050061) and 10% fetal bovine serum was used for culture medium. After 3 days culture, CD8+T cells were harvested and washed with culture medium and continue to culture in the presence of rhIL-2. One day before assay, these CD8+T cells were washed with culture medium twice and rested overnight with culture medium. After resting, T cells were stimulated with the mixtures of VHH-VHH fusion, which binds to CD8 and IL-2R gamma (SEQ ID NO: 131-133), and VHH-VHH fusion, which binds to CD8 and IL-2R beta (SEQ ID NO: 134, 137-141), for 30 minutes at 37 degrees C. rhIL-2 (Peprotech, #200-02) was used as a positive control. These cells were immediately fixed with CytoFix (BD Biosciences, #554655) for 20 minutes at room temperature. Cells were washed with staining buffer (Biolegend, #420201) and chilled permeabilization buffer (BD Biosciences, #558050) were subsequently added to the cells and incubated on ice for 30 minutes. Cells were washed twice with staining buffer and Alexa Fluor (registered trademark) 647 Mouse Anti-Stat5 (pY694) (BD Biosciences, #562076) were added and incubated for at least 60 minutes at room temperature. Cells were washed once with staining buffer before analysis. Data acquisition was performed on an LSRFortessa X-20 (Becton Dickinson) (FIG. 25).

As shown in FIG. 25, the mixtures of VHH-VHH fusions of anti-CD8/IL2R gamma, which is Vhh2g-VhhC8.2, Vhh2g30-VhhC8.2 or Vhh2g40-VhhC8.2, and VHH-VHH fusions of anti-CD8/IL2R beta, which is VhhC8.4-Vhh2b, VhhC8.4-Vhh2b1, VhhC8.4-Vhh2b3, VhhC8.4-Vhh2b5, VhhC8.4-Vhh2b6 or VhhC8.4-Vhh2b7, induced significant STAT5 activation in activated human CD8+T cells. These data suggested various IL-2R beta/gamma binding domains can be used for CD8-dependent IL-2 receptor activation.

### Example 43: The Combinations of VHH-VHH Fusion Proteins that Target CD8 as a Target Scaffold Protein and IL-2R Beta/Gamma as Target Receptor Proteins Induce CD8+T Cell Proliferation but not in CD4+T Cells

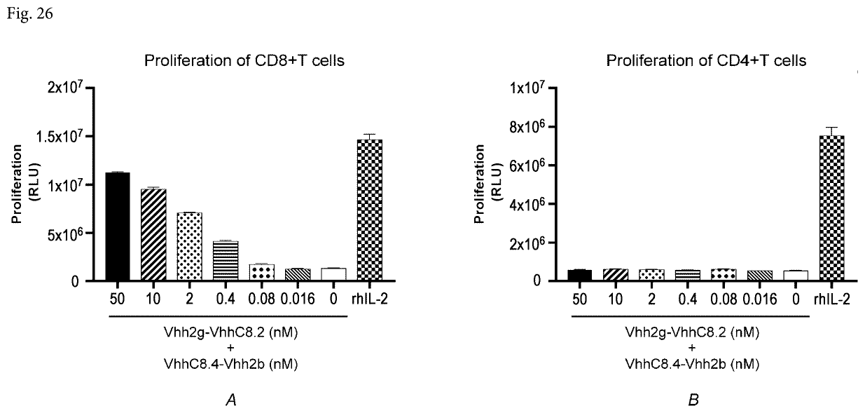

Human CD8+T cells or human CD4+T cells were isolated from human PBMCs (STEMCELL, #70025.2) using EasySep™ Human CD8+T cell isolation kit (STEMCELL, #19053) or EasySep™ Human CD4+T cell isolation kit (STEMCELL, #17952). After isolation, CD8+ or CD4+T cell were treated with plate coated anti-CD3/CD28 Abs (Biolegend, #317347, #302943, 5 micro g/mL each) and rhIL-2 (Peprotech, #200-02) for 3 days at 37 degrees C./5% CO₂. CTS Optimizer T cell expansion SFM (GIBCO, #A1048501) with penicillin/streptomycin (GIBCO, #15140122), GlutaMax (GIBCO, #35050061) and 10% fetal bovine serum was used for culture medium. After 3 days culture, CD8+ or CD4+ T cell were harvested and washed with culture medium twice and rested overnight with culture medium. After resting, T cells were stimulated with the mixtures of Vhh2g-VhhC8.2 (SEQ ID NO: 131) and VhhC8.4-Vhh2b (SEQ ID NO: 134) from 50 nM concentration for 3 days at 37 degrees C./5% CO₂. rhIL-2 (Peprotech, #200-02) was used as a positive control. After 3 days culture, cell proliferation was evaluated by CellTiter-Glo 2.0 (Promega, #G924C). (FIGS. 26A and 26B).

As shown in FIGS. 26A and B, the mixtures of Vhh2g-VhhC8.2 and VhhC8.4-Vhh2b induced significant CD8+T cells proliferation, but not for CD4+T cells. These data indicated these Abs can induce not only STAT5 activation but also functional signaling, like proliferation.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-15. (canceled)

16. A method for producing a protein complex, wherein
the protein complex comprises a first binding domain, a second binding domain, a third binding domain and a fourth binding domain, wherein the first binding domain is capable of binding to a scaffold protein, the second binding domain is capable of binding to a first receptor protein, the third binding domain is capable of binding to the scaffold protein, and the fourth binding domain is capable of binding to a second receptor protein,
wherein the first binding domain and the third binding domain are capable of non-competitively binding to the scaffold protein,
wherein the first receptor protein and the second receptor protein are each receptor subunits that can associate to form a receptor complex, and
wherein the protein complex is capable of inducing receptor signaling of the receptor complex,
the method comprising the steps of:
(i) Identifying a first binding domain and a third binding domain capable of non-competitively binding to the scaffold protein,
(ii) connecting, optionally via a polynucleotide encoding a linker, a polynucleotide encoding the first binding domain with a polynucleotide encoding the second binding domain,
(iii) connecting, optionally via a polynucleotide encoding a linker, a polynucleotide encoding the third binding domain with a polynucleotide encoding the fourth binding domain, and
(iv) expressing the polynucleotides of (ii) and (iii) to produce the protein complex.

17. The method of claim 16, wherein the first binding domain and the third binding domain are capable of binding the scaffold protein biparatopically.

18. The method of claim 16, wherein the first binding domain is connected to the second binding domain, optionally via a linker, and/or the third binding domain is connected to the fourth binding domain, optionally via a linker.

19. The method of claim 18, wherein the first binding domain is connected to the second binding domain via a linker, and the third binding domain is connected to the fourth binding domain via a linker, wherein each linker is 20 amino acids or less, preferably 15 amino acids or less, or more preferably 10 amino acids or less.

20. The method of claim 16, wherein the first binding domain and the third binding domain are each selected from the group consisting of a neutralizing binding domain and a non-neutralizing binding domain, wherein the first binding domain is a neutralizing binding domain and the third binding domain is a non-neutralizing binding domain or vice versa.

21. The method of claim 16, wherein the first binding domain and the third binding domain each are capable of binding subunits of the scaffold protein which can associate to form the scaffold protein.

22. The method of claim 16, wherein the scaffold protein is a membrane-bound protein, a soluble protein, an insoluble protein deposition, a cell surface marker, an immune cell surface marker, a T cell exhaustion marker, tumor-specific marker, tumor-associated marker, or a protein whose expression is characteristic of a disease or disorder.

23. The method of claim 16, wherein the scaffold protein is selected from the group consisting of PD1, PDL1, CSF2RB, CD4, CD5, CD6, CD7, CD8, CD9, CCR4, CD8, CD25, CD27, CD38, CD39, CD45, CD62L, CCR6, CD69, CD103, CD73, CD127, GITR, LRRC32, ICOS, TIGIT, RBD, MUC1, CEA, CTLA4, IL2R alpha, CXCR5, Neuropilin-1, TIM3, LAG3, TNF alpha, CD19, CD20, CD22, CD30, CD33, Glycoprotein NMB, CD56, CD70, CD79A, CD79B, CD138, PSCA, PSMA, BCMA, FcRH5, GPRC5D, FAP, LRRC15, E-selectin, EphB2, Melanotransferrin, HER2, TROP2, Nectine-4, EGFRvIII, IL13RA2, TMEFF2, Muc16, EpCAM, FcRH2, AFP, PSA, Amyloid beta, MBP and ASGPR.

24. The method of claim 16, wherein the first and second receptor proteins are each independently selected from the group consisting of IL2R beta, IL2R gamma, IL4R, IL13RA1, IL7R, IL9R, IL21R, TSLPR, IL3RA, CSF2RB, IL5RA, CSF2RA, IL6R, gp130, IL11RA, IL12RB1, IL12RB2, IL27RA, IL31RA, OSMR, CNTFR, LIFR, IL10RA, IL10RB, IL20RA, IL20RB, IL22RA1, IL28RA, IFNAR1, IFNAR2, IFNGR1, IFNGR2, IL1R1, IL1RAP, IL18R1, IL18RAP, ST2, IL17RA, IL17RC, TLR3, TLR4, TLR7, TLR9, CSF1R, TNFR1, TNFR2, LTBR, HVEM, FAS, CD28, cMET, DR3, DR4, DR5, NGFR, RANK, FN14, CD40, 4-1BB, OX40, GITR, TGFBR1, TGFBR2, ACVRL1, ACVR2A, BMPR2, ACVR2B, ACVR1B, ACVR1C, ACVR1, AMHR2, BMPR1A, BMPR1B, BMPR2, TRA, TRB, CD3E, CD16, TREM2, FGFR1, FGFR2, FGFR3, FGFR4, Fzd1, Fzd2, Fzd3, Fzd4, Fzd5, Fzd6, Fzd7, Fzd8 Fzd9, Fzd10, LRP5, LRP6 and LGR5.

25. The method of claim 16, wherein the receptor complex is IL-2R.

26. The method of claim 16, wherein the scaffold protein is PD1, the first receptor protein is IL2R beta, and the second receptor protein is IL2R gamma.

27. The method of claim 16, wherein the scaffold protein, the first receptor protein, and the second receptor protein is a combination chosen from any of (i) to (vi) below:
(i) the scaffold protein is PDL1, the first receptor protein is IL2R beta, and the second receptor protein is IL2R gamma;
(ii) the scaffold protein is TNFa, the first receptor protein is IL2R beta, and the second receptor protein is IL2R gamma;
(iii) the scaffold protein is CD25, the first receptor protein is IL2R beta, and the second receptor protein is IL2R gamma;
(iv) the scaffold protein is PDL1, the first receptor protein is CSF2RA, and the second receptor protein is CSF2RB;
(v) the scaffold protein is MUC1 or CEA, the first receptor protein is a Fzd receptor, and the second receptor protein is a Lrp; or
(vi) the scaffold protein is CD8, the first receptor protein is IL2R beta, and the second receptor protein is IL2R gamma.

28. The method of claim 16, wherein each binding domain is an antigen binding domain comprising VH and VL, sdAb, VHH, scFv, Fab, scFab, Fab′, Fab′-SH, F(ab′)2, diabody, triabody, Fv, aptamer, Affibody, a cytokine or variant thereof, a ligand, or a split cytokine.

29. The method of claim 16, wherein the protein complex further comprises a half-life extending domain comprising an Fc domain, FcRn-binding domain, an albumin-binding domain, an albumin or variant thereof, or polyethylene glycol.
